(19) United States
(12) Patent Application Publication
(10) Pub. No.: US 2007/0048860 A1
(43) Pub. Date: Mar. 1, 2007

(54) Carcinoembryonic antigen (CEA) peptides

(75) Inventors: Jeffrey Schlom, Potomac, MD (US); James Hodge, Kensington, MD (US); Sam Zaremba, Rockville, MD (US); Maria Elena Salazar, Frederick, MD (US)

Correspondence Address: OFFICE OF TECHNOLOGY TRANSFER;NATIONAL INSTITUTES OF HEALTH, C/O HELLER EHRMAN WHITE & MCAULIFFE LLP, 1717 RHODE ISLAND AVENUE, NW, WASHINGTON, DC, 20036-3001, US

(73) Assignee: The Government of the USA, as represented by the Secretary, Department of Health and Human Services, Rockville, MD (US)

(21) Appl. No.: 11/321,868

(22) Filed: Dec. 30, 2005

Related U.S. Application Data
(63) Continuation-in-part of application No. 10/406,317, filed on Apr. 4, 2003.
Application No. 10/406,317 is a division of application No. 09/856,988, filed on Sep. 24, 2001, now Pat. No. 6,969,609.
(63) Continuation-in-part of application No. 10/725,373, filed on Dec. 3, 2003.
Application No. 10/725,373 is a continuation of application No. 09/529,121, filed on Jun. 13, 2000, now Pat. No. 6,756,038.
(63) Continuation-in-part of application No. PCT/CA01/01092, filed on Jul. 27, 2001.
(60) Provisional application No. 60/111,582, filed on Dec. 9, 1998.
(60) Provisional application No. 60/061,589, filed on Oct. 10, 1997.
(60) Provisional application No. 60/222,043, filed on Jul. 31, 2000.

(51) Int. Cl. A61K 39/12; C12N 15/00

(52) U.S. Cl. 435/320.1; 424/204.1

(57) ABSTRACT

The present invention relates to novel modified CEA agonist (or antagonist) peptides, polypeptides and proteins containing a modified epitope therein, nucleic acids coding therefor, vectors comprising said nucleic acids, mixtures and compositions of the aforementioned agents, and their advantageous use in generating CEA-specific immune responses and/or in the treatment of cancers and the present invention further relates to the foregoing combined with one or more costimulatory molecules.

### FIELD OF THE INVENTION

The present invention relates to novel modified CEA agonist (or antagonist) peptides, polypeptides and proteins containing a modified epitope therein, nucleic acids coding therefor, vectors comprising said nucleic acids, mixtures and compositions of the aforementioned agents, and their advantageous use in generating CEA-specific immune responses and/or in the treatment of cancers and the present invention further relates to the foregoing combined with one or more costimulatory molecules.

### BACKGROUND OF THE INVENTION

A major challenge of modern cancer immunotherapy is the identification of cytotoxic T lymphocyte (CTL) epitopes from defined tumor-associated antigens (TAA) that promote lysis of tumor cells. The majority of antigens on human cancers are not tumor specific and are overexpressed in malignant cells as opposed to cells of normal tissues. Therefore, immunity to cancer in humans may rest mostly on the development of an effective immune response mainly directed to self-molecules qualitatively common to all cell types.

Human carcinoembryonic antigen (CEA) is a 180 kD glycoprotein expressed on the majority of colon, rectal, stomach and pancreatic tumors (1), some 50% of breast carcinomas (2) and 70% of lung carcinomas (3). CEA is also expressed in fetal gut tissue, and to a lesser extent on normal colon epithelium. The immunogenicity of CEA has been ambiguous, with several studies-reporting the presence of anti-CEA antibodies in patients (4-7) while other studies have not (8-10). CEA was first described as a cancer specific fetal antigen in adenocarcinoma of the human digestive tract in 1965 (Gold, P. and Freeman, S. O. (1965) Exp. Med. 121:439-462). Since that time, CEA has been characterized as a cell surface antigen produced in excess in nearly all solid tumors of the human gastrointestinal tract. The gene for the human CEA protein has been cloned. (Oikawa et al (1987) Biochim. Biophys. Res. 142:511-518; European Application No. EP 0346710).

Recently, the first evidence was reported of a human CTL response to CEA (11). This CAP1 peptide showed the highest level of T2 cell binding among the various CEA peptides tested with stimulation of the T cells resulting in the generation of cytotoxic T cell lines. We have identified a 9-mer peptide, designated CAP1 (with the sequence YLSGANLNL) (SEQ. ID NO: 1A), on the basis of binding to HLA-A2, and the ability to generate specific CTL from peripheral blood mononuclear cells (PBMC) from carcinoma patients immunized with a recombinant vaccinia virus expressing CEA (rV-CEA). For example, peripheral blood lymphocytes (PBLs) from 5 patients showed signs of T cell response to CAP1 peptide after immunization with rV-CEA. Two other laboratories have since generated CAP1 specific CTL in vitro employing peptide pulsed dendritic cells as antigen presenting cells (APC) (12). It has also recently been reported (13) that CAP1 specific CTL can be generated from PBMC from carcinoma patients immunized with the avipox recombinant ALVAC-CEA. Several groups have also reported the generation of anti-CEA antibodies and CEA specific proliferative T cell responses following immunization with either an anti-Id to an anti-CEA monoclonal antibody (MAb) (14), recombinant CEA protein (15), or rV-CEA (16).

Several investigators have introduced CTL to tumor associated and viral antigens by in vitro stimulation of PBMC with an immunodominant peptide. Recent work with the gp100 melanoma antigen (17-19), an HIV polymerase peptide (20) and the papilloma virus tumor antigen E6 (21) demonstrated enhanced immunogenicity after modifications to the peptide sequences. In these studies, replacements were at anchor positions and were intended to increase binding to murine or human MHC antigens. This approach was based on a demonstrated correlation between immunogenicity and peptide binding affinity to class I MHC (major histocompatibility complex) molecules for viral antigen epitopes (22).

Previous investigators have also worked with fragments of CEA. Thus, Shively (1989), in a European patent publication (EP No. 0343946 A2) reports a number of CEA fragments that include a unique epitope (as defined by its reactivity with an antibody). The latter CEA fragment is 177 amino acid residues long and contains the 9-mer sequence of CAP1. However, no shorter CEA fragments that include the CAP1 sequence were described.

In sum, the use of rV-CEA alone as an agent for boosting the CEA-specific immune response of rV-CEA suffers from the drawback of stimulating an immune response to vaccinia virus. However, the novel combination of rV-CEA and CAP1 suggested itself to us as a “second generation protocol” for the treatment of cancer patients.

It is an accepted principle that when an immunogenic peptide is modified in a conserved manner (e.g., a hydrophobic amino acid is substituted with a hydrophobic amino acid) the modified peptide is likely to have similar immunogenic activity based upon the maintenance of the molecule's shape, charge and hydrophobic character.

More specifically, a study by Madden (33) has identified specific amino acid preferences in peptides for MHC-complexing, a precursor step to T cell recognition. Madden as well as other investigators (31) suggest that specific amino acid positions in peptides are available for T cell recognition.

Skipper et al., (40) describes the identification and characterization of a naturally-occurring peptide epitope of tyrosinase, wherein the peptide sequence differs from that which is predicted from the DNA. This modified peptide is recognized by tyrosinase-specific human cytotoxic T-lymphocytes (“CTL”) more effectively than the direct translation product and is the only one of the two peptides to be presented by HLA-A2.1 molecules on the cell surface. The modification is a substitution of an asparagine with an aspartic acid. The authors propose that the asparagine is N-glycosylated in the endoplasmic reticulum during protein synthesis and is deamidated post-translationally.

In the case of CAP1, the primary and secondary anchors at positions 2, 9, and 1 are already occupied by preferred amino acids and so a different approach was taken to improve peptide immunogenicity by attempting to enhance its ability to bind to the TCR. It appeared to us that by altering amino acid residues expected to contact the TCR one could generate an analog of CAP1 with substitutions at non-MHC anchor positions. Such an analog might then represent a T cell enhancer agonist capable of stimulating CTL more efficiently than the native peptide. Previous results supported the concept that some peptide analogs could act as T cell antagonists by inhibiting responses to the antigenic peptide (23-29). Such inhibition was shown to be TCR specific and could not be explained by competition for peptide binding to the MHC protein. Analogously, a peptide enhancer agonist would be an analog that increased the effector function without accompanying increases in MHC binding. We therefore sought to increase CAP1 immunogenicity by analyzing panels of analogs containing single amino acid substitutions to residues we predicted would interact with the T cell receptor (TCR) of CAP1-specific CTL. The present invention relates to the construction of a novel T cell enhancer agonist for the CAP1 peptide, the first such example for a human CTL epitope.

Additional background of the invention is as follows. The extent of the primary response of T cells, which involves their activation, expansion, and differentiation, is paramount to a successful immune response to an antigen. The initiation of an immune response requires at least two signals for the activation of naive T cells by antigen presenting cells (APC) (B1-B5). The first signal is antigen specific, delivered through the T-cell receptor via the peptide/major histocompatibility complex, and causes the T cell to enter the cell cycle. The second, or “costimulatory,” signal is required for cytokine production and proliferation. At least three distinct molecules normally found on the surface of professional APC have been proposed as capable of providing the second signal critical for T-cell activation: B7.1 (CD80), Intercellular adhesion molecule-1 (ICAM-1; CD54), and Leukocyte function-associated antigen-3 (LFA-3; human CD58; murine CD48) (B2, B6, B7). The T-cell ligands for these costimulatory molecules are distinct. B7-1 interacts with the CD28 and CTLA4 molecules, ICAM-1 interacts with the CD11a/CD18 (LFA-1/2 integrin) complex, and LFA-3 interacts with the CD2 (LFA-2) molecules. It is not known whether these costimulatory molecules perform equivalent functions or carry out specialized functions at specific stages of an induced immune response (B2). These molecules have been individually shown to costimulate T-cell proliferation in vitro (B6). However, because they may be expressed simultaneously on APC, it has been difficult to examine relative potencies of individual costimulatory molecules during the induction of T-cell proliferation (B2).

As it has been proposed that both antigen and costimulatory molecules must be expressed in proximity to each other to properly co-engage the T cell and costimulatory receptors (B8, B9), the admixture of several recombinant viruses could be utilized to explore the potential cooperation of costimulatory molecules. The disadvantage of this approach, however, is that the admixture of three or more viruses has a statistically diminished probability of co-infecting the same cell, thereby making a multi-gene construct much more desirable for use with multiple costimulatory molecule genes.

WO 91/02805, published Mar. 7, 1991, discloses a recombinant retrovirus vector construct which directs the expression of a target antigen, an MHC protein and other proteins involved in immune interactions which are missing or under-represented in a target cell.

Akagi, et al. 1997, J. Immunotherapy Vol. 20 (1):38-47 disclose an admixture of a recombinant vaccinia virus containing a modified MUC1 gene (rV-MUC1), and a recombinant vaccinia virus containing the gene for the murine costimulatory molecule B7 (rV-B7).

Cavallo, P. et al. 1995, Eur. J. Immunol., 25:1154-1162 disclose that transfection of B7-1 cDNA into three ICAM-1⁺ tumor cell lines is sufficient to induce rejection in syngeneic mice.

Chen, L. et al. 1994, J. Exp. Med., 179:523-532 disclose a recombinant retrovirus vector containing cDNA for murine B7 and the use of the vector in transducing various tumors.

Damle, N. K. et al 1992, J. Immunol Vol 148 (No. 7): 1985-1992 disclose the use of an antigen presenting cell (APC)-independent in vitro culture system consisting of immobilized combinations of monoclonal antibodies directed at the TCR/CD3 complex and soluble Ig chimeras (RG) of four distinct APC-associated costimulatory molecules to compare the abilities of these molecules to costimulate T cell proliferation.

Dubey, C. et al 1995, J Immunol 155: 45-57 disclose a study of the relative contribution of ICAM-1: LFA-1 and B7: CD28/CTLA-4 costimulatory pathways in nave T cell activation, using either anti-CD28 antibody or fibroblast cell lines transfected with I-E^{k}, which express either no costimulatory molecules, ICAM-1 alone, B7-1 alone, or ICAM-1 and B7-1 together.

Fenton, R. G. et al, 1998 Vol. 21, No. 2, pp 95-108, disclose transfection of the costimulatory molecule B7-1 gene into three HLA-A2-expressing human melanoma cell lines, and their capacity to stimulate primary human T cells. The three melanoma lines also expressed detectable levels of the costimulatory molecules ICAM-1 (CD54) and LFA-3 (CD58).

Gjorloff Wingren, A. et al 1995, Critical Reviews in Immunol 15 (3 & 4): 235-253 disclose that with co-transfection of HLA-DR, B7 and LFA-3 into CHO cells, these molecules cooperate in activation of both nave and memory T cells and allow responses at picomolar concentrations of the antigen, staphylococcal enterotoxin B (SEB).

Goldbach-Mansky, R. et al 1992, International Immunol. 4(No. 12): 1351-1360 disclose that CD4⁺ T cells respond to staphylococcal enterotoxin B (SEB) in the presence of the LFA-3, ICAM-1 and B7 positive erythroleukemic cell line K562, murine L cells, and human B7 transfected L cells.

Hodge, J. W. et al 1994, Cancer Research 54:5552-5555 disclose the construction and characterization of recombinant vaccinia viruses containing the murine B7.1 and B7.2 genes.

Hodge, J. W. et al 1995, Cancer Research 55: 3598-3603 Cancer Research 55:3598-3603 disclose an admixture of recombinant vaccinia murine B7.1 (rV-B7) plus recombinant vaccinia expressing the human carcinoembryonic antigen gene (rV-CEA) and the use of this admixture for anti-tumor activity.

Parra, et al 1993, Scand J. Immunol 38: 508-514, Parra, E. et al 1994, J. Immunol 153: 2479-2487, and Parra, et al. 1997, J. Immunol., 458:637-642 disclose CHO cells transfected with the human HLA-DR4 molecule (CHO-DR4); HLA-DR4 and B7 (CHO-DR4/B7), HLA-DR4 and LFA-3 (CHO-DR4/LFA3); HLA-DR4 and ICAM-1 (CHO-DR4/ICAM-1); or DR4, B7 and LFA-3 (CHO-DR4/B7/LFA-3) genes.

Thomas, R. et al. 1993 J. Immunol. 151:6840-6852 disclose that freshly obtained dendritic cells (DC) express similar densities of HLA-DR and the accessory molecules LFA-3, ICAM-1 and B7 as monocytes.

Uzendoski, K et al. May 1997, Human Gene Therapy 8:851-860 disclose the construction, characterization and immunological consequences of a recombinant vaccinia virus expressing the murine costimulatory molecule, ICAM-1.

WO 96/10419, published Apr. 11, 1996, of PCT/US95/12624 discloses subject matter relating to a single recombinant viral vector which has incorporated one or more genes or portion thereof encoding an immunostimulatory molecule and one or more genes or portion thereof encoding an antigen of a disease state.

Robinson et al U.S. Pat. No. 5,738,852 discloses a retroviral vector containing a polynucleotide sequence encoding a target antigen of an infectious agent and a polynucleotide sequence encoding a B7 costimulatory molecule.

Further background of the invention is as follows. The prospects of cancer immunotherapy rely upon the identification of tumor associated antigens which can be recognized by the immune system. Specifically, target antigens eliciting T cell-mediated responses are of critical interest. This comes from evidence that cytotoxic T lymphocytes (CILs) can induce tumor regression both in animal models (Kast W. et al (1989) Cell 59:6035; Greendberg P. (1991) Adv. Immunol. 49:281) and in humans (Boon T. et al. (1994) Annu. Rev. Immunol 12 : 337).

Human carcinoembryonic antigen (CEA) is a 1801D glycoprotein expressed on the majority of colon, rectal, stomach and pancreatic tumors (Muaro et al., (1985) Cancer Res. 45: 5769), some 50% of breast carcinomas (Steward et al. (1974) Cancer 33: 1246) and 70% of lung carcinomas (Vincent, R. G. and Chu, T. M. (1978) J Thor. Cardiovas. Surg. 66: 320). CEA is also expressed in fetal gut tissue and to a lesser extent on normal colon epithelium. The immunogenicity of CEA has been ambiguous, with several studies reporting the presence of anti-CEA antibodies in patients (Gold et al., (1973) Nature New, Biology 239: 60; Pompecki, R. (1980) Eur. J. Cancer 16 : 973; Ura et al. (1985) Cancer Lett. 25 : 283; Fuchs et al. (1988) Cancer Im7mmol. Inamunother. 26: 180) while other studies have not (LoGerfo et al. (1972) Int. J Cancer 9: 344; MacSween, J. M. (1975) hat J. Cancer 15 : 246; Chester K. A. and Begent, H. J. (1984) Clin. Exp. Immunol 58 : 685). CEA was first described as a cancer specific fetal antigen inadenocarcinoma of the human digestive tract in 1965 (Gold, P. and Freeman, 5.0. (1965) Exp. Med. 121: 439). Since that time, CEA has been characterized as a cell surface antigen produced in excess in nearly all solid tumors of the human gastrointestinal tract. The gene for the human CEA protein has been cloned (Oikawa et al (1987) Biochim. Biophys. Res. 142: 511-518; European Application No. EP0346710).

Notwithstanding the aforementioned ambiguous demonstration of CEA's immunogenicity, a phase I clinical trail using a vaccinia-CEA vaccine (“rV-CEA”) did demonstrate that CEA-specific cytolytic T-lymphocyte (CTL) response could be elicited in humans (Tsang, K. Y. et al. (1995) J. Natl. Cancer Instit 87:982-990; Tsang K. Y. et al. (1997) Clin. Cancer Res. 3: 2439-2449). As a consequence of the studies, a CEA immunodominant CTL epitope was identified. This 9-mer (i.e. YLSGANLNL) was shown to bind to a HLA-A2 class I molecule and has been designated carcinoembryonic antigen peptide-1 (“CAP-1”). Several subsequent studies have also demonstrated the ability of the CAP-1 epitope/peptide per se to elicit CEA-specific human CTL responses (Alters, S. E. et al. (1998) J. linmunotlzer.

21: 17-26; Tsang, K. Y. et al. (1997) Supra; Zaremba, S. et al. (1997) Cancer Res.

57: 4570-4577). Moreover, stable CTL lines derived by culture of peripheral blood mononuclear cells (PBMCs) from rV-CEA vaccinated patients with CAP 1 and interleukin (IL)-2 have recently been described (Tsang, K. Y. (1997) Supra).

It is an accepted principle that when an immunogenic peptide is modified in a conserved manner (i.e. a hydrophobic amino acid is substituted with a hydrophobic amino acid) the modified peptide is likely to have similar immunogenic activity based upon the maintenance of the molecule's shape, charge and hydrophobic character. More specifically, a study by Madden (Madden et al. (1993) Cell 75 : 693) has identified specific amino acid preferences in peptides for MHC-complexing, a precursor step to T cell recognition. Madden as well as other investigators (Rammensee et al., (1995) Immunogenetics 41: 178) have suggested that specific amino acid positions in peptides are available for T cell recognition.

Skipper et al. ((1996) J Exp, Med. 183: 527) described the identification and characterization of a naturally-occurring peptide epitope of tyrosinase wherein the peptide sequence differed from that which is predicted from the DNA. This modified peptide was recognized by tyrosinase-specific human cytotoxic T-lymphocytes (“CTL”) more effectively than the direct translation product, and was the only one of the two peptides to be presented by HLA-A2.1 molecules on the cell surface. The modification was a substitution of an asparagine with an aspartic acid. The authors proposed that the asparagine was N-glycosylated in the endoplasmic reticulum during protein synthesis and subsequently deamidated post-translationally.

With respect to the CEA epitope CAP1, the primary and secondary anchors positions for HLA binding at positions 2, 9, and 1 (of the epitope) are already occupied by preferred amino acids. As such, Schlom and colleagues attempted to increase CAP1 immunogenicity via the generation of epitope analogs containing single amino acid substitutions to residues predicted to interact with the T cell receptor (“TCR”) of CAP1-specific CTL. One such analog epitope (YLSGADLNL; designated CAP-1-6D) was identified which demonstrated an increased immunogenicity to that of the natural epitope, but not a concomitant increase in MHC binding per se (i.e. it behaved as an agonist; Zaremba, S. et al (1997) Supra).

### SUMMARY OF THE INVENTION

The present invention relates to the identification of peptides which are single or double amino acid changes from the CAP-1 peptide sequence. The CAP-1 peptide has been identified as a highly immunogenic epitope of the carcinoembryonic antigen (referred to herein as “CEA”), which is capable of stimulating CEA-specific cytolytic T-cell (“CTL”) responses. CEA is a cell surface antigen found in abundance on several types of cancer cells. Thus, peptides of CEA capable of stimulating a cytolytic CTL response, such as CAP-1 are potential immunogens for use in cancer immunotherapy.

Some of the peptides of the present invention are agonists of CAP-1 and CEA; that is, they facilitate the interaction between the MHC-complex of the antigen-presenting cell and the T-cell receptor (“TCR”) complex of the T-cell. Thus, these peptides can serve as immunogens to treat and/or vaccinate patients with CEA-expressing cancers. Also, these peptides may be used to stimulate T-cells in culture for adoptive transfer of T-cells to cancer patients. Four such peptides have amino acid sequences:

(1) YLSGADLNL (Agonist CAP1-6D) (SEQ. ID NO: 2A);

(2) YLSGADINL (Agonist CAP1-6D, 71) (SEQ. ID NO: 3A);

(3) YLSGANINL (Agonist CAP1-71) (SEQ. ID NO: 4A); and

(4) YLSGACLNL (agonist CAP1-6C) (SEQ. ID NO: 5A).

The underlined amino acids identify the amino acids changes from the CAP-1 peptide sequence. Peptides CAP1-6D and CAP1-6D, 71 are especially preferred peptides according to the present invention and have enhanced activity pared to CAP-1 activity. Peptides CAP1-71 and CAP1-6C have activity similar to CAP-1.

Other peptides according to the present invention function as antagonists of CEA; that is, they reduce or eliminate CEA-specific T-cell activation and killing which occur through interactions of the MHC-peptide complex and TCR complex.

The present invention encompasses kits comprising an agonist peptide and a vector comprising a gene encoding CEA or a recombinantly produced CEA protein. Moreover, the kit may include an immunostimulatory molecule.

The present invention also encompasses kits comprising an antagonist peptide alone or in combination with an immunosuppressive agent.

Another object of the present invention is a pharmaceutical composition comprising one or more agonist peptides alone or in combination with an immunostimulatory molecule and a pharmaceutically acceptable carrier.

Another object of the present invention is a pharmaceutical composition comprising one or more antagonist peptides alone or in combination with an immunosuppressing agent and a pharmaceutically acceptable carrier.

The present aspect of the present invention is a nucleic acid sequence encoding at least one agonist peptide or encoding at least one antagonist peptide.

Another aspect of the invention is a vector comprising a nucleic acid sequence encoding at least one agonist peptide or a nucleic acid sequence encoding at least one antagonist peptide and host cells comprising such vectors.

Another aspect of the present invention relates to the use of these peptides in cancer immunotherapy. The agonist peptides are useful in stimulating a cytolytic immune response to CEA, resulting tumor reduction and/or prevention. Accordingly, the present invention also relates to a method of treating cancer patients with the peptides as well as a cancer vaccine. The antagonist peptides are useful in methods of controlling autoimmune response to CEA or CAP-1.

Yet another aspect of the present invention is an agonist-pulsed antigen presenting cell.

The present invention further provides a recombinant vector comprising foreign or exogenous genes or portions thereof encoding multiple costimulatory molecules. Genes or functional portions thereof encoding costimulatory molecules having utility in the present invention include but are not limited to a B7 family member, ICAM-1, LFA-3,4-1BBL, CD59, CD40, CD70, VCAM-1, OX-40L, functional portions and homologs thereof. The vector of the invention may further provide a foreign gene encoding at least one target antigen or immunological epitope thereof in combination with the foreign genes encoding multiple costimulatory molecules. The foreign gene encoding at least one target antigen or immunological epitope thereof may be derived from cells, tissues or organisms such as viruses, bacteria, protozoans, parasites, yeast, tumor cells, preneoplastic cells, hyperplastic cells, tissue specific cells, or synthetic antigens. The vector may further provide a foreign gene encoding at least one or a combination of cytokines, chemokines and flt-3L.

The recombinant vector for use in the present invention group consisting of bacterial vectors, virus vectors, nucleic acid based vectors and the like. The recombinant virus vectors include but are not limited to poxvirus, adenovirus, herpes virus, alphavirus, retrovirus, picornavirus, iridovirus and the like. The poxvirus include but are not limited to the orthopox, avipox, suipox and capripox.

The present invention provides a recombinant virus comprising foreign genes or portions thereof encoding multiple costimulatory molecules for providing an enhanced immune response to a target cell, target antigen or immunological epitope thereof which is greater than a response provided by a recombinant virus comprising a foreign gene or genes encoding single or double costimulatory molecules. The recombinant virus of the invention may further provide a foreign gene encoding at least one target antigen or immunological epitope thereof in combination with the foreign genes encoding multiple costimulatory molecules. The recombinant virus may further provide a foreign gene encoding other classes of immunostimulatory molecules such as cytokines including but not limited to IL-2, IL-12, GM-CSF and the like, chemokines such as MIP1, MIP2, RANTES and the like, and Flt-3L which stimulates DC proliferation.

The present invention further provides a recombinant poxvirus comprising foreign genes or portions thereof encoding multiple costimulatory molecules for providing an enhanced immune response to a target cell, target antigen or immunological epitope thereof which is greater than a response provided by a recombinant poxvirus comprising a foreign gene or genes encoding single or double costimulatory molecules. The recombinant poxvirus of the invention may further provide a foreign gene encoding at least one target antigen or immunological epitope thereof in combination with the foreign genes encoding multiple costimulatory molecules.

The present invention also provides a recombinant poxvirus comprising a nucleic acid sequence encoding and expressing multiple costimulatory molecules, said nucleic acid sequence comprising a nucleic acid sequence encoding at least one molecule from the B7 family of costimulatory molecules, a nucleic acid sequence encoding an ICAM-1 costimulatory molecules, and a nucleic acid sequence encoding an LFA-3 costimulatory molecule. The recombinant virus further provides a multiplicity of poxvirus promoters which regulate expression of each foreign gene.

The present invention provides a recombinant virus produced by allowing a plasmid vector comprising foreign DNA encoding multiple costimulatory molecules to undergo recombination with a parental virus genome to produce a recombinant virus having inserted into its genome the foreign DNA. The recombinant virus produced by recombination may further contain a foreign gene encoding at least one target antigen or immunological epitope thereof provided by the plasmid vector.

The present invention also provides a recombinant poxvirus produced by allowing a plasmid vector comprising foreign DNA encoding the costimulatory molecule, LFA-3, ICAM-1 and at least one molecule from the B7 family to undergo recombination with a parental poxvirus genome to produce a recombinant poxvirus having inserted into its genome the foreign DNA and a multiplicity of poxvirus promoters capable of controlling the expression of the foreign DNA. The recombinant poxvirus produced by recombination may further contain a foreign gene encoding at least one target antigen or immunological epitope thereof provided by the plasmid vector.

An object of the invention is to provide an immunogen for enhancement of immune responses against target cells, target antigens or immunological epitopes thereof comprising a recombinant vector having foreign nucleic acid sequences encoding multiple costimulatory molecules. The vector may further comprise a foreign nucleic acid sequence encoding at least one target antigen or immunological epitope thereof.

Another object of the invention is to provide an immunogen for enhancement of immune responses against target cells, target antigens or immunological epitopes thereof comprising a recombinant virus vector having foreign nucleic acid sequences encoding three or more costimulatory molecules. The recombinant virus vector may further comprise a foreign nucleic acid sequence encoding at least one or more target antigens or immunological epitopes thereof.

Yet another object of the invention is to provide an immunogen for enhancement of immune responses against target cells, target antigens or immunological epitopes thereof comprising a recombinant poxvirus vector comprising a foreign nucleic acid sequence encoding the costimulatory molecules LFA-3, ICAM-1 and at least one molecule from the B7 family and a foreign nucleic acid sequence encoding at least one target antigen or immunological epitope thereof.

The vector of the present invention provides a vaccine for eliciting and enhancing immune responses against target cells, target antigens or epitopes thereof for protection and/or treatment of disease states. The vector vaccine comprises foreign nucleic acid sequences encoding multiple costimulatory molecules. The vector vaccine may also comprise foreign nucleic acid sequences encoding one or more target antigens or immunological epitopes thereof for producing a monovalent or polyvalent vaccine against a disease.

The present invention provides pharmaceutical compositions comprising a vector having foreign nucleic acid sequences encoding multiple costimulatory molecules and a pharmaceutically acceptable carrier. The vector may further comprise a foreign nucleic acid sequence encoding at least one target antigen or immunological epitope thereof. The vector may additionally comprise a nucleic sequence encoding a cytokine, chemokine, flt-3L, or combination thereof.

The present invention provides a pharmaceutical composition comprising a recombinant virus vector which comprises foreign or exogenous genes or functional portions thereof encoding three or more costimulatory molecules, a foreign gene encoding at least one target antigen or immunological epitope thereof, and a pharmaceutically acceptable carrier.

The present invention also provides pharmaceutical compositions comprising a recombinant poxvirus comprising foreign genes or portions thereof encoding multiple costimulatory molecules and a pharmaceutically acceptable carrier. The recombinant poxvirus may further comprise a foreign nucleic acid sequence encoding at least one target antigen or immunological epitope thereof.

Another aspect of the invention is a pharmaceutical composition comprising a recombinant poxvirus comprising foreign genes or portions thereof encoding three or more costimulatory molecules and may further comprise a foreign gene or portion thereof encoding at least one target antigen or immunological epitope thereof, and a pharmaceutically acceptable carrier or immunological epitope thereof.

The present invention also provides a pharmaceutical composition comprising a first vector comprising foreign genes or functional portions thereof encoding multiple costimulatory molecules and a second vector comprising foreign genes encoding at least one target antigen or immunological epitope thereof and a pharmaceutically acceptable carrier.

The present invention provides host cells infected, transfected or transduced with a first vector comprising foreign genes encoding multiple costimulatory molecules causing expression of the multiple costimulatory molecules in the host cells. The first vector or a second vector may further provide a foreign gene encoding at least one target antigen or immunological epitope thereof to the host cell.

The present invention provides antigen-presenting cells (APCs) or tumor cells infected, transfected or transduced with a first vector comprising foreign or exogenously provided genes encoding multiple costimulatory molecules causing expression or overexpression of the multiple costimulatory, molecules. The first vector or a second vector may further provide a foreign gene encoding at least one target antigen or immunological epitope thereof to the host cell.

The present invention further provides host cells infected with a recombinant poxvirus causing expression of the multiple costimulatory molecules, and optionally causing expression of a target antigen or immunological epitope thereof.

Another aspect of the invention is a dendritic cell (DC) and precursor thereof infected, transfected or genetically engineered to overexpress genes encoding multiple exogenous costimulatory molecules. The DCs and precursors thereof may further be engineered to express foreign genes encoding at least one target antigen or immunological epitope thereof.

Yet another aspect of the invention is a DC and precursors thereof genetically engineered to overexpress genes encoding at least three exogenous costimulatory molecules. The DCs and precursor thereof may further be engineered to express foreign genes encoding at least one target antigen or immunological epitope thereof.

The present invention further provides a DC and precursors thereof genetically engineered to overexpress genes encoding at least one B7 molecule, ICAM-1 and LFA-3. The DCs and precursor thereof may further be engineered to express foreign genes encoding at least one target antigen or immunological epitope thereof.

The present invention provides methods and a plasmid vector for recombination with a parental virus designed to produce a recombinant virus capable of expressing foreign nucleic acid sequences encoding multiple costimulatory molecules comprising (a) a multiplicity of viral promoters, (b) the foreign nucleic acid sequences encoding the multiple costimulatory molecules, (c) DNA sequences flanking the constructs of elements (a) and (b), the flanking sequences at both the 5′ and 3′ ends being homologous to a region of a parental virus genome where elements (a) and (b) are to be inserted. The plasmid vector may further provide a foreign nucleic acid sequence encoding at least one target antigen or immunological epitope thereof. The plasmid vector may also provide a gene encoding a selectable marker.

The present invention also provides methods and a plasmid vector for recombination with a parental poxvirus designed to produce a recombinant poxvirus capable of expressing foreign nucleic acid sequences encoding the costimulatory molecules LFA-3, ICAM-1 and at least one B7 molecule which comprises (a) a multiplicity of poxviral promoters, (b) the foreign nucleic acid sequences encoding the LFA-3, ICAM-1 and at least one B7 molecule, (c) DNA sequences flanking the construct of elements (a) and (b), the flanking sequences at both 5′ and 3′ ends being homologous to a region of a parental poxvirus genome where elements (a) and (b) are to be inserted. The plasmid vector may further provide a foreign nucleic acid sequence encoding at least one target antigen or immunological epitope thereof. The plasmid vector may also provide a gene encoding a selectable marker.

One aspect of the invention is a method of enhancing immunological responses in a mammal to at least one target cell, target antigen or immunological epitope thereof comprising administration of a first vector comprising foreign nucleic acid sequences encoding multiple costimulatory molecules, each costimulatory molecule expressed in a cell in the mammal in an amount effective to enhance at least one immunological response in the mammal. Genes or functional portions thereof encoding costimulatory molecules having utility in the present invention include but are not limited to a B7 family member, ICAM-1, LFA-3, 4-1 BBL, CD59, CD40, CD70, VCAM-1, OX-40L and homologs and portions thereof. A foreign nucleic acid sequence encoding at least one target antigen or immunological epitope thereof may further be provided in the method by the first vector or by a second vector.

In addition to genes or portion thereof encoding multiple costimulatory molecules, a foreign or exogenous nucleic acid sequence or functional portions thereof encoding at least one or a combination of other classes of immunostimulatory molecules may also be provided by the first vector, by the second vector, or by a third vector. Other classes of immunostimulatory molecules includes cytokines such as IL-2, IL-12, GM-CSF and the like, chemokines such as MIP1, MIP2, RANTES and the like and Flt-3L.

An aspect of the invention is a method of enhancing an antigen-specific T cell immune response in a mammal to a target cell, target antigen or immunological epitope thereof comprising administration of a foreign recombinant poxvirus comprising nucleic acid sequences encoding multiple costimulatory molecules LFA-3, ICAM-1 and at least one B7 molecule, each costimulatory molecule expressed in a cell in the mammal in an amount effective to enhance at least one T-cell immune response in which the enhancement is greater than the additive sum of enhancement provided by administration of single or double costimulatory molecules.

In another method of enhancing immunological responses. APCs or tumor cells expressing foreign or exogenously provided genes encoding multiple costimulatory molecules are provided to a mammal in an effective amount to enhance immunological responses. The APC or tumor cell may further express foreign genes encoding at least one target antigen or immunological epitope thereof for enhancement of immune responses. A target antigen or immunological epitope thereof may be administered to the mammal prior to, concurrently with or subsequent to the administration of the APC or tumor cell. In addition, or alternatively, APCs or tumor cells are pulsed with at least one target antigen or immunological epitope thereof prior to administration to the mammal.

The present invention provides methods of enhancing humoral responses in a mammal to a target cell, target antigen or immunological epitope thereof comprising administration of a recombinant vector comprising foreign nucleic acid sequences encoding multiple costimulatory molecules to a mammal in an amount effective to enhance an humoral response. The vector may further comprise nucleic acid sequences encoding at least one target antigen or immunological epitope thereof. The invention further provides an isolated antibody or functional portion thereof against a target cell, target antigen or immunological epitope thereof produced by the method.

The present invention also provides antibody specific for a target antigen or immunological epitope thereof produced in response to administration of a recombinant poxvirus comprising foreign genes encoding B7, ICAM-1 and LFA-3 and genes encoding one or more target antigens or epitopes thereof.

The present invention further encompasses novel modified CEA agonist polypeptides/proteins comprising a modified epitope containing the sequence YLSGADLNL, nucleic acids coding therefore, vectors (such as recombinant virus and/or bacteria) and/or cells (such as antigen-presenting cells) comprising said nucleic acids, and mixtures and/or compositions of the aforementioned. All of these aforementioned agents, mixtures and compositions are characterized by their ability to induce or elicit an immune response against a CEA protein or fragment thereof, a

CEA agonist polypeptide containing said modified epitope, a normal or modified CEA epitope, or cells binding or expressing the aforementioned CEA protein/fragment, CEA agonist polypeptide, or normal/modified CEA epitope.

Accordingly, in one embodiment of the invention a CEA agonist polypeptide/protein is provided comprising a modified epitope of CEA, wherein said modified epitope contains the sequence YLSGADLNL.

In a further embodiment of the invention, the CEA agonist polypeptide/protein has the amino-acid sequence of SEQ ID NO: 1 (FIG. 62).

As previously noted, embodiments of the invention encompass nucleic acids coding for the aforementioned CEA agonist polypeptides/proteins. Accordingly, embodiments of the invention consist/comprise the nucleic acid sequence of SEQ ID NO: 2 (FIG. 62).

In further embodiments of the invention, the nucleic acid is a DNA selected from the group consisting of viral nucleic acid, plasmid, bacterial DNA, naked/free DNA, and RNA. In yet further embodiments, the viral nucleic acid is selected from the group consisting of adenovirus, alphavirus and poxvirus. In still yet further embodiments, the poxvirus is selected from the group consisting of avipox, suipox and orthopox. In still yet further embodiments, the poxviral nucleic acid is selected from the group consisting of TROVAC, NYVAC, ALVAC, MVA, Adeno-Associated Virus (AAV), Wyeth; and PoxvacTC.

Additional embodiments of the invention are contemplated encompassing nucleic acids comprising a sequence encoding for the CEA agonist polypeptide/protein in addition to a second sequence encoding at least one member selected from the group comprising cytokines, lymphokines, and co-stimulatory molecules.

Embodiments of the invention further contemplate vectors comprising the nucleic acid (s) of the invention. In particular embodiments, these vectors may be either recombinant viruses or bacteria. In further embodiments, the recombinant viruses are selected from the group consisting of adenovirus, alphavirus and poxvirus. In yet further embodiments, the poxvirus is selected for the group consisting of avipox, orthopox and suipox; particular embodiments encompass ALVAC, NYVAC, TROVAC, MVA, Wyeth and Poxvac-TC.

The invention further provides for cells comprising the aforementioned nucleic acid (s) of the invention, wherein said cells express the CEA agonist polypeptide/protein of the invention. In further embodiments, the cells expressing the CEA agonist polypeptide/protein also express a MHC HLA class 1 molecule. In yet further embodiments, the cells expressing the polypeptide are antigen-presenting cells.

Embodiments of the invention further encompass mixtures and/or compositions of the aforementioned CEA agonist polypeptides/proteins, nucleic acids, vectors, and cells. These mixtures and/or compositions, may optionally include adjuvants.

The invention further provides a method of inducing an immune response in an animal directed against: (i) a CEA protein or fragment thereof; and/or (ii) a CEA agonist polypeptide/protein of the invention; and/or (iii) a CEA epitope; and/or (iv) a modified CEA epitope; and/or (v) cells expressing a CEA protein or fragment thereof, CEA agonist polypeptide/protein of the invention, CEA epitope, modified CEA epitope; and/or; (vi) cells binding a CEA protein or fragment thereof, CEA agonist polypeptide/protein of the invention, CEA epitope, modified CEA epitope, comprising administering to said animal a CEA agonist polypeptide/protein, nucleic acid, vector, cell, or mixture and/or composition of the invention in an amount sufficient to induce an immune response.

The invention further contemplates a method of inhibiting a CEA epitope expressing carcinoma cell in a patient comprising administering to said patient an effective amount of a CEA agonist polypeptide/protein, nucleic acid, vector, cell, or mixture and/or composition of the invention.

The invention in yet a further aspect provides for a treatment for cancer comprising any one of the aforementioned methods for inducing immune responses and/or inhibiting carcinoma cells expressing a CEA epitope.

Other features and advantages of the present invention will become apparent from the following detailed description. It should be understood, however, that the detailed description and the specific examples while indicating particular embodiments of the invention are given by way of illustration only, since various changes and modifications within the spirit and scope of the invention will become apparent to those skilled in the art from this detailed, description.

The present invention relates to the preparation and use of peptides that can act as agonists and antagonists of human carcinoembryonic antigen (CEA). More specifically, the agonist peptide according to the present invention can be used as an immunogen, either alone, or in prime and boost protocols with other immunogens such as rV-CEA, for a variety of neoplastic conditions. These may include colorectal cancer, lung cancer, pancreatic cancer, and breast cancer. Thus, the present invention also relates to the production and use of vaccines against cancer. Peptide agonists according to the present invention can also be used to facilitate propagation of T cells, for example, from vaccinated patients, for adoptive transfer studies. Peptide antagonists according to the present invention find utility in suppressing autoimmune responses, such as those involving T cells, when such responses occur in vaccinated patients. Thus, the present invention also relates to the production and use of vaccines against autoimmune diseases, especially those mediated by lymphocytes and other antigen presenting cells.

Other aspects of the present invention relate to a recombinant vector comprising foreign genes encoding multiple costimulatory molecules and optionally a foreign gene encoding a target antigen. The invention further relates to a recombinant virus comprising foreign genes encoding at least three costimulatory molecules and optionally a foreign gene encoding at least one target antigen or immunological epitope thereof. More specifically, the present invention relates to a recombinant poxvirus comprising foreign genes encoding at least the costimulatory molecules: one molecule from the B7 family, LFA-3 and ICAM-1 and optionally a foreign gene encoding at least one target antigen or immunological epitope thereof and uses thereof as immunogens and vaccines. The invention further relates to antigen presenting cells transfected, infected or transduced by a recombinant vector comprising foreign genes encoding multiple costimulatory molecules and optionally a foreign gene encoding at least one target antigen or immunological epitope thereof.

Other aspects of the present invention relate to immunology, in particular to novel biologically active modified CEA agonist polypeptides/proteins containing a modified epitope therein, nucleic acids coding therefor, vectors and/or cells comprising said nucleic acid, mixtures and/or compositions of the aforementioned, and their use as immunogenic agents and/or in treatments of cancer.

Another aspects of the present invention is a vector containing foreign DNA coding at least three costimulatory molecules, alone or in combination with foreign DNA encoding at least one target antigen or immunological epitope thereof which allows functional expression of each foreign DNA in an infected host cell.

These and other objects, features and many of the attendant advantages of the invention will be better understood upon a reading of the detailed description of the invention.

### BRIEF DESCRIPTION OF ASPECTS OF THE DRAWINGS

FIG. 1. Genomic structure of plasmid pT5032 comprising nucleic acid sequences encoding murine LFA-3, ICAM-1 and B7.1, flanked by portions of the Hind III M region of the vaccinia genome.

FIG. 2. Genomic structure of plasmid pT5047 comprising nucleic acid sequences encoding murine LFA-3, ICAM-1, B7.1, and the lacZ gene, flanked by portions of the Hind III J region of the vaccinia genome.

FIG. 3. Genomic structure of plasmid pT5031 comprising nucleic acid sequences encoding murine LFA-3, ICAM-1 and B7.1 and a nucleic acid sequence encoding CEA, flanked by portions of the Hind III M region of the vaccinia genome.

FIGS. 4A through 4C. Genomic structure of recombinant vaccinia viruses expressing three murine costimulatory molecules with (FIG. 4C) or without (FIGS. 4A and B) a tumor-associated antigen. FIG. 4A shows the genomic structure of recombinant vaccinia, vT171. FIG. 4B shows the genomic structure of recombinant vaccinia vT199. FIG. 4C shows the genomic structure of recombinant vT172. Hind III M and Hind III J are the sites of insertion in the poxvirus of the foreign genes. Promoters 30K, I3, sE/L, 7.5K, 40K and C1 are promoters. Bam HI and Hind III restriction sites in the inserted sequences are shown, with the distance of each site (in kilobase pairs) from the 5′ end of the insertion (0) listed above each site in parentheses (not drawn to scale).

FIG. 5. Genomic structure of plasmid pT8001 comprising nucleic acid sequences encoding murine B7.1, LFA-3, ICAM-1 and the lacZ gene, flanked by portions of the BamHI J region of the fowlpox genome.

FIG. 6. Genomic structure of plasmid pT5049 comprising a nucleic acid sequence encoding the tumor associated antigen, CEA, and murine B7.1, LFA-3, and ICAM-1, in combination with the lacZ gene, flanked by portions of the BamHI J region of the fowlpox genome.

FIGS. 7A through 7D. Genomic structure of recombinant foxlpox viruses expressing three murine costimulatory molecules with (FIGS. 7B, 7C and 7D) or without (FIG. 7A) a tumor-associated antigen (TAA). FIG. 7A shows the genomic structure of recombinant fowlpox vT222. FIG. 7B shows the genomic structure of recombinant fowlpox vT194. FIG. 7C shows the genomic structure of recombinant fowlpox expressing MUC-1, B7.1, ICAM-1 and LFA-3. FIG. 7D shows the genomic structure of recombinant fowlpox expressing a tumor-associated antigen, B7.1, ICAM-1 and LFA-3. BamHI J is the site of insertion in the fowlpox virus genome of the foreign genes. sE/L, I3, 7.5K, C1, 40K and 30 K are poxviral promoters. P1-P5 denote five different poxvirus promoters. BamHI and HindIII restriction sites in the inserted sequences are shown, with the distance of each site (in kilobase pairs) from the 5′ end of the insertion (0) listed above each site in parentheses (not drawn to scale).

FIG. 8. Genomic structure of plasmid pT5064 comprising nucleic acid sequences encoding human LFA-3, human ICAM-1, human B7.1 and the lacZ gene, flanked by portions of the HindIII J region of the vaccinia genome.

FIGS. 9A through 9C Genomic structure of recombinant poxvirus expressing three human costimulatory molecules LFA-3, ICAM-1 and B7.1 along with the lacZ gene with (FIGS. 9B, C) or without (FIG. 9A) a tumor associated antigen, HindIII J is the site of insertion in the vaccinia virus genome of the foreign genes. BamHI J is the site of insertion in the fowlpox virus genome. 30K, I3, sE/L, 40K and C1 are poxviral promoters. BgIII and HindIII restriction sites in the inserted sequences are shown, with the distance of each site (in kilobase pairs) from the 5′ end of the insertion (O) listed above each site in parentheses (not drawn to scale).

FIG. 10. Genomic structure of plasmid pT8016 comprising nucleic acid sequences encoding CEA (6D) and human LFA-3, ICAM-1, B7.1, and the E. coli lacZ gene, flanked by portions of the Hind III J region of the vaccinia genome.

FIG. 11. Genomic structure of recombinant vaccinia virus vT238 expressing CEA (6D) and three human costimulatory molecules. HindIII J is the site of insertion in the poxvirus genome of the foreign genes. 40K, 30K, I3, sE/L, and C1 are poxviral promoters.

FIG. 12. Genomic structure of plasmid pT8019 comprising nucleic acid sequences encoding murine LFA-3, ICAM-1, B7.1, and the E. coli lacZ gene, flanked by portions of the BamHI J region of the fowlpox genome.

FIGS. 13A and 13B. Genomic structure of recombinant fowlpox viruses expressing murine or human costimulatory molecules. FIG. 13A shows the genomic structure of recombinant fowlpox vT251. FIG. 13B shows the genomic structure of recombinant fowlpox vT232. BamHI J is the site of insertion in the poxvirus genome of the foreign genes. 30K, I3, sE/L and C1 are poxviral promoters.

FIG. 14. Genomic structure of plasmid pT5072 comprising nucleic acid sequences encoding human LFA-3, ICAM-1, B7.1, and the E. coli lacZ gene, by portions of the BamHI J region of the fowlpox genome.

FIG. 15. Genomic structure of plasmid pT8020 comprising nucleic acid sequences encoding MUC-1, murine LFA-3, ICAM-1, B7.1, and the E. coli lacZ gene, flanked by portions of the BamHI J region of the fowlpox genome.

FIGS. 16A through 16D. Genomic structure of recombinant fowlpox viruses expressing murine or human costimulatory molecules with at least one tumor-associated antigen. FIG. 16A shows the genomic structure of recombinant fowlpox vT250. FIG. 16B shows the genomic structure of recombinant fowlpox vT242. FIG. 16C shows the genomic structure of recombinant fowlpox vT236. FIG. 16D shows the genomic structure of recombinant fowlpox vT257. BamHI J is the site of insertion in the poxvirus genome of the foreign genes. 40K, 7.5K, 30K, I3, sE/L, and C1 are poxviral promoters.

FIG. 17. Genomic structure of plasmid pT2186 comprising nucleic acid sequences encoding MUC-1, human LFA-3, ICAM-1, B7.1, and the E. coli lacZ gene, flanked by portions of the BamHI J region of the fowlpox genome.

FIG. 18. Genomic structure of plasmid pT2187 comprising nucleic acid sequences encoding CEA (6D), human LFA-3, ICAM-1, B7.1, and the E. coli lacZ gene, flanked by portions of the BamHI J region of the fowlpox genome.

FIG. 19. Genomic structure of plasmid pT5080 comprising nucleic acid sequences encoding PSA, PSMA, human LFA-3, ICAM-1, B7.1, and the E. coli lacZ gene, flanked by portions of the BamHI J region of the fowlpox genome.

FIG. 20. Genomic structure of plasmid pT5085 comprising nucleic acid sequences encoding murine LFA-3, ICAM-1, B7.1, and the E. coli lacZ gene, flanked by portions of the deletion III region of the MVA genome.

FIGS. 21A and 21B. Genomic structure of recombinant MVA viruses expressing murine or human costimulatory molecules with or without tumor-associated antigens. FIG. 21A shows the genomic structure of recombinant MVA vT264. FIG. 21B shows the genomic structure of recombinant MVA vT260. Deletion III is the site of insertion in the poxvirus genome of the foreign genes. 40K, 7.5K, 30K, I3, sE/L, and C1 are poxviral promoters.

FIG. 22. Genomic structure of plasmid pT5084 comprising nucleic acid sequences encoding PSA, PSMA, human LFA-3, ICAM-1, B7.1, and the E. coli lacZ gene, flanked by portions of the deletion III region of the MVA genome.

FIG. 23. Costimulatory molecule surface expression following infection with recombinant viruses. MC38 tumor cells were infected for 5 hours at 5 MOI (multiplicity of infection; pfu/cell) with the indicated virus. After infection, cells were immunostained with FITC-labeled monoclonal antibodies (MAb) specific for the costimulatory molecule. Shaded areas are fluorescence intensity of the specific MAb while unshaded areas are the fluorescence intensity of the appropriate isotype control antibody (see Materials and Methods).

FIGS. 24A and 24B. Effect of multiple costimulatory molecules on T-cell proliferation. Naive murine T cells, in the presence of varying concentrations of Con A to provide the first signal, were co-cultured with MC38 stimulator cells infected with either recombinant vaccinia (FIG. 24A) or recombinant fowlpox (FIG. 24B) vectors. Recombinant vectors were wild-type (i.e. V-Wyeth or WT-FP [open squares]), rV-LFA-3 (closed triangle), rV-ICAM-1 or rF-ICAM-1 (closed circles), rV-B7-1 or rF-B7-1 (closed diamonds), and rV-B7-1/ICAM-1/LFA-3 or rF-CEA/B7-1/ICAM-1/LFA-3 (closed squares). Uninfected MC38 cells are open circles. Proliferation assay is as described in Materials and Methods.

FIGS. 25A through 25D. Specificity of costimulation delivered via recombinant vaccinia viruses. T cells, in the presence of Con A, were co-cultured with MC38 stimulator cells infected with V-Wyeth (FIG. 25A), rV-B7-1 (FIG. 25B), rV-ICAM-1 (FIG. 25C), and rV-LFA-3 (FIG. 25D), as denoted by open circles. Infected stimulator cells in the presence of costimulatory molecule-specific MAb are denoted by closed circles, and isotype control antibody is denoted by closed triangles.

FIG. 26. Relative capacity of B7-1, ICAM-1, LFA-3 and the coexpression of all three costimulatory molecules to deliver the second signal for T-cell proliferation. In the presence of Con A (2.5 .mu.g/ml), 100,000 T cells were co-cultured with 10,000 MC38 cells. The stimulator MC38 cells expressing one or all of the costimulatory molecules were added to the wells in various ratios in combination with V-Wyeth-infected stimulator cells to a total of 10⁴ MC38 cells/well. MC38 cells were infected with V-Wyeth (open square). rV-LFA-3 (closed triangles), rV-ICAM-1 (closed circles), rV-B7-1 (closed diamonds), or rV-B7-1-ICAM-1-LFA-3 (closed squares). Cells were co-cultured for 48 hours. During the final 18 hours, ³H-Thymidine was added to measure T-cell proliferation. Inset panel depicts proliferation values obtained from a culture in which 3% of the MC38 stimulator cells were infected with the erectors shown. Thus, in this experiment, the final ratio of stimulator cells to T cells was 0.003. Note the relatively poor effect of rVB7.1/ICAM under these conditions as compared to rV-B7/ICAM/LFA-3.

FIGS. 27A through 27D. Effect of costimulation on specific T-cell populations. Murine CD4⁺ (FIG. 27A) or CD8⁺ T cells (FIG. 27B) were co-cultured with uninfected MC38 cells (open circle), or cells infected with V-Wyeth (open squares), rV-LFA-3 (closed triangles), rV-ICAM-1 (closed circles), rV-B7-1 (closed diamonds) or rV-B7-1/ICAM-1/LFA-3 (closed squares) at a 10:1 ratio for 48 hours in the presence of various concentrations of Con A. During the final 18 hours, ³H-Thymidine was added to measure T-cell proliferation. FIGS. 27C and 27D show the proliferative responses of purified CD4⁺ and CD8⁺ cells, respectively, when co-cultured in the presence of vector-infected MC38 stimulator cells at a low Con A concentration (0.625 .mu.g/ml).

FIGS. 28A through 28D. Effect of costimulation on cytokine production. Murine CD4⁺ (FIGS. 28A and 28C) or CD8⁺ (FIGS. 28B and 28D) T cells were purified as described in Materials and Methods and co-cultured with the indicated MC38 vector-infected stimulator cells for 24 hours in the presence of 2.5 .mu.g/ml Con A. Supernatant fluids were analyzed for production of IL-2 (FIGS. 28A and 28B) and IFN-.gamma. (FIGS. 28C and 28D) by capture ELISA.

FIGS. 29A through 29C. Effect of costimulation on cytokine RNA expression. FIG. 29A: murine CD4⁺ or CD8⁺ T cells were co-cultured with MC38 stimulator cells infected with V-Wyeth (lane A), rV-B7-1 (lane B), rV-ICAM-1 (lane C), rV-LFA-3 (lane D) or rV-B7-1/ICAM-1/LFA-3 (lane E) at a T-cell to stimulator cell ratio of 10:1 for 24 hours in the presence of 2.5 .mu.g/ml Con A. Following culture, T-cell RNA was analyzed by multiprobe RNAse protection assay. The quantitative representation of results from the autoradiograph is normalized for expression of the housekeeping gene L32 in FIG. 29B (CD4⁺ cells) and FIG. 29C (CD8⁺ cells). Order of histogram bars (from left to right) is MC38/V-Wyeth, MC38/B7-1, MC38/ICAM-1, MC38/LFA-3, and MC38/B7-1/ICAM-1/LFA-3.

FIG. 30. C57BL/6 mice (5/group) were administered HBSS (closed squares) or vaccinated with 10⁷ pfu rV-CEA (closed triangles) or rV-CEA/TRICOM (closed circle). One hundred days later, mice were inoculated with 1.times.10⁶ MC38 carcinoma cells expressing CEA and survival was monitored. All mice other than the rV-CEA/TRICOM group developed tumors and were sacrificed when tumors exceeded 20 mm in length or width, or when the mice were moribund. FIG. 30: In a second experiment, C57BL/6 mice (5/group) were vaccinated with 10⁷ pfu rV-CEA, rV-CEA/B7.1, rV-CEA/TRICOM or HBSS buffer. Lymphoproliferative responses from pooled splenic T cells were analyzed 22 days following vaccination. Values represent the stimulation index of the mean cpm of triplicate sames vs. media. Standard deviation never exceeded 10%. Antigens used were Con A (5 .mu.g/ml), CEA (100 .mu.g/ml) and ovalbumin (100 .mu.g/ml).

FIG. 31 shows a schematic of an in vitro costimulation assay of dendritic cells.

FIGS. 32A and 32B show the proliferative response of naive CD4⁺ (FIG. 32A) or naive CD8⁺ (FIG. 32B) T cells stimulated with progenitor DCs infected with rV-B7/ICAM-1/LFA-3 or DCs (noninfected, ie. CD34⁺ cells treated with GM-CSF+IL-4 for 6 days) in the presence of Con A.

FIGS. 33A and 33B show the proliferative response of nave CD4⁺ (FIG. 33A) or nave CD8⁺ (FIG. 33B) T cells stimulated with progenitor DCs infected with rV-B7/ICAM-1/LFA-3 or DCs infected with rV-B7/ICAM-1/LFA-3 or V-Wyeth (control).

FIG. 34 shows the mixed lymphocyte reaction (MLR) of Balb/C splenocytes vs. irradiated C57b1/6 dendritic cells infected with 25 MOI of V-Wyeth or rV-TRICOM. ³H-thymidine pulsed on day 3, harvest on day 4, .quadrature.DC (uninfected), .box-solid. DC (V-Wyeth infected), .quadrature.DC (rV-TRICOM infected).

FIG. 35 shows the proliferative response of responder T cells (CAP-M8 T-cell line specific for CEA peptide 8) at various APC ratios harvested on day 5 after stimulation with peptide-pulsed DCs infected with rV-TRICOM and rested 2 days with 10 .mu./ml IL2 (no APC or peptide). Peptide 8-(EAQNTTYL) in assay at 1 .mu.g/ml final concentration. ³H-thymidine added on day 2, T cells harvested on day 3. 0=DC(v-Wyeth)-pep and .DELTA.=DC (rV-TRICOM)-pep results are at baseline.

FIGS. 36A and 36B. Efficiency of poxviral infection of murine dendritic cells (DC). DC were infected with 25 MOI rV-TRICOM or 50 MOI rF CEA/TRICOM for 5 h. DC infected with TRICOM vectors exhibit enhanced capacity to stimulate nave T-cells. All DC populations were co-cultured for 48 h with T-cells at a ratio of 10:1 in the presence of different concentrations of Con A to provide signal-1. ³H-thymidine was added during the final 18 h. FIG. 36A: Uninfected DC (closed squares), mock-infected DC (closed diamonds), or DC infected with V-WT (closed inverse triangles), rV-B7.1 (open triangles) or rV-TRICOM (open circles). FIG. 36B: DC (closed squares), mock-infected DC (closed diamonds), or DC infected with WT-FP (closed inverse triangles), rF-B7.1 (open triangles) or rF-TRICOM (open circles).

FIGS. 37A through 37F. Enhanced allostimulatory activity by DC infected with vaccinia (FIGS. 37A, C, E) or fowlpox (FIGS. 37B, D, F) vectors. Uninfected DC (closed squares); mock-infected DC (closed diamonds); or DC infected with wild-type poxviral vectors (V-WT or F-WT, closed inverse triangles), rV-B7.2 or rF-B7.1 (open triangles), or rV-TRICOM or rF-TRICOM (open circles) were co-cultured with allogeneic (FIGS. 37A-D) or syngeneic T cells (FIGS. 37E-F) for 5 days. ³H-thymidine was added during the final 18 h.

FIGS. 38A through 38F. Effect of vaccinia infection of DC on peptide-specific T-cell proliferation. Uninfected DC (closed squares), or DC infected with V-WT (closed inverse triangles), rV-B7.1 (open triangles) or rV-TRICOM (open circles) were co-cultured with OVA peptide-specific T cells (FIGS. 38A, C, E) or CAP-M8 peptide-specific T cells (FIGS. 38B, D, F). Experimental conditions included a fixed effector:stimulator cell ration of 10:1 in the presence of various concentrations of the appropriate peptides (FIGS. 38A-D), negative control peptides (open squares, either VSVN (FIG. 38A), or FLU-NP (FIG. 38B), or a fixed peptide concentration of 1 .mu.M in the presence of various effector:stimulator cell ratios (FIGS. 38E and F).

FIGS. 39A and 39B. Effect of rV-TRICOM infection with DC matured with TNF-.alpha. or CD40. DC (closed squares), or DC cultured with either 100 ng/ml TNF-.alpha. (open triangles), or 5 .mu.g/ml CD40 mAb (open circles) for the final 24 h of culture were used to stimulate CAP-M8-specific effector T cells (FIG. 39A). The proliferation of CAP-M8 T cells in response to these DC populations after infection with 25 MOI rV-TRICOM (FIG. 39B). For all panels, the T-cell:DC ratio was 10:1, while the CAP-M8 peptide concentration was 1 .mu.g/ml. Closed circles denote proliferation of CAP-M8 T cells stimulated with all DC populations in the presence of 1 .mu.g/ml VSVN peptide.

FIGS. 40A through 40H: Effect of vaccinia infection of DC on induction of CTL activity. DC (FIG. 40B), or DC infected with V-WT (FIG. 40C), or rV-TRICOM (FIG. 40D) were pulsed with 10 .mu.M OVA peptide for 2 h. DC populations were administered intravenously to mice (1.times.10⁵ cells/mouse). Control mice were immunized subcutaneously with 100 .mu.g OVA peptide in Ribi/Detox adjuvant (FIG. 40A). Fourteen days later spleens were harvested, restimulated for 6 days with the corresponding peptide, and assessed for lytic ability against EL-4 cells pulsed with either OVA (closed squares) or VSVN peptides (open squares). Inset numbers depict CTL activity as expressed in lactic units. Also shown is the effect of vaccinia infection of DC on induction of CTL activity. DC (FIG. 40F), or DC infected with V-WT (FIG. 40G), or rV-TRICOM (FIG. 40H) were pulsed with 10 .mu.M CAP-M8 peptide for 2 h. DC populations were administered intravenously to mice (1.times.10⁵ cells/mouse). Control mice were immunized subcutaneously with 100 .mu. CAP-M 8 peptide in Ribi/Detox adjuvant (FIG. 40E). Fourteen days later spleens were harvested, restimulated for 6 days with the corresponding peptide, and assessed for lytic ability against EL-4 cells pulsed with either CAP-M8 (closed squares) or FLU-NP peptides (open squares). Inset numbers depict CTL activity as expressed in laic units.

FIGS. 41A through 41C: Effect of multiple immunizations with vaccinia-infected DC on induction of CTL activity. DC (closed squares) or DC infected with V-WT (closed inverse triangles) or rV-TRICOM (open circles) were pulsed with 10 .mu.M CAP-M8 peptide for 2 h. DC populations were administered intravenously to mice (1.times.10⁵ cells/mouse) 1, 2 or 3 times at 7 day intervals. Control mice were immunized subcutaneously with 100 .mu.g CAP-M8 peptide in Ribi/Detxo adjutant (crosses). Fourteen days after the final immunization, spleens were harvested, restimulated for 6 days with CAP-M8, and assessed for lytic ability against EL-4 cells pulsed with CAP-M8 or control peptide VSVN (not shown).

FIGS. 42A and 42B. Effect of vaccinia and fowlpox TRICOM-infected splenocytes on T cell proliferation. Nave murine T cells were co-cultured with autologous splenocytes infected with either recombinant vaccinia or fowlpox vectors. Co-culture was performed in varying concentrations of Con-A as Signal-1. Recombinant vectors were wild type (i.e. V-WT, FP-WT, open diamond), rV-B7-1 or rF-B7-1, (open circles) or rV-TRICOM or rF-TRICOM (closed squares). Uninfected splenocytes are shown as open triangles.

FIGS. 43A through 43D. Effect of TRICOM vector infected splenocytes on allogeneic T cells. Nave Balb/C T cells were co-cultured with C57B1/6 splenocytes infected with recombinant vaccinia (FIGS. 43A and C) or fowlpox (FIGS. 43B and D) vectors for either 2 days (FIGS. 43A and B) or 5 days (FIGS. 43C and 43D). Recombinant vectors were V-WT or FP-WT, open diamonds, rV-B7-1 or rF-B7-1 (open circles), or rV-TRICOM or rF-TRICOM (closed squares). Uninfected splenocytes are indicated as open triangles. Proliferation induced by DC is indicated as closed squares.

FIGS. 44A through 44F. Effect of rV-TRICOM-infected splenocytes on specific T cell populations. Nave murine T cells were fractionated with CD3⁺, CD4⁺, and CD8⁺ subpopulations. T cells were co-cultured with either uninfected autologous BMDC or splenocytes infected with recombinant vaccinia vectors. Varying Con-A concentrations (FIGS. 44A-C) or varying number of stimulator cells (FIGS. 44D-F) provided the first signal. T cell proliferation in response to mature BMDC is indicated by open squares, and to uninfected splenocytes by open triangles. Recombinant vectors were wild-tape (V-WT, open diamonds) or rV-TRICOM (closed squares).

FIGS. 45A through 45F. Effect of rV-TRICOM-infected bone marrow cells on specific T cell populations. Nave murine T cells were fractionated into CD3⁺, CD4⁺, and CD8⁺ subpopulations. T cells were co-cultured with either uninfected autologous BMDC or splenocytes infected with recombinant vaccinia vectors. Varying Con-A concentrations (FIGS. 45A-C) or varying number of stimulator cells (FIGS. 45D-F) provided the first signal. T cell proliferation in response to mature BMDC is indicated by open squares, and to uninfected splenocytes by open triangles. Recombinant vectors were wild-tape (V-WT, open diamonds) or rV-TRICOM (closed squares).

FIGS. 46A through 46D. Effect or rV-TRICOM-infected splenocytes or bone marrow (BM) cells on peptide-specific memory CD8⁺ T cells. CAP-M8-specific T cells were co-cultured with autologous splenocytes (FIGS. 46A and B) or bone marrow cells (FIGS. 46C and D) infected with recombinant vaccinia vectors. The analysis was carried out using two sets of conditions: a) a 10:1 fixed ratio of responder:stimulator cells that were cultured in the presence of several concentrations of CAP-M8 peptide (FIGS. 46A and 46C), or b) a fixed concentration of peptide (1 uM) at various responder:stimulator ratios (FIGS. 46B and 46D). Recombinant vectors were wild type (open diamonds), and rV-TRICOM (closed squares). Uninfected splenocytes are shown as open triangles. BM are shown as open squares.

FIG. 47. Shows production of IFN-.gamma. bad human T cells isolated from peripheral blood mononuclear cells (PBMC) using rF-TRICOM-infected human dendritic cells pulsed with CEA peptides, CAP-1 or CAP1, 6D.

FIG. 48. Shows production of IFN-.gamma. by human T cells using rF-TRICOM-infected human dendritic cells pulsed with PSA peptide. PSA-3.

FIG. 49. Shows production of IFN-.gamma. by human T cells isolated from PBMC using rF-TRICOM-infected human dendritic cells pulsed with Flu peptide 58-66.

FIG. 50. Shows production of IFN-.gamma. by human T cells isolated from PBMC using rF-TRICOM- or rF-B7.1-infected human dendritic cells pulsed with Flu peptide 58-66 at various effector:APC ratios.

FIG. 51. Shows production of IFN-.gamma. by human T cells from donor 868 using rF-TRICOM-infected human dendritic cells pulsed with HPV peptide (11-20) after one or two in vitro stimulation (IVS).

FIG. 52. Shows production of IFN-.gamma. by human T cell line using rF-TRICOM- or rF-B7.1-infected human dendritic cells pulsed with HPV peptide (11-20).

FIG. 53. Shows production of IFN-.gamma. be a human T cell line using rF-TRICOM- or rF-B7.1-infected human dendritic cells pulsed with various concentrations of HPV peptide (11-20).

FIG. 54. Shows production of IFN-gamma. by human T cells using rF-TRICOM or rF-B7.1-infected human dendritic cells pulsed with HPV E7 peptide 11-20 at various effector:APC ratios.

FIG. 55A-D: Effect of single amino acid substitutions in CEA CAP1 peptide on lysis by CEA CTL T-Vac8 C1R-A2 cells were labeled with ¹¹¹In and incubated for 1 hour in round bottom wells (2,000/well) with each substituted peptide at 1 (solid), 0.1 (open) and 0.01 (hatched).mu.g/ml. T-Vac8 CTL were added at E:T=1.45:1 and isotope release was measured after 4 hours. Spontaneous release was determined for each peptide at 1 .mu.g/ml. All assays were performed in triplicate. FIGS. 55A-D depict substitutions at positions p5 through p8, respectively. Amino acids are designated by the single letter code; the amino acid encoding the native CAP1 sequence is indicated in each figure and alone the right-hand margin.

FIG. 56A-B: CAP1 and analogs show different sensitivity to CL-A CTL T-Vac8 cytotoxicity FIG. 56A-B target cells were labeled with ⁵¹Cr and incubated in round-bottomed 96 well plates (10,000/well) with CAP1 (large circle) or substituted peptides CAP1-6D (quadrature) or CAP1-71 (diamond) at the indicated concentrations. After 1 hour. T-Vac8 CTL were added at E:T=2.5:1 and isotope release was determined after 4 hours. All assays were done in triplicate. NCA571 (DELTA) is a 9-mer peptide obtained after optimal alignment of CEA with the related gene NCA (11).

FIG. 57: Effect of single amino acid substitutions in CAP1 peptide on binding to and stability of HLA-A2 complexes T2 cells were collected in serum free medium then incubated overnight (10⁶ well) with peptides CAP1 (large circle). CAP1-6D (quadrature), or CAP1-71 (diamond) at the indicated concentrations. Cells were collected and assayed for cell surface expression of functional HLA-A2 molecules by staining with conformation sensitive MAb BB7.2, HLA specific antibody W6/32 (not shown) and isotype control Ab MOPC-195 (not shown). Mean fluorescent intensity was determined on a live, gated cell population. Figure insert: Cells were incubated with peptide at 100 .mu.g/ml overnight, then washed free of unbound peptide and incubated at 37 C. At the indicated times, cells were stained for the presence of cell surface peptide-HLA-A2 complexes. The error bars indicate SEM for two experiments.

FIG. 58A-B: CTL generated from apparently health individuals with CAP1-6D peptide recognize CAP1 and CAP1-6D CTL lines (designated T-N1 and T-N2) were generated with CAP1-6D and were assayed for peptide specificity. T-N1 was assayed after 5 cycles of stimulation at an effector to target ratio of 20:1 (FIG. 58A). T-N2 was assayed after 10 cycles at an effector to target ratio of 15:1 (FIG. 58B). ⁵¹Cr-labeled C1R-A2 targets (5.000/well) were incubated with the indicated amount of CAP1 (small circle) or CAP1-6D (quadrature) peptide. After 4 hours the amount of isotope release was determined in a gamma counter. Values were determined from triplicate cultures.

FIG. 59A-B: CAP1-6D, but not CAP1 generated T cell lines from apparently healthy donors recognize tumor cells expressing endogenous CEA CAP1-6D generated T-N2 CTL (FIG. 59A) and T cells generated with native CAP1 (FIG. 59B), were assayed after 9 cycles of in vitro stimulation against tumor targets SW480 and SW1463 (CEA⁺, HLA-A2⁺, large circle and respectively), SKmel24 (CEA⁻, -A2⁺, quadrature) and K562 (diamond). Tumor cells were cultured for 72 hours in the presence of .gamma.-IFN to up regulate HLA. Cells were trypsinized and labeled with ⁵¹Cr and incubated (5,000 cells/well) with T-N2 CTL at increasing effector to target ratios. Cultures were incubated for 4 hours and the amount of isotope release determined in a gamma, counter. Values were determined from triplicate cultures.

FIG. 60: MHC-class 1 A2.1 restriction of CTL line (T-N2) derived from CAP1-6D agonist CTL line T-N2 was used as an effector for the human colon carcinoma SW837 target cell. SW837 is CEA positive and HLA-A2.1 negative. SW837 were infected at an MOI of 110:1 with either a recombinant vaccinia containing the A2.1 transgene (quadrature) or wild type vaccinia (DEL-TA).

FIG. 61A-B: CTL generated with CAP1-6D lose CEA positive, HLA-A2 positive tumors: Effect of IFN upregulation The T-N1 CTL generated with CAP1-6D were assayed against various tumor cell lines: SW480 (CEA⁺ and HLA-A2⁺, large circle), SW1116 (CEA.⁺ but -A2⁻, quadrature) and CaOV3 (CEA⁻ but -A2⁺, diamond). Tumor cells were cultured 72 hours in the absence (FIG. 61A) or presence (FIG. 61B) of .gamma.-IFN, trypsinized and labeled with ⁵¹Cr then incubated (5,000 cells/well) with T-N1 CTL at increasing effector to target ratios. Cultures were incubated for 4 hours and the amount of isotope release determined in a gamma counter. Values were determined from triplicate cultures.

FIG. 62: depicts the nucleic acid and amino acid sequence of an embodiment of the invention encompassing modified CEA.

FIG. 63 depicts 3 schematic representations encompassing the genomic structure of particular recombinant fowlpox, vaccinia and MVA constructs expressing modified CEA.

FIG. 64 depicts a schematic representation of the restriction map profile of an ALVAC(2)-CEA (modified)/human B7.1 construct.

FIG. 65 depicts the nucleic acid sequence of the H6-promoted CEA (modified)/human B7.1 insertion cassette used in the production of the construct of FIG. 64.

FIG. 66 depicts the results of animmunoprecipitation analysis of HeLa cells infected with various ALVAC recombinant constructs.

FIG. 67 depicts the results of a western blot analysis of HeLa cells infected with various ALVAC recombinant constructs.

### DETAILED DESCRIPTION OF ASPECTS OF THE INVENTION

The invention is an peptide agonist of the native CEA epitope, CAP-1 (SEQ. ID NO: 1A), as well as antagonists of SEQ. ID NO: 1A. The agonist is characterized by its ability to elicit antigen specific cytotoxic T lymphocytes which inhibit the growth or kill carcinoma cells expressing CEA or CEA epitopes. An antagonist of the present invention serve to inhibit or prevent CEA specific immune responses. Such peptides may be used to shut off any unwanted immune responses to CAP-1 or CEA. One example for such use of an antagonist is 10 control any possible autoimmune response that may occur during cancer immunotherapy, where the therapy has killed off tumor cells and begins to attack normal cells expressing CEA. In accordance with the present invention an antagonist would advantageously prevent extensive damage to normal tissue.

The peptide agonists of the present invention comprise about 8-13 amino acids, preferably 9-10 amino acids. In a preferred embodiment, the agonist peptide of the present invention comprises at least one amino acid substitution at a non-anchor position. In one embodiment, the agonist comprises a sequence with a substitution at position 6 compared to the native CAP-1 (SEQ. ID NO: 1A). In another embodiment the agonist comprises a sequence with an amino acid substitution at position 7 compared to the native CAP-1 (SEQ. ID NO: 1A). In vet another embodiment the agonist comprises a sequence with an amino acid substitution at position 6 and at position 7 compared to the native CAP-1. The substituted amino acid sequences to enhance the interaction of the TCR complex on the cytotoxic T lymphocytes with the peptide-MHC antigen ligand complex. Such enhanced interaction results in greater effector function by the cytotoxic T lymphocytes.

An example of a substitution includes Asp and Cys at position 6 or an Ile at position 7.

In one embodiment, the peptide agonist comprises the following amino acid sequence:

Amino Acid

| | |
|---|---|
| Position | 1 2 3 4 5 6 7 8 9 |

| | |
|---|---|
| Native CAP-1 | |
| Peptide | Y L S G A N L N L (SEQ. ID NO: 1) |
| Agonist | Y L S G A D L N L (SEQ. ID NO: 2) |
| Agonist | Y L S G A D I N L (SEQ. ID NO: 3) |
| Agonist | Y L S G A N I N L (SEQ. ID NO: 4) |
| Agonist | Y L S G A C L N L (SEQ. ID NO: 5) |

The agonist peptide of the present invention may be obtained by recombinant DNA technology or by chemical peptide synthesis.

The agonist peptide may be formulated into a pharmaceutical composition in combination with a pharmaceutically acceptable carrier for use as an immunogen in a mammal, preferably a human. The composition may further comprise one or more other constituents to enhance the immune response which include but are not limited to immunostimulatory molecules such as interleukin 2, interleukin 6, interleukin 12, interferon gamma, tumor necrosis factor alpha, GM-CSF, B7.1, B7.2, ICAM-1, LFA-3, CD72, and cyclophosphamide.

The agonist peptide is administered to a mammal in an amount effective in generating a CEA specific immune response, preferably a cellular immune response. The efficacy of the mutant ras peptide as an immunogen may be determined by in vivo or in vitro parameters as are known in the art. These parameters include but are not limited to antigen specific cytotoxicity assays, regression of tumors expressing CEA or CEA epitopes, inhibition of cancer cells expressing CEA or CEA epitopes, production of cytokines and the like.

At least one or more agonist peptides may be administered in a dose of about 0.05 mg to about 10 mg per vaccination of the mammal, preferably about 0.1 mg to about 5 mg per vaccination. Several doses may be provided over a period of weeks as indicated. In one embodiment a dose is provided every month for 3 months. The agonist peptide may be administered alone or in combination with adjuvants incorporated into liposomes (U.S. Pat. Nos. 5,643,599; 5,464,630; 5,059,421; 4,885,172), with cytokines, biological response modifiers, or other reagents in the art that are known to enhance immune response. Adjuvants include but are not limited to RIBI Detox™, QS21, alum and incomplete Freund's adjuvant. In one embodiment, the mutant ras peptide is administered in combination with Detox™ (RIBI Immunochem Research, Hamilton, Mont.). RIBI Detox™ contains as active ingredients the cell wall skeleton from Mycobacterium phlei and monophosphloryl lipid A from Salmonella minnesota R595 prepared as an oil-in-water emulsion with squalene and tween 80.

The agonist peptides mats also be conjugated to helper peptides or to large carrier molecules to enhance the immunogenicity of the peptide. These molecules include but are not limited to influenza peptide, tetanus toxoid, tetanus toxoid CD4 epitope, Pseudomonas exotoxin A, poly-L-lysine, a lipid tail, endoplasmic reticulum (ER) signal sequence and the like.

The peptides of the present invention many also be conjugated to an immunoglobulin molecule using art accepted methods. The immunoglobulin molecule may be specific for a surface receptor present oil tumor cells but absent or in very low amounts on normal cells. The immunoglobulin may also be specific for a specific tissue. Such a peptide-immunoglobulin conjugate allows for targeting of the peptide to a specific tissue and/or cell.

Another effective form of the agonist peptide for generating an peptide specific immune response in a mammal is an agonist peptide-pulsed antigen presenting cell. The antigen presenting cells include but is not limited to dendritic cells, B lymphocytes, monocytes, macrophages and the like. In a preferred embodiment, the agonist peptide-pulsed antigen presenting cell is a dendritic cell.

The invention also provides a method of generating CEA and agonist peptide specific cytotoxic T lymphocytes in vivo or in vitro by stimulation of lymphocytes from a source with an effective amount of a agonist alone or in combination with a immunostimulatory molecule and/or adjuvant or in a liposome formulation. The sources of lymphocytes include but are not limited to peripheral blood, tumor tissues, lymph nodes and effusions such as pleural fluid or ascites fluid and the like.

The CEA and agonist peptide specific cytotoxic T lymphocytes of the present invention are immunoreactive with CEA agonist or peptide. The cytotoxic T lymphocytes inhibit the occurrence of tumor cells and cancer and inhibit the growth or kill expressing tumor cells expressing CEA or epitopes thereof or agonist expressing tumor cells. The cytotoxic T lymphocytes, in addition to being antigen specific, are MHC class I restricted. In one embodiment the cytotoxic T lymphocytes are MHC class I HLA-A2 restricted. The cytotoxic T lymphocytes have a CD8⁺ phenotype.

Selected patients bearing carcinoma cells expressing CEA or CEA epitopes are vaccinated subcutaneously up to three times at monthly intervals with DETOX™ adjuvant admixed with the appropriate peptide agonist may also be vaccinated carcinoma patients with autologous peripheral blood mononuclear cells pre-pulsed ex vivo with a peptide agonist alone or in combination with a peptide agonist. Anti-CEA T cell responses are evaluated as measured by proliferation assays.

Vaccination with CEA agonist peptides of the present invention induces highly specific and systemic anti-CEA cellular immune responses. Moreover, the development of such MHC class I-restricted agonist peptides has important implications for both active (i.e., vaccination) and passive (i.e., ex vivo expansion for cellular adoptive transfer) immunotherapies, which may be used for the induction and propagation of specific CD8⁺ CTL responses in cancer patients.

Patients with solid tumors expressing CEA or epitopes thereof, including but not limited to colon cancer, lung cancer, pancreas cancer, endometrial cancer, breast cancer, thyroid cancer, melanoma, oral cancer, laryngeal cancer, seminoma, hepatocellular cancer, bile duct cancer, acute myeloblastic leukemia, basal cell carcinoma, squamous cell carcinoma, prostate cancer and the like benefit from immunization with the agonist peptides. Patients amenable to treatment using the agonist peptides of the present invention are those patients having tumors with CEA or CEA epitopes.

Peptides may be chemically synthesized under GMP conditions and purified by HPLC to >95% purity and lyophilized. Pharmaceutical compositions are formulated by reconstituting the peptide with a pharmaceutically acceptable carrier such as sodium chloride. In one example, each milliliter of solution contains 1500 .mu.g of a agonist peptide plus 9.0 mg sodium chloride.

When the agonist peptide is administered with an adjuvant it is desirable to mix the peptide with the adjuvant shortly before administration to a patient.

The agonist peptide may be administered to a patient by various routes including but not limited to subcutaneous, intramuscular, intradermal, intraperitoneal, intravenous and the like. In one embodiment the agonist peptide is administered subcutaneously. The peptide may be administered at one or more sites to a patient. In one embodiment, the peptide, alone or in combination with an adjuvant, is administered into three sites subcutaneously, over the deltoids, the thighs and the abdomen.

In another method of generating an immune response, agonist peptide-pulsed antigen presenting cells are administered to the patient in an amount effective to generate an antigen specific immune response. The antigen presenting cells include but are not limited to dendritic cells, B lymphocytes, monocytes, macrophages and the like. In one embodiment, dendritic cells are isolated from a patient by methods described in Romani, N. et al (1994). The isolated dendritic cells are cultured in vitro with an agonist peptide for a period of about 0.5 to about 3 hours and washed to remove non-bound peptide. The agonist peptide-pulsed dendritic cells are transferred back into the patient at a concentration of about 10⁶ to about 10⁹ dendritic cells. Such a concentration is effective in generating an immune response in the patient including the generation of agonist peptide specific cytotoxic T lymphocytes which are able to inhibit the growth or kill tumor cells.

The criteria for determining an anti-tumor response in the immunized patient is as follows:

1. Complete Remission (CR): Complete disappearance of all evidence of tumor and return of abnormal tests to normal levels for a minimum of 4 weeks.

2. Partial Response (PR): Decrease by at least 50% in the sum of the products of the perpendicular diameters of all measured lesions in the absence of progression of any lesion nor the appearance of any new lesions for at least 4 weeks.

3. Stable Disease (SD): Change in measurable disease too small to meet the requirements for partial response or progression and the appearance of no new lesions for a period of at least 12 weeks. There may be no worsening of symptoms.

4. Progressive Disease (PD) or Relapse: Any one of the criteria below must be met to be considered progressive disease:

Development of any new area of malignant disease (measurable or palpable),

Increase (>25%) in any pretreatment area of measurable malignant disease.

The immunological response to immunization with the agonist peptides are assessed by in-vitro T cell proliferation assay and/or by in-vitro T cell cytotoxic assay before and after vaccination.

The present invention includes in vitro immunization for T cell proliferation and generation of cytotoxic T cell lines to the tumor specific agonist peptide. In vitro cultivation of peptide specific T cells from peripheral blood mononuclear cells (PBMC), lymph node tissue (LNT), or tumor infiltrating lymphocytes (TIL) with agonist peptide and IL-2 generates CEA and agonist peptide specific T cells. These T cells are tested for cytotoxicity against agonist peptide primed APC (autologous EBV transformed B cells or autologous tumor cells) has described herein. Generated T cell clones are characterized phenotypically by flow cytometry for express of CD3, CD4, and CD8. Agonist peptide specific cytotoxic lymphocytes may be adoptively transferred to a patient in order to inhibit or kill CEA or CEA epitopes expressing tumor cells. Patients may then be reimmunized with agonist peptide preferably in adjuvant.

Generally, between about 1.times.10⁵ and 2.times.10¹¹ cytotoxic T cells per infusion are administered in, for example, one to three infusions of about 200 to about 250 ml each over a period of 30 to 60 minutes. After completion of the infusions, the patient may be treated with a biological response modifier such as interleukin 2 (IL-2). In the case of IL-2, recombinant IL-2 is administered intravenously in a dose of 720,000 IU per kilogram of body weight every eight hours. After adoptive transfer of the antigen specific cytotoxic T cells into the patient, the patient may be additionally treated with the agonist peptide used to prime the cytotoxic T cells, to further expand the T cell number in vivo.

The invention encompasses a DNA sequence and variants thereof which encode an agonist peptide.

In one embodiment the DNA sequence encoding the agonist peptide is a variant of the DNA sequence comprising:

2 (SEQ. ID No: 6A) TAC CTT TCG GGA GCG AAC CTC AAC CTC (SEQ. ID No: 1A) Tyr Leu Ser Gly Ala Asn Leu Asn Leu.

One variant of SEQ. ID No: 6A includes but is not limited to a codon ATC (Ile) in place of the codon. CTC (Leu at position 7). Another variant of SEQ. ID No: 6A includes but is not limited to a codon, TGT (Cys) in place of the codon, AAC (Asn at position 6).

In another embodiment, the DNA sequence encoding the agonist peptide comprises:

3 (SEQ. ID No: 7A) TAC CTT TCG GGA GCG GAC CTC AAC CTC (SEQ. ID No: 2A) Tyr Leu Ser Gly Ala Asp Leu Asn Leu and variants thereof.

In yet another embodiment, the DNA sequence encoding the agonist peptide comprises:

4 (SEQ. ID No: 8A) TAC CTT TCG GGA GCG GAC ATC AAC CTC (SEQ. ID No: 3A) Tyr Leu Ser Gly Ala Asp Ile Asn Leu or variants thereof.

Included in the ambit of the invention are conservative substitutions based on codon degeneracy provided the modification results in a functionally equivalent agonist peptide or a peptide with enhanced immunogenicity.

The invention further provides vectors and plasmids comprising a DNA sequence encoding an agonist peptide. The vectors include but are not limited to E. coli plasmid, a Listeria vector and recombinant viral vector. Recombinant viral vectors including but not limited to orthopox virus, avipox virus, capripox virus, suipox virus, vaccinia, baculovirus, human adenovirus, SV40, bovine papilloma virus, and the like comprising the DNA sequence encoding an agonist peptide.

Recombinant agonist peptide can be obtained using a baculovirus expression system in accordance with the method of Bei et al J. Clin. Lab. Anal. 9:261-268 (1995). Recombinant viral vectors can be constructed by methods known in the art such as U.S. Pat. No. 5,093,258; WO96/10419 Cepko et al Cell 37:1053-1062 (1984); Morin et al Proc. Natl. Acad. Sci USA 84:4626-4630 (1987); Lowe et al Proc. Natl. Acad. Sci USA 84:3896-3900 (1987); Panicali & Paoletti, Proc. Natl. Acad. Sci USA 79:4927-4931 (1982); Mackett et al. Proc. Natl. Acad. Sci USA 79:7415-7419 (1982); WO 91/19803; Perkus et al Science 229:981-984 (1985); Kaufman et al Int. J. Cancer 48:900-907 (1991); Moss Science 252:1662 (1991); Smith and Moss BioTechniaues November/December, p. 306-312 (1984); U.S. Pat. No. 4,738,846; Sutter and Moss Proc. Natl. Acad. Sci USA 89:10847-10851 (1992); Sutter et al Virology (1994); and Baxby and Paoletti Vaccine 10:8-9 (1992).

Host cells which may express the DNA encoding the agonist peptide carried by vectors or plasmids are prokaryotic and eukaryotic host cells and include but are not limited to E. coli, Listeria, Bacillus species, COS cells, Vero cells, chick embryo, fibroblasts, tumor cells, antigen presenting cells and the like. When the host cell is an antigen presenting cell, the host cell is an antigen presenting cell, the host cell should additionally express an MHC class I molecule.

We recently reported (11) evidence of CTL responses to CEA in patients immunized with rV-CEA. The 9-mer peptide CAP1 was employed to expand CTL in vitro because of: (a) its strong binding to HLA-A2, and (b) its non-identity to other members of the CEA gene family expressed on normal tissues. CTLs were generated from post-immunization PBMC of patients while preimmulization blood of the same patients failed to proliferate. In addition, CAP1 pulsed dendritic cells stimulated in vitro growth of -A2 restricted CTL from peripheral blood of unimmunized cancer patients (12). Finally when CTL were generated in vitro by stimulation with dendritic cells encoding full-length CEA mRNA, cytotoxicity against CAP1 was higher than activity against six other -A2 binding CEA peptides (S. Nair and E. Gilboa, personal communication or unpublished observation). Such results encourage the notion that CAP1 is an immunodominant epitope of the CEA molecule.

The present invention is intended to improve the immunogenicity of the CAP1 peptide by introducing amino acid substitutions at non-anchor positions to form the agonist peptides of the present invention. When using T-Vac8 CTL as an effector, the analog CAP1-6D sensitized target cells for lysis far better than CAP1 itself. Further studies showed that cytolytic activity of a second -A2 restricted, CAP1 specific CTL, T-Vac24, was as good or greater with CAP1-6D than with CAP1. These demonstrations of enhanced reactivity could not be explained by improved presentation by class I MHC. Finally, CAP1-6D could be used to stimulate CTL in vitro from PBMC of both carcinoma patients and normal donors. Prior to the present invention, attempts to stimulate anti-CAP1 CTL from normal donors using this same methodology have been unsuccessful. The present invention relates to stimulation of normal donors with CAP1-6D as opposed to native CAP1 where stimulation with the native sequence failed to produce specific cytotoxic activity. In contrast, stimulation with CAP1-6D produced several CTL with specific anti-CAP1 peptide reactivity as well as anti-tumor reactivity. Thus, the analog peptide CAP1-6D is capable of selecting a population of CAP1 specific human CTL more efficiently than native CAP1. Such an agonist might find applications in the design of T cell directed vaccines against CEA-expressing carcinoma.

The present invention also relates to the more efficient generation and expansion of tumor specific T cells for adoptive immunotherapy. In recent years, much progress has been achieved in characterizing the tumor associated antigen peptides that can be presented to CTL by class I HLA antigens. In instances where mutations generate neo-antigens such as point mutated ras (35, 36), p53 (37, 38) or β-catenin (39) vaccination strategies target the novel sequence under the assumption that the immune system is not “tolerant” to an antigen it has never seen. More recently it has been proposed that neo-antigens may also arise through post-translational deamidations (29, 40). However, in many instances the intended targets of tumor therapy are not neoantigens but rather normal oncofetal or differentiation antigens that are overexpressed or ectopically expressed by malignant cells. Such is the case for CEA (41). In such situations, models invoking “tolerance” predict that the immune system has encountered these antigens and is less able to respond to them. This classical picture has been challenged in recent years by numerous reports of immunity elicited to overexpressed differentiation antigens, oncogenes, and tumor suppressor genes (37, 38, 42-44). Nonetheless, it is often experimentally difficult to generate and expand T cells with desired anti-tumor activity and it is therefore desirable to devise new strategies for generating CTL.

Some class II binding-peptides have been described in which substitutions enhance responses of murine and human Th clones without increasing the binding to class II antigens (29, 45-47). Among human class I peptides, however, the only substitutions described for the generation of CTL were those that increase binding to HLA (17-20). The substitutions in those studies were directed to residues at the primary or secondary anchor positions that define the binding motifs to class I MHC antigens. Even substitutions directed to a non-anchor position (19) achieved their enhancing effect by increasing binding to HLA-A2. The analog CAP1-6D in the present report represents what appears to be a different class of substituted CTL peptides, agonists that enhance recognition of the peptide-MHC ligand by the T cell receptor and produce greater effector function without increases in binding. To our knowledge this is the first such enhancer agonist peptide described for a human CTL.

The increased lytic susceptibility of targets in the presence of CAP1-6D is unlikely to be due to better antigen presentation. Binding experiments show that HLA-A2 presents the native CAP1, and the analogs CAP1-6D and CAP1-71 approximately equally. Another possibility is that CAP1-6D shows increased activity because it is presented by more than one allele and T-Vac8 is promiscuous towards peptide-MHC complexes. However, T-Vac8, T-Vac24, and CTL derived from nonimmunized patients showed better lysis with CAP1-6D. Since HLA-A2 is the only class I MHC on the targets employed, the improved lysis cannot be accounted for by recruitment of another class I MHC.

Since anti-CAP1 CTL from multiple donors demonstrate agonist cross reactivity it is possible that CAP1-6D could be used to stimulate growth of CTL from numerous -A2 individuals. We are encouraged by the quite distinct differences between T-Vac8 and T-Vac24 in magnitude of response to the agonist; this implies that each effector utilizes different TCR gene segments and that nonetheless they can recognize both the native sequence and the CAP1-6D substitution. The ability of CAP1-6D to act as an agonist with T cells expressing different T cell receptors clearly magnifies its therapeutic potential. Thus, the present invention also relates to stimulation with the agonist and subsequent generation of T cells that recognize the normal sequence in non-immunized individuals. Such individuals have presumably never encountered the modified sequence and since the agonist is more efficient at triggering a T cell response, such agonists might be capable of selecting CTL more readily than immunogens based on the native sequence.

For peptide-derived CTL to be useful therapeutic reagents it is essential to demonstrate that they can lyse tumor cells that express endogenous antigen (48, 49). Previously (11), we had shown that tumor cells process CEA and present antigens recognized by CTL generated by stimulation with CAP1. In accordance with the present invention, CTL grown from the normal donors by stimulation with CAP1-6D are also capable of recognizing allogeneic CEA-positive, HLA-A2 positive tumor cells. These T cells fail to recognize -A2 negative tumor cells or -A2 positive cells that lack CEA expression.

We have also shown that CTL selected with the CAP1-6D agonist can be maintained subsequently by stimulation with the native CAP1 sequence. This is an important finding since CTL in patients, whether established in vivo through active immunization, or transferred adoptively after ex vivo expansion, will likely only encounter the native sequence. This allows the CTLs to be maintained over an extended duration in vivo.

One of the original reasons for selecting and testing CAP1 was its non-identity with other reported sequences in the human genome. It was therefore predicted that any immune responses attained would be unlikely to damage normal tissues bearing other antigens. For this reason a similar search of protein databases was undertaken for the peptides CAP1-6D and CAP1-71 and revealed that they are not reported as human sequences elsewhere in the Genebank (Genetics Computer Group, Madison, Wis.). However, two similar sequences, YLNVQDLNL (SEQ. ID No: 9A) and YLHDPEFNL (SEQ. ID No: 10A), are reported for antigens from African swine fever virus and measles virus, respectively. These sequences fit the consensus motif for HLA-A2 and therefore allow infected individuals to express cross-reacting antigens to CAP1. One interesting possibility is that the presence of anti-CAP1 CTL in some patients represents an example of epitope mimicry (50).

Two recent reports suggest that modified asparagine residues might enhance the immunogenicity of class I MHC peptides. Skipper et al. (40) used CTL generated in mixed lymphocyte tumor cell cultures to identify antigens in extracts of melanoma cells. One antigenic peptide was identical at 8 of 9 positions to a sequence from tyrosinase, with an asparagine to aspartic acid replacement at position 3. When tested using synthetic peptides, the CTL were more active against the aspartic acid peptide than against the peptide containing the genetically predicted asparagine. These authors speculate that post-translational deamidations can generate antigenic peptides from normal differentiation antigens. Recently. Chen et al., (51) reported generating murine CTL to a stabilized succinimide derivative of an asparagine-containing antigenic peptide. Although these CTL could kill targets pulsed with the natural asparagine peptide, they did so with less sensitivity. They raise the possibility that deamidation of proteins in vivo and in vitro can produce transient succinimide intermediates that represent altered self-ligands capable of eliciting an immune response. At the other extreme, Kersh and Allen (52) replaced a TCR contact asparagine with aspartic acid in a hemoglobin peptide and abolished responsiveness to a murine Th clone. Presently we cannot exclude the possibility that the enhanced reactivity of CAP1-6D is due to deamidation of the native sequence which in turn primes the response that we detect with CAP1. However, our repeated inability to raise anti-CAP1 CTL from pre-immunized PBMC of the same patients from whom we generated post-immunization CTL, argues against this. Also, putative deamidations could not account for the recognition of other analogs such as CAP1-6C or CAP1-71 by T-Vac8 CTL. Instead it seems more reasonable that T cell receptors from both T-Vac8 and T-Vac24, as well as the new lines described here, can recognize some deviation from the native CAP1 sequence.

In summary, synthesis of analogs of an immunodominant CEA peptide with amino acid substitutions at positions predicted to potentially interact with the T cell receptor allowed us to identify an enhancer agonist. This agonist was recognized by two different CEA CTL and increases the activity of one of them by 2-3 orders of magnitude. The agonist was also able to stimulate growth of CTL from peripheral blood of non-immunized normal donors with far greater facility than the native peptide sequence. Most important, the CTL generated using the enhancer agonist was able to recognize and lyse targets presenting the native sequence, including tumor cell lines expressing endogenous CEA. In accordance with the present invention, characterization of this enhancer agonist peptide facilitates more aggressive anti-tumor immunotherapies when employed as an immunogen in vivo, or for the ex vivo expansion of autologous anti-tumor CTL. The synthetic approach employed according to the present invention is also useful in improving immunogenicity of other peptide CTL epitopes.

Methods and Materials

Peptides

A panel of single amino acid substitutions to positions p5 through p8 of the CEA peptide CAP1 were made by f-moc chemistry using pin technology (Chiron Mimotopes, Victoria, Australia). CAP1 (YLSGANLNL) and CAP1.about.6D (YLSGADLNL), greater than 96% pure, were also made by Multiple Peptide Systems (San Diego, Calif.). Additional peptides CAP1-71 and NCA571 were synthesized on an Applied Biosystems 432A synthesizer and were greater than 90% pure, by C18 reverse-phase HPLC.

Cell Lines

T-Vac8 (53) and T-Vac24 (11) are human CTL specific for the CEA peptide CAP1. These cell lines were generated by in vitro stimulation of PBMC using CAP1 and IL-2, according to previously published methods (11). Briefly, post-immunization PBMC were from HLA-A2+ individuals with advanced carcinoma that had been administered rV-CEA in a Phase 1 trial. PBMC were isolated on gradients of lymphocyte separation medium (Organon Teknika Durham, N.C.) and 2.times.10⁵ cells were placed in wells of sterile 96 well culture plates (Coming Costar, Cambridge, Mass.) along with 50 .mu.g/ml peptide. After 5 days incubation at 37 C. in a humidified atmosphere containing 5% CO₂, supernatants were removed and replaced with medium containing 10 U/ml human IL-2 (a gift of the Surgery Branch, NCl). Cultures were fed with IL-2 every 3 days for 11 days and then restimulated with irradiated (4000 rad) autologous PBMC (5.times.10⁵) and peptide. Fresh IL-2 was provided every third day and subsequent restimulations were done every 2 weeks. CTL are maintained in complete RPMI (GIBCO/BRL, Grand Island, N.Y.) medium with glutamine (GIBCO/BRL), penicillin, streptomycin and 10% pooled human AB serum (Gemini Bioproducts, Inc., Calabasas, Calif.).

Cell line C1R-A2 (provided by Dr. W. Biddison, National Institute of Neurological Disorders and Stroke, National Institutes of Health, Bethesda, Md.) is maintained in complete RPMI with 10% fetal bovine serum (FBS, Biofluids Inc., Rockville, Md.), glutamine, non essential amino acids and pyruvate (Biofluids) and 1 mg/ml G418. Cell line 174.CEM-T2 (provided by Dr. P. Creswell, Yale University School of Medicine, New Haven. Conn.) is defective in endogenous peptide processing and is maintained in Iscove's-(GIBCO/BRL) with 10% FBS. Both C1R-A2 and T2 lines present exogenous peptides with HLA-A2.

CEA positive tumor cell lines SW480, SW1463. SW1116 and SW 837 were obtained from the American Type Culture Collection (ATCC, Rockville. Md.) and passaged weekly in respective culture medium described in the ATCC catalog. The CEA negative melanoma line SKmel24 (provided by Dr. S. Rosenberg, National Cancer Institute, National Institutes of Health, Bethesda, Md.) was passaged weekly in RPMI 1640, 10% FBS and 10 .mu.g/ml gentamicin (Life Technologies). The CEA negative ovarian tumor CaOV3 was provided by Dr. R. Freedman (MD Anderson Cancer Center, Houston Tex.) and was cultured in RPMI with 15% FBS, glutamine, 12 .mu.g/ml insulin (Sigma, St. Louis. Mo.), 10 .mu.g/ml hydrocortisone (Biofluids) and 10 .mu.g/ml gentamicin. All tumor lines were trypsinized with Trypsin/versene (Biofluids) for 5-10 minutes prior to labeling with isotope for CTL assays. The highly sensitive natural killer (NK) target K562 was obtained from ATCC and passaged weekly with RPMI 1640, 10% FBS.

Generation of CTL

T cell lines T-N1 and T-N2 were generated from PBMC of two normal HLA-A2 positive donors by in vitro stimulation with peptide as follows. For the first stimulation cycle, T cells were positively selected by panning on CD3+ MicroCellector flasks (Applied Immune Sciences. Santa Clara, Calif.). CD3+ cells (3.times.10⁶) were cultured with 10⁶ 174.CEM-T2 cells that were previously infected with vaccinia virus expressing human B7 at a multiplicity of infection of 10, pulsed with 50 .mu.g/ml CAP1 or CAP1-6D peptide and 2 .mu.g/ml human β2 microglobulin (Intergen, Purchase, N.Y.), and irradiated (10,000 rad). Cultures were incubated at 37 C. in a humidified atmosphere containing 5% Co₂, in T25 flasks in RPMI with 10% human serum, 2 mM glutamine, and 10 .mu.g/ml gentamicin in a total volume of 10 ml with 2.times.10⁷ irradiated (2500 rads) autologous PBMC as feeder cells. After 24 hours in culture 10 U/ml huIL-2 and 0.1 ng/ml rIL-12 (R & D Systems, Minneapolis, Minn.) were added. After 9 days in culture, cells were restimulated using irradiated (10,000 rads) autologous EBV-B cells preincubated with 25 .mu.g/ml peptide at a ratio of 2.5:1 stimulator cells to T cells, and IL-2 and IL-12 were again added 24 hours later. Peptide concentration was halved with each subsequent stimulation cycle until a final concentration of 3.12 .mu.g/ml was achieved.

In addition, CTL were generated from post-immunization PBMC of cancer patient Vac8 by stimulation with CAP1-6D according to already published procedures (11).

CTL Assay

Target cells were labeled with ⁵¹Cr or ¹¹¹In, then incubated at 2,000-10,000 per well with or without peptides in round bottom microtiter plates (Corning Costar). One hour later, T cells were added. Supernatants were harvested (Skatron, Inc., Sterling Va.) after 4 hour and isotope release was measured. All assays were performed in triplicate and percent specific release was calculated according to: 1 (observed release-spontaneous release)/(maximum release-spontaneous release)×100

where spontaneous release is obtained by omitting the T cells, and maximum release is obtained by adding 1% Triton .times.100.

Binding Assay

Binding of peptides to HLA-A2 was evaluated by incubation with processing defective 174.CEM-T2 cells and measuring the stability of cell surface peptide-A2 complexes (30). Briefly, cells were harvested and washed with serum-free RPMI then incubated overnight at 1-2.times.10⁶ cells/well with various concentrations of peptides. The next day, cells were collected, washed in PBS with Ca²+, Mg²+ and 5% FBS, then divided into aliquots for single color flow cytometric analysis. Cells were incubated 1 hour on ice without antibody, with anti-A2 antibody A2,69 (One Lambda, Inc., Canoga Park. Calif.) or with isotype-matched control antibody UPC-10 (Organon Teknika) then washed and stained 1 hour with fluorescein-isothiocyanate (FITC) goat anti-mouse Ig (Southern Biotechnology Associates, Birmingham, Ala.). Cell surface staining was measured in a Becton Dickinson flow cytometer (Mountain View, Calif.) and the mean fluorescence intensity (MFI) for 10,000 live cells was plotted against peptide concentration.

TCR Chain Usage

T-N1 CTL were cultured as described for 5 cycles of antigenic stimulation using the CAP1-6D analog. The line was then split and duplicate cultures were maintained either with CAP1 or CAP1-6D for 5 additional stimulation cycles. Ficoll-purified T cells (5.times.10⁵) were stained with a panel of 19 anti-Vβ and 2 anti-V.alpha. murine monoclonal antibodies to human .alpha.β T cell receptor variable regions. Cells were incubated with 10 .mu.g/ml of purified antibodies for 30 minutes at 4 C. The unlabeled monoclonals used were: Vβ3.1 clone 8F10, Vβ5(a) clone 1C1, Vβ5(b) clone W112, Vβ5(c) clone LC4, Vβ6.7 clone OT145, Vβ8(a) clone 16G8, Vβ12 clone S511, Vβ13 clone BAM13, V.alpha.2 clone F1 and V.alpha.12.1 clone 6D6 (T Cell Diagnostics, Woburn, Mass.) and Vβ18 (Immunotech, Westbrook, Me.). Cells were stained with 10 .mu.g/ml of FITC-labeled goat anti-mouse IgG antibody (Southern Biotechnology Associates) for 30 minutes in the dark. Directly labeled monoclonals were: FITC-labeled Vβ11, Vβ21.3, Vβ13.6, Vβ14, Vβ16, Vβ17, Vβ20 and Vβ22 and PE-labeled Vβ9 and Vβ23 (Immunotech). Cells were fixed with 1% paraformaldehyde, washed with FACSFlow buffer (Becton Dickinson) and analyzed using a Becton Dickinson flow cytometer.

The present invention further is a recombinant vector comprising foreign genes encoding multiple costimulatory molecules, in combination, or the functionally active portions of each costimulatory molecule. Multiple costimulatory molecules as used herein are at least three or more costimulatory molecules. As used herein a functionally active portion is that portion of the molecule responsible for binding to its respective ligand, triggering an appropriate costimulatory signal for immune-cell activation. One method of determining functional activity is to access the induction of nave T-cell proliferation by delivering the costimulatory molecule to a target cell in vitro as described herein. A functional portion of a costimulatory molecule stimulates at least 20% increase in T cell proliferation.

The term foreign gene or foreign nucleic acid sequence or functional portion thereof as used herein is a gene, nucleic acid sequence or functional portion thereof that is exogenously provided by a recombinant vector to a host cell or organism. The exogenous gene or portion thereof which is provided to the host cell or host organism may be one which is not endogenously present in the host cell or organism or may be endogenously present and functional or non-functional. In the case in which a functional endogenous gene is present in the host cell or organism, the foreign or exogenously provided gene or functional portion thereof results in overexpression of the gene product.

The recombinant vectors of the present invention have utility in providing enhanced immunological response to cells of the immune system including but not limited to T lymphocytes, B lymphocytes, NK cells, antigen-presenting cells (APCs) and the like. The enhancement of the immunological response using the recombinant vectors expressing multiple costimulatory molecules is synergistic as compared to the use of a single costimulatory molecule or the use of two costimulatory molecules in enhancing immunological responses. The immunological response may be a cellular and/or humoral immune response and may be directed to a specific target antigen or epitope thereof or may be a generalized immune enhancing or upregulating effect as demonstrated by increased cytokine release, increase proliferation by immune cells, increased mitogen responsiveness and the like. The enhancement in an immune response preferably includes hyperstimulation or high intensity T cell stimulation (HITS) as a result of stimulation using the recombinant vectors of the present invention or cells transfected, transduced or induced by the recombinant vector of the present invention.

The foreign genes encoding the costimulatory molecules may be obtained from a variety of sources. The selection of the source of foreign genes encoding the costimulatory molecules may depend on the species to be immunized or treated using the recombinant vector.

The foreign genes encoding the costimulatory molecules may be murine-derived, human-derived, simian-derived, other mammalian homologs and may be chemically synthesized based on mammalian genes. The foreign genes encoding the costimulatory molecules may also be avian-derived or chemically synthesized based on avian costimulatory molecule genes. The recombinant vectors of the present invention are useful as immunogens and as vaccines in stimulating an enhancement of immunological responses to target cells, target antigens and immunological epitopes thereof. Such level of enhancement of a immune response using the present recombinant vectors comprising genes encoding multiple costimulatory molecules has not been obtainable using a single or double costimulatory molecule.

Genes or functional portions thereof encoding costimulatory molecules having utility in the present invention include but are not limited to B7.1, B7.2, ICAM-1, LFA-3, 4-1BBL, CD59, CD40, CD70, VCAM-1, OX-40L, mammalian homologs and the like. The recombinant vector of the present invention comprises genes encoding at least three costimulatory molecules for synergistic enhancement of immune responses which is not obtainable by the use of a single or a double costimulatory molecule. Genes encoding various combinations of costimulatory molecules are an ambit of the invention for use in the recombinant vector and may include such combinations as B7.1, B7.2, ICAM-1, LFA-3; B7.1, B7.2, ICAM-1, LFA-3; B7.1, B7.2, ICAM-1, 4-1BBL; B7.1, B7.2, ICAM-1, LFA-3, 4-1BBL; CD59, VCAM-1; and B7.1, B7.2; CD59, CD40, 4-1 BBL, CD70 and VCAM-1. B7.1, B7.2; OX-40L, 4-1BBL; and the like depending on the desired immune response and the disease or condition to be treated. Based on the dramatic synergistic immune responses achieved using a recombinant vector encoding three costimulatory molecules as compared to the use of a recombinant vector encoding one or two costimulatory molecules, a recombinant vector encoding four, five or more costimulatory molecules will result in a synergistic immune response or immune response equal to/or greater than that using a recombinant vector encoding three costimulatory molecules.

B7 represents a family of costimulatory molecules which are members of the Ig gene superfamily. The members include murine B7.1 (CD80) and B7.2 (CD86). B7.1 and B7.2 are the natural ligands of CD28/CTLA-4 (CD152). The gene sequence of murine B7.1 is disclosed in Freeman et al (J. Immunol. 143:2714-2722, 1989) and in GENBANK under Accession No. X60958. The gene sequence of murine B7.2 is disclosed in Azuma et al (Nature 366:76-79, 1993) and in GENBANK under Accession No. L25606 and MUSB72X.

The human homologs of the murine B7 costimulatory molecules and functional portions thereof are an ambit of the present invention and have particular utility in recombinant vectors for human clinical use. The human homolog of the murine B7 costimulatory molecules include CD80, the homolog of murine B7.1, and CD86, the homolog of B7.2. The gene sequence of human B7.1 (CD80) is disclosed in GENBANK under Accession No. M27533, and the gene sequence of human B7.2 (CD86) is disclosed under Accession No. U04343 and AF099105. A license may be required to practice this invention.

For use in the present invention, a recombinant vector may contain a foreign nucleic acid sequence encoding at least one molecule from the B7 costimulatory molecule family, or a combination of B7 costimulatory molecules or functional portions thereof in addition to other costimulatory molecules. The combination of B7 costimulatory molecules includes but is not limited to two or more B7.1 molecules, two or more B7.2 molecules, B7.1 and B7.2 and the like. In one embodiment the recombinant vector contains a foreign nucleic acid sequence encoding the B7.1 molecule in combination with foreign nucleic acid sequences encoding LFA-3 and ICAM-1.

Intercellular adhesion molecule-1 (murine ICAM-1, CD54) and the human homolog, CD54, also acts as a costimulatory molecule. Its ligand is leukocyte function-associated antigen-1 (LFA-1, CD11a/CD 18) which is expressed on the surface of lymphocytes and granulocytes. The gene for murine ICAM-1 is disclosed in GenBank under Accession No. X52264 and the gene for the human ICAM-1 homolog, (CD54), is disclosed in Accession No. J03132. In one embodiment, the recombinant vector of the present invention contains a foreign nucleic acid sequence encoding at least one murine ICAM-1 molecule, human homolog, other mammalian homolog or functional portion thereof in addition to foreign nucleic acid sequences encoding two or more additional costimulatory molecules.

The costimulatory molecule leukocyte function antigen 3, murine LFA-3 (CD48), and its human homolog LFA-3 (CD58), a glycosyl-phosphatidylinositol-linked glycoprotein, is a member of the CD2 family within the immunoglobulin gene superfamily. The natural ligand of LFA-3 is CD2 (LFA-2) which is expressed on thymocytes. T cells, B cells and NK cells. The gene for murine LFA-3 is disclosed in GenBank under Accession No. X53526 and the gene for the human homolog is disclosed in Accession No. Y00636.

The T cell antigen 4-1BBL is a costimulatory molecule that relays costimulatory signals in antigen-stimulated primary T cell cultures and in lectin-driven activation of thymocytes (Hurtado, J. C. et al J. Immunol. 158(6):2600-2609, 1997). 4-1BBL belongs to the tumor necrosis factor receptor superfamily, a group of cysteine-rich cell surface molecules (Vinay, D. S. et al., Seminars in Immunology, 1998, Vol. 10, pp. 481-489). The gene for the murine 4-1BBL is disclosed in GenBank under Accession No. U02567. The gene for the human homolog, hu4-1BBL is disclosed in GenBank under Accession No. U03397.

OX-40L is a type 11 membrane protein with limited homology to TNF and is stimulatory to OX-40⁺ T cells in vitro. The murine and human OX-40L cDNAs have 68% homology at the nucleotide level and 46% at the amino acid level. Human OX-40L stimulates human T cells exclusively, while murine OX-40L stimulates both human and mouse T cells. APC express OX-40L and can transmit the OX-40L: OX40R signal during presentation of antigen to CD4⁺ T cells. OX-40L signaling is important for differentiation of human dendritic cells and leads to increased production of IL-12, TNF-.alpha., IL-1B, and IL-6. (Weinberg, A. D. et al 1998 Seminars in Immunology, Vol. 10:471-480). OX-40L is a potent costimulatory molecule for sustaining primary CD4⁺ T cell responses, used in combination with B7-1 (Gramaglia, I, et al 1998 J. Immunology, Vol. 161:6510-7.

Vectors having utility in the present invention are capable of causing expression of at least three or more foreign genes, preferably five or more foreign genes. Vectors having utility in the present invention include any vector capable of causing functional expression of at least three foreign costimulatory molecules gene products in a host cell. In addition to the genes encoding at least three costimulatory molecules, the vector is also capable of causing the expression of at least one foreign gene encoding at least one target antigen or immunological epitope thereof as well as a selectable marker.

Vectors of the present invention include but are not limited to bacterial vectors such as Salmonella, viral vectors, nucleic acid based vectors and the like. Viral vectors include but are not limited to poxvirus, Herpes virus, adenovirus, alphavirus, retrovirus, picornavirus, iridovirus, and the like. Poxviruses having utility in the present invention include replicating and non-replicating vectors. Such poxviruses include but are not limited to orthopox such as vaccinia, raccoon pox, rabbit pox and the like, avipox, suipox, capripox and the like. Poxviruses may be selected from the group consisting of vaccinia-Copenhagen, vaccinia-Wyeth strain, vaccinia-MVA strain, NYVAC, fowlpox, TROVAC, canarypox, ALVAC, swinepox, and the like. In one embodiment, the recombinant vector is a vaccinia virus. In another embodiment, the recombinant vector is fowlpox.

A preferred vector of the present invention is a recombinant virus, preferably a poxvirus. The recombinant poxviruses having utility in the present invention have a number of attributes, including (i) efficient delivery of genes to multiple cell types, including APC and tumor cells; (ii) high levels of protein expression; (iii) optimal presentation of antigens to the immune system; (iv) the ability to elicit cell-mediated immune responses as well as antibody responses; (v) transient, rather than permanent, genetic modification of cells, and (vi) the ability to use combinations of poxviruses from different genera, as they are not immunologically cross-reactive. Parental poxviruses useful in constructing the recombinant poxvirus of the present invention include but are not limited to orthopox virus such as replicating vaccinia virus (Perkus et al Science 229:981-984, 1985; Kaufman et a Int. J. Cancer 48:900-907, 1991, Moss Science 252:1662, 1991), highly attenuated vaccinia viruses such as MVA, modified vaccinia Ankara (Sutter and Moss, Proc. Nat'l Acad. Sci. U.S.A. 89:10847-10851; Sutter et al Virology 1994), vaccinia-Copenhagen and NYVAC: avipoxviruses (15) such as fowlpox virus (15), canary poxviruses, such as ALVAC and the like (Baxby and Paoletti, Vaccine 10:8-9, 1992; Rinns, M. M. et al (Eds) Recombinant Poxviruses CRC Press, Inc, Boca Raton 1992; Paoletti, E. Proc. Nat'l Acad. Sci. USA 93:11349-11353, 1996), and suipoxvirus, capripoxvirus and the like.

In one embodiment, the parental poxvirus is a vaccinia virus. In a particular embodiment, the vaccinia virus is a Wyeth strain or derivative thereof. A derivative of the Wyeth strain includes but is not limited to vTBC33 which lacks a functional K1L gene and the like. In yet another embodiment, the virus is Dry-Vax available as a smallpox vaccine from the Centers for Disease Control, Atlanta, Ga. In another embodiment, the parental poxvirus is a strain of fowlpox, for example POXVAC-TC (Schering-Plough Corporation), and the like.

The recombinant vector of the present invention is able to infect, transfect or transduce host cells in a host. The host includes but is not limited to mammals, birds, fish and the like. The host cells are any cell amenable to infection, transfection or transduction by the recombinant vector and capable of expressing the foreign genes from the recombinant vector at functional levels. The host cells include but are not limited to professional APC and antigen presenting precursor cells such as monocytes, macrophages, DC, Langerhans cells and the like. The recombinant vector of the present invention may also infect tumor cells or other cell types such as fibroblasts or muscle cells. Infection of the host cells allows expression of each foreign, exogenous costimulatory molecule and expression of the foreign nucleic acid sequence encoding target antigen(s) if present in the recombinant vector. The host cells express, or are engineered to express, the appropriate MHC (HLA) Class I or II molecules for appropriate antigenic presentation to CD4⁺ and/or CD8⁺ T cells. As such virtually any mammalian cell may be engineered to become an appropriate antigen presenting cell expressing multiple costimulatory molecules.

The recombinant vector of the present invention comprises at least one expression control element operably linked to the nucleic acid sequence. The expression control elements are inserted in the vector to control and regulate the expression of the nucleic acid sequence (Ausubel et al, 1987, in “Current Protocols in Molecular Biology, John Wiley and Sons, New York, N.Y.). Expression control elements are known in the art and include promoters. Promoters useful in the present invention are poxviral promoters as are known in the art which include but are not limited to 30K, I3, sE/L, 7.5K, 40K, C1 and the like. The nucleic acid sequence of the 30K promoter is disclosed in GenBank Accession No. M35027 at base numbers 28,012 through 28,423 (antisense). The nucleic acid sequence of 13 is disclosed in GenBank Accession No. J03399 at base numbers 1100 through 1301 (antisense). The nucleic acid sequence of the 7.5K promoter is disclosed in GenBank Accession No. M35027 at base numbers 186550 through 186680. The nucleic acid sequence of the 40K promoter is disclosed in GenBank Accession No. M13209 at base numbers 9700 through 9858 (antisense). The nucleic acid sequence of the C1 promoter is disclosed in GenBank Accession No. M59027 at base numbers 1 through 242 and in U.S. Pat. No. 5,093,258. The sequence of the sE/L promoter is disclosed in Reference 16. Other poxvirus promoters may be used, such as, those described by Davison and Moss (J. Mol. Biol. 210:749-769. (1989). Any of these promoters can be synthesized by using standard methods in the art. The selection of an appropriate promoter is based on its timing and level of expression. Early or early/late promoters are preferred. In a preferred embodiment, the promoter or combination of promoters utilized allow for optimal expression of each costimulatory molecule in an infected host to provide a synergistic immune response. In a preferred embodiment, each foreign gene encoding a costimulatory molecule is controlled by a separate and distinct promoter.

In the case of nucleic acid-based vectors, the constructs may be either nucleic acid (DNA or RNA) or associated with/or encapsulated in a lipid carrier. Optionally, the lipid carrier molecule and/or construct may provide targeting and/or expression in a particular target cell type or types. Naked DNA vectors may be prepared by methods described in U.S. Pat. No. 5,827,703. For the transcriptional initiation region, or promoter element, any region may be used with the proviso that it provides the desired level of transcription of the DNA sequence of interest. The transcriptional initiation region may be native to or homologous to the host cell and/or to the DNA to be transcribed, or foreign or heterologous to the host cell and/or the DNA sequence to be transcribed. Efficient promoter elements for transcription initiation of naked DNA include but are not limited to the SV40 (simian virus 40) early promoter, the RSV (Rous sarcoma virus) promoter, the adenovirus major late promoter, the human CMV (cytomegalovirus) immediate early I promoter, and the like. Nucleic acid-based vectors may be delivered to a host using a syringe, a catheter, or a needle-free injection device such as a gene gun.

In an embodiment of the invention, a recombinant vector is provided comprising a foreign nucleic acid sequence encoding a first costimulatory molecule or functional portion thereof under control of a first promoter, a foreign nucleic acid sequence encoding a second costimulatory molecule or functional portion thereof under control of a second promoter, and a foreign nucleic acid sequence encoding a third costimulatory molecule or functional portion thereof under control of a third promoter. The recombinant vector may further provide a foreign nucleic acid sequence encoding a target antigen or immunological portion thereof under control of a fourth promoter.

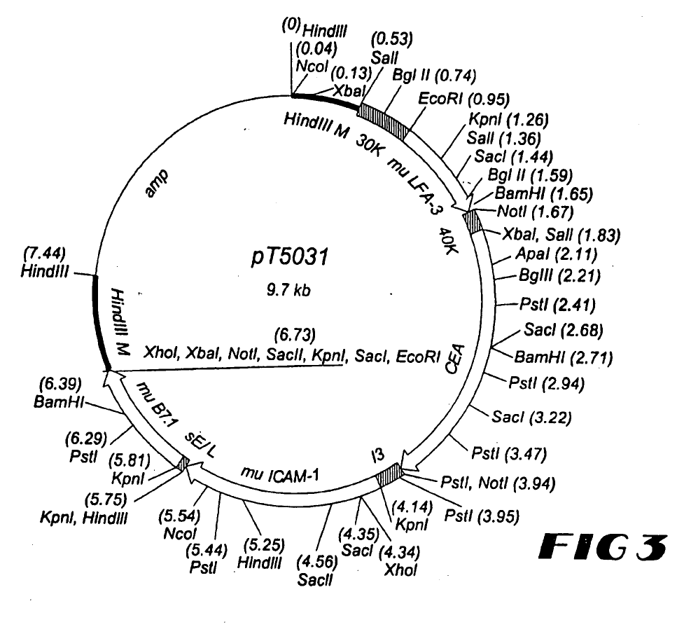

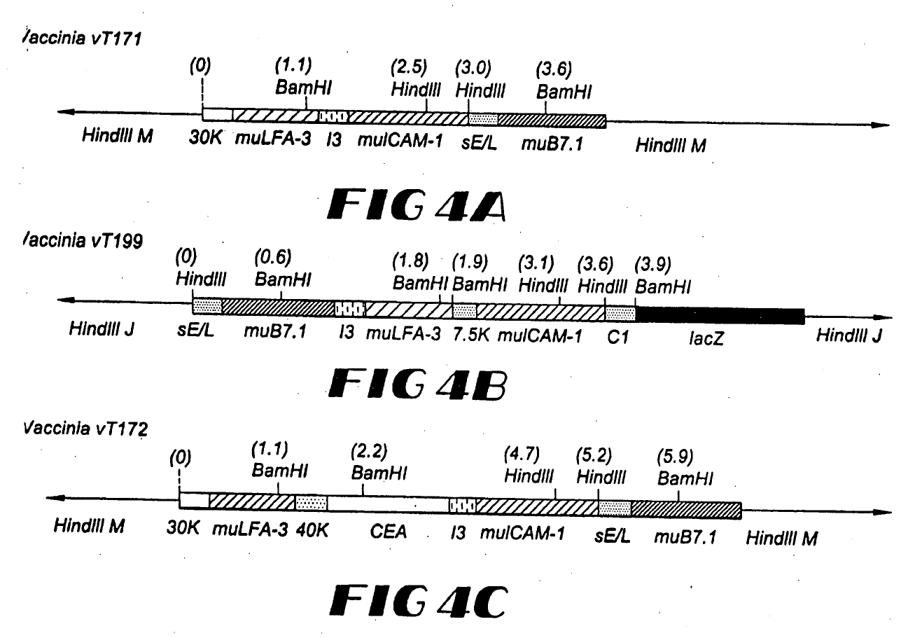

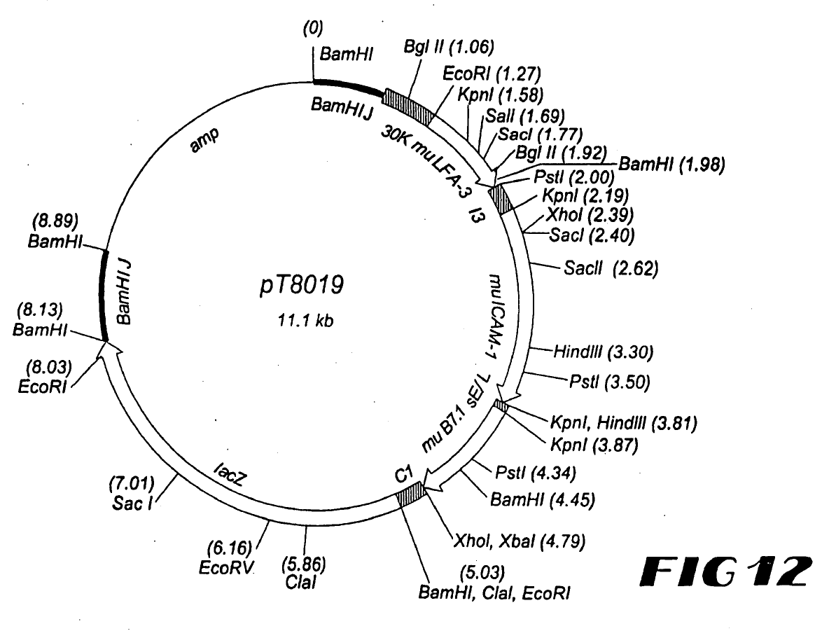

In one embodiment of the present invention, a recombinant poxvirus is provided comprising a nucleic acid sequence encoding LFA-3 or functional portion thereof under control of a 30K poxviral promoter, a nucleic acid sequence encoding ICAM-1 or portion thereof under control of an I3 poxviral promoter, and a nucleic acid sequence encoding B7.1 or portion thereof under control of an sE/L poxviral promoter. One example of such a recombinant poxvirus construct is vaccinia vT171 as depicted in FIG. 11A. The recombinant poxvirus may further provide a nucleic acid sequence encoding a tumor associated antigen or immunological portion thereof. One embodiment of the invention is recombinant vaccinia vT172 as depicted in FIG. 4C.

In another embodiment of the present invention, a recombinant poxvirus is provided comprising a nucleic acid sequence encoding B7.1 under control of a sE/L poxviral promoter, a nucleic acid sequence encoding LFA-3 or portion thereof under control of the I3 poxviral promoter, and a nucleic acid sequence encoding ICAM-1 or portion thereof under control of the 7.5K poxvirus promoter. Optionally the construct further comprises a nucleic acid sequence encoding at least one target antigen or immunological epitope thereof and/or a nucleic acid sequence encoding a selectable marker. One embodiment of such a recombinant poxvirus construct is vaccinia vT199 as depicted in FIG. 4B containing a lacZ gene as the selectable marker.

In an embodiment of the invention a recombinant fowlpox virus comprises a nucleic acid sequence encoding B7.1 or portion thereof under control of the sE/L poxviral promoter, a nucleic acid sequence encoding LFA-3 or portion thereof under control of the I3 poxviral promoter, and a nucleic acid sequence encoding ICAM-1 or portion thereof under control of the 7.5K poxviral promoter. An example of this embodiment is fowlpox vT222 as depicted in FIG. 4A. A recombinant fowlpox virus may further comprise a nucleic acid sequence encoding a target antigen, CEA, under control of the 40K poxviral promoter and a nucleic acid sequence encoding the selectable marker, lacZ under control of the C1 poxviral promoter. An example of this embodiment is fowlpox vT194 as depicted in FIG. 4B.

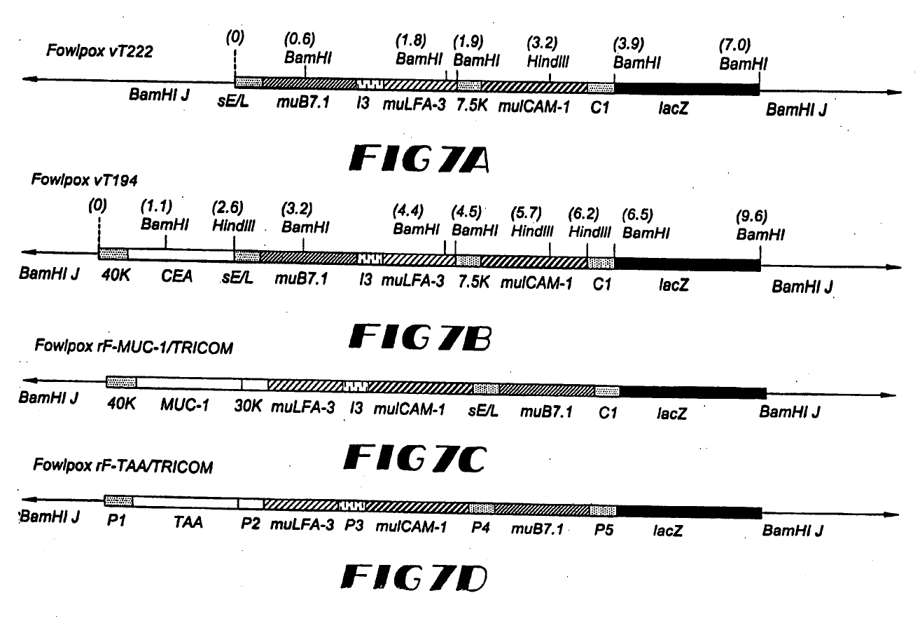

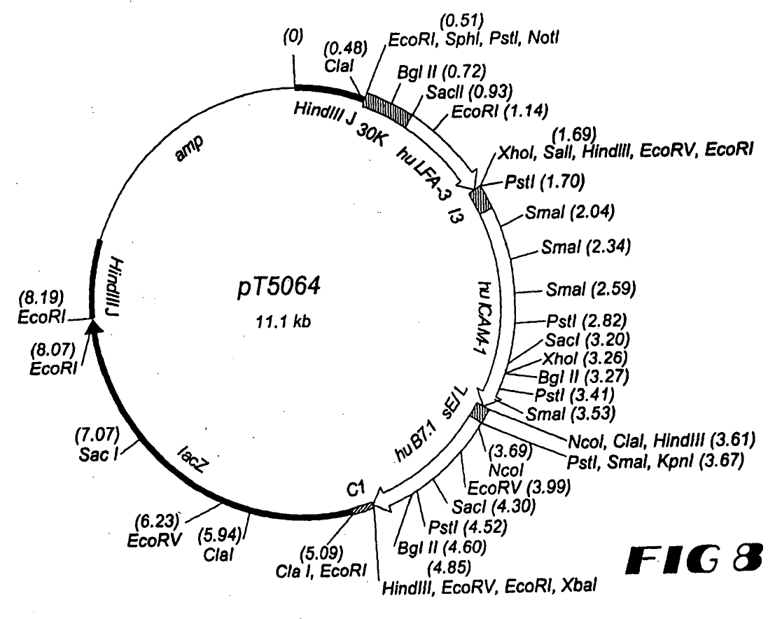

In another embodiment, a recombinant fowlpox virus comprises a nucleic acid sequence encoding the tumor-associated antigen MUC-1 or portion thereof under the control of the 40K promoter, a nucleic acid sequence encoding LFA-3 or portion thereof under the control of the 30K promoter, a nucleic acid sequence encoding ICAM-1 or portion thereof under the control of the I3 promoter, and a nucleic acid sequence encoding B7.1 or portion thereof under the control of the sE/L promoter, as depicted in FIG. 14C. The recombinant fowlpox virus may comprise a nucleic acid sequence encoding any tumor-associated antigen or portion thereof and nucleic acid sequences encoding LFA-3, ICAM-1 and B7.1, under the control of a multiplicity of promoters, as depicted in FIG. 7D.

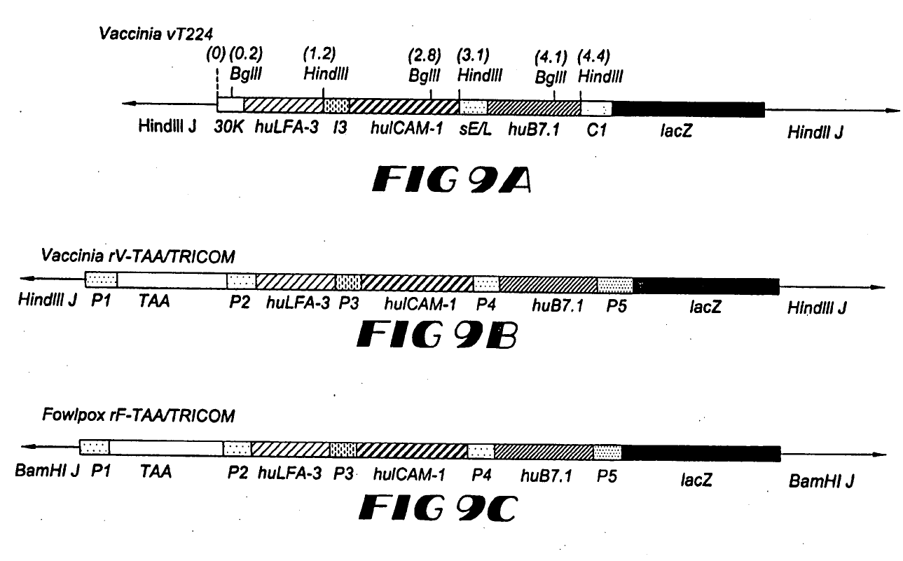

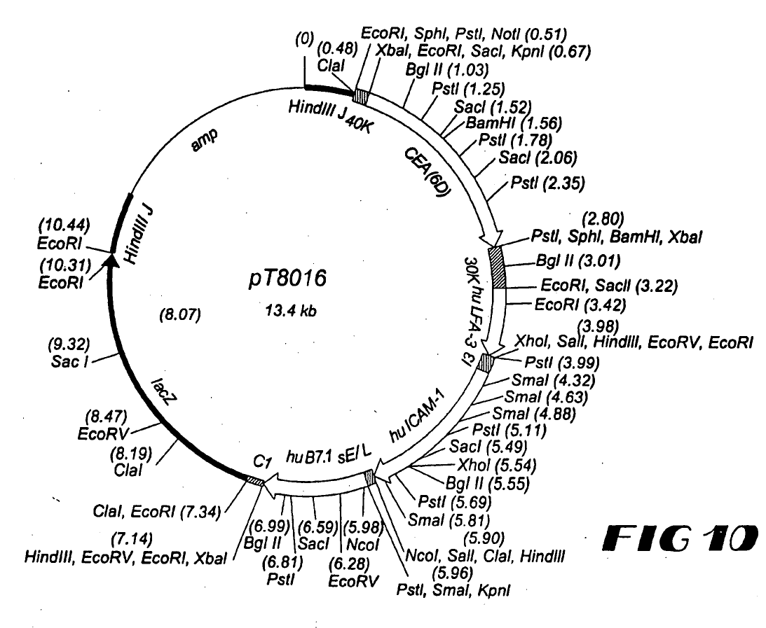

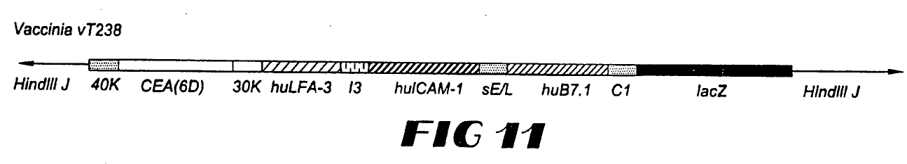

Another embodiment of the present invention is a recombinant vector comprising nucleic acid sequences encoding the human homologs of the costimulatory molecules LFA-3, B7 and ICAM-1. The recombinant vector may further provide the appropriate promoters to allow; expression of each sequence in an infected host cell. One embodiment of the recombinant vector is vT224 depicted in FIG. 9.

The present invention provides plasmid vectors comprising a foreign nucleic acid sequence encoding multiple costimulatory molecules. In one embodiment, foreign nucleic acid sequences are selected that encode at least three or more costimulatory molecules selected from the group consisting of B7, ICAM-1, LFA-3,4-1BBL, CD59, CD40, CD70, VCAM-1, OX-40L and the like. In one embodiment of the present invention, plasmid vectors comprising a foreign nucleic acid sequence encoding at least one B7 costimulatory molecule, a foreign nucleic acid sequence encoding an ICAM-1 costimulatory molecule and a foreign nucleic acid sequence encoding a LFA-3 costimulatory molecule are provided. The plasmid vectors of the present invention further provide at least one promoter sequence for controlling the expression of the costimulatory molecules. In a preferred embodiment each nucleic acid sequence encoding a costimulatory molecule is controlled by a separate discrete promoter sequence. For use in making a recombinant poxvirus, the plasmid vectors of the present invention further provide flanking viral nucleic acid sequences from a non-essential region of a poxvirus genome. The flanking viral nucleic acid sequences direct insertion of the foreign sequences into a parental poxviral genome via homologous recombination. The plasmid vectors of the present invention may further comprise one or more selectable markers for selection and identification of recombinant progeny containing the inserted foreign DNA as are known in the art including but not limited to the vaccinia K1L host range gene, the E. coli lacZ gene, antibiotic resistance genes, the gene encoding β-glucuronidase and the like.

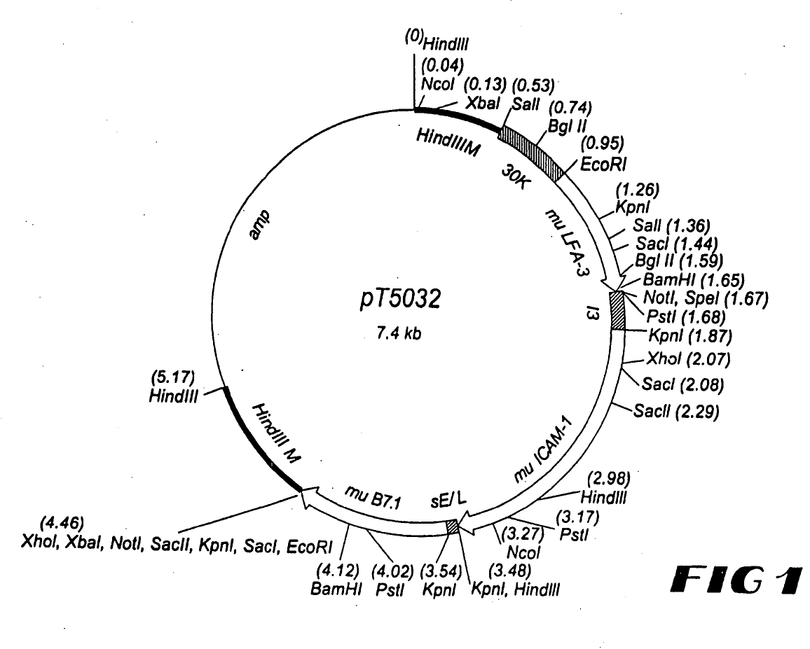

In an embodiment, a plasmid vector of the present invention comprises a nucleic acid sequence encoding LFA-3 under control of the 30K promoter, a nucleic acid sequence encoding ICAM-1 under control of the I3 promoter and a nucleic acid sequence encoding B7 under control of the sE/L promoter, flanked by portions of the Hind III M region of the vaccinia genome. In one embodiment, the plasmid vector is as depicted in FIG. 1 as pT5032.

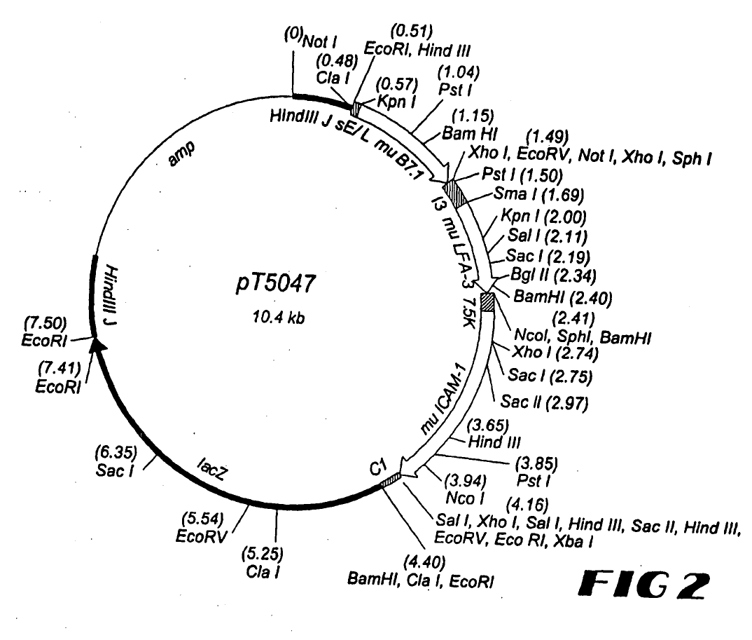

Another embodiment of the plasmid vector of the present invention comprises a nucleic acid sequence encoding B7 under control of the sE/L promoter, a nucleic acid sequence encoding LFA-3 under control of the I3 promoter and a nucleic acid sequence encoding ICAM-1 under control of the 7.5K promoter. The plasmid vector may further comprise a lacZ gene or portion thereof driven by a distinct promoter sequence. These sequences are flanked by portions of the Hind III J region of the vaccinia genome. A particular embodiment of the plasmid vector is depicted as pT5047 in FIG. 2.

In another embodiment of the plasmid vector comprises in combination with the nucleic acid sequences encoding B7, ICAM-1, and LFA-3, a nucleic acid sequence encoding at least one target antigen or immunological epitope thereof. A promoter is provided for controlling the expression of the target antigen. A particular embodiment of the plasmid vector is depicted as pT5031 in FIG. 3 and comprises a nucleic acid sequence encoding the target antigen. CEA.

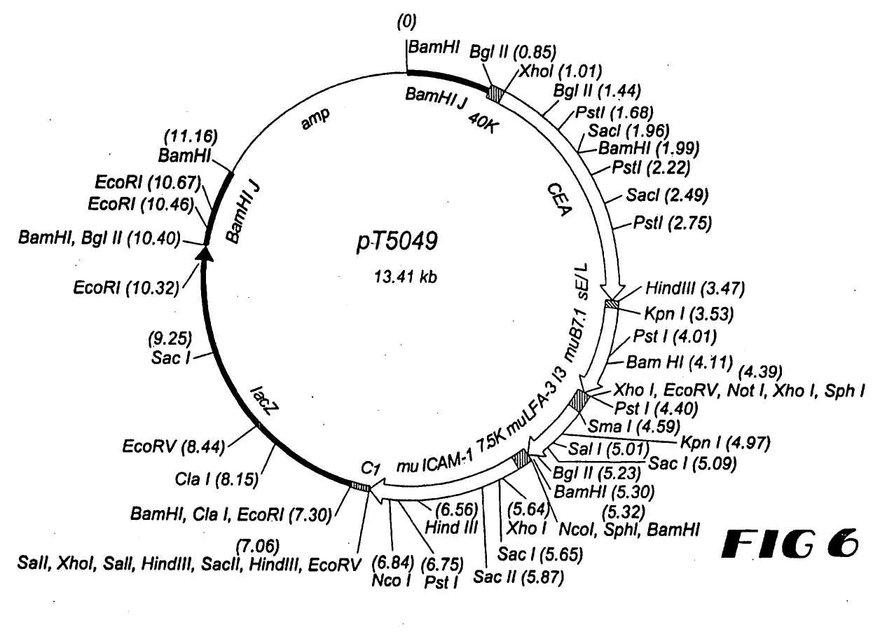

In another particular embodiment the plasmid vector comprises a nucleic acid sequence encoding the tumor associated antigen, CEA, under control of the 40K promoter, a nucleic acid sequence encoding B7 under control of the sE/L promoter, a nucleic acid sequence encoding LFA-3 under control of a I3 promoter and a nucleic acid sequence encoding ICAM-1. The plasmid vector may further comprise a lacZ gene under control of a C1 promoter as depicted in FIG. 6 as pT5049. Plasmid pT5049, was deposited with the American Type Culture Collection, 10801 University Boulevard, Manassas, Va. 20110 on Nov. 13, 1998 as ATCC Accession No. 203481 under the terms of the Budapest Treaty.

In yet another embodiment of the plasmid vector, the vector comprises a nucleic acid sequence encoding huLFA-3 under control of the 30K promoter, a nucleic acid sequence encoding hulCAM-1 under control of an I3 promoter and huB7.1 under control of the sE/L promoter. A particular embodiment of the plasmid vector is depicted as pT5064 in FIG. 8, which was deposited with the ATCC on Nov. 13, 1998 as ATCC Accession No. 203482 under the terms of the Budapest Treaty.

The plasmid vector of the present invention may be provided in kit form for use in methods of generating recombinant vectors. The kit may further provide a parental virus, and other reagents used in the recombination process.

The present invention further provides methods of generating recombinant poxviruses comprising nucleic acid sequences encoding multiple costimulatory molecules. One method of generation of recombinant poxviruses is accomplished via homologous recombination in vivo between parental poxvirus genomic DNA and a plasmid vector that carries the heterologous sequences to be inserted as disclosed in U.S. Pat. No. 5,093,258. Plasmid vectors for the insertion of foreign sequences into poxviruses are constructed by standard methods of recombinant DNA technology (A36). The plasmid vectors contain one or more chimeric foreign genes, each comprising a poxvirus promoter linked to a protein coding sequence, flanked by viral sequences from a non-essential region of the poxvirus genome. The plasmid is transfected into cells infected with the parental poxvirus using art accepted transfection methods, and recombination between poxvirus sequences on the plasmid and the corresponding DNA in the parental viral genome results in the insertion into the viral genome of the chimeric foreign genes from the plasmid. Recombinant viruses are selected and purified using any of a variety of selection or screening systems as are known in the art (A14). Insertion of the foreign genes into the vaccinia genome is confirmed by polymerase chain reaction (PCR) analysis. Expression of the foreign genes is demonstrated by Western blot analysis. An alternative method of generation of recombinant poxviruses is accomplished by direct ligation (Pleiderer et al J. Gen. Virol. 76:2957-2962, 1995; Merchlinsky et al Virol. 238:444-451, 1997).

Use of the recombinant vector comprising nucleic acid sequences encoding multiple costimulatory molecules in combination with a nucleic acid sequence encoding at least one target antigen or epitope thereof is useful in enhancing an immune response against the target antigen or epitope thereof, and enhance the immune response against cells expressing the target antigen or epitope thereof. The magnitude of the immune response against the target antigen, epitope, or cells expressing target antigen obtained using the recombinant vector of the present invention is significantly greater than that achieved using systems employing a single or a double costimulatory molecule.

The recombinant vector encodes at least three or more costimulatory molecules in combination with a nucleic acid sequence encoding a target antigen or immunological epitope thereof for providing a synergistic immunological response to the target antigen or epitope thereof. In one embodiment, a recombinant poxvirus provides a nucleic acid sequence encoding B7, ICAM-1 and LFA-3, along with a nucleic acid sequence encoding at least one target antigen or immunological epitope thereof. In some instances it may be beneficial to provide more than one nucleic acid sequence to provide multiple target antigens or immunological epitopes thereof for the purpose of having a multivalent vaccine.

The target antigen, as used herein, is an antigen or immunological epitope on the antigen which is crucial in immune recognition and ultimate elimination or control of the disease-causing agent or disease state in a mammal. The immune recognition may be cellular and/or humoral. In the case of intracellular pathogens and cancer, immune recognition is preferably a T lymphocyte response.

The target antigen may be derived or isolated from a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036). Hepatitis C (U.S. Pat. No. 5,709,995), EBV, Cytomegalovirus (CMV) and the like. Target antigen may be derived or isolated from pathogenic bacteria such as from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like.

Target antigen may be derived or isolated from pathogenic yeast including Aspergillus, invasive Candida (U.S. Pat. No. 5,645,992). Nocardia, Histoplasmosis, Cryptosporidia and the like.

Target antigen may be derived or isolated from a pathogenic protozoan and pathogenic parasites including but not limited to Pneumocystis carinii. Trypanosoma, Leishmania (U.S. Pat. No. 5,965,242), Plasmodium (U.S. Pat. No. 5,589,343) and Toxoplasma gondii.

Target antigen includes an antigen associated with a preneoplastic or hyperplastic state. Target antigen may also be associated with, or causative of cancer. Such target antigen may be tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D (A46) and the like (GenBank Accession No. M29540), MART-1 (Kawakami et al., J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et a Cancer Res. 53:227-230, 1993), tyrosinase (K won et al PNAS 84:7473-7477, 1987. TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBO J, 11:527-535, 1992), TAG72, KSA, CA-125, PSA, HER-2/neu/c-erb/B2, (U.S. Pat. No. 5,550,214), BRC-1, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each.

Possible human tumor antigens and tissue specific antigens as well as immunological epitopes thereof for targeting using the present invention include but are not limited to those exemplified in Table 1.

TABLE I

| | | | |
|---|---|---|---|
| | | | SEQ. |
| Target | Restriction | Immunological | ID |
| antigens | element | Peptide epitope | No. |

Human target tumor antigens recognized by T cells

| | | | |
|---|---|---|---|
| SP 100 | HLA-A2 | KTWQQYWZY | |
| | HLA-A2 | ITDQVPPSV | 2 |
| | HLA-A2 | YLEPGPVTA | 3 |
| | HLA-A2 | LLDGTATLRL | 4 |
| | HLA-A2 | VLYRYGSFSV | 5 |
| MART1-s | HLA-A2 | AAGIGILTV | 6 |
| Melam A | HLA-A2 | ILTVILGVL | 7 |
| TRP-1 | HLA-A3 | MSLQRQPLR | 8 |
| (GP75) | | | |
| Tyrosinase | HLA-A2 | MLLAVLYCL | 9 |
| | HLA-A2 | YMNGTMSQV | 10 |
| | HLA-B44 | SEIWRDIDP | 11 |
| | HLA-A24 | AFLPWHRLF | 12 |
| | HLA-DR4 | QNILLSNAPLGPQFT | 13 |
| | HLA-DR4 | SYLQDSDPDSFQD | 14 |
| MAGB-1 | HLA-A1 | EADPTGHSY | 15 |
| | HLA-Cw16 | SAYGEPRKL | 16 |
| MAGB-3 | HLA-A1 | EVDPIGHLY | 17 |
| | HLA-A2 | FLWGPRALV | 18 |
| BAGE | HLA-Cw16 | AARAVFLAL | 19 |
| GAGE-1.2 | HLA-Cw6 | YRPRPRRY | 20 |
| N-acetyl | HLA-A2 | VLPDVFCRC | 21 |
| glucos- | | | |
| aminyl- | | | |
| transferase | | | |
| V | | | |
| p15 CEA | HLA-A24 | AYGLDFYIL | 22 |
| | | YLSGANILNL | 23 |
| | | (CAP1) | |
| | | YLSGADILNL | 24 |
| | | (CAP1-6D) | |
| | | | 37 |
| Z,899 | HLA-A24 | SYLDSGIHF | 25 |
| MUM-1 | HLA-B44 | EEKLIVVLF | 26 |
| CDK4 | HLA-A2 | ACDPESGFIFV | 27 |
| HER-2/neu | HLA-A2 | HSAVVGHL | 28 |
| (Breast | HLA-A2 | KIFGSLAFL | 29 |
| and ovarian | | | |
| carcinoma) | | | |
| Human | HLA-A2 | YMLDLQPETT | 30 |
| papillomavirus | | | |
| E6, E7 | | | |
| (cervical | | | |
| carcinoma) | | | |
| MOC-1 | Non-MHC | PDTRPAPGSTAPPAFIGV- | 31 |
| | restricted | TSA (and portions | |
| | MHC | thereof) | |
| | restricted | | |
| (Breast, | A2, A3 | FLTPKKLQCVDLHVISND- | 32 |
| ovarian and | | VCAQVHPQKVTK | |
| pancreatic | | FLTPKKLQCV | 33 |
| carcinoma) | | KLQCVDLHV | 34 |
| PSA | | VLSNDVCAQV | 35 |
| | | QVHPQKVTK | 36 |

For organisms which contain a DNA genome, a gene encoding a target antigen or immunological epitope thereof of interest is isolated from the genomic DNA. For organisms with RNA genomes, the desired gene may be isolated from cDNA copies of the genome. If restriction maps of the genome are available, the DNA fragment that contains the gene of interest is cleaved by restriction endonuclease digestion by methods routine in the art. In instances where the desired gene has been previously cloned, the genes may be readily obtained from the available clones. Alternatively, if the DNA sequence of the gene is known, the gene can be synthesized by any of the conventional techniques for synthesis of deoxyribonucleic acids.

Genes encoding an antigen of interest can be amplified by cloning the gene into a bacterial host. For this purpose, various prokaryotic cloning vectors can be used. Examples are plasmids pBR322, pUC and pEMBL.

The genes encoding at least one target antigen or immunological epitope thereof can be prepared for insertion into the plasmid vectors designed for recombination with a virus by standard techniques. In general, the cloned genes can be excised from the prokaryotic cloning vector by restriction enzyme digestion. In most cases, the excised fragment will contain the entire coding region of the gene. The DNA fragment carrying the cloned gene can be modified as needed, for example, to make the ends of the fragment compatible with the insertion sites of the DNA vectors used for recombination with a virus, then purified prior to insertion into the vectors at restriction endonuclease cleavage sites (cloning sites) as described herein.

Diseases may be treated or prevented by use of the present invention and include diseases caused by viruses, bacteria, yeast, parasites, protozoans, cancer cells and the like. The recombinant vector comprising multiple costimulatory molecules may be used as a generalized immune enhancer and as such has utility in treating diseases of no known etiological cause.

Preneoplastic or hyperplastic states which may be treated or prevented using a recombinant vector of the present invention include but are not limited to preneoplastic or hyperplastic states such as colon polyps, Crohn's disease, ulcerative colitis, breast lesions and the like.

Cancers which may be treated using the recombinant vector of the present invention include but are not limited to primary or metastatic melanoma, adenocarcinoma, squamous cell carcinoma, adenosquamous cell carcinoma, thymoma, lymphoma, sarcoma, lung cancer, liver cancer, non-Hodgkins lymphoma, Hodgkins lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

The present invention provides a pharmaceutical composition comprising a recombinant vector comprising foreign genes encoding multiple costimulatory molecules in a pharmaceutically acceptable carrier. At least three genes encoding a costimulatory molecule form part of the recombinant vector and may be selected from the group of genes encoding B7, ICAM-1, LFA-3, 4-1BBL, CD59, CD40, CD70, VCAM-1, OX-40L and the like. The recombinant vector may further comprise a nucleic acid sequence encoding at least one target antigen or immunological epitope thereof. In another embodiment, the pharmaceutical composition comprises a first recombinant vector comprising foreign genes encoding multiple costimulatory molecules, a second recombinant vector comprising nucleic acid sequences encoding at least one target antigen or immunological epitope thereof and a pharmaceutically acceptable carrier. Administration of the pharmaceutical composition provides host cells with the foreign genes encoding multiple costimulatory molecules.

In one embodiment, a pharmaceutical composition comprises a recombinant poxvirus containing foreign genes encoding multiple costimulatory molecules in a pharmaceutically acceptable carrier. The recombinant poxvirus may further comprise a nucleic acid sequence encoding at least one target antigen or immunological epitope thereof or alternatively, a second recombinant poxvirus may be provided encoding at least one target antigen or immunological epitope thereof.

The present invention provides a pharmaceutical composition comprising a recombinant poxvirus comprising a nucleic acid sequence encoding B7.1 to B7.2, a nucleic acid sequence encoding ICAM-1, and a nucleic acid sequence encoding LFA-3 and a pharmaceutically acceptable carrier. In addition to the B7, ICAM-1, LFA-3 construct, the recombinant poxvirus of the pharmaceutical composition may additionally comprise a nucleic acid sequence encoding at least one target antigen or immunological epitope thereof or the nucleic acid sequence encoding at least one target antigen or immunological epitope thereof may be provided in the composition by a second recombinant poxvirus.

The pharmaceutical composition may also comprise exogenously added immunostimulatory molecules as are known in the art including the costimulatory molecules B7, ICAM-1, LFA-3, 4-1BBL, CD59, CD40, CD70, VCAM-1, OX-40L and the like and/or cytokines and chemokines including but not limited to IL2, GM-CSF, TNF.alpha., IFN.gamma., IL-12, RANTES, MIP-1.alpha., Flt-3L (U.S. Pat. Nos. 5,554,512; 5,843,423) and the like for additional synergy or enhancement of an immune response. The cytokines and chemokines themselves may be provided in the composition or, alternatively, the cytokines and chemokines may be provided by a recombinant viral vector encoding the cytokine or chemokine.

The present invention also encompasses methods of treatment or prevention of a disease caused by pathogenic microorganisms or by cancer disclosed herein.

In the method of treatment, the administration of the recombinant vector of the invention may be for either “prophylactic” or “therapeutic” purpose. When provided prophylactically, the recombinant vector of the present invention is provided in advance of any symptom. The prophylactic administration of the recombinant vector serves to prevent or ameliorate any subsequent infection or disease. When provided therapeutically, the recombinant lector is provided at or after the onset of a symptom of infection or disease. Thus the present invention may be provided either prior to the anticipated exposure to a disease-causing agent or disease state or after the initiation of the infection or disease.

The term “unit dose” as it pertains to the inoculum refers to physically discrete units suitable as unitary dosages for mammals, each unit containing a predetermined quantity of recombinant vector calculated to produce the desired immunogenic effect in association with the required diluent. The specifications for the novel unit dose of an inoculum of this invention are dictated by and are dependent upon the unique characteristics of the recombinant virus and the particular immunologic effect to be achieved.

The inoculum is typically prepared as a solution in tolerable (acceptable) diluent such as saline, phosphate-buffered saline or other physiologically tolerable diluent and the like to form an aqueous pharmaceutical composition.

The route of inoculation may be scarification, intravenous (I.V.), intramuscular (I.M.), subcutaneous (S.C.), intradermal (I.D.), intraperitoneal (I.P.), intratumor and the like, which results in eliciting a protective response against the disease causing agent. The dose is administered at least once. Subsequent doses may be administered as indicated.

In one embodiment, heterologous prime-boost regimens are employed. In one example, the host is immunized at least once with a first vector such as a nucleic acid-based vector. Subsequent immunizations are performed with a poxvirus vector. In another example, the host is first immunized with a first poxvirus vector and then with a second poxvirus vector of a different genus.

In providing a mammal with the recombinant vector of the present invention, preferably a human, the dosage of administered recombinant lector will vary depending upon such factors as the mammal's age, weight, height, sex, general medical condition, previous medical history, disease progression, tumor burden and the like.

In general, it is desirable to provide the recipient with a dosage of recombinant virus in the range of about 10⁵ to about 10¹⁰ plaque forming units, although a lower or higher dose may be administered.

The genetic definition of tumor-associated and tumor-specific antigens allows for the development of targeted antigen-specific vaccines for cancer therapy. Insertion of a tumor antigen gene in the genome of recombinant pox viruses in combination with genes encoding multiple costimulatory molecules is a powerful system to elicit a specific immune response in terms of prevention in individuals with an increased risk of cancer development (preventive immunization), to shrink tumors prior to surgery, to prevent disease recurrence after primary surgery (anti-metastatic vaccination), or to expand the number of cytotoxic lymphocytes (CTL) in vivo, thus improving their effectiveness in eradication of diffuse tumors (treatment of established disease). Recombinant viruses of the present invention can elicit an immune response ex vivo in autologous lymphocytes (CD8⁺), either cytotoxic T lymphocytes and/or CD4⁺ helper T cells or NK cells prior to being transferred back to the tumor bearing patient (adoptive immunotherapy).

In cancer treatments, the recombinant vectors can be introduced into a mammal either prior to any evidence of cancers such as an adenocarcinoma or to mediate regression of the disease in a mammal afflicted with a cancer such as adenocarcinoma.

Depending on the disease or condition to be treated and the method of treatment, the recombinant vector may or may not comprise a nucleic acid sequence encoding a target antigen or immunological epitope thereof in addition to the genes encoding multiple costimulatory molecules. The target antigen or immunological epitope thereof may be provided endogenously by the host cell infected with the recombinant vector as, for instance, a tumor cell may endogenously express a tumor associated antigen or epitope thereof and may not require the addition of a foreign gene encoding an exogenous tumor associated antigen. In the case in which a tumor associated antigen is absent, not expressed or expressed at low levels in a host cell, a foreign gene encoding an exogenous tumor associated antigen may be provided. Further, genes encoding several different tumor associated antigens may be provided. The foreign gene encoding an exogenous tumor associated antigen may be provided by the same recombinant vector comprising genes encoding multiple costimulatory molecules or may be provided by a second recombinant vector in an admixture with the first recombinant vector.

Examples of methods for administering the recombinant vector into mammals include, but are not limited to, exposure of tumor cells to the recombinant virus ex vivo, or injection of the recombinant vector into the affected host by intravenous, S.C., I.D. or I.M. administration of the virus. Alternatively the recombinant vector or combination of recombinant vectors may be administered locally by direct injection into the cancerous lesion or tumor or topical application in a pharmaceutically acceptable carrier. The quantity of recombinant vector carrying the nucleic acid sequence of one or more tumor associated antigens (TAAs) in combination with nucleic acid sequences encoding multiple costimulatory molecules to be administered is based on the titer of virus particles. A preferred range of the immunogen to be administered is 10⁵ to 10¹⁰ virus particles per mammal, preferably a human. If the mammal to be immunized is already afflicted with cancer or metastatic cancer, the vaccine can be administered in conjunction with other therapeutic treatments.

In one method of treatment, autologous cytotoxic lymphocytes or tumor infiltrating lymphocytes may be obtained from blood, lymph nodes, tumor and the like from a patient with cancer. The lymphocytes are grown in culture and target antigen-specific lymphocytes are expanded by culturing in the presence of specific target antigen and either antigen presenting cells expressing multiple foreign costimulatory molecules or target antigen pulsed APCs of the present invention. The target antigen-specific lymphocytes are then reinfused back into the patient.

After immunization the efficacy of the vaccine can be assessed by production of antibodies or immune cells that recognize the antigen, as assessed by specific lytic activity or specific cytokine production or by tumor regression. One skilled in the art would know the conventional methods to assess the aforementioned parameters.

The present invention encompasses methods of enhancing antigen-specific T-cell responses by administration of an effective amount of a recombinant vector encoding multiple foreign costimulatory molecules and a target antigen into a mammal alone, or infecting a target cell with the vector, target antigen or immunological epitope thereof. In one embodiment of the method, a recombinant vector encoding at least one molecule from the B7 family, ICAM-1 and LFA-3 is administered alone, or admixed with a target cell, target antigen or immunological epitope thereof. This immunization approach augments or enhances immune responses generated by the target antigen, providing a synergistic response compared to the use of single or double costimulatory molecules. The method of administering a recombinant vector containing genes encoding multiple costimulatory molecules results in increased target antigen-specific lymphoproliferation, enhanced cytolytic activity, enhanced cytokine secretion and longer lasting immunity to the target antigen as compared to the use of recombinant vector encoding a single or double costimulatory molecule. The recombinant vector may further comprise a nucleic acid sequence encoding at least one target antigen or immunological epitope thereof for synergistic enhancement of target-antigen-specific immune responses. Alternatively, the nucleic acid sequence encoding at least one target antigen or immunological epitope thereof is provided by a second recombinant vector, distinct from the vector encoding the multiple costimulatory molecules. In one embodiment of the method of enhancing antigen-specific T-cell responses, mammals, preferably humans, are immunized with an rV-HIV-1 epitope/B7-1/ICAM-1/LFA-3 construct. The efficacy of the treatment may be monitored in vitro and/or in vivo by determining target antigen-specific lymphoproliferation, target antigen-specific cytolytic response, clinical responses and the like.

The method of enhancing antigen-specific T-cell responses may be used for any target antigen or immunological epitope thereof. Of particular interest are tumor associated antigens, tissue specific antigens and antigens of infectious agents.

In addition to administration of the recombinant vector to the patient, other exogenous immunomodulators or immunostimulatory molecules, chemotherapeutic drugs, antibiotics, antifungal drugs, antiviral drugs and the like alone or in combination thereof may be administered depending on the condition to be treated. Examples of other exogenously added agents include exogenous IL-2, IL-6, alpha-, beta- or gamma-interferon, GM-CSF, tumor necrosis factor, Flt-3L, cyclophosphamide, cisplatinum, gancyclovir, amphotericin B, 5 fluorouracil and the like.

The present invention provides for host cells expressing multiple, exogenous foreign costimulatory molecules in which the molecules are provided by a recombinant vector having foreign nucleic acid sequences encoding multiple costimulatory molecules. The host cells may also express one or more endogenous target antigens or immunological epitopes thereof or may be engineered to express one or more exogenous, foreign target antigens or immunological epitopes thereof which may also be provided by the recombinant vector encoding multiple costimulatory molecules or by a second recombinant vector.

The host cells of the present invention, wraith utility in stimulating an antigen-specific immune response may be any cell capable of infection using the recombinant virus of the present invention and capable of expressing multiple, exogenous costimulatory molecules and may further be genetically engineered to express one or more exogenous target antigens or immunological epitopes thereof. Such host cells included but are not limited to tumor cells, antigen presenting cells, such as PBMC, dendritic cells, cells of the skin or muscle, and the like. Antigen presenting cells include, but are not limited to, monocytes, macrophages, dendritic cells, progenitor dendritic cells, Langerhans cells, splenocytes, B-cells, tumor cells, muscle cells, epithelial cells and the like.

In one embodiment, the host cells are tumor cells in which the tumor cells are exposed to the recombinant vector in situ or in vitro to cause expression of multiple foreign or exogenous costimulatory molecules on the tumor cells. The tumor cells may express an endogenous target antigen or the tumor cells may be further genetically engineered to express a target antigen such as TAA or immunological epitope thereof. Tumor cells expressing both the TAA along with multiple immunostimulatory molecules are administered to a mammal in an effective amount to result in tumor reduction or elimination in the mammal afflicted with a cancer.

The present invention also provides progenitor dendritic cells, dendritic cells (DC), DC subpopulations, and derivatives thereof overexpressing multiple costimulatory molecules in which multiple costimulatory molecules are exogenously provided by a recombinant vector having nucleic acid sequences encoding multiple costimulatory molecules. The progenitor DC and DC of the present invention express higher levels of costimulatory molecules, than levels endogenously expressed by a nontreated progenitor DC or DC. The APCs such as progenitor dendritic cells and dendritic cells may also express one or more endogenous target antigens or immunological epitopes thereof or exogenous target antigen may also be provided by the recombinant vector encoding multiple costimulatory molecules or by a second recombinant vector. The present invention further provides methods of using the multiple costimulatory molecule-overexpressing APCs, such as multiple costimulatory molecule-overexpressing progenitor dendritic cells and multiple costimulatory molecule-overexpressing dendritic cells in activating T cells in vivo or in vitro for vaccination and immunotherapeutic responses against one or more target cells, target antigens and immunological epitopes thereof.

The APCs such as progenitor dendritic cells, dendritic cells, DC subpopulations and derivatives thereof isolated from a source are infected, transfected or transduced with a recombinant vector comprising exogenous genes encoding at least three costimulatory molecules for a time period sufficient to allow functional overexpression of the multiple costimulatory molecules. Such multiple costimulatory molecule-overexpressing antigen presenting progenitor dendritic cells and dendritic cells may also be pulsed or incubated wraith at least one target cell, target cell lysate, target cell membrane, target antigen, or immunological epitope thereof, or with RNA or DNA of at least one target cell and administered to a species in an amount sufficient to activate the relevant T cell responses in vivo. In another embodiment, the antigen presenting progenitor dendritic cells and dendritic cells additionally express at least one foreign target antigen or immunological epitope thereof.

Host cells expressing multiple, exogenous costimulatory molecules may be provided in a dose of 10³ to 10⁹ cells. Routes of administration that may be used include intravenous, subcutaneous, intralymphatic, intratumoral, intradermal, intramuscular, intraperitoneal, intrarectal, intravaginal, intranasal, oral, via bladder instillation, via scarification, and the like.

In one embodiment, the multiple costimulatory molecule-overexpressing antigen presenting progenitor dendritic cells or dendritic cells are exposed to a target cell, target cell lysates, target cell membranes, target antigen or immunological epitope thereof or with DNA or RNA from at least one target cell in vitro and incubated with primed or unprimed T cells to activate the relevant T cell responses in vitro. The activated T cells alone or in combination with the progenitor DC or DC are then administered to a species such as a human for vaccination or immunotherapy against a target cell, target antigen or immunological epitope thereof. In one method of use, the progenitor dendritic cells or dendritic cells are advantageously used to elicit an immunotherapeutic growth inhibiting response against cancer cells.

In another embodiment, the multiple costimulatory molecule-overexpressing antigen-presenting cell, preferably a precursor DC or DC is fused with a target cell expressing a relevant target antigen or immunological epitope thereof to form a heterokaryon of APC and target cell by methods known in the art (Gong, J. et al Proc. Natl. Acad. Sci. USA 95:6279-6283, 1998). Such a fusion cell or chimeric APC/target antigen cell expresses both multiple costimulatory molecules and target antigen or immunological epitopes thereof. In a preferred embodiment the target cell is a hyperplastic cell, premalignant or malignant cell. The chimeric APC/target antigen cell may be used both in vivo and in vitro to enhance immune responses of T and B lymphocytes.

Progenitor dendritic cells are obtained from bone marrow, peripheral blood and lymph nodes from a patient. The patient may have been previously vaccinated, or treated with a compound such as Flt-3L to enhance the number of antigen-presenting cells. Dendritic cells are obtained from any tissue such as the epidermis of the skin (Langerhans cells) and lymphoid tissues such as found in the spleen, bone marrow, lymph nodes, and thymus as well as the circulatory system including blood and lymph (veiled cells). Cord blood is another source of dendritic cells.

Dendritic cells may be enriched or isolated for use in the present invention using methods known in the art such as those described in U.S. Pat. No. 5,788,963. Once the progenitor dendritic cells, dendritic cells and derivatives thereof are obtained, they are cultured under appropriate culture conditions to expand the cell population and/or maintain the cells in a state for optimal infection, transfection or transduction by a recombinant vector and for optimal target antigen uptake, processing and presentation. Particularly advantageous for maintenance of the proper state of maturity of dendritic cells in in vitro culture is the presence of both the granulocyte/macrophage colony stimulating factor (GM-CSF) and interleukin 4 (IL-4). Subpopulations of dendritic cells may be isolated based in adherence and/or degree of maturity based on cell surface markers. The phenotype of the progenitor DC, DC and subpopulations thereof are disclosed in Banchereau and Steinman Nature 392:245-252, 1998.

In one embodiment GM-CSF and IL-4 are each provided in a concentration of about 500 units/ml for a period of about 6 days (A41, A42). In another embodiment, TNF.alpha. and/or CD40 is used to cause precursor DC or DC to mature.

The progenitor dendritic cells or dendritic cells may be obtained from the individual to be treated and as such are autologous in terms of relevant HLA antigens or the cells may be obtained from an individual whose relevant HLA antigens (both class I and II, e.g. HLA-A, B, C and DR) match the individual that is to be treated. Alternatively, the progenitor dendritic cell is engineered to express the appropriate, relevant HLA antigens of the individual receiving treatment.

The progenitor dendritic cells and dendritic cells may be further genetically modified to extend their lifespan by such methods as EBV-transformation as disclosed in U.S. Pat. No. 5,788,963.

The dendritic cells and precursors thereof may be provided in the form of a pharmaceutical composition in a physiologically acceptable medium. The composition may further comprise a target cell, target cell lysate, target cell membrane, target antigen or immunological epitope thereof. The composition may additionally comprise cytokines and/or chemokines such as IL4 and GM-CSF for additional synergistic enhancement of an immune response.

In another embodiment, the APC of the present invention overexpressing multiple costimulatory molecules is useful in methods of evaluating efficacy of a vaccine by determination of antigen-specific lymphocyte proliferation and function. In such a method, lymphocytes are recovered from an individual nacho has been vaccinated with a target cell lysate, target cell membrane, target antigen or immunological epitope thereof. The lymphocytes are cultured in vitro with an APC of the present invention in the presence of the target cell, target cell lysate, target cell membrane, target antigen or immunological epitope thereof and an enhancement of antigen-specific lymphocyte numbers and functions determined by methods known in the art. An enhancement in numbers and/or functions is indicative of efficacy of the vaccine. The method is particularly useful in determining efficacy of peptide vaccines in stimulating an appropriate immune response.

In another embodiment, the APCs of the present invention expressing exogenous multiple costimulatory molecules are useful in a method of screening for novel immunogenic peptides from a multiplicity of peptides. In the method of screening, antigen presenting cells infected with a recombinant vector encoding multiple costimulatory molecules or functional portions thereof are pulsed with a multiplicity of peptides to form a peptide-pulsed antigen presenting cell. The peptide-pulsed antigen presenting cell is incubated with lymphoid cells and the immunoreactivity of the lymphoid cells measured. An enhancement of immunoreactivity of the lymphoid cells in the presence of the peptide-pulsed APC is indicative of an antigen specific response to the peptide. The peptide eliciting the enhanced response can be identified by eluting from tumor, by analysis of HLA binding, etc. The source of the multiplicity of peptides may be a combinatorial library which expresses a multiplicity of random peptides. The enhanced immunoreactivity may be a humoral or cell-mediated immune response and may be measured using standard techniques known in the art such as antigen-induced proliferation, cytotoxicity, antibody secretion, signal transduction, and the like. The novel peptides identified may be used as immunogens, vaccines or diagnostic agents. The proteins that contain the peptides may be identified by subtraction libraries and differential display gene technologies.

The recombinant vectors of the present invention as well as host cells infected, transfected or induced by the recombinant vector of the present invention are useful in methods of stimulating an enhanced humoral response both in vivo and in vitro. Such an enhanced humoral response may be monoclonal or polyclonal in nature. The enhancement of humoral responses using multiple costimulatory molecules is synergistic as compared to a humoral response using a single or double costimulatory molecule. The synergistic enhancement of a humoral response may be determined by increased proliferation and/or cytokine secretion by CD4⁺ T cells, increased proliferation or antibody production by B cells, increased antibody dependent cellular toxicity (ADCC), increased complement-mediated lysis, and the like. Antibody elicited using the recombinant vectors of the present invention or using host cells infected, transfected or induced by the recombinant vector of the present invention are expected to be higher affinity and/or avidity and higher titer than antibody elicited by standard methods. The antibody elicited by methods using the recombinant vector may recognize immunodominant target epitopes or nondominant target epitopes.

This invention further comprises an antibody or antibodies elicited by immunization with the recombinant vector of the present invention. The antibody has specificity for and reacts or binds with the target antigen or immunological epitope thereof of interest. In this embodiment of the invention the antibodies are monoclonal or polyclonal in origin.

Exemplary antibody molecules are intact immunoglobulin molecules, substantially intact immunoglobulin molecules or those portions of an immunoglobulin molecule that contain the antigen binding site, including those portions of immunoglobulin molecules known in the art as F(ab), F(ab′), F(ab′)₂ and F(v). Polyclonal or monoclonal antibodies may be produced by methods known in the art. (Kohler and Milstein (1975) Nature 256, 495-497, Campbell “Monoclonal Antibody Technology, the Production and Characterization of Rodent and Human Hybridomas” in Burdon et al. (eds.) (1985) “Laboratory Techniques in Biochemistry and Molecular Biology,” Volume 13, Elsevier Science Publishers, Amsterdam). The antibodies or antigen binding fragments may also be produced by genetic engineering. The technology for expression of both heavy and light chain genes in E. coli is the subject of the PCT patent applications: publication number WO 901443, WO 901443 and WO 9014424 and in Huse et al. (1989) Science 246:1275-1281.

In one embodiment the antibodies of this invention are used in immunoassays to detect the novel antigen of interest in biological samples.

In one embodiment, the antibodies of this invention generated by immunization with a recombinant vaccinia virus expressing a TAA and expressing B7-1, ICAM-1 and LFA-3 are used to assess the presence of the a TAA from a tissue biopsy of a mammal afflicted with a cancer expressing TAA using immunocytochemistry. Such assessment of the delineation of the a TAA antigen in diseased tissue can be used to prognose the progression of the disease in a mammal afflicted with the disease or the efficacy of immunotherapy. In this embodiment, examples of TAAs include but are not limited to CEA, PSA, and MUC-1. Conventional methods for immunohistochemistry are described in (Harlow and Lane (eds) (1988) In “Antibodies A Laboratory Manual”. Cold Spinning Harbor Press, Cold Spring Harbor, N.Y.; Ausubel et al., (eds) (1987). In Current Protocols In Molecular Biology, John Wiley and Sons (New York, N.Y.).

In another embodiment the antibodies of the present invention are used for immunotherapy. The antibodies of the present invention may be used in passive immunotherapy.

In providing a patient with the antibodies or antigen binding fragments to a recipient mammal, preferably a human, the dosage of administered antibodies or antigen binding fragments will vary depending upon such factors as the mammal's age, weight, height, sex, general medical condition, previous medical condition and the like.

The antibodies or antigen-binding fragments of the present invention are intended to be provided to the recipient subject in an amount sufficient to prevent, lessen or attenuate the severity, extent or duration of the disease or infection.

Anti-idiotypic antibodies arise normally during the course of immune responses, and a portion of the anti-idiotype antibody resembles the epitope that induced the original immune response. In the present invention, the immunoglobulin gene or portion thereof of an antibody whose binding site reflects a target antigen of a disease state, is incorporated into the genome or portion thereof of a virus genome, alone or in combination with a gene or portion thereof of multiple immunostimulatory molecules, the resulting recombinant virus is able to elicit cellular and humoral immune response to the antigen.

The description of the specific embodiments will so fully reveal the general nature of the invention that others can readily modify and/or adopt for various purposes such specific embodiments without departing from the generic concept, and therefor such adaptations and modifications are intended to be comprehended within the meaning and range of equivalents of the disclosed embodiments.

The invention further includes CEA agonist polypeptides/proteins comprising a modified CEA epitope wherein said modified CEA epitope comprises the sequence YLSGADLNL, nucleic acids coding therefor, vectors and/or cells comprising said nucleic acids (collectively designated as “agents” of the invention), and mixtures/compositions of the aforementioned. All of the aforementioned agents and mixtures/compositions of the invention have the ability to induce or elicit an immune response against a CEA protein fragment thereof, a CEA agonist polypeptide/protein of the invention, a CEA epitope and/or modified CEA epitope, and/or cells binding or expressing the aforementioned. An “immune response” is defined as any response of the immune system, for example, of either a cell mediated (i.e. cytotoxic T-lymphocyte mediated) or humoral (i.e. antibody mediated) nature. As is known to those skilled in the art, various assays/methodologies exist for the assessment and/or monitoring of immunological responses.

Within the context of cell-mediated immune responses, tumor associated antigen proteins (such as CEA) are processed by intracellular proteases into smaller epitope peptides which are subsequently transported to the cell surface tightly bound in a cleft on an MHC HLA class I molecule. T cells recognize these small epitope peptides only when presented in association with MHC HLA Class I molecules on the surface of appropriate cells. Analogously, in the context of humoral immune responses proteins can be processed into smaller epitope peptides which are subsequently presented on cell surfaces (i.e. antigen presenting cells) in association with MHC HLA class II molecules. Said complexes are recognized by appropriate cells of the humoral immune system.

As is well known to those skilled in the art, short peptides (i.e. epitopes) composed of amino acid sequences of about 8 to 12 amino acids derived from antigens are capable of binding directly within the cleft of an HLA class I molecule without intracellular processing. As previously noted, a number of such epitope peptides derived from CEA have been identified. Moreover, some of these CEA specific antigen epitopes have demonstrated the capacity to induce/elicit immune responses wherein appropriate CEA expressing target cells are lysed. The CEA agonist polypeptides/proteins of the present invention (comprising a modified CEA epitope) elicit an improved immune response (hence, deemed to be CEA “agonist(s)”) by comparison to that observed when normal/unmodified CEA (comprising normal epitopes) is employed as an immunogen.

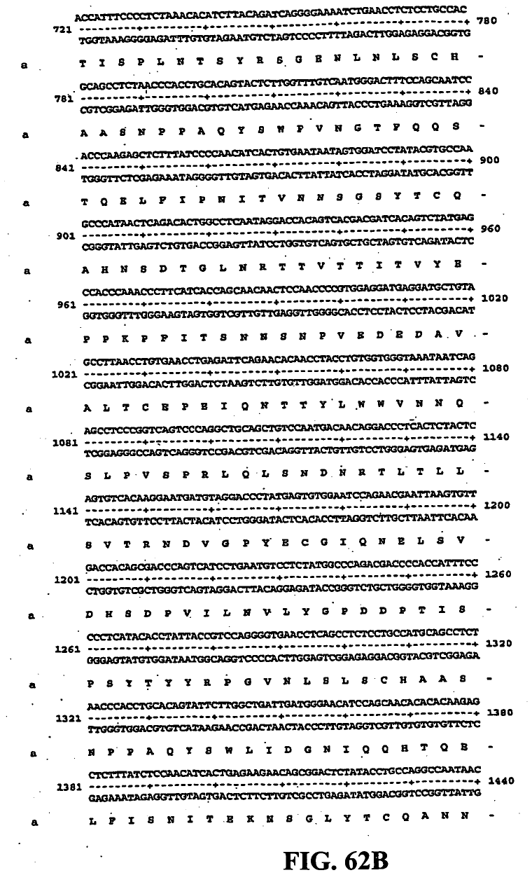

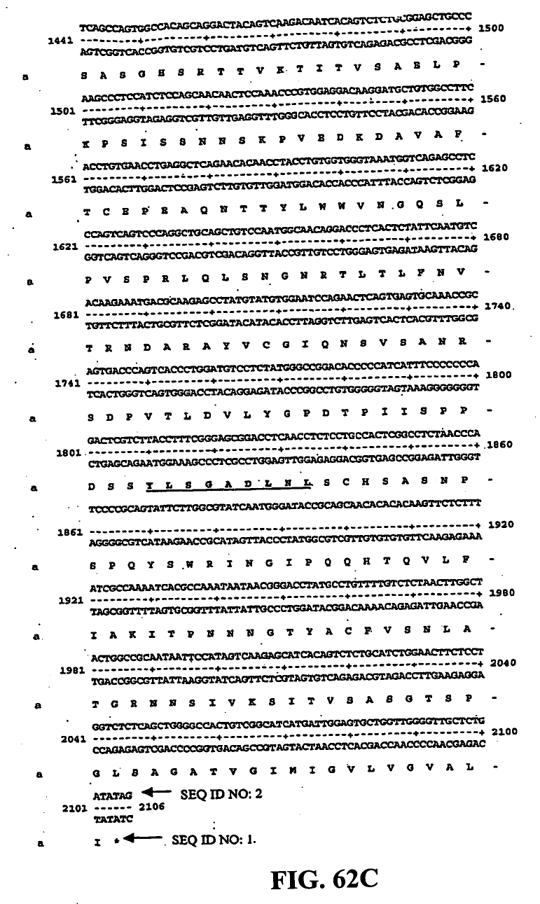

As encompassed by this invention, the CEA agonist polypeptides/proteins comprise a modified epitope containing the amino acid sequence YLSGADLNL (designated “CAP16D”). The counterpart sequence of the naturally occurring epitope in unmodified CEA is YLSGANLNL (designated “CAP1”; Zaremba. S. et al., (1997) Cancer Res. 57: 4570). In a particular embodiment of the invention, the CEA agonist polypeptide/protein has the amino acid sequence of SEQ ID NO:1 (FIG. 62). It is further recognized that CEA agonist polypeptide (s)/protein (s) embodiments of the invention encompass both precursor and mature forms of polypeptide/protein (for example, see Oikawa, S. et al. (1987) Biochefn. Biophys. Res. Conzmun. 142: 511-518).

The CEA agonist polypeptides/proteins of the invention may be prepared using a variety of methods known to one skilled in the art. Accordingly, recombinant DNA methods known to those skilled in the art can be utilized to provide these polypeptides. Nucleic acid sequences which encode for the CEA agonist polypeptides/proteins of the invention may be incorporated in a known manner into appropriate expression vectors (i.e. recombinant expression vectors). Possible expression vectors include (but are not limited to) cosmids, plasmids, or modified viruses (e.g. replication defective retroviruses, adenoviruses and adeno-associated viruses, lentiviruses, herpes viruses, poxviruses), so long as the vector is compatible with the host cell used. The expression “vector is compatible with the host cell” is defined as contemplating that the expression vector (s) contain a nucleic acid molecule of the invention (hereinafter described) and attendant regulatory sequence (s) selected on the basis of the host cell (s) to be used for expression, said regulatory sequence (s) being operatively linked to the nucleic acid molecule.

“Operatively linked” is intended to mean that the nucleic acid is linked to regulatory sequence (s) in a manner which allows expression of the nucleic acid. Suitable regulatory sequences may be derived from a variety of sources, including bacteria), fungal, or viral genes. (For example, see the regulatory sequences described in Goeddel, Gene Expression Technology: Methods in Enzymology 185, Academic Press, San Diego, Calif. (1990). Selection of appropriate regulatory sequence (s) is dependent on the host cell (s) chosen, and may be readily accomplished by one of ordinary skill in the art. Examples of such regulatory sequences include the following: a transcriptional promoter and enhancer, RNA polymerase binding sequence, or a ribosomal binding sequence (including a translation initiation signal).

Depending on the host cell chosen and the expression vector employed, other additional sequences (such as an origin of replication, additional DNA restriction sites, enhancers, and sequences confeffing inducibility of transcription) may be incorporated into the expression vector.

The aforementioned expression vectors of the invention may also contain a selectable marker gene which facilitates the selection of host cells transformed or transfected with a recombinant molecule of the invention. Examples of selectable marker genes are genes encoding a protein such as G418 and hygromycin (which confer resistance to certain drugs), p-galactosidase, chloramphenicol acetyltransferase, or firefly luciferase.

Transcription of the selectable marker gene is monitored by changes in the concentration of the selectable marker protein such asp-galactosidase, chloramphenicol acetyltransferase, or firefly luciferase. Transformant cells can be selected with appropriate selection molecules if the selectable marker gene encodes a protein conferring antibiotic resistance (i.e. G418 in context of neomycin resistance). As is known to one skilled in the art, cells that have incorporated the selectable marker gene will survive awhile cells which do not have any such incorporated detectable marker will die. This makes it possible to visualize and assay for expression from recombinant expression vectors of the invention. It will also be appreciated that selectable markers can be introduced on a separate vector from the nucleic acid of interest.

The recombinant expression vectors may also contain genes which encode a fusion moiety which provides increased expression of the polypeptides of the invention; increased solubility of the polypeptides of the invention; and/or aids in the purification of a target recombinant protein by acting as a ligand in affinity purification. For example, a proteolytic cleavage site may be added to the target recombinant polypeptide to allow separation of the recombinant polypeptide peptide (s) from the fusion moiety subsequent to purification of the fusion protein.

The CEA agonist polypeptides/proteins of the invention may also be prepared by chemical synthesis using techniques well known in the chemistry of proteins such as solid phase synthesis (Merrifield (1964) J; Am. Chen. Assoc. 65: 2149) or synthesis in homogenous solution (Methods of Organic Chemistry. E. Wansch (Ed.) Vol. 15, pts. I and II, Thieme, Stuttgart (1987)).

Additional embodiments of the invention encompass nucleic acids coding for the CEA agonist polypeptides/proteins hereinbefore described. As defined herein, “nucleic acid (s)” encompass (but is not limited to) viral nucleic acid (s), plasmid (s), bacterial DNA, naked/free DNA and RNA. The nucleic acids encompass both single and double stranded forms. As such, these nucleic acids comprise the relevant base sequences coding for the aforementioned polypeptides. For purposes of definitiveness, the “relevant base sequence's coding for the aforementioned polypeptides” further encompass complementary nucleic acid sequences.

In one embodiment of the invention, the nucleic acid has the sequence denoted by SEQ ID NO: 2 (FIG. 62). In further embodiments of the invention, the nucleic acid comprises this sequence (i.e. SEQ ID NO: 2 (FIG. 62)). It is further recognized that additional embodiments of the invention may consist of and/or comprise nucleic acid sequences coding for precursor or mature CEA agonist polypeptide (s)/protein (s) (for example, see Oikawa, S. et al., (987) Supra).

Bacterial DNA useful in embodiments of the invention are known to those of ordinary skill in the art. These bacteria include, for example, Shigella, Salnioiiella, Vibrio claolerae, Lactobacillus, Bacille Calmette Guerin (BCG), and Streptococcus.

In bacterial DNA embodiments of the invention, nucleic acid of the invention may be inserted into the bacterial genome, can remain in a free state, or be parried on a plasmid.

Viral nucleic acid embodiments of the invention may be derived from a poxvirus or other virus such as adenovirus or alphavirus. Preferably the viral nucleic acid is incapable of integration in recipient animal cells. The elements for expression from said nucleic acid may include a promoter suitable for expression in recipient animal cells.

Embodiments of the invention encompass poxviral nucleic acid selected from the group consisting of avipox, orthopox, and suipox nucleic acid. Particular embodiments encompass poxviral nucleic acid selected from vaccinia, fowlpox, canary pox and swinepox; specific examples include TROVAC, NYVAC, ALVAC,

MVA, Wyeth and Poxvac-TC (described in more detail below).

It is further contemplated that nucleic acids of this invention may further comprise nucleic acid sequences, encoding at least one member chosen from the group consisting cytokines, lymphokines, and co-stimulatory molecules. Examples include (but are hot limited to) interleukin 2, interleukin 12, interleukin 6, interferon gamma, tumor necrosis factor Alpha, GM-CSF, B7.1, B7.2, ICAM-1, LFA-3, and Cd72.

Standard techniques of molecular biology for preparing and purifying nucleic acids well known to those skilled in the art can be used in the preparation of aspects of the invention (for example, as taught in Current Protocols in Molecular Biology, F. M. Ausubel et al. (Eds.), John Wiley and Sons, Inc, N.Y.: U.S.A. (1998), Chpts. 1, 2 and 4; Molecular Cloning: A Laboratory Manual (2nd EdJ, J. Sambrook, E. F. Fritsch and T. Maniatis (Eds.), Cold Spring Harbor Laboratory Press, N.Y., U.S.A. (1989), Chpts. 1, 2 3 and 7).

Aspects of this invention further encompass vector comprising, the aforementioned nucleic acids. In certain embodiments, said vectors may be recombinant viruses or bacteria.

Adenovirus vectors and methods, for their construction have been described (e.g. U.S. Pat. Nos. 5,994,132, 5,932,210,6,057,158 and Published PCT Applications WO9817783, WO 9744475, WO 9961034, WO 9950292, WO 9927101, WO 9720575, WO 9640955, WO 9639534, all of which are herein incorporated by reference). Alphavirus vectors have also been described in the art and can be used in embodiments of this invention (e.g. U.S. Pat. Nos. 5,792,462, 5,739,026,5,843,723, 5,789,245, and Published PCT Applications WO 9210578, WO 9527044, WO 9531565, WO 9815636 all of which are herein incorporated by reference), as have lentivirus vectors (e.g. U.S. Pat. Nos. 6,013,516, 5,994,136 and Published PCT Applications WO 9617816, WO 9712622, WO 9817815, WO 9839463, WO9846083, WO 9915641, WO 9919501, WO 9930742, WO 9931251, WO 9851810, WO 0000600 all of which are herein incorporated by reference). Poxvirus vectors that can be used include, for example, avipox, orthopox or suipox poxvirus (as described in U.S. Pat. Nos. 5,364,773, 4,603,112, 5,762,938, 5,378,457, 5,494,807, 5,505,941, 5,756,103, 5,833,975 and 5,990,091 all of which are herein incorporated by reference). Poxvirus vectors comprising a nucleic acid coding for a CEA agonist polypeptide/protein of the invention can be obtained by homologous recombination as is known to one skilled in the art, as such, the nucleic acid coding for the CEA agonist polypeptide/protein is inserted into the viral genome under appropriate conditions for expression in mammalian cells (as described below).

In one embodiment of the invention the poxvirus vector is ALVAC (1) or ALVAC (2) (both of which have been derived from canarypox virus). ALVAC (1) (or ALVAC (2)) does not productively replicate in non-avian hosts, a characteristic thought to improve its safety profile. ALVAC (1) is an attenuated canarypox virus based vector that was a plaque-cloned derivative of the licensed canarypox vaccine, Kanapox (Tartaglia et al. (1992) Virology 188:217; U.S. Pat. Nos. 5,505,941, 5,756,103 and 5,833,975 all of which are incorporated herein by reference). ALVAC (1) has some general properties which are the same as some general properties of

Kanapox. ALVAC-based recombinant viruses expressing extrinsic immunogens have also been demonstrated efficacious as vaccine vectors (Tartaglla et al, In AIDS Research Reviews (vol. 3) Koff W., Wong-Staol F. and Kenedy R. C. (eds.), Marcel Deldcer N.Y., pp. 361-378 (1993a); Tartaglia, J. et al. (1993b) J. Vivol. 67: 2370). For instance, mice immunized with an ALVAC (1) recombinant expressing the rabies virus glycoprotein were protected from lethal challenge with rabies virus (Tartaglia, J. et al, (1992) supra) demonstrating the potential for ALVAC (1) as a vaccine vector. ALVAC-based recombinants have also proven efficacious in dogs challenged with canine distemper virus (Taylor, J. et al. (1992) Virology 187: 321) and rabies virus (Perkus, M. E. et al., In Combined Vaccines and Simultaneous Administration: Current Issues and Perspective, Annals of the New York Academy of Sciences (1994)), in cats challenged with feline leukemia virus (Tartaglia, J. et al., (1993b) supra), and in horses challenged with equine influenza virus (Taylor, J. et al., In Proceedings of the Third International Symposium on Avian Influenza, Univ. of Wisconsin-Madison, Madison, Wis., pp. 331-335 (1993)). ALVAC (2) is a second-generation ALVAC vector in which vaccinia transcription elements E3L and K3L have been inserted within the C6 locus (U.S. Pat. No. 5,990,091, incorporated herein by reference). The E3L encodes a protein capable of specifically binding to dsRNA. The K3L ORF has significant homology to E1 F-2. Within ALVAC (2) the E3L gene is under the transcriptional control of its natural promoter, whereas K3L has been placed under the control of the early/late vaccine B6 promoter. The E3L and K3L genes act to inhibit PKR activity in cells infected with ALVAC (2), allowing enhancement of the level and persistence of foreign gene expression. Additional viral vectors encompass natural host-restricted poxviruses.

Fowlpox virus (FPV) is the prototypic virus of the Avipox genus of the Poxvirus family. Replication of avipox viruses is limited to avian species (Matthews, R. E. F. (1982) Irzte3 virology 17: 42) and there are no reports in the literature of avipoxvirus causing a productive infection in any non-avian species including man. This host restriction provides an inherent safety barrier to transmission of the virus to other species and makes use of avipox virus based vectors in veterinary and human applications an attractive proposition.

FPV has been used advantageously as a vector expressing immunogens from poultry pathogens. The hemagglutinin protein of a virulent avian influenza virus was expressed in an FPV recombinant. After inoculation of the recombinant into chickens and turkeys, an immune response was induced which was protective against either a homologous or a heterologous virulent influenza virus challenge (Taylor, J. et al. (1968) Vaccine 6: 504). FPV recombinants expressing the surface glycoproteins of Newcastle Disease Virus have also been developed (Taylor, J. et al. (1990) J : Virol. 64: 1441; Edbauer, C. et al. (1990) Virology 179: 901); U.S. Pat. No. 5,766,599 incorporated herein by reference).

A highly attenuated strain of vaccinia, designated MVA, has also been used as a vector for poxvirus-based vaccines. Use of MVA is described in U.S. Pat. No. 5,185,146.

Other attenuated poxvirus vectors have been prepared via genetic modification to wild type strains of vaccinia. The NYVAC vector, for example, is derived by deletion of specific virulence and host-range genes from the Copenhagen strain of vaccinia (Tartaglia, J. et al. (1992), supra; U.S. Pat. Nos. 5,364,773 and 5,494,807 incorporated herein by reference) and has proven useful as a recombinant vector in eliciting a protective immune response against expressed foreign antigens.

Recombinant viruses can be constructed by processes known to those skilled in the art (for example, as previously described for vaccinia and avipox viruses; U.S. Pat. Nos. 4,769,330; 4,722,648; 4,603,112; 5,110,587; and 5,174,993—all of which are incorporated herein by reference).

In further embodiments of the invention, live and/or attenuated bacteria may also be used as vectors. For example non-toxicogenic Vibrio cholera mutant strains may be useful as bacterial vectors in embodiments of this invention; as described in U.S. Pat. No. 4,882,278 (disclosing a strain in which a substantial amount of the coding sequence of each of the two ctxA alleles has been deleted so that no functional cholera toxin is produced), WO92111354 (strain in which their gA locus is inactivated by mutation; this mutation can be combined in a single strain with ctxA mutations), and WO 94/1533 (deletion mutant lacking functional ctxA and attRS1 DNA sequences). These strains can be genetically engineered to express heterologous antigens, as described in WO 94/19482. (All of the aforementioned issued patent/patent applications are incorporated herein by reference). Attenuated Salmonella typhiyizurium strains, genetically engineered for recombinant expression of heterologous antigens and their use as oral immunogens are described, for example, in WO 92/11361.

As noted, those skilled in the art will readily recognize that other bacterial strains useful as bacterial vectors in embodiments of this invention include (but are not limited to) Shigella flexfzeri. Streptococcus gordoyaii, and Bacille Calmette Guerin (as described in WO 88/6626, WO 90/0594, WO 91/13157, WO 92/1796, and WO92/21376; all of which are incorporated herein by reference). In bacterial vector embodiments of this invention, a nucleic acid coding for a CEA agonist polypeptide/protein may be inserted into the bacterial genome, can remain in a free state, or be carried on a plasmid.

It is further contemplated that the invention encompasses vectors which comprise nucleic acids coding for at least one member from the group consisting of cytokines, lymphokines and immunostimulatory molecules. Said nucleic acid sequences can be contiguous with sequences coding for CEA agonist polypeptide/proteins, or encoded on distinct nucleic acids.

Cells comprising the aforementioned nucleic acids coding for CEA agonist polypeptides/proteins encompass further embodiments of the invention. These cells encompass any potential cell into which the aforementioned nucleic acid might be introduced and/or transfected (for example, bacteria. COS cells, Vero cells, chick embryo fibroblasts, tumor cells, and antigen presenting cells). The choice of process for the introduction and/or transfection into cells is dependent upon the intrinsic nature of the nucleic acid (i.e. free DNA, plasmid, incorporated into a recombinant virus), as will be known to one skilled in the an (for example, as taught in Current Protocols in Molecular Biology, F. M. Ausubel et al. (Eds.), John Wiley and Sons, Inc., N.Y., U.S.A. (1998), Chpt. 9; Molecular Cloning: A Laboratory Manual (2nd Ed.), J. Sambrook, E. F. Fritsch and T. Maniatis (Eds.), Cold Spring Harbor Laboratory Press, N.Y., U.S.A. (1989), Chpt's. 1, 2, 3 and 16).

It is well documented that the class I and class II proteins of the major histocompatibility complex (MHC) perform a central immunological function in focusing T-lymphocytes of the immune system (i.e. CD8+ and CD4° T lymphocytes). MHC class I proteins are expressed in nearly all nucleated cell types throughout the human body; MHC class II molecules are expressed mainly on antigen-presenting cells (APCs; namely, mononuclear phagocytes. Langerhans dendritic cells, and B lymphocytes). These distinct classes of cell surface molecules (i.e. class I and class II) present peptides/epitopes (derived from intracellular processing of protein antigens) to T lymphocytes (CD8+ and CD4+ T lymphocytes respectively) thus initiating both cellular and humoral immune responses. Generally, epitopes/peptides derived from alloantigens, tumor antigens or viruses will be presented in association, with MHC class I molecules; extracellular antigens/proteins will be presented in association with MHC class II molecules.

However, in some contexts endogenous antigens can also be presented in association with MHC class II molecules. These general immunological principles are well described in the art-as, for example, in Encyclopedia of Immunology (2nd Ed.), Peter J. Delves (Ed.-in-Chief), Academic Press, San Diego. U.S.A., pp. 174-8,191-8, 1108-13,1690-709 (1998). As such, embodiments of the invention contemplate cells into which has been introduced/transfected a nucleic acid coding for a CEA agonist polypeptide/protein wherein said cells express said polypeptide/protein.

As further conceived herein, embodiments of the invention also encompass cells into which has been introduced/transfected a nucleic acid coding for CEA agonist polypeptide/protein wherein said cells also express a MHC HLA molecule (i.e. class I and/or class II). In further embodiments, these cells are antigen presenting cells, possibly selected from the group consisting of mononuclear phagocytes, Langerhans dendritic cells (“dendritic cell (s)”), and B lymphocytes.

Aspects of this invention contemplate mixtures of the CEA agonist polypeptide (s)/protein (s), nucleic acids coding therefor, vectors comprising said nucleic acids, or cells comprising said nucleic acids, and at least one members selected from the group consisting of cytokines, lymphokines, immunostimulatory molecules, and nucleic acids coding therefor. Additional embodiments of this invention further encompass pharmaceutical compositions comprising the CEA agonist polypeptide/protein, nucleic acids coding therefor, erectors cells or mixtures for administration to subjects in a biologically compatible form suitable for administration in vivo. By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of eliciting an immune response in a human. A therapeutically effective amount of a substance may vary according to factors such as the disease state/health, age, sex, and weight of the recipient, and the inherent ability of the particular polypeptide, nucleic acid coding therefor, or recombinant virus to elicit a desired immune response. Dosage regima may be adjusted to provide the optimum therapeutic response. For example, several divided doses may be administered daily or on at periodic intervals, and/or the dose may be proportionally reduced as indicated by the exigences of the therapeutic situation.

The compositions described herein can be prepared bay per se known methods for the preparation of pharmaceutically acceptable compositions which can be administered to subjects, such that an effective quantity of the active substance (i.e. CEA agonist polypeptide/protein, nucleic acid coding therefor) is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic wraith physiological fluids. In this regard, reference can be made to U.S. Pat. No. 5,843,456. These compositions may further comprise an adjuvant (as described below).

Methods of inducing or eliciting an immune response in an animal directed against: a CEA protein or fragment thereof; and/or a CEA agonist polypeptide/protein of the invention; and/or a CEA epitope; and/or a modified CEA epitope; and/or cells expressing a CEA protein or fragment thereof, CEA agonist polypeptide/protein of the invention, CEA epitope, modified CEA epitope; and/or cells binding a CEA protein or fragment thereof, CEA agonist polypeptide/protein of the invention, CEA epitope, modified CEA epitope, comprising the step of; administering to said animal a CEA agonist polypeptide/protein or fragment thereof, a nucleic acid coding therefor, a vector or cell comprising said nucleic acid; mixtures thereof or pharmaceutical compositions of the aforementioned (hereinafter collectively referred to as “immunizing agent's)”, “agent's)’, or “immunogen (s)”) are also within the scope of this invention. As previously noted, an “immune response” is defined as any response of the immune system, for example, of either a cell-mediated (i.e. cytotoxic T-lymphocyte mediated) or humoral (i.e. antibody mediated) nature. These immune responses can be assessed by a number of in vivo or in vitro assays well known to one skilled in the art (for example, (but not limited to) antigen specific cytotoxicity assays, production of cytotoxins, regression of tumors expressing CEA/CEA-epitopes, inhibition of cancer cells expressing CEA/CEA epitopes).

Further embodiments of the invention encompass methods inhibiting a CEA epitope expressing carcinoma cell, in a patient comprising administering to said patient an effective amount of an immunogen of the invention. Patients with solid tumors expressing CEA (or epitopes thereof) include (but are not limited to) those suffering from colon cancer, lung cancer, pancreas cancer, endometrial cancer, breast cancer, thyroid cancer, melanoma, oral cancer, laryngeal cancer, seminoma, hepatocellular cancer, bile duct cancer, squamous cell carcinoma, and prostate cancer. As such, methods of treating patients with cancer per se encompassing the aforementioned methods of inducing an immune response and/or inhibiting a CEA epitope expressing carcinoma cell are contemplated aspects/embodiments of the invention.

As known to one of ordinary skill in the art, an animal may be immunized with an immunogen of the invention by any conventional route. This may include, for example, immunization via a mucosal surface (e.g., ocular, intranasal, oral, gastric, pulmonary, intestinal, rectal, vaginal, or urinary tract) or via a parenteral route (e.g., subcutaneous, intradermal, intramuscular, intravenous, or intraperitoneal). Preferred routes depend upon the choice of the immunogen (i.e. polypeptide vs. nucleic acid, recombinant/virus, composition formulation, etc.). The administration can be achieved in a single dose or repeated at intervals. The appropriate dosage is dependant on various parameters understood by the skilled artisans, such as the immunogen itself (i.e. polypeptide vs. nucleic acid (and more specifically type thereof)), the route of administration and the condition of the animal to be vaccinated (weight, age and the like). As such, embodiments of this invention encompass methods of eliciting immune responses in animals comprising administering an effective amount of a CEA agonist polypeptide/proteins of the invention, a nucleic acid coding therefore, vector or cells or recombinant virus comprising said nucleic acid, mixtures thereof, or pharmaceutical compositions of the aforementioned.

As previously noted, nucleic acids (in particular plasmids and/or free/naked DNA and/or RNA coding for the CEA agonist polypeptide/protein of the invention) can be administered to an animal for purposes of inducing/eliciting an immune response (for example, U.S. Pat. No. 5,589,466; McDonnell and Askari, NEJM 334: 42-45 (1996), Kowalczyk and Ertl, Cell Mot Life Sci 55: 751-770 (1999)).

Typically, this nucleic acid is a form that is unable to replicate in the target animal's cell and unable to integrate in said animal's genome. The DNA/RNA molecule encoding the CEA agonist polypeptide/protein is also typically placed under the control of a promoter suitable for expression in the animal's cell. The promoter can function ubiquitously or tissue specifically. Examples of non-tissue specific promoters include the early Cytomegalovirus (CMV) promoter (described in U.S. Pat. No. 4,168,062) and the Rous Sarcoma Virus promoter. The desmin promoter is tissue-specific and drives expression in muscle cells. More generally useful vectors have been described (i.e., WO94/21 797).

For administration of nucleic acids coding for a CEA agonist polypeptide/protein of the invention, said nucleic acid can encode a precursor or mature form of the polypeptide/protein. When it encodes a precursor form the precursor form can be homologous (for example, see Oikawa, S. et al. (1987) Supra) or heterologous. In the latter case, a eucaryotic leader sequence can be used, such as the leader sequence of the tissue-type plasminogen factor (tPA).

For use as an immunogen, a nucleic acid of the invention can be formulated according to various methods known to a skilled artisan. First, a nucleic acid can be used in a naked/free form, free of anti delivery vehicles (such as anionic liposomes, cationic lipids, microparticles, (e.g., gold microparticles), precipitating agents (e.g., calcium phosphate) or any other transfection-facilitating agent. In this case the nucleic acid can be simply diluted in a physiologically acceptable solution (such as sterile saline or sterile buffered saline) with or without a carrier. When present, the carrier preferably is isotonic, hypotonic, or weakly hypertonic, and has a relatively low ionic strength (such as provided by a sucrose solution (e.g., a solution containing 20% sucrose)).

Alternatively, a nucleic acid can be associated with agents that assist in cellular uptake. It can be, i.e., (i) complemented with a chemical agent that modifies the cellular permeability (such as bupivacaine: see, for example, WO 94/16737), (ii) encapsulated into liposomes, or (iii) associated with cal cationic lipids or silica, gold, or tungsten microparticles.

Cationic lipids are well known in the art and are commonly used for gene delivery. Such lipids include Lipofectin (also known as DOTMA (N-[1-(2,3dioleyloxy)propyl]-N,N,N-trimethylammonium chloride), DOTAP (1,2-bis(oleyloxy)-3-(trimethylammonio)propane). DDAB (dimethyldioctadecylammonium bromide), DOGS (dioctadecylamidologlycyl spermine) and cholesterol derivatives such as DC-Chol (3beta-(N—(N′,N′-dimethyl aminomethane)carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleylphosphatidylethanolamine), as for example, described in WO 90/11092.

Other transfection-facilitating compounds can be added to a formulation containing cationic liposomes. A number of them are described in, for example, WO 93/18759, WO 93/19768, WO 94/25608, and WO 95/2397. They include, i.e., Spennine derivatives useful for facilitating the transport of DNA through the nuclear membrane (see, for example, WO 93/18759) and membrane-permeabilizing compounds such as GALA, Gramicidine 5, and cationic bile salts (see, for example, WO93/19768).

Gold or tungsten microparticles can also be used for gene delivery (as described in WO 91/359 and WO 93/17706). In this case, the microparticle-coated polynucleotides can be injected viaintradermal orintraepidermal routes using a needleless injection device (“gene gun”), such as those described, for example, in U.S. Pat. No. 4,945,b50, U.S. Pat. No. 5,015,580, and WO 94/24263.

Anionic and neutral liposomes are also well-known in the art (see, for example, Liposomes: A Practical Approach, RPC New Ed, IRL Press (1990), for a detailed description of methods for making liposomes) and are useful for delivering a large range of products, including nucleic acids.

Particular embodiments of the aforementioned methods (i.e. to induce/elicit immune responses and/or to inhibit a CEA epitope expressing carcinoma cell in a patient) encompass prime-boost protocols for the administration of immunogens of the invention. More specifically, these protocols encompass (but are not limited to) a “priming” step with a particular/distinct form of immunogen (i.e. nucleic acid (for example, plasmid, bacterial/viral/free or naked) coding for an immunogen, or vector (i.e. recombinant virus, bacteria) comprising said nucleic acid) followed by at least one “boosting” step encompassing the administration of an alternate (i.e. distinct from that used to “prime”) form of the immunogen (i.e. protein or fragment thereof (for example, epitope-peptide), nucleic acid coding for an immunogen (or fragment thereof), or vector comprising said nucleic acid). Examples of “prime-boost” methodologies are known to those skilled in the art (as taught, for example, in PCT published applications WO 00/00216, WO 98/58956, WO 98/56919, WO 97/39771). One advantage of said protocols is the potential to circumvent the problem of generating neutralizing immune responses to viral vectors per se wherein is inserted incorporated nucleic acids encoding the immunogen or fragments thereof (see for example, R. M. Conty et al. (2000) Clin. Cancer Res. 6: 34-41).

As is well known to those of ordinary skill in the art, the ability of an immunogen to induce/elicit an immune response can be improved if, regardless of administration formulation (i.e. recombinant virus, nucleic acid, polypeptide), said immunogen is coadministered with an adjuvant. Adjuvants are described and discussed in “Vaccine Design—the Subunit and Adjuvant Approach” (edited by Powell and Newman, Plenum Press, New York, U.S.A., pp. 61-79 and 141-228 (1995)). Adjuvants typically enhance the immunogenicity of an Immunogen but are not necessarily immunogenic in and of themselves. Adjuvants may act by retaining the immunogen locally near the site of administration to produce a depot effect facilitating a slow, sustained release of immunizing agent ale cells of the immune system. Adjuvants can also attract cells of the, immune system to an immunogen depot and stimulate such cells to elicit immune responses. As such, embodiments of this invention encompass compositions further comprising adjuvants.

Desirable characteristics of ideal adjuvants include:

1) lack of toxicity:

2) ability to stimulate a long-lasting immune response;

3) simplicity of manufacture and stability in long-term storage;

4) ability to elicit both cellular and humoral responses to antigens administered by various routes, if required:

5) synergy with other adjuvants;

6) capability of selectively interacting with populations of antigen presenting cells (APC);

7) ability to specifically elicit appropriate TR1 or TH2 cell-specifib immune responses; and

8) ability to selectively increase appropriate antibody isotype levels (for example, IgA) against antigens/immunogens.

However, many adjuvants are toxic and can cause undesirable side effects, thus making them unsuitable for use in humans and many animals. For example, some adjuvants may induce granulomas, acute and chronic inflammations (i.e. Freund's complete adjuvant (FCA)), cytolysis (i.e. saponins and pluronic polymers) and pyrogenicity, arthritis and anterior uveitis (i.e. muramyl dipeptide (MDP) and lipopolysaccharide (LPS)). Indeed, only, aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established.

Notwithstanding, it does have limitations. For example, alum is ineffective for influenza vaccination and inconsistently elicits a cell mediated immune response with other immunogens. The antibodies elicited by alum-adjuvanted antigens are mainly of the IgG1 isotype in the mouse, which may not be optimal for protection in vaccination contexts.

Adjuvants may be characterizedas “intrinsic” or “extrinsic”. Intrinsic adjuvants (such as lipopolysaccharides) are integral and normal components of agents which in themselves are used as vaccines (i.e. killed or attenuated bacteria), Extrinsic adjuvants are typically nonintegral immunomodulators generally linked to antigens in a noncovalent manner, and are formulated to enhance the host immune response.

In embodiments of the invention, adjuvants can be at least one member chosen from the group consisting of cytokines, lymphokines, and co-stimulatory molecules.

Examples include (but are not limited to) interleukin 2, interleukin 12, interleukin 6, interferon gamma, tumor necrosis factor alpha, GM-CSF, 87.1, 87.2, ICAM-1, LFA3, and CD72. Particular embodiments specifically encompass the use of GM-CSF as an adjuvant (as taught, for example, in U.S. Pat. Nos. 5,679,356, 5,904,920, 5,637,483, 5,759,535, 5,254,534, European Patent Application EP 211684, and published PCT document WO97/28816 all of which are herein incorporated by reference).

A variety of potent extrinsic adjuvants have been described. These include (but are not limited to) saponins complexed to membrane protein antigens (immune stimulating complexes), pluronic polymers with mineral oil, killed mycobacteria and mineral oil, Freund's complete adjuvant bacterial products such as muramyl dipeptide (MDP) and lipopolysaccharide (LPS), as well as lipid A, and liposomes.

The use of saponins per se as adjuvants is also well known (Lacaille-Dubois, M. and Wagner, H (1996) Phytomedicine 2: 363). For example, Quil A (derived from the bark of the South American tree, Quillaia Saponaria Molina) and fractions thereof has been extensively described (i.e. U.S. Pat. No. 5,057,540; Kensil, C. R.

(1996) Crit Rev Ther Drug Carrier Syst 12: 1: and European Patent EP 362279). The haemolytic saponins Q521 and QS17 (HPLC purified fractions of Quil A) have been described as potent systemic adjuvants (U.S. Pat. No. 5,057,540: European Patent EP 362279). Also described in these references is the use of QS7 (a non-haemolytic fraction of Quil-A) which acts as a potent adjuvant for systemic vaccines. Use of QS21 is further described in Kensil et al. ((1991) J. Immunol 146 :431).

Combinations of 0521 and polysorbate or cyclodextrin are also known (WO9910008). Particulate adjuvant systems comprising fractions of Quil A (such as QS21 and QS7) are described in WO 9633739 and WO 9611711.

Another preferred adjuvant/immunostimulant is an immunostimulatory oligonucleotide containing unmethylated CpG dinucleotides (“CpG”). CpG is an abbreviation, or cytosine-guanosine dinucleotide motifs present in DNA. CpG is known in the art as being an adjuvant when adminstered by both systemic and mucosal routes (WO 9002555; European Patent EP468520; Davies et al. (1998). J. Immunol. 160: 87:McCluskie and Davis (1998) J. Immunol 161 : 4463). In a number of studies, synthetic oligonucleotides derived from BCG gene sequences have also been shown to be capable of inducing immunostimulatory effects (both in vitro and in vivo: Krieg, (1995) Nature 374: 546). Detailed analyses of immunostimulatory oligonucleotide sequences has demonstrated that the CG motif must be in a certain sequence context, and that such sequences are common in bacterial DNA but are rare in vertebrate DNA. (For example, the immunostimulatory sequence is often: purine, purine, C, G, pyrimidine, pyrimidine, wherein the CG motif is not methylated; however other unmethylated CpG sequences are known to be immunostimulatory and as such may also be used in the present invention.) As will be evident to one of normal skill in the art, said CG motifs/sequences can be, incorporated into nucleic acids of the invention per se, or reside on distinct nucleic acids.

A variety of other adjuvants are taught in the art, and as such are encompassed by embodiments of this invention. U.S. Pat. No. 4,855,283 granted to Lockhoff et al. (incorporated herein by reference) teaches glycolipid analogues and their use as adjuvants. These include N-glycosylamides, N-glycosylureas and Nglycosylcarbamates, each of which is substituted in the sugar residue by an amino acid, as immuno-modulators or adjuvants. Furthermore, Lockhoff et al. ((1991) Cheni. mt. Ed. Engl. 30: 1611) have reported that N-glycolipid analogs displaying structural similarities to the naturally-occurring glycolipids (such as glycophospholipids and glycoglycerolipids) are also capable of eliciting strong immune responses in both herpes simplex virus vaccine and pseudorabies virus vaccine.

U.S. Pat. No. 4,258,029 granted to Moloney (incorporated herein by reference) teaches that octadecyl tyrosine hydrochloride (OTH) functions as an adjuvant when complexed with tetanus toxoid and formalin inadtivated type I, II and III poliomyelitis virus vaccine. Nixon-George et al. ((1990) J. Immunol. 14: 4798) have also reported that octadecyl esters of aromatib amino acids complexed with a recombinant hepatitis B surface antigen enhanced the host immune responses against hepatitis B virus.

Adjuvant compounds may also be chosen from the polymers of acrylic or methacrylic acid and the copolymers of maleic anhydride and alkenyl derivative.

Adjuvant compounds are the polymers of acrylic or methacrylic acid which are cross-linked, especially with polyalkenyl ethers of sugars or polyalcohols. These compounds are known by the term carbomer (Pharmeuropa Vol. 8 No. 2, June 1996). Preferably, a solution of adjuvant according to the invention, especially of carbomer, is prepared in distilled water, preferably in the presence of sodium chloride, the solution obtained being at acidic pH. This stock solution is diluted by adding it to the desired quantity (for obtaining the desired final concentration), or a substantial part thereof of water charged with NaCl, prefer ably physiological saline (NaCl 9 g/l) all at once in several portions with concomitant or subsequent neutralization (pH 7.3 to 7.4), preferably with NaOH. This solution at physiological pH will be used as it is for mixing with the immunizing agent; said mixture being amenable to storage in the freeze-dried, liquid or frozen form.

Persons skilled in the art can also refer to U.S. Pat. No. 2,909,462 (incorporated herein by reference) which describes adjuvants encompassing acrylic polymers cross-linked with a polyhydroxylated compound having at least 3 hydroxyl groups (preferably not more than 8), the hydrogen atoms of the at least three hydroxyls being replaced by, unsaturated aliphatic radicals having at least 2 carbon atoms. The preferred radicals are those containing from 2 to 4 carbon atoms (e.g. vinyls, allyls and other ethylenically unsaturated groups). The unsaturated radicals may themselves contain other substituents such as methyl. The products sold under the name Carbopol (BF Goodrich, Ohio, USA) are particularly appropriate. They are cross-linked with allyl sucrose or with allyl pentaerythritol. Among them, there may be mentioned Carbopol (for example, 974P, 934P and 971 P). Among the copolymers of maleic anhydride and alkenyl derivative, the copolymers EMA (Monsanto; which are copolymers of maleic anhydride and ethylene, linear or crosslinked, (for example cross-linked with divinyl ether)) are preferred. Reference may be made to J. Fields et al. ((1960) Nature 186: 778) for a further description of these chemicals (incorporated (herein by reference).

In further aspects of this invention, adjuvants useful for parenteral administration of immunizing agent include aluminum compounds (such as aluminum hydroxide, aluminum phosphate, and aluminum hydroxy phosphate; but might also be a salt of calcium, iron or zinc, or may be an insoluble suspension of acylated tyrosine, or acylated sugars, cationically or anionically derivatised polysaccharides, or polyphosphazenes). The antigen can be precipitated with, or adsorbed onto, the aluminum compound according to standard protocols well known to those skilled in the art.

Other adjuvants encompassed by embodiments of this invention include lipid A (in particular 3-de-0-acylated monophosphoryl lipid A (3D-MPL). 3D-MPL is a well known adjuvant manufactured by Ribilrmnunochem, Mont. Chemically it is often supplied as a mixture of 3-de-0-acylated monophosphoryl lipid A with 4, 5, or 6 acylated chains. It can be prepared by the methods taught in GB 21222048. A preferred form of 3D-MPL is in the form of a particulate formulation having a particle size less than 0.2 pm in diameter (European Patent EP689454).

Adjuvants for mucosal immunization may include bacterial toxins (e.g., the cholera toxin (CT), the E. coli heat-labile toxin (LT), the Clostriliuill difficile toxin

A and the pertussis toxin (PT), or combinations, subunits, toxoids, or mutants thereof). For example, a purified preparation of native cholera toxin subunit B (CTB) can be of use. Fragments, homologs, derivatives, and fusion to any of these toxins are also suitable, provided that they retain adjuvant activity. A mutant having reduced toxicity may be used. Mutants have been described (e.g., in WO 95/17211 (Arg-7-Lys CT mutant), WO 96/6627 (Arg 192-Gly LT mutant), and WO 95134323 (Arg-9-Lys and Glu-129-Gly PT mutant)). Additional LT mutants include, for example Ser-63-Lys, Ala-69-Gly, Glu-110-Asp, and Glu-112-Asp mutants. Other adjuvants (such as a bacterial monophosphoryl lipid A (MPLA)) of various sources (e.g., E. coli, Salmonella minnesota, Salmonella typhimurium, or Shigella flexneri) can also be used in the mucosal administration of immunizing agents.

Adjuvants useful for both mucosal and parenteral immunization include polyphosphazene (for example, WO 95/2415), DC-chol (3b-(N—(N′,N′-dimethyl aminomethane)-carbamoyl) cholesterol (for example. U.S. Pat. Nos. 5,283,185 and O 96/14831) and QS-21 (for example, WO 88/9336).

Adjuvants/immunostimulants as described herein may be formulated together with carriers, such as for example liposomes, oil in water emulsions, and/or metallic salts including aluminum salts (such as aluminum hydroxide). For example, 3D-MPL may be formulated with aluminum hydroxide (as discussed in EP 689454) or oil in water emulsions (as discussed in WO 9517210); QS21 may be advantageously formulated with cholesterol containing liposomes (as discussed in WO 9633739), in oil water emulsions (as discussed in WO 9517210) or alum (as discussed in WO 9815287). When formulated into vaccines, immunostimulatory oligonucleotides (i.e. CpGs) are generally administered in free solution together with free antigen (as discussed in WO 9602555; McCluskie and Davis (1998) Supra), covalently conjugated to an antigen (as discussed in WO 9816247), or formulated with a carrier such as aluminum hydroxide or alum (as discussed in Davies et al. Supra Brazolot-Millan et al (1998) Proc. Natl. Acad. Sci. 95: 15553).

Combinations of adjuvants/immunostimulants are also within the scope of this invention. For example, a combination of a monophosphoryl lipid A and a saponin derivative (as described in WO 9400153, WO 9517210, WO 9633739, WO9856414, WO 9912565, WO 9911214) can be used, or more particularly the combination of QS21 and 3D-MPL (as described in WO 9400153). A combination of an immunostimulatory oligonucleotide and a saponin (such as QS21), or a combination of monophosphoryl lipid A (preferably 3D-MPL) in combination with an aluminum salt also form a potent adjuvant for use in the present invention.

The following non-limiting examples are illustrative of the present invention:

### EXAMPLES

### First Set of Examples

CAP1 Substituted Peptides

Several factors were considered in deciding which positions to examine for effects on T cell activity. Sequencing and mapping experiments have defined a binding motif in which position 2 and the C-terminal (position 9 or 10) are critical for peptide presentation by BLA-A2 (for review, see 31). In addition, Tyr at position 1 has been identified as an effective secondary anchor (20, 32). Since the CEA peptide CAP1 already has the preferred amino acids at these three positions these residues were not altered. Instead, we focused attention on residues predicted to interact with the TCR in the hope of finding analogs that would stimulate human CAP1-specific cytotoxic T cells. X-ray crystallographic studies of several peptides bound to soluble HLA-A2 suggest that all binding peptides assume a common conformation in the peptide binding groove (33). When five model peptides were examined, residues 5 through 8 protrude away, from the binding groove and are potentially available for binding to a TCR. Therefore a panel of 80 CAP1 analog peptides was produced in which the residues at positions 5 through 8 (p5-p8) were synthesized with each of the 20 natural amino acids. The peptides are designated CAP1-pAA, where p refers to the position in the peptide and AA refers to the replacement amino acid, using the single letter amino acid code. i.e., CAP1-6D in which position 6 is occupied by aspartic acid.

Enhanced CTL Sensitivity of Targets to CAP1-6D Analog

The effects of these amino acid substitutions on potential TCR recognition was studied using a CAP1 specific, HLA-A2 restricted human CTL line designated T-Vac8. Briefly, T-Vac8 was generated as described in Materials and Methods by in vitro peptide stimulation of PBMC from a patient that had been administered rV-CEA. For initial screening, T-Vac8 was used in a cytotoxicity assay to measure ¹¹¹In release from labeled C1R-A2 cells incubated with each member of the peptide panel (at three peptide concentrations). Spontaneous release from the targets (in the absence of T-Vac8) was determined for each individual peptide.

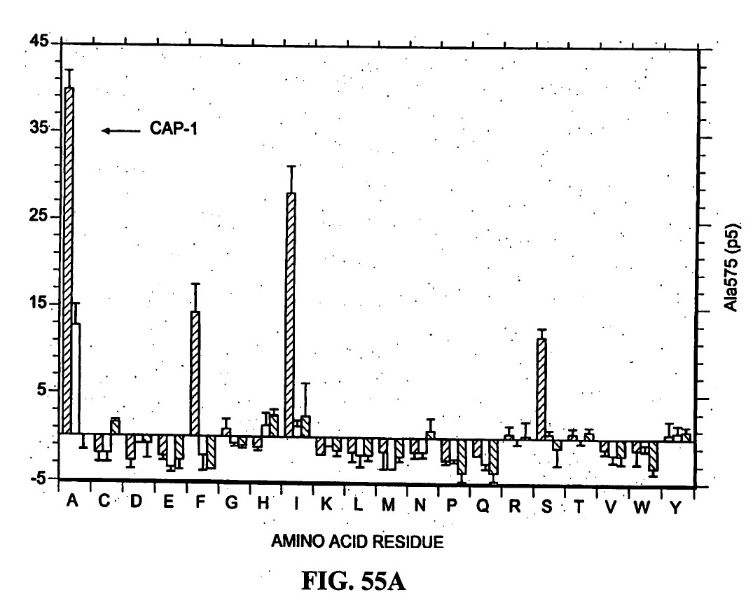

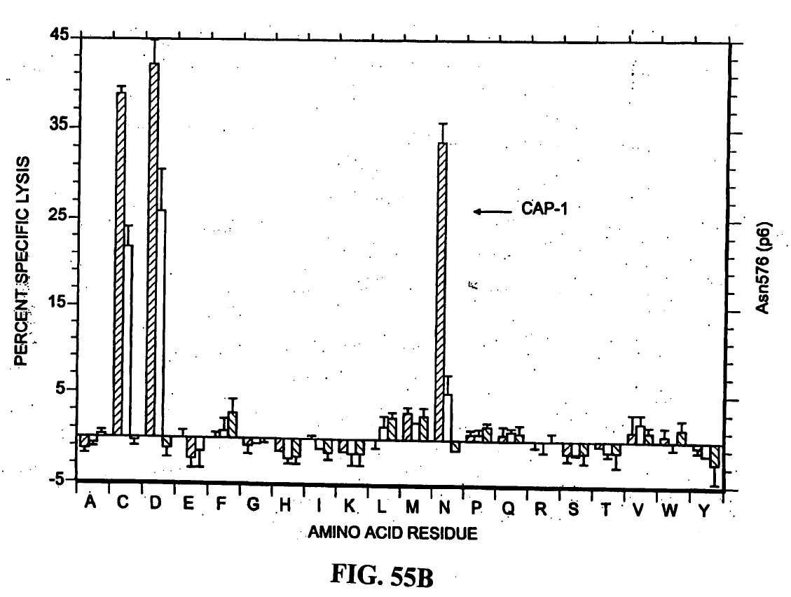

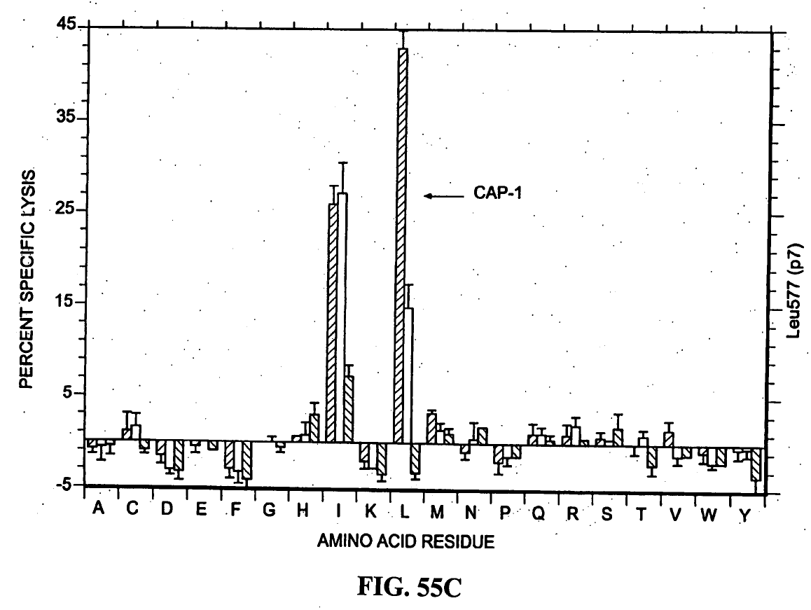

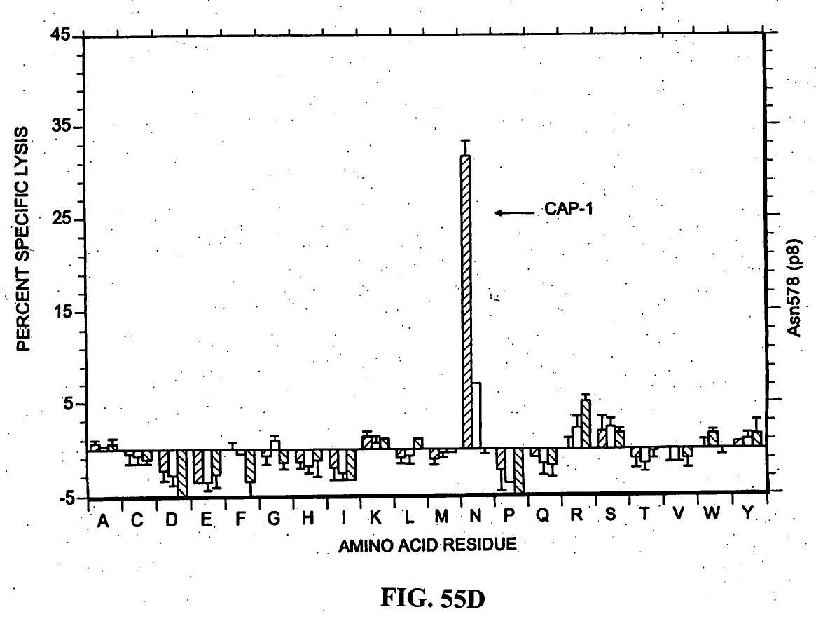

The results are presented in FIGS. 55A-D. Of the 80 single amino acid substitutions, most failed to activate cytotoxicity of T-Vac8. However, six independent substitutions preserved reactivity. At position 5, three analogs CAP1-5F, CAP1-5I and CAP1-5S provided stimulation, albeit at reduced levels compared to CAP1 itself. At position 6 the substitutions CAP1-6C and CAP1-6D activated T-Vac8 cytotoxicity and seemed to be equal to or better than CAP1 since they were more active at the intermediate (0.1 .mu.g/ml) peptide concentration. At position 7 analog CAP1-71 also appeared to be active. Finally, at position 8, no analogs were able to sensitize targets to lysis by T-Vac8. The two most active analogs (CAP1-6D and CAP1-71) were then analyzed in detail, omitting CAP1-6C due to concern for disulfide formation under oxidizing conditions.

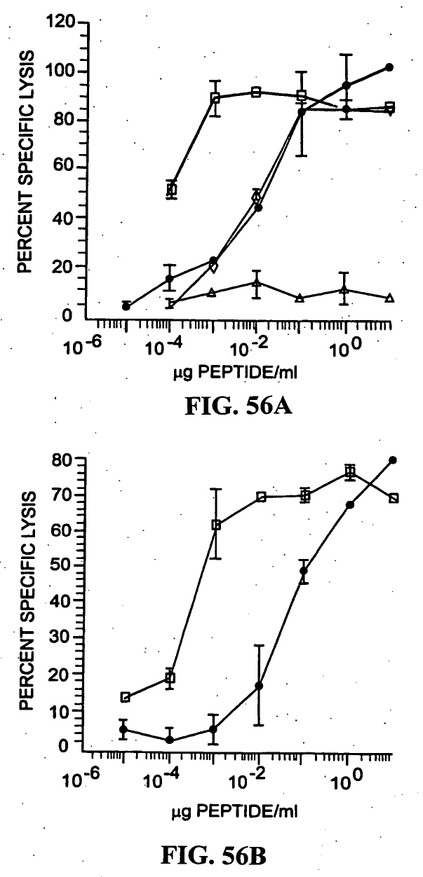

Purer preparations (90-96% pure) of native CAP1 and the analogs CAP1-6D and CAP1-71 were synthesized and compared in a CTL assay over a wider range of peptide concentrations, using two different cell lines as targets (FIGS. 56A-B). Employing T2 cells analog CAP1-6D was at least 10² times more effective than native CAP1. CAP1-6D lytic activity was at ½ maximum at 10⁻⁴ .mu.g/ml (FIG. 56A). In contrast, the CAP1-71 analog and the native CAP1 sequence were comparable with each other over the entire range of peptide titration and showed half maximal lysis at 10⁻² .mu.g/ml. Employing the C1R-A2 cells as targets, CAP1-6D was similarly between 10² and 10³ more effective in mediating lysis than CAP1 (FIG. 56B).

The CAP1-6D peptide was also tested using a second CEA-specific T cell line, T-Vac24 (11). This line was generated from rV-CEA post vaccination PBMC of a different carcinoma patient by in vitro stimulation with the native CAP1 peptide; in contrast to predominantly CD8+ T-Vac8, T-Vac24 has a high percentage of CD4+ CD8+ double positive cells (11). In a 4 hr 111In release assay employing T-Vac24, CAP1-6D was slightly more effective (30% lysis) than the native CAP1 sequence (20% lysis); although the differences were not as pronounced as with T-Vac8, the increased sensitivity to the analog was seen in three separate experiments. The analog peptide clearly engaged the lytic apparatus of a second CAP1 specific CTL.

Analogs and Native Peptide Show Identical Presentation by HLA-A2

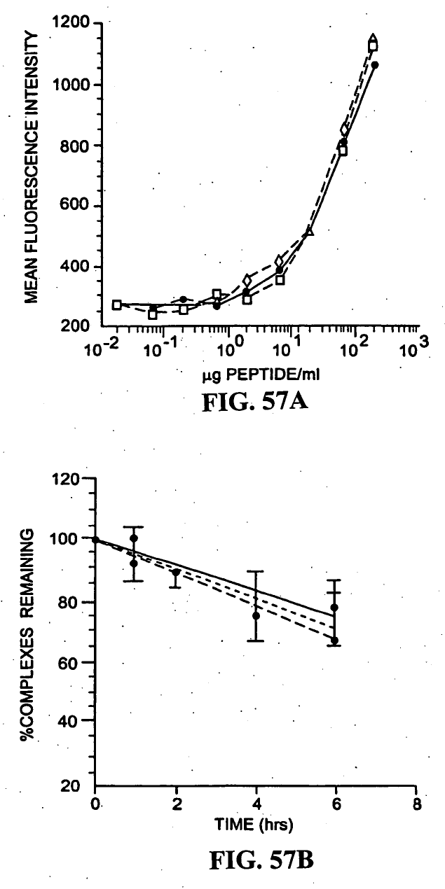

The increased effectiveness of CAP1-6D in CTL assays could be due to better presentation by the target. The most active CAP1 analogs were tested for binding to HLA-A2 by measuring cell surface HLA-A2 in the transport-defective human cell line T2. When compared over a 4-log range of concentrations, native CAP1 and the two analogs CAP1-6D and CAP1-71 all presented equally on T2 cells (FIG. 57). In addition, dissociation experiments indicate that the HLA-A2 complexes that form with the 3 peptides show no appreciable differences in stability (FIG. 57—insert). When peptide-pulsed T2 cells were washed free of unbound peptide, the half lives of cell surface peptide-A2 complexes were 12.5 hrs (CAP1), 9.7 hrs (CAP1-6D), and 10.8 hrs (CAP1-71). If anything, the complex formed with the agonist peptide seems slightly less stable. Since there are no differences in binding to HLA-A2, the improved, effectiveness of CAP1-6D in the CTL assays appears to be due to better engagement by the T cell receptor, a behavior characteristic of an enhancer agonist peptide.

Human CTL Generated With CAP1-6D also Recognize Native CAP1 The CAP1-6D agonist might be useful in both experimental and clinical applications if it can stimulate growth of CEA-specific CTL from patients with established carcinomas. In one experiment, post rV-CEA immunization PBMC from cancer patient Vac8 (the same rV-CEA patient from whom T-Vac8 CTL were established) were stimulated in vitro with CAP1-6D and after 5 rounds of stimulation were assayed for CTL activity against targets coated with CAP1 or CAP1-6D. This new line demonstrated peptide-dependent cytotoxic activity against target cells coated with either CAP1-6D or native CAP1 (Table 1A).

Post immunization PBMC from patients Vac8 and Vac24 were already shown to produce CTL activity when stimulated with CAP1 while preimmunization PBMC were negative (11, 34). Moreover, previous attempts to stimulate CTL activity from healthy, non-immunized donors with the CAP1 peptide were unsuccessful. To test if the agonist peptide is indeed more immunogenic than native CAP1 we attempted to generate CTL from healthy, non-immunized donors using CAP1-6D. HLA-A2+ PBMC from apparently healthy individuals were stimulated in vitro either with CAP1 or the CAP1-6D agonist. After 4 cycles of in vitro stimulation, cell lines were assayed for specificity against C1R-A2 cells pulsed with either CAP1 or CAP1-6D.

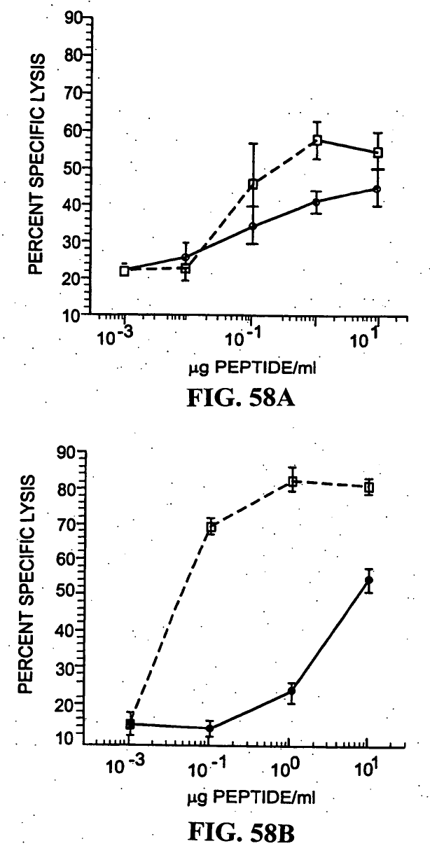

While stimulations with CAP1 or the CAP1-6D peptide produced T cell lines, peptide specific lysis was only obtained in the lines generated with CAP1-6D. Two independent T cell lines from different donors were derived using CAP1-6D and were designated T-N1 and T-N2 (FIG. 58A and FIG. 58B respectively). Both CTL lines lyse C1R-A2 targets pulsed with native CAP1 peptide. However, more efficient lysis is obtained using the CAP1-6D agonist. T-N1 CTL recognizes CAP1-6D at a 3-10 fold lower peptide concentration than CAP1 and T-N2 recognizes the agonist 100 fold better than CAP1. In contrast, attempts to generate a CTL cell line from normal donors by stimulation with CAP1 resulted in lines with no peptide-dependent lysis and loss of the lines in early stimulation cycles. Thus the attempts to generate T cell lines using the two peptides demonstrated the ability of CAP1-6D to act as an agonist not only at the effector stage, in the lysis of targets, but also in selecting T cells that are presumably in low precursor frequencies.

To determine whether CTL established with the agonist could be maintained on the native CAP1 sequence, T-N1 was cultured for 5 cycles as described using CAP1-6D, then divided into duplicate cultures maintained on the agonist or on CAP1. T-N1 continued to grow when stimulated with either peptide and responded to both peptides in CTL assays. Phenotypic analysis of the TCR usage in T-N1 indicates that the majority of cells (71%) utilize Vβ12, with a minor population that utilize Vβ5.3 (Table 2A). The same pattern of TCR Vβ usage was observed after switching the cells to CAP1 for 5 more stimulation cycles. This Vβ usage pattern was distinct from that of T-Vac8. These data indicate that the agonist can select T cells that are probably in low precursor frequency but that once selected, such CTL could be maintained with the native CAP1.

CTL Generated with CAP1-6D Specifically Lysed CEA.⁺, HLA-A2⁺ Tumor Cells

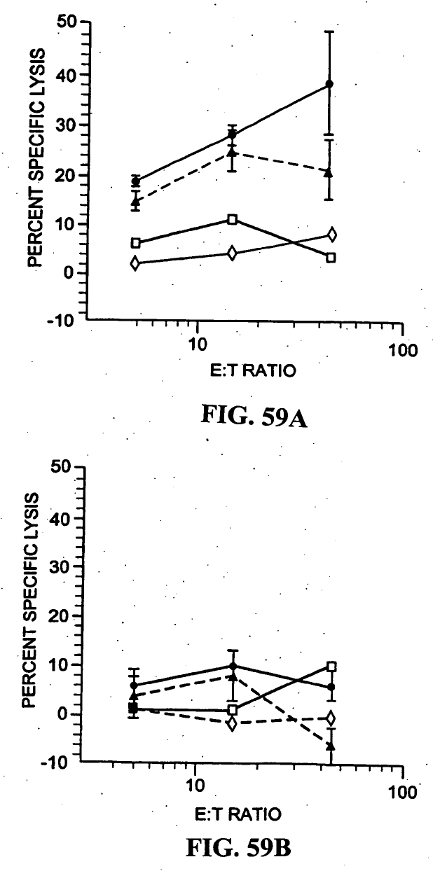

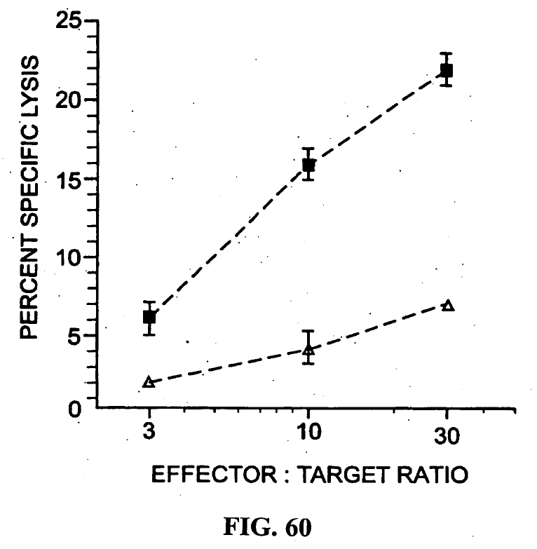

Studies were conducted to determine the ability of CTL generated with the enhancer agonist to lyse human tumor cells endogenously expressing CEA. T-N1 and T-N2 were tested against a panel of tumor cells that are CEA⁺/A2⁺ (SW480 and SW1463), CEA+/A2⁻ (SW1116) or CEA⁻/A2⁺ (CaOV3 and SKmel24). A T cell line (T-N2) from the normal donor was tested for the ability to lyse tumor targets endogenously expressing CEA. T-N2 CTL generated with the agonist lysed tumor cells expressing both CEA and HLA-A2 while exhibiting no titratable lysis of CEA⁻/A2⁺ SKmel24 melanoma cells (FIG. 59A). No significant lysis of K562 was observed. In contrast, cell lines generated by stimulation with native CAP1 showed no detectable antitumor activity (FIG. 59B). The HLA-A2.1 restriction of the T-N2 response to CEA positive tumor targets was further demonstrated by the specific lysis of a CEA positive BLA-A2.1 negative tumor cell, SW837 after infection with a vaccinia-A2.1 construct (rV-A2.1). No lysis was observed when SW837 targets were infected with the control wild type vaccinia without the A2.1 transgene (FIG. 60).

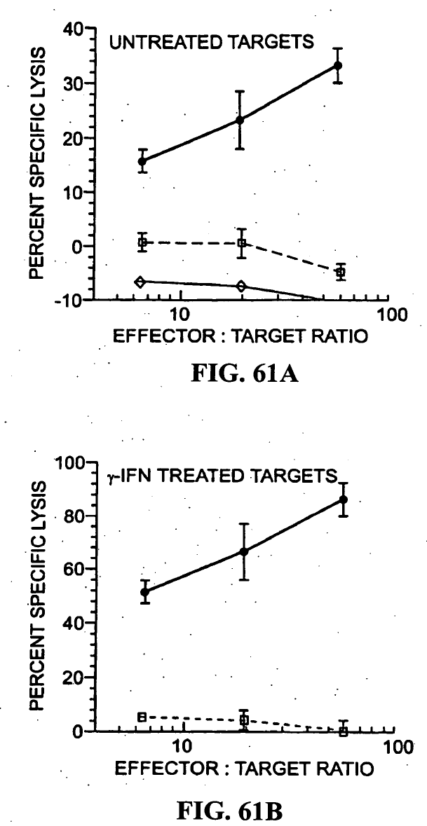

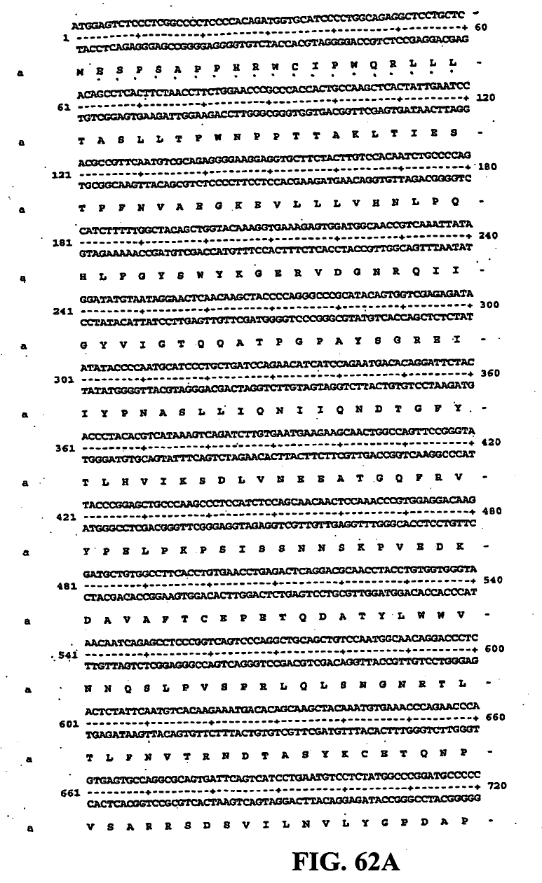

The ability of a CTL line (T-N1) derived from a second donor to kill carcinoma targets expressing endogenous CEA is shown in FIGS. 61A and 61B. T-N1 specifically lysed SW480 tumor cells. This is dramatically enhanced to 79% lysis by pretreatment of the tumor cells wraith IFN-.gamma., a treatment that increases the cell surface density of both BLA-A2 and CEA. The specificity of T-N1 killing is demonstrated by its inability to lyse CEA⁻/A2⁺ tumors such as the ovarian derived tumor CaOV3, the melanoma tumor SKmel24; or the NK target K562. Finally, restriction by HLA-A2 is demonstrated by the failure of T-N1 to lyse CEA⁺/A2⁻ SW1116 tumor cells (FIG. 61A), even after IFN-.gamma. treatment (FIG. 61B)

TABLE 1 A
CTL generated by stimulation with the CAP1-6D analog
from PBMC of an HLA-A2 patient immunized with rVCEA

| | % Lysis | | |
|---|---|---|---|
| Effector/target ratio | no peptide | CAP1 | CAP1-6D |
| 25:1 | 10% | 41% | 40% |
| 6.25:1 | 0.5% | 38% | 46% |

T cells were assayed after 5 in vitro stimulations. Cytotoxic activity was determined in 4 hour release assay with peptide at 25 μg/ml.

TABLE 2 A
TCR usage of CTL line established on CAP1-6D agonist

| | |
|---|---|
| T-N1^{b} | T-N1^{c} |

| TCR usage^{a} | % positive | MFI | % positive | MFI |
|---|---|---|---|---|
| Vβ12 | 71 | 83 | 70 | 83 |
| Vβ5.3 | 18 | 47 | 20 | 57 |
| Vβ3.1 | 6 | 48 | 8 | 46 |
| Vβ8 | 3 | 30 | 6 | 26 |
| Vβ13.6 | 2 | 19 | 3 | 39 |
| Vβ12.1 | 3 | 43 | 3 | 40 |

^{a}Determined by FACS analysis using a panel of 19 Vβ and 2 Vc antibodies (see Materials and Methods). Only positively staining antibodies are shown.
^{b}CTL line selected and maintained on agonist CAP1-6D as described in the Materials and Methods section.
^{c}CTL line selected on agonist CAP1-6D for 5 stimulation cycles, and maintained on CAP1 for an additional 10 cycles.

### Second Set of Examples

### Example 1

Generation of Recombinant Vaccinia, rV-TRICOM(mu1) No. vT171

The origin of vaccinia parental virus is the New York City Board of Health strain and was obtained by Wyeth from the New York City Board of Health and passaged in calves to create the Smallpox Vaccine Seed. Flow Laboratories received a lyophilized vial of the Smallpox Vaccine Seed, Lot 3197, Passage 28 from Drs. Chanock and Moss (National Institutes of Health). This seed virus was ether-treated and plaque-purified three times.

For the generation of rV-TRICOM (mu1), a plasmid vector, designated pT5032 was constructed to direct insertion of the foreign sequences into the M2L (30K) gene, which is located in the Hind III M region of the vaccinia genome. The murine LFA-3 gene is under the transcriptional control of the vaccinia 30K (M2L) promoter (34), the murine ICAM-1 gene is under the control of the vaccinia I3 promoter (18), and the murine B7.1 gene is under the control of the synthetic early/late (sE/L) promoter (32). These foreign sequences are flanked by DNA sequences from the Hind III M region of the vaccinia genome (see FIG. 1). These flanking sequences include the vaccinia K1L host range gene (33). A derivative of the Wyeth strain of vaccinia was used as the parental virus in the construction of recombinant vaccinia virus. This parental virus, designated vTBC33, lacks a functional K1L gene and thus cannot efficiently replicate on rabbit kidney RK₁₃ cells (38). The generation of recombinant vaccinia virus was accomplished via homologous recombination between vaccinia sequences in the vTBC33 vaccinia genome and the corresponding sequences in pT5032 in vaccinia-infected RK₁₃ cells transfected with pT5032. Recombinant virus, designated vT171, was selected by growth on RK₁₃ cells (ATCC, CCL 37). Plaques were picked from the cell monolayer and their progeny were further propagated. Two rounds of plaque isolation and replating on RK₁₃ cells resulted in the purification of the desired recombinant. The genomic structure of recombinant vT171 is depicted in FIG. 4A.

### Example 2

Generation of Recombinant Vaccinia, rV-TRICOM(mu2) No. vT199

For the generation of rV-TRICOM(mu2), a plasmid vector, designated pT5047, was constructed to direct insertion of the foreign sequences into the thymidine kinase (TK) gene, which is located in the Hind III J region of the vaccinia genome. The murine B7.1 gene is under the control of the sE/L promoter, the murine LFA-3 gene is under the transcriptional control of the I3 promoter, and the murine ICAM-1 gene is under the control of the vaccinia 7.5K promoter (39). In addition, the E. coli lacZ gene, under the control of the fowlpox virus C1 promoter (A15) is included as a screen for recombinant progeny. These foreign sequences are flanked by DNA sequences from the Hind III J region of the vaccinia genome (see FIG. 2). A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT5047 in vaccinia-infected Hu143TK cells (Bacchetti and Graham 1977) transfected with pT5047. Recombinant virus was identified using selection for TK virus in the presence of bromodeoxyuridine (BudR) in combination with a chromogenic assay, performed on viral plaques in situ, that detects expression of the lacZ gene product in the presence of halogenated indolyl-beta-D-galactoside (Bluo-gal), as described previously (31). Viral plaques expressing lacZ appeared blue against a clear background. Positive plaques, designated vT199, were picked from the cell monolayer and their progeny were replated under the selective conditions described above. In other recombinant viruses selected and purified in this manner, the only plaques that appeared under these selective conditions were blue, and these blue plaques were readily isolated and purified. However, in the case of vT199, only white plaques were observed at the second round of plaque-purification; no blue plaques were apparent. A new set of blue plaques were picked and replated; again, only white plaques were observed at the second round of plaque-purification. A final attempt, using yet another set of blue plaques, yielded both blue and white plaques after the second round of plaque-purification. Blue plaques were selected and replated. To additional rounds of plaque-purification (a total of four rounds) yielded recombinant viruses uses that were 100% blue. The genomic structure of recombinant vT199 is depicted in FIG. 4B.

### Example 3

Generation of Recombinant Vaccinia rV-TAA/TRICOM(mu)

For the generation of rV-TAA/TRICOM(mu), a plasmid vector is constructed to direct insertion of the foreign sequences into the vaccinia genome. The TAA gene, the murine LFA-3 gene, the murine ICAM-1 gene, and the murine B7.1 gene are under the control of a multiplicity of promoters. These foreign sequences are flanked by DNA sequences from the vaccinia genome, into which the foreign sequences are to be inserted. The generation of recombinant vaccinia virus is accomplished via homologous recombination between vaccinia sequences in the vaccinia genome and the corresponding sequences in the plasmid vector in vaccinia-infected cells transfected with the plasmid vector. Recombinant plaques are picked from the cell monolayer under selective conditions and their progeny are further propagated. Additional rounds of plaque isolation and replating result in the purification of the desired recombinant virus.

### Example 4

Generation of Recombinant Vaccinia rV-MUC-1/TRICOM(mu)

For the generation of rV-MUC-1/TRICOM(mu), a plasmid vector is constructed to direct insertion of the foreign sequences into the vaccinia genome. The MUC-1 gene, the murine LFA-3 gene, the murine ICAM-1 gene, and the murine B7.1 gene are under the control of a multiplicity of promoters. These foreign sequences are flanked by DNA sequences from the vaccinia genome into which the foreign sequences are to be inserted. The generation of recombinant vaccinia virus is accomplished via homologous recombination between vaccinia sequences in the vaccinia genome and the corresponding sequences in the plasmid vector in vaccinia-infected cells transfected with the plasmid vector. Recombinant plaques are picked from the cell monolayer under selective conditions and their progeny are further propagated. Additional rounds of plaque isolation and replating result in the purification of the desired recombinant virus.

### Example 5

Generation of Recombinant Vaccinia rV-CEA/TRICOM(mu) No. vT172

For the generation of rV-CEA/TRICOM(mu), a plasmid vector, designated pT5031, was constructed to direct insertion of the foreign sequences into the M2L (30K) gene, which is located in the Hind III M region of the vaccinia genome (see FIG. 3). The CEA gene is under the control of the 40K promoter (13), the murine LFA-3 gene is under the control of the 30K promoter, the murine ICAM-1 gene is under the control of the I3 promoter, and the murine B7.1 gene is under the control of the sE/L promoter. These foreign sequences are flanked by DNA sequences from the Hind III M region of the vaccinia genome, including the vaccinia K1L host range gene. vTBC33, described above, was used as the parental virus in the construction of the recombinant vaccinia virus. The generation of recombinant vaccinia virus was accomplished via homologous recombination between vaccinia sequences in the vTBC33 vaccinia genome and the corresponding sequences in pT5031 in vaccinia-infected RK₁₃ cells transfected with pT5031. Recombinant virus, designated vT172, was selected by growth on RK₁₃ cells as described above. Plaques were picked from the cell monolayer and their progeny were further propagated. Two rounds of plaque isolation and replating on RK₁₃ cells resulted in the purification of the desired recombinant. The genomic structure of recombinant vT172 is depicted in FIG. 4C.

### Example 6

Generation of Recombinant Fowlpox, rF-TRICOM(mu) No. vT222

The origin of parental fowlpox virus used for the generation of recombinants was plaque-purified from a vial of a USDA-licensed poultry vaccine, POXVAC-TC, which is manufactured by Schering-Plough Corporation. The starting material for the production of POXVAC-TC was a vial of Vineland Laboratories' chicken embryo origin Fowlpox vaccine, obtained by Schering-Plough. The virus was passaged twice on the chorioallantoic membrane of chicken eggs to produce a master seed virus. The master seed virus was passaged 27 additional times in chicken embryo fibroblasts to prepare the POXVAC-TC master seed. To prepare virus stocks for the generation of POXVAC-TC product lots, the POXVAC-TC master seed was passaged twice on chicken embryo fibroblasts. One vial of POXVAC-TC, Serial # 96125, was plaque-purified three times on primary chick embryo dermal cells.

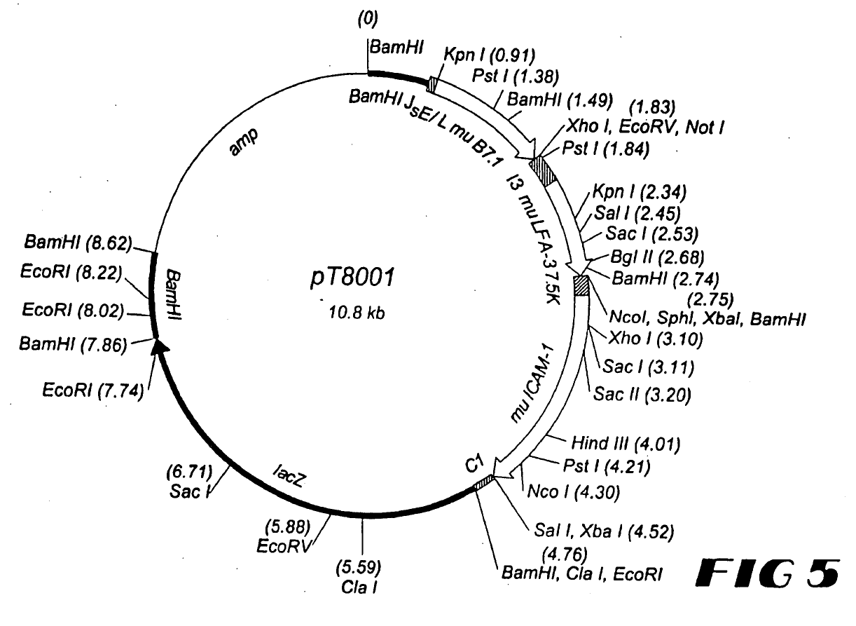

For the generation of rF-TRICOM(mu), a plasmid vector, designated pT8001, was constructed to direct insertion of the foreign sequences into the BamHI J region of the fowlpox genome. The murine B7.1 gene is under the control of the sE/L promoter, the murine LFA-3 gene is under the control of the I3 promoter, the murine ICAM-1 gene is under the control of the 7.5K promoter, and the lacZ gene is under the control of the C1 promoter. These foreign sequences are flanked by DNA sequences from the BamHI J region of the fowlpox genome (see FIG. 5). A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8001 in fowlpox-infected primary chick embryo dermal cells transfected with pT8001. Recombinant virus was identified using the chromogenic assay for the lacZ gene product described above. Viral plaques expressing lacZ appeared blue against a clear background. Positive plaques, designated vT222, were picked from the cell monolayer and their progeny were replated. Six rounds of plaque isolation and replating in the presence of Bluo-Gal resulted in the purification of the desired recombinant. The genomic structure of recombinant vT222 is depicted in FIG. 7A.

### Example 7

Generation of Recombinant Fowlpox rF-TAA/TRICOM(mu)

For the generation of rF-TAA/TRICOM(mu), a plasmid vector is constructed to direct insertion of the foreign sequences into the BamHI J region of the fowlpox genome. The TAA gene, the murine LFA-3 gene, the murine ICAM-1 gene, and the murine B7.1 gene are under the control of a multiplicity of promoters. In addition, the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired virus.

### Example 8

Generation of Recombinant Fowlpox rF-MUC-1/TRICOM(mu)

For the generation of rF-MUC-1/TRCOM(mu), a plasmid vector is constructed to direct insertion of the foreign sequences into the BamHI J region of the fowlpox genome. The MUC-1 gene, the murine LFA-3 gene, the murine ICAM-1 gene, and the murine B7.1 gene are under the control of a multiplicity of promoters. In addition, the lacZ gene is under the control of C1 promoter. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant virus.

### Example 9

Generation of Recombinant Fowlpox, rF-CEA/TRICOM(mu) No. vT 194

For the generation of rF-CEA/TRICOM(mu), a plasmid vector, designated pT5049, was constructed to direct insertion of the foreign sequences into the BamHI J region of the fowlpox genome. The CEA gene is under the control of the vaccinia 40K promoter, the murine B7-1 gene is under the control of the sE/L promoter, the murine LFA-3 gene is under the transcriptional control of the I3 promoter, the murine ICAM-1 gene is under the transcriptional control of the vaccinia 7.5K promoter, and the lacZ gene is under the control of the C1 promoter. These foreign sequences are flanked by DNA sequences from the BamHI J region of the fowlpox genome (see FIG. 6). A plaque-purified isolate from the POXVAC-TC (Schering Corporation) strain of fowlpox was used as the parental virus for this recombinant vaccine. The generation of recombinant fowlpox virus was accomplished via homologous recombination between fowlpox sequences in the fowlpox genome and the corresponding sequences in pT5049 in fowlpox-infected primary chick embryo dermal cells transfected with pT5049. Recombinant virus was identified using the chromogenic assay for the lacZ gene product described above. Viral plaques expressing lacZ appeared blue against a clear background. Positive plaques, designated vT194, were picked from the cell monolayer and their progeny were replated. Five rounds of plaque isolation and replating in the presence of Bluo-Gal resulted in the purification of the desired recombinant. The genomic structure of recombinant fowlpox vT194 is depicted in FIG. 7B.

### Example 10

Generation of Recombinant Vaccinia, rV-TRICOM (hu) No. vT224

For the generation of rV-TRICOM(hu), a plasmid vector, designated pT5064, was constructed to direct insertion of the foreign sequences into the thymidine kinase (TK) gene, which is located in the Hind III J region of the vaccinia genome. The human LFA-3 gene is under the control of the 30K promoter, the human ICAM-1 gene is under the control of the I3 promoter, and the human B7.1 gene is under the control of the sE/L promoter. In addition, the E. coli lacZ gene, under the control of the C1 promoter, is included as a screen for recombinant progeny. These foreign sequences are flanked by DNA sequences from the Hind III J region of the vaccinia genome (see FIG. 8). A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT5064 in vaccinia-infected CV-1 cells (ATTC, CCL 70) transfected with pT5064. Recombinant virus was identified using the chromogenic assay for the lacZ gene product described above. Viral plaques expressing lacZ appeared blue against a clear background. Positive plaques, designated vT224, were picked from the cell monolayer and their progeny were replated. Five rounds of plaque isolation and replating in the presence of Bluo-Gal resulted in the purification of the desired recombinant. The genomic structure of recombinant vT224 is depicted in FIG. 9A.

### Example 11

Generation of Recombinant Vaccinia rV-TAA/TRICOM(hu)

For the generation of rV-TAA/TRICOM(hu), a plasmid vector is constructed to direct insertion of the foreign sequences into the thymidine kinase (TK) gene, which is located in the Hind III J region of the vaccinia genome. The TAA gene, the human LFA-3 gene, the human ICAM-1 gene, the human B7.1 gene, and the E. coli lacZ gene are under the control of a multiplicity of poxvirus promoters. These foreign sequences are flanked by DNA sequences from the Hind III J region of the vaccinia genome. A plaque-purified isolate from the Wyeth (New York City Board of Health) strain of vaccinia is used as the parental virus for this recombinant vaccine. The generation of recombinant vaccinia virus is accomplished via homologous recombination between vaccinia sequences in the Wyeth vaccinia genome and the corresponding sequences in the plasmid vector in vaccinia-infected CV-1 cells (ATTC, CCL 70) transfected with the plasmid. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant.

### Example 12

Generation of Recombinant Fowlpox rF-TAA/TRICOM(hu)

For the generation of rF-TAA/TRICOM(hu), a plasmid vector is constructed to direct insertion of the foreign sequences into the BamHI J region of the fowlpox genome. The TAA gene, the human LFA-3 gene, the human ICAM-1 gene, the human B7.1 gene, and the E. coli lacZ gene are under the control of a multiplicity of poxvirus promoters. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant virus.

### Example 13

Generation of Recombinant Vaccinia Virus, rV-CEA (6D)/TRICOM(hu) No. vT238

For the generation of rV-CEA(6D)/TRICOM(hu), a plasmid vector, designated pT8016, was constructed to direct insertion of the foreign sequences into the thymidine kinase (TK) gene, which is located in the Hind III J region of the vaccinia genome. The CEA gene was altered by in vitro mutagenesis to express full-length protein containing one modified epitope. This mutation changed the encoded amino acid at position 576 from asparagine to aspartic acid. The modified gene, designated CEA(6D), was designed to enhance the immunogenicity of CEA (Zaremba et al, 1997, Cancer Res. 57:4570-4577). The CEA(6D) gene is under the control of the 40K promoter. The human LFA-3 gene is under the control of the 30K promoter, the human ICAM-1 gene is under the control of the I3 promoter, and the human B7.1 gene is under the control of the sE/L promoter. In addition, the E. coli lacZ gene, under the control of the C1 promoter, is included as a screen for recombinant progeny. These foreign sequences are flanked by DNA sequences from the Hind III J region of the vaccinia genome (see FIG. 10). A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT8016 in vaccinia-infected CV-1 cells (American Type Culture Collection (ATCC), Rockville, Md., CCL 70) transfected with pT8016. Recombinant virus was identified using the chromogenic assay for the lacZ gene product described above. Viral plaques expressing lacZ appeared blue against a clear background. Positive plaques, designated vT238, were picked from the cell monolayer and their progeny were replated. Six rounds of plaque isolation and replating in the presence of Bluo-Gal resulted in the purification of the desired recombinant. The genomic structure of recombinant vaccinia virus vT238 is shown in FIG. 11.

### Example 14

Generation of Recombinant Fowlpox Virus, rF-TRICOM(mu) No. vT251

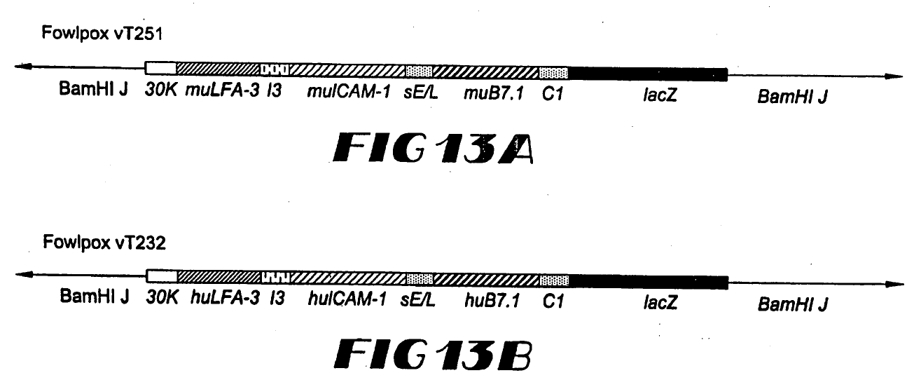

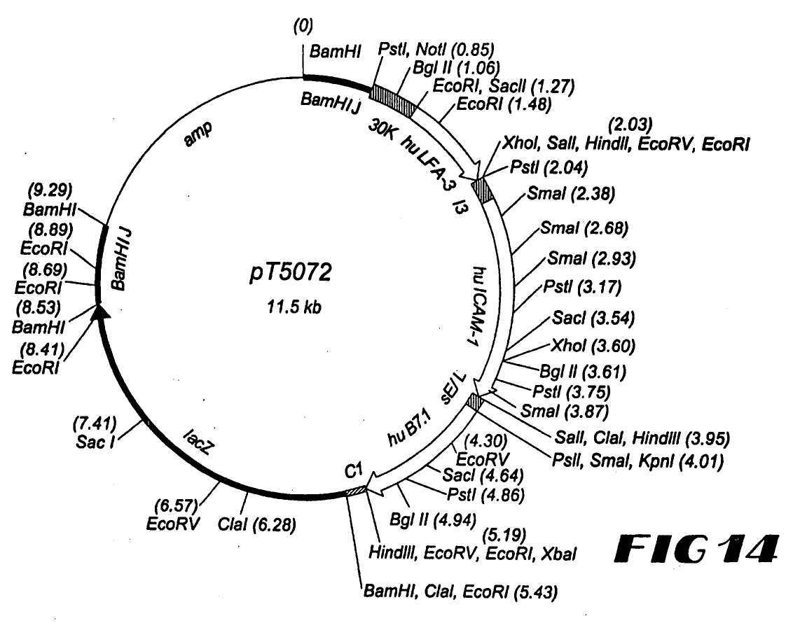

For the generation of rF-TRICOM(mu), a plasmid vector, designated pT8019, was constructed to direct insertion of the foreign sequences into the BamHI J region of the fowlpox genome. The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the BamHI J region of the fowlpox genome (see FIG. 12). A plaque-purified isolate form the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8019 in fowlpox-infected primary chick embryo dermal cells transfected with pT8019. Recombinant virus was identified using the chromogenic assay for the lacZ gene product described above. Viral plaques expressing lacZ appeared blue against a clear background. Positive plaques, designated vT251, were picked from the cell monolayer and their progeny were replated. Three rounds of plaque isolation and replating in the presence of Bluo-Gal resulted in the purification of the desired recombinant. The genomic structure of recombinant vaccinia virus vT251 is shown in FIG. 13A.

### Example 15

Generation of Recombinant Fowlpox Virus, rF-TRICOM(hu) No. vT232

For the generation of rF-TRICOM(hu), a plasmid vector, designated pT5072, was constructed to direct insertion of the foreign sequences into the BamHI J region of the fowlpox genome. The human LFA-3 gene is under the control of the 30K promoter, the human ICAM-1 gene is under the control of the I3 promoter, the human B7.1 gene is under the control of the sE/L promoter, and the lacZ gene is under the control of the C1 promoter. These foreign sequences are flanked by DNA sequences from the BamHI J region of the fowlpox genome (see FIG. 14). A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT5072 in fowlpox-infected primary chick embryo dermal cells transfected with pT5072. Recombinant virus was identified using the chromogenic assay for the lacZ gene product described above. Viral plaques expressing lacZ appeared blue against a clear background. Positive plaques, designated vT232 were picked from the cell monolayer and their progeny were replated. Four rounds of plaque isolation and replating in the presence of Bluo-Gal resulted in the purification of the desired recombinant. The genomic structure of recombinant vaccinia virus vT232 is shown in FIG. 13B.

### Example 16

Generation of Recombinant Fowlpox Virus, rF-MUC-1/TRICOM(mu) No. vT250

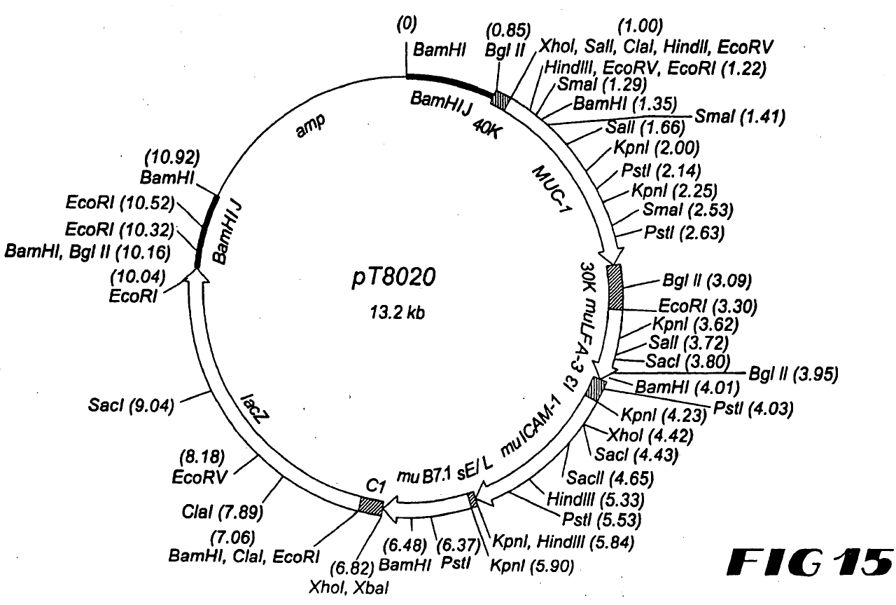

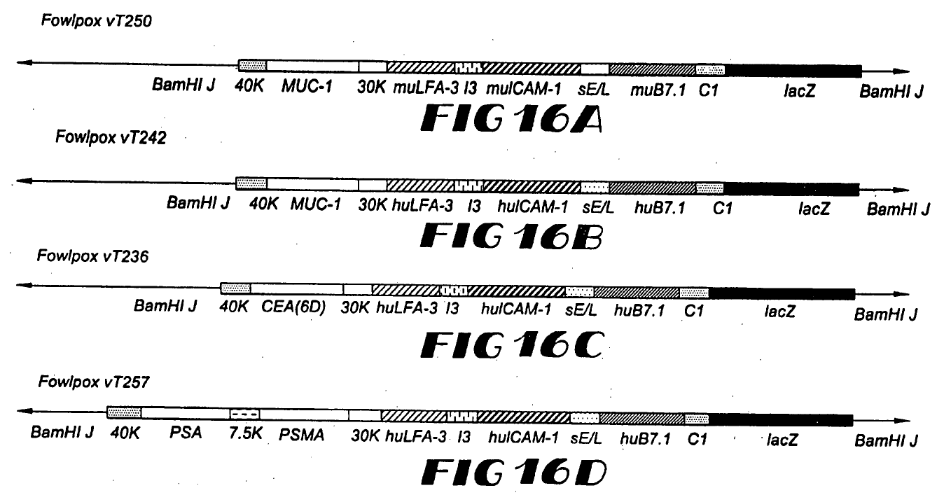

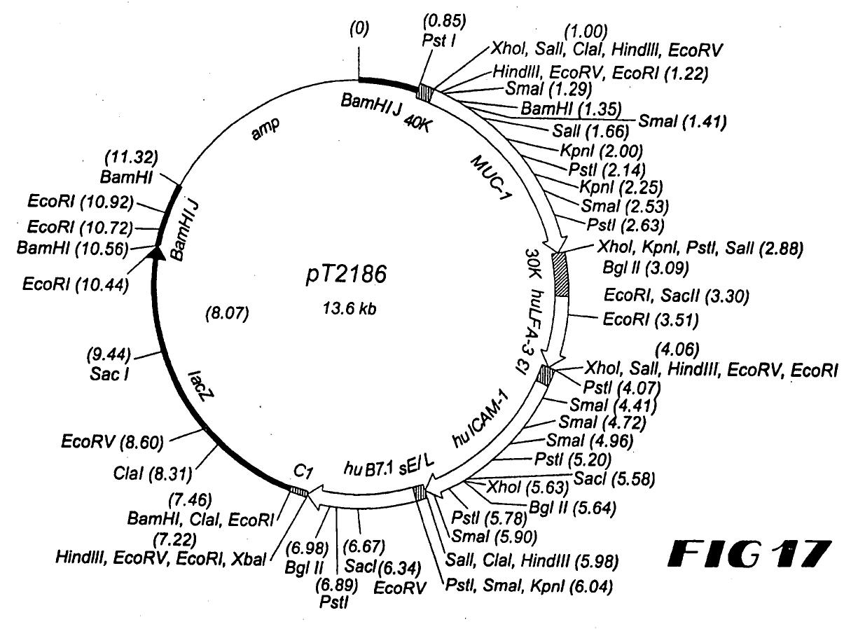

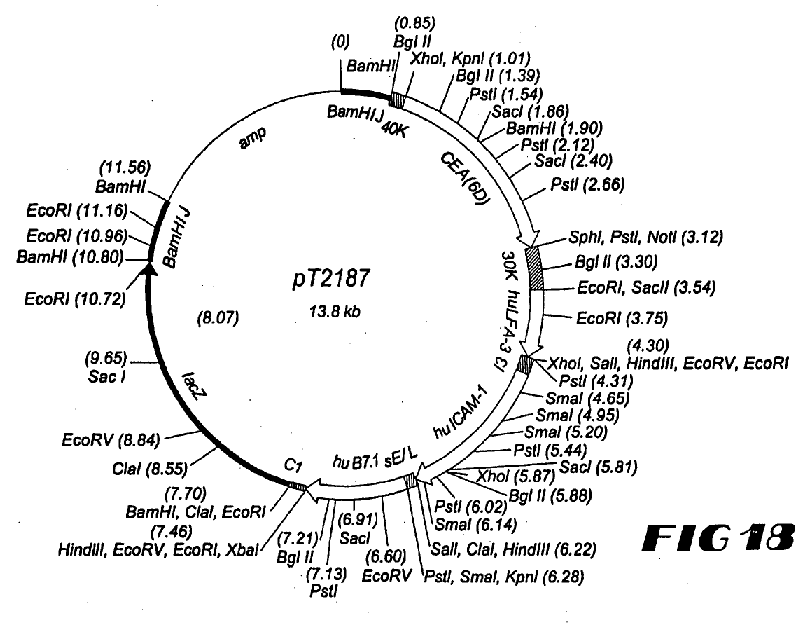

For the generation of rF-MUC-1/TRICOM(mu), a plasmid vector, designated pT8020, was constructed to direct insertion of the foreign sequences into the BamHI J region of the fowlpox genome. A truncated version of the MUC-1 gene was used, consisting of the signal sequence, ten copies of the tandem repeat sequence, and the 3′ unique coding sequence. (SEQ ID NO: 41 OF THE SECOND SEQUENCE LISTING HEREIN). The nucleotide sequence of the tandem repeat region was altered to minimize homology between the repeats without changing the amino acid sequence. The gene was contained on an 1881 bp fragment which includes the truncated coding sequence, 6 nucleotides of the 5′ untranslated region, and 186 nucleotides of the 3′ untranslated region (Gendler et al, 1990, J. Biol. Chem. 265:15286-15293). The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the BamHI J region of the fowlpox genome (see FIG. 15). A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8020 in fowlpox-infected primary chick embryo dermal cells transfected with pT8020. Recombinant virus was identified using the chromogenic assay for the lacZ gene product described above. Viral plaques expressing lacZ appeared blue against a clear background. Positive plaques, designated vT250, were picked from the cell monolayer and their progeny were replated. Four rounds of plaque isolation and replating in the presence of Bluo-Gal resulted in the purification of the desired recombinant. The genomic structure of recombinant vaccinia virus vT250 is shown in FIG. 16A.

### Example 17

Generation of Recombinant Fowlpox Virus, rF-MUC-1/TRICOM(hu) No. vT242

For the generation of rF-MUC-1/TRICOM(hu), a plasmid vector, designated pT2186 was constructed to direct insertion of the foreign sequences into the BamHI J region of the fowlpox genome. A truncated version of the MUC-1 gene was used, as described in Example 16 above. The MUC-1 gene is under the control of the 40K promoter. The human LFA-3 gene is under the control of the 30K promoter, the human ICAM-1 gene is under the control of the I3 promoter, the human B7.1 gene is under the control of the sE/L promoter, and the lacZ gene is under the control of the C1 promoter. These foreign sequences are flanked by DNA sequences from the BamHI J region of the fowlpox genome (see FIG. 17). A plaque-purified isolate from the POXVAC-TC (Schering-Plough Corporation) strain of fowlpox was used as the parental virus for this recombinant vaccine. The generation of recombinant fowlpox virus was accomplished via homologous recombinant between fowlpox sequences in the fowlpox genome and the corresponding sequences in pT2186 in fowlpox-infected primary chick embryo dermal cells transfected with pT2186. Recombinant virus was identified using the chromogenic assay for the lacZ gene product described above. Viral plaques expressing lacZ appeared blue against a clear background. Positive plaques, designated vT242, were picked from the cell monolayer and their progeny were replated. Four rounds of plaque isolation and replating in the presence of Bluo-Gal resulted in the purification of the desired recombinant. The genomic structure of recombinant vaccinia virus vT242 is shown in FIG. 16B.

### Example 18

Generation of Recombinant Fowlpox Virus, rF-CEA(6D)/TRICOM(hu) No. vT236

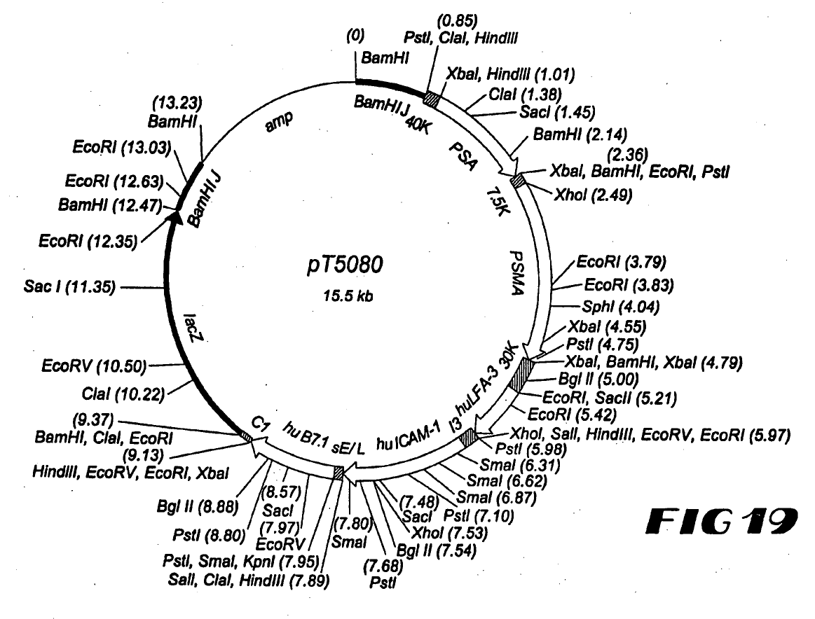

For the generation of rF-CEA(6D)/TRICOM(hu), a plasmid vector, designated pT2187, was constructed to direct insertion of the foreign sequences into the BamHI J region of the fowlpox genome. The CEA(6D) gene is under the control of the 40K promoter. The human LFA-3 gene is under the control of the 30K promoter, the human ICAM-1 gene is under the control of the I3 promoter, the human B7.1 gene is under the control of the sE/L promoter, and the lacZ gene is under the control of the C1 promote. These foreign sequences are flanked by DNA sequences from the BamHI J region of the fowlpox genome (see FIG. 18). A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT2187 in fowlpox-infected primary chick embryo dermal cells transfected with pT2187. Recombinant virus was identified using the chromogenic assay for the lacZ gene product described above. Viral plaques expressing lacZ appeared blue against a clear background. Positive plaques, designated vT236, were picked from the cell monolayer and their progeny were replated. Eight rounds of plaque isolation and replating in the presence of Bluo-Gal resulted in the purification of the desired recombinant. The genomic structure of recombinant vaccinia virus vT236 is shown in FIG. 16C.

### Example 19

Generation of Recombinant Fowlpox Virus, rF-PSA/PSMA/TRICOM(hu) No. vT257

For the generation of rF-PSA/PSMA/TRICOM(hu), a plasmid vector, designated pT5080, was constructed to direct insertion of the foreign sequences into the BamHI J region of the fowlpox genome. The gene encoding PSA was isolated by polymerase chain reaction amplification of cDNA derived from RNA from the human LNCaP cell line (CRL 1740, American Type Culture Collection (ATCC), Rockville, Md.). The gene was contained on a 1346 bp fragment which includes the entire coding sequence for PSA, 41 nucleotides of the 5′ untranslated region, and 552 nucleotides of the 3′ untranslated region (Lundwall and Lilja, 1987, FEBS Lett. 214:317-322). The gene encoding PSMA was isolated by polymerase chain reaction amplification of cDNA derived from RNA from the human LNCaP cell line. The gene was contained on a 2298 bp fragment which includes the entire coding sequence for PSMA, 26 nucleotides of the 5′ untranslated region, and 19 nucleotides of the 3′ untranslated region (Israeli et al, 1993 Cancer Res. 53:227-230). The PSA gene is under the control of the 40K promoter and the PSMA gene is under the control of the 7.5K promoter. The human LFA-3 gene is under the control of the 30K promoter, the human ICAM-1 gene is under the control of the I3 promoter, the human B7.1 gene is under the control of the sE/L promoter, and the lacZ is under the control of the C1 promoter. These foreign sequences are flanked by DNA sequences from the BamHI J region of the fowlpox genome (see FIG. 19). A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT5080 in fowlpox-infected primary chick embryo dermal cells transfected with pT5080. Recombinant virus was identified using the chromogenic assay for the lacZ gene product described above. Viral plaques expressing lacZ appeared blue against a clear background. Positive plaques, designated vT257, were picked from the cell monolayer and their progeny were replated. Five rounds of plaque isolation and replating in the presence of Bluo-Gal resulted in the purification of the desired recombinant. The genomic structure of recombinant vaccinia virus vT257 is shown in FIG. 16D.

### Example 20

Generation of Recombinant rMVA Virus, rMVA-TRICOM(mu) No. vT264

Modified Vaccinia Ankara (MVA) is an attenuated derivative of the Ankara strain of vaccinia virus (Meyer et al. 1991, J. Gen. Virol. 72:1031-1038). The seed stock from the MVA vaccine used as smallpox vaccine in humans was obtained from Dr. Anion Mayr (Institute for Medical Microbiology, Munich). The seed stock was plaque-purified two times on primary chick embryo dermal cells.

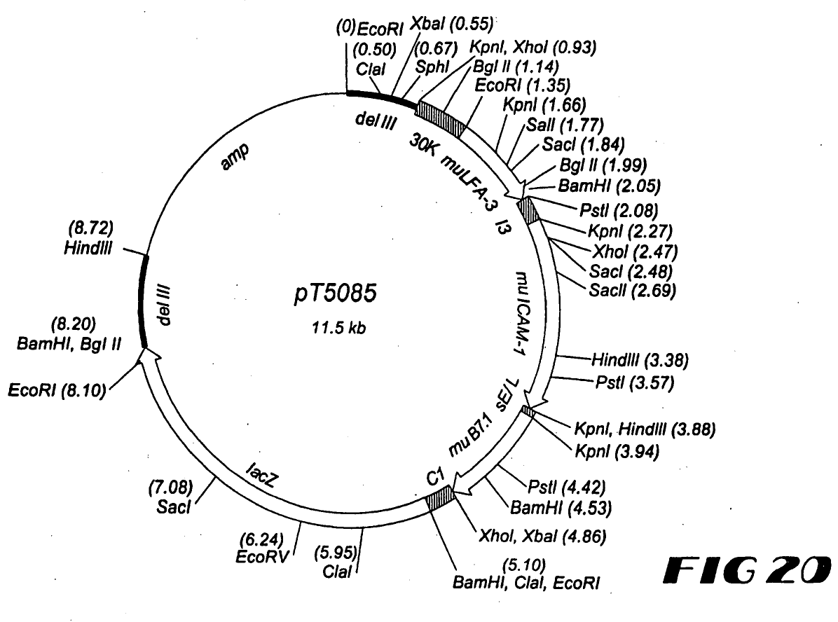

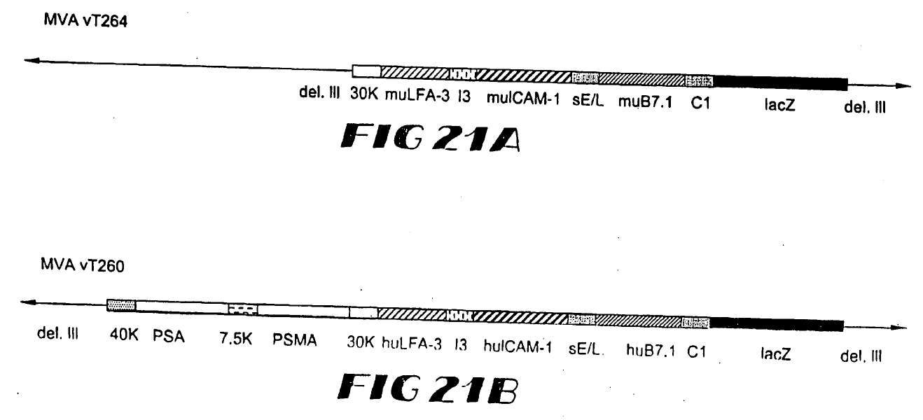

For the generation of rMVA-TRICOM(mu), a plasmid vector, designated pT5085, was constructed to direct insertion of the foreign sequences into the deletion III region of the MVA genome (Meyer et al, 1991, J. Gen. Virol. 72:1031-1038). The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the deletion III region of the MVA genome (see FIG. 20). A plaque-purified isolate from the MESA vaccine seed stock was used as the parental virus for this recombinant vaccine. The generation of recombinant MVA was accomplished via homologous recombinant between MVA sequences in the MVA genome and the corresponding sequences in pT5085 in MVA-infected primary chick embryo dermal cells transfected with pT5085. Recombinant virus was identified using the chromogenic assay for the lacZ gene product described above. Viral plaques expressing lacZ appeared blue against a clear background. Positive plaques, designated vT264 were picked from the cell monolayer and their progeny were replated. Four rounds of plaque isolation and replating in the presence of Bluo-Gal resulted in the purification of the desired recombinant. The genomic structure of recombinant MVA vT264 is shown in FIG. 21A.

### Example 21

Generation of Recombinant MVA Virus, rMVA-PSA/PSMA/TRICOM(hu) No. vT260

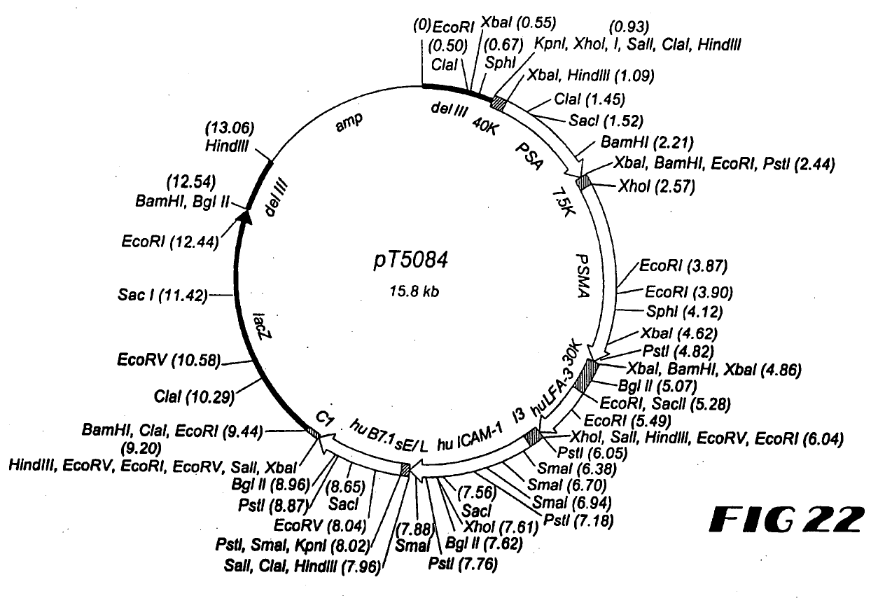

For the generation of rMVA-PSA/PSMA/TRICOM(hu), a plasmid vector, designated pT5084, was constructed to direct insertion of the foreign sequences into the deletion III region of the MVA genome. The PSA gene is under the control of the 40K promoter and the PSMA gene is under the control of the 7.5K promoter. The human LFA-3 gene is under the control of the 30K promoter, the human ICAM-1 gene is under the control of the I3 promoter, the human B7.1 gene is under the control of the sE/L promoter, and the lacZ gene is under the control of the C1 promoter. These foreign sequences are flanked by DNA sequences from the deletion III region of the MVA genome (see FIG. 22). A plaque-purified isolate from the MVA vaccine seed stock was used as the parental virus for this recombinant vaccine. The generation of recombinant MVA was accomplished via homologous recombination between MVA sequences in the MVA genome and the corresponding sequences in pT5084 in MVA-infected primary chick embryo dermal cells transfected with pT5084. Recombinant virus was identified using the chromogenic assay for the lacZ gene product described above. Viral plaques expressing lacZ appeared blue against a clear background. Positive plaques, designated vT260, were picked from the cell monolayer and their progeny were replated. Four rounds of plaque isolation and replating in the presence of Bluo-Gal resulted in the purification of the desired recombinant. The genomic structure of recombinant MVA vT260 is shown in FIG. 21B.

### Example 22

Recombinant Poxviruses

The individual recombinant vaccinia viruses containing either the gene encoding murine costimulatory molecule B7-1 (designated rV-B7-1) or the gene encoding murine Intercellular adhesion molecule-1 (designated rV-ICAM-1) have been described (10, 11). The recombinant vaccinia virus containing the gene for murine CD48 [designated rV-LFA-3; murine CD48 is the homologue of human LFA-3 (CD58) (6)] was constructed in a similar fashion to rV-B7-1 and rV-ICAM-1, and has been described (12). In each of these single recombinant vaccinia viruses, the gene encoding the costimulatory molecule was put under the control of the vaccinia virus early/late 40K promoter (A15), and the transgene was inserted into the Hind III M region of the genome of the Wyeth strain of vaccinia virus as described (A13). Recombinant fowlpox viruses were constructed by the insertion of foreign sequences into the BamHI J region of the genome of the POXVAC-TC (Schering Corporation) strain of fowlpox virus as described (A14). In recombinant viruses containing a single foreign gene, the gene is under control of the vaccinia 40K promoter. rV-B7-1/ICAM-1 is a recombinant vaccinia virus that contains the murine B7-1 gene under control of the synthetic early/late (SE/L) promoter (A16) and the murine ICAM-1 gene under control of the 40K promoter. rV-B7-1/ICAM-1/LFA-3 is a recombinant vaccinia virus that contains the murine LFA-3 gene under control of the vaccinia 30K (M2L) promoter (A17), the murine ICAM-1 gene under control of the vaccinia I3 promoter (A18), and the murine B7-1 gene under control of the synthetic early/late (sE/L) promoter. rF-CEA/B7-1/ICAM-1/LFA-3 is a recombinant fowlpox virus that contains the human carcinoembryonic antigen (CEA) gene under control of the 40K promoter, the murine B7-1 gene under control of the sE/L promoter, the murine LFA-3 gene under control of the I3 promoter, and the murine ICAM-1 gene under control of the vaccinia 7.5K promoter (A 19). Non-recombinant vaccinia virus was designated V-Wyeth, while non-recombinant fowlpox virus was designated WT-FP.

### Example 23

Expression of Recombinant Costimulatory Molecules

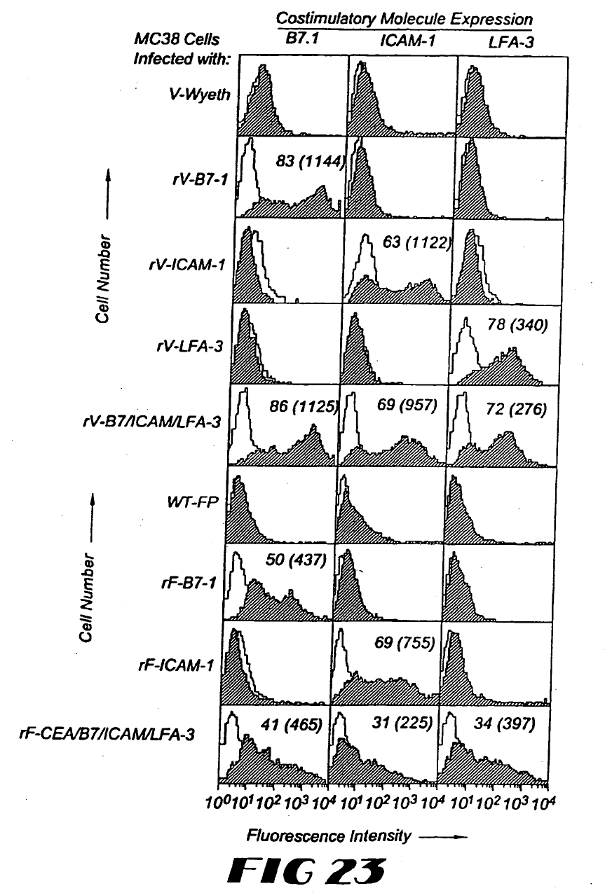

To confirm that each of the recombinant vectors could express the appropriate transgene(s), the murine adenocarcinoma cell line MC38 was infected with the various recombinant vaccinia or fowlpox constructs, and cell-surface expression of the transgene(s) was demonstrated by flow cytometry (FIG. 23). Uninfected cells (data not shown) and cells infected with wild-type vaccinia failed to express any of the three costimulatory molecules. This observation was confirmed by PCR (data not shown). In contrast, cells infected with rV-B7-1 became strongly positive for B7-1 protein; cells infected with rV-ICAM-1 became positive for ICAM-1; and cells infected with rV-LFA-3 became positive for LFA-3 protein. Similar analysis of a construct containing two costimulatory molecules (rV-B7-1/ICAM-1) showed expression of B7-1 (78% positive with a mean fluorescent intensity (MFI) of 1012) and ICAM-1 (70% positive with a MFI of 690). Moreover, cells infected with the vaccinia multiple-gene construct rV-B7-1/ICAM-1/LFA-3 co-expressed all three costimulatory molecules. To determine if the recombinant fowlpox viruses expressed their recombinant proteins, MC38 cells were infected with the fowlpox constructs in a similar manner (FIG. 23). Again, cells infected with wild-type fowlpox virus WT-FP failed to express any costimulatory molecule. Cells infected with rF-B7-1 became positive for B7-1 protein, and cells infected with rF-ICAM-1 became positive for ICAM-1 protein. A rF-LFA-3 vector was not constructed. However, cells infected with the fowlpox multiple-gene construct rF-CEA/B7-1/ICAM-1/LFA-3 co-expressed all three costimulatory molecules.

Characterization of Recombinant Viruses: Fluorescent Analysis of Protein Surface Expression

The MC38 murine colonic adenocarcinoma cell line has been described (20). Confluent MC38 cells were infected with vaccinia constructs (V-Wyeth, rV-B7-1, rV-ICAM-1, rV-LFA-3, rV-B7-1/ICAM-1/LFA-3) or fowlpox constructs (WT-FP, rF-B7-1, rF-ICAM-1, rF-CEA/B7-1/ICAM-1/LFA-3) at 5 MOI (multiplicity of infection; PFU/cell) for 5 hours. CEA was used in one rF construct as a marker gene only. After infection, cells were harvested and immunostained with FITC conjugated monoclonal antibodies (MAb) specific for murine CD80 (B7-1), CD54 (ICAM-1), or CD48 (LFA-3; PharMingen, San Diego, Calif.). Cell fluorescence was analyzed with a FACSCAN cytometer (Becton Dickinson. Mountain View, Calif.) with the Lysis II software.

In vitro Costimulation Analysis

Female C57BL/6 mice (6-8 weeks old) were obtained from Taconic Farms (Gennantown, N.Y.). Naive T cells were isolated from spleens mechanically dispersed through 70 ml cell strainers (Falcon, Becton Dickinson. Franklin Lakes, N.J.) to isolate single cell suspensions, and erythrocytes and dead cells were removed by centrifugation over Ficoll-Hypaque gradients (density=1.119 g/ml) (Sigma, St. Louis, Mo.). Populations consisting of approximately 95% T cells were obtained by passage of splenic mononuclear cells over two nylon wool columns sequentially (Robbins Scientific Corp., Sunnyvale, Calif.). For certain experiments, T cells were further fractionated into CD4⁺ and CD8⁺ populations by negative selection utilizing anti-CD4 or anti-CD8 paramagnetic beads (MiniMACS, Miltenyi Biotec, Auburn. Calif.). T cells were added at 10⁵/well in 96-well flat-bottomed plates (Costar. Cambridge, Mass.). Stimulator cells consisted of uninfected MC38 cells or cells infected for 5 hours with 5 MOI of vaccinia constructs (V-Wyeth, rV-B7-1, rV-ICAM-1, rV-LFA-3, rV-B7-1/ICAM-1/LFA-3) or fowlpox constructs (WT-FP, rF-B7-1, rF-ICAM-1, rF-CEA/B7-1/ICAM-1/LFA-3) fixed with 2% paraformaldehyde and added at 10⁴/well. Cells in all wells were cultured in a total volume of 200 .mu.l of complete media (CM) [RPMI 1640 with fetal calf serum (10%), glutamine (2 mM), sodium pyruvate (1 mM), Hepes (7 mM), gentamicin (50 .mu.g/ml), 2-mercaptoethanol (50 .mu.M), and non-essential amino acids (0.1 mM), (Biofluids, Rockville, Md.)] in the presence of several dilutions (5 to 0.625 .mu.g/ml for 2 days) of Concanavalin-A (Con A, Sigma). Control wells received T cells, stimulator cells and media only. For indicated experiments, plate-bound anti-CD3 (1.5 .mu.g/well-0.012 .mu.g/well) was substituted for Con A. Cells were labeled for the final 12-18 h of the incubation with 1 Ci/well ³H-Thymidine (New England Nuclear, Wilmington, Del.) and harvested with a Tomtec cell harvester (Wallac Incorporated, Gaithersburg, Md.). The incorporated radioactivity was measured by liquid scintillation counting (Wallac 1205 Betaplate, Wallac, Inc.) The results from triplicate wells were averaged and are reported as mean CPM.+−.SEM. For indicated experiments, the in vitro costimulation analysis was performed in the presence of either a MAb specific for the expressed costimulatory molecule or the matching isotype control antibody (Armenian hamster IgG, polyclonal). Antibodies used to block T-cell proliferation were Hamster anti-murine CD80 (B7-1; clone 16-10A1), Hamster anti-murine CD54 (ICAM-1; clone 3E2), or Hamster anti-murine CD48 (BCM-1 clone HM48-1), all from PharMingen. All antibodies were used at 25 .mu.g/ml final concentration.

Determination of Costimulatory Molecule Capacity

T cells and stimulator cells were prepared as described above. Fixed stimulator cells expressing one or more costimulatory molecules were added to wells in various ratios in combination with V-Wyeth-infected/fixed stimulator cells to a total of 10⁴/well. T cells (10⁵/well) were then added, and cells were cultured in a total volume of 200 .mu.l of CM in the presence of 2.5 .mu.g/ml Con A for 2 days and labeled for the final 12-18 h of the incubation with 1 .mu.Ci/well ³H-Thymidine. The incorporated radioactivity was measured by liquid scintillation counting as before.

Cytokine Analysis

CD4⁺ and CD8⁺ T-cell populations were prepared as described above and added at 2.5.times.10⁶/well in a 6-well plate (Costar). Stimulator cell populations were prepared as above and added at 2.5.times.10⁵/well. Cells were cultured in a total volume of 5 ml of CM in the presence of 2.5 .mu.g/ml Con A for 24 hours. Supernatant fluids were collected and analyzed for murine IL-2, IFN.gamma., TNF-.alpha., GM-CSF, and IL-4 by capture ELISA as described previously (A21). Sensitivity of detection was 30, 100, 20, 20, and 20 pg/ml, respectively.

RNA populations from stimulated cells were also analyzed by multiprobe RNAse protection assay (mpRPA). Defined riboprobes for murine cytokines were purchased from PharMingen. Assays were performed as described previously (22). Protected probe-tagged duplexes were separated by electrophoresis on 6% polyacrylamide gels. Dried gels were exposed to Biomax film (Kodak) at −70 C. for 24-72 hours. Radioactivity contained in the bands was quantified using a Storm system phosphoimager (Molecular Dynamics, Sunnyvale. Calif.). The net CPM for a given band was calculated by the following formula [cpm of cytokine gene minus cpm of background] and was expressed as a percent of the housekeeping gene transcript L32.

### Example 24

B7-1, ICAM-1, and LFA-3 Cooperate Synergistically to Enhance T-cell Proliferation

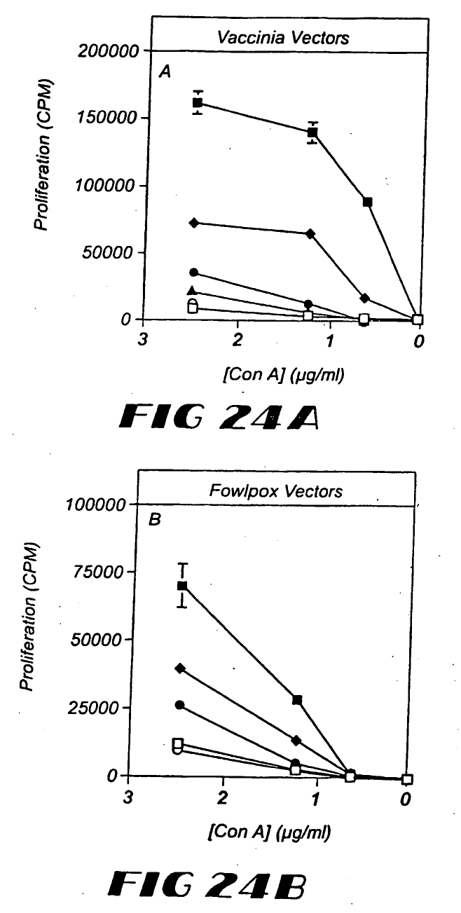

The B7-1, ICAM-1 and LFA-3 molecules have been shown individually to costimulate T-cell proliferation. However, because they may be expressed simultaneously on APC, it has been difficult to examine relative roles of individual costimulatory molecules during the induction of T-cell proliferation (2). To analyze the contribution of B7-1, ICAM-1 and/or LFA-3 molecules to the induction of naive T-cell proliferation, a modified in vitro model (23, 24) was employed where the first signal for T-cell activation was delivered via a pharmacological reagent (Con A). A panel of stimulator cells that differed only in costimulatory molecules was created using the MC38 cell line infected with various recombinant vaccinia (FIG. 24A) or fowlpox (FIG. 24B) viruses engineered to express costimulatory molecules. The second, or “costimulatory,” signal was delivered to the T cell via one or more costimulatory molecules expressed on the surface of these “stimulator” MC38 cells. As shown in FIG. 24A, both uninfected MC38 cells and MC38/V-Wyeth induced marginal proliferation of T cells at all levels of Con A examined. MC38/LFA-3 induced a small (2.1-fold) but significant (P<0.05) increase in T-cell proliferation. Delivery of signal-2 via MC38/ICAM-1 induced a 3.5-fold increase in T-cell proliferation at 2.5 .mu.g/ml Con A. MC38/B7-1 induced a 7.8-fold and a 16-fold increase in proliferation at 2.5 and 1.25 .mu.g/ml Con A respectively. However, MC38/B7-1/ICAM-1/LFA-3 (MC38 cells co-expressing all three costimulatory molecules) induced a 17.5-fold increase in T-cell proliferation at 2.5 .mu.g/ml Con A, and a 34-fold increase at 1.25 .mu.g/ml Con A. Moreover, at low Con A levels (0.625 .mu.g/ml), expression of ICAM-1 and LFA-3 did not induce T-cell proliferation. While B7-1 induced measurable proliferation (20,000 CPM) at 0.625 .mu.g/ml Con A, the co-expression of all three costimulatory molecules induced an even greater level of proliferation (100,000 CPM) (FIG. 24A). These experiments were repeated four times with similar results.

MC38 stimulator cells were also prepared by infection with recombinant fowlpox vectors (FIG. 24B). Again, uninfected MC38 or MC38/WT-FP induced marginal proliferation of T cells at all levels of Con A examined. MC38/rF-ICAM-1 supported a 2-fold increase, MC38/rF-B7-1 supported a 3.2-fold increase, and MC38/rF-B7-1/ICAM-1/LFA-3 supported a 6-fold increase in T-cell proliferation at 2.5 .mu.g/ml Con A. Similar results were obtained when this experiment was repeated two additional times. Similar results were also observed when the first signal was delivered via immobilized anti-CD3 (data not shown). The differences noted in proliferation supported by MC38/rV-B7-1/ICAM-1/LFA-3 and MC38/rF-CEA/B7-1/ICAM-1/LFA-3 (17.5-fold vs. 6-fold) are most likely due to the levels of expressed recombinant protein(s) following a 5-hour infection period (FIG. 23). Specifically, approximately 70% of the cells infected with rV-B7-1/ICAM-1/LFA-3 express the costimulatory molecules, while approximately 40% of cells infected with rF-CEA/B7-1/ICAM-1/LFA-3 are positive. Those positive cells infected with the rF vectors express recombinant B7-1 and ICAM-1 at levels of 50% of those cells infected with rV-B7-1/ICAM-1/LFA-3 with the conditions used.

### Example 25

Specificity of Costimulatory Molecule Contribution on T-Cell Proliferation

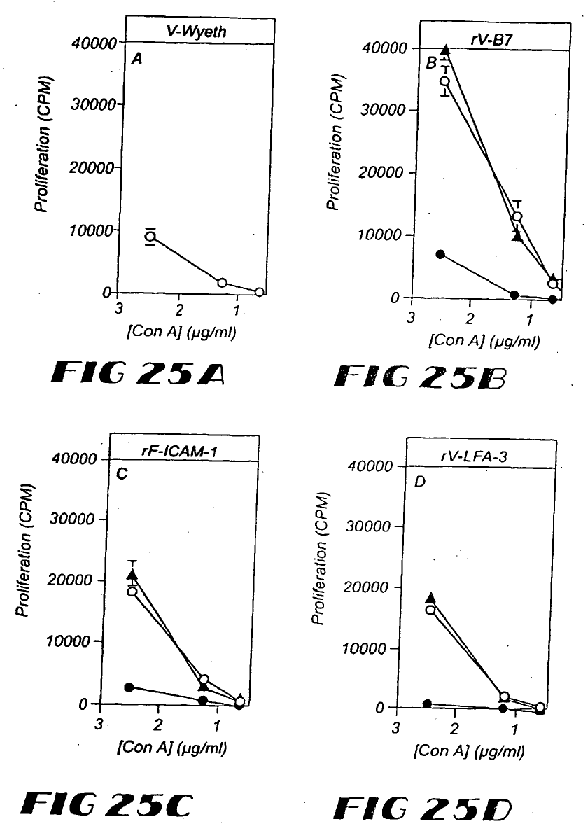

To further confirm the specificity of the proliferative contribution of B7-1, ICAM-1, or LFA-3, MC38 stimulator cells were again prepared by infection with V-Wyeth, rV-B7-1, rV-ICAM-1, or rV-LFA-3 and co-cultured with naive murine T cells and Con A in the presence or absence of MAb specific for the given costimulatory molecule. As shown in FIG. 3B, MC38/B7-1 enhanced T-cell proliferation 4.5-fold more than that of MC38/V-Wyeth (FIG. 25A). This increased proliferation was inhibited 83% by the addition of a blocking MAb for murine B7-1. Similarly, MC38/ICAM-1 (FIG. 25C) increased proliferation 2.25-fold, which was then reduced by 88% in the presence of anti-murine ICAM-1 MAb. Finally, MC38/LFA-3 (FIG. 25D) increased proliferation 2.1-fold, which was then reduced by 98% in the presence of anti-murine CD48 MAb. For each group, incubation with the appropriate isotype control antibody (as specified in Materials and Methods) failed to block the noted proliferation. This experiment was repeated two additional times with similar results.

### Example 26

Determination of Costimulatory Molecule Capacity

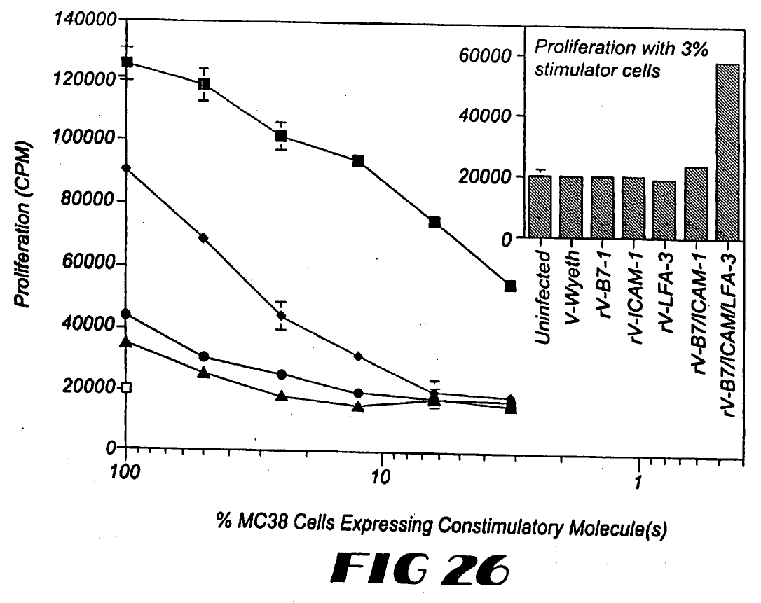

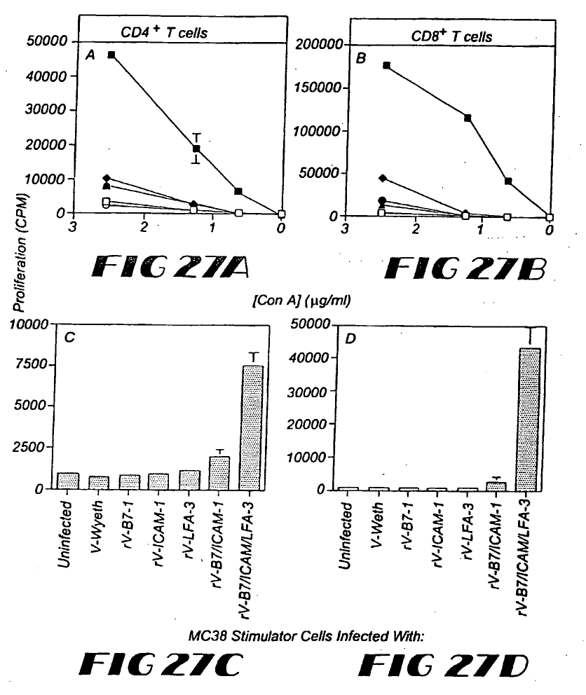

Modification of the in vitro costimulation assay allowed a quantitative estimation of the relative capacity of B7-1, ICAM-1, and/or LFA-3 to deliver the second signal for T-cell proliferation. To that end, stimulator cells (MC38 cells infected with the various recombinant vaccinia viruses) were titered out by dilution with varying amounts of MC38 cells infected with V-Wyeth and co-cultured with a constant number of T cells in the presence of 2.5 .mu.g/ml Con A. The MC38 to T-cell ratio in these experiments remained constant at 1:10. As seen in FIG. 4, MC38/LFA-3 (closed triangles) enhanced proliferation of T cells over that of MC38/V-Wyeth (open square) out to a concentration of 40% (i.e., of the stimulator cells in the well. 40% were infected with rV-LFA-3 and the remaining 60% were infected with V-Wyeth). MC38/ICAM-1 (closed circles) or MC38/B7-1 (closed diamonds) supported increased proliferation out to a concentration of 13% and 6%, respectively. In contrast, MC38/B7-1/ICAM-1/LFA-3 enhanced proliferation when less than 3% of stimulator cells contained the triad vector (extrapolated to less than 1% via linear least squares analysis). Given the titration curves of these individual costimulatory molecules, it appeared that the extent of T-cell proliferation mediated by ICAM-1 and B7-1 is 3-fold and 6-fold, respectively, more potent than that mediated by LFA-3 alone. Clearly the strongest proliferation, however, is mediated by B7-1/ICAM-1/LFA-3. It should be noted (FIG. 26) that at relatively low stimulator cell concentrations (i.e., when 3%-6% of the MC38 cells are acting as stimulator cells), expression of LFA-3, ICAM-1, and even B7-1 alone does not enhance T-cell activation, while the three costimulatory molecules expressing stimulator cells substantially enhance T-cell activation. The data in FIG. 26 (insert) shows proliferation results obtained when 3% of the MC38 stimulator cells were infected with the vectors denoted. Since each well contained 104 total MC38 cells and 10⁵ naive T cells, the actual stimulator to T-cell ratio in these cultures was 0.003. Note that the MC38 cells infected with the two-gene construct (rV-B7-1/ICAM-1) induced little, if an, proliferation of T cells under these conditions, while MC38/B7-1/ICAM-1/LFA-3 increased proliferation substantially (p<0.0001).

### Example 27

Costimulation of CD4⁻ and CD8⁺ T Cells

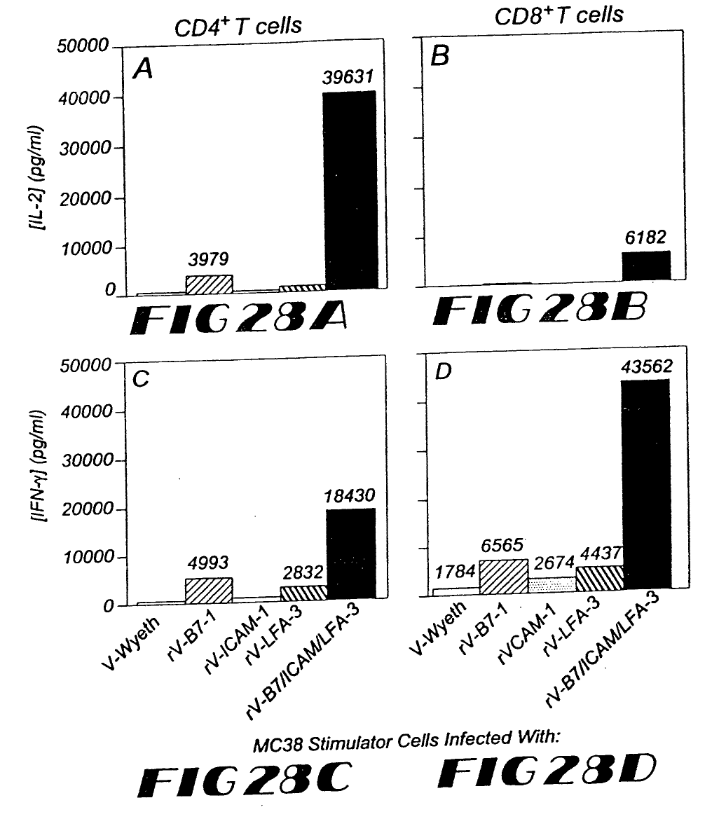

To further characterize the T-cell response to costimulatory molecules expressed singly or in combination, the ability of B7-1, ICAM-1, and LFA-3 to costimulate purified CD4⁻ and CD8⁺ T cells was tested. FIG. 5 shows the proliferation of purified CD4⁺ (FIG. 27A) and CD8⁺ (FIG. 27B) cells activated with suboptimal concentrations of Con A. The stratification of stimulator cell effects on proliferation was similar for both CD4⁻ and CD8⁺ cells: MC38/LFA-3 stimulated the weakest proliferation, followed by MC38/ICAM-1 and MC38/B7-1. MC38/B7-1/ICAM-1/LFA-3 were the most potent stimulator cells for CD4⁺ and CD8⁺ T cells. These experiments were repeated three additional times with similar results. It should be noted that at very low concentrations of Con A (0.625 .mu.g/ml, FIG. 5, panels C and D), there was no significant enhancement in activation of CD4⁺ or CD8⁺ T cells when either ICAM-1, LFA-3, B7-1, or the B7-1/ICAM-1 combination was used to provide the second signal. However, substantial activation of both T-cell subsets was observed when the vaccinia virus coexpressing the triad of costimulatory molecules was employed. Similar results were noted when the first signal was delivered via immobilized anti-CD3 (data not shown).

It has been reported that B7-1 costimulation prolongs IL-2 mRNA half life and upregulation of IL-2 transcription, resulting in production of considerable amounts of secreted IL-2 (4, 25). Additionally, T-cell costimulation with LFA-3 has been reported to have an effect on a variety of cytokines, notably IL-2 and IFN-.gamma. (6). To determine qualitative and quantitative effects of costimulation by single or multiple costimulatory molecules on cytokine production, purified CD4⁺ and CD8⁺ T cells were again co-cultured with various stimulator cells expressing B7-1, ICAM-1 and LFA-3 alone or in combination in the presence of 2.5 .mu.g/ml Con A. Supernatant fluids were analyzed for IL-2, IFN-.gamma., TNF-.alpha., GM-CSF, and IL-4 after 24 hours. Uninfected MC38 (data not shown) and MC38/V-Wyeth induced a marginal quantity of IL-2 from CD4⁺ cells (FIG. 28A), while MC38/B7-1 induced 3,979 pg/ml. However, T-cell stimulation with MC38/B7-1/ICAM-1/LFA-3 induced a 10-fold greater amount of IL-2. Similarly, MC38/B7-1 induced a marginal quantity of IL-2 from CD8+ cells (FIG. 28B), while MC38/B7-1/ICAM-1/LFA-3 induced a 20-fold greater amount (6,182 pg/ml). IFN-.gamma. production by stimulated T cells was also examined. MC38/B7-1 and MC38/LFA-3 induced only moderate amounts of IFN-.gamma. from CD4⁺ cells (FIG. 28C). In contrast stimulation of CD4⁺ cells with MC38/B7-1/ICAM-1/LFA-3 induced 4-fold more IFN-.gamma. than stimulation with any other construct. Stimulation of CD8⁺ cells with MC38/B7-1/ICAM-1/LFA-3 induced the greatest amount of IFN-.gamma. greater than 6-fold more than CD8⁺ cells stimulated with any of the other constructs (FIG. 28D). Stimulation of either cell type with any construct failed to mediate significant changes (p>0.05) in the levels of secreted TNF-.alpha. GM-CSF, or IL-4 (data not shown). It appears that the predominant culmination of stimulation via the triad construct (rV-B7-1/ICAM-1/LFA-3) was IL-2 secretion from CD4⁺ cells and IFN-.gamma. secretion from CD8⁺ T cells. These experiments were repeated three additional times with similar results. Studies were also carried out comparing stimulator cells infected with the two-gene construct (rV-B7-1/ICAM-1) vs. the multi-gene construct (rV-B7-1/ICAM-1/LFA-3) for their ability to enhance cytokine production by T cells. Only small differences were observed between the two in IFN-.gamma. production by either CD4⁺ or CD8⁺ cells, or in IL-2 production by CD8⁺ cells. But a substantial difference was seen in the stimulation of IL-2 production by CD4⁺ cells (5000 pg/ml employing MC38/B7-1/ICAM-1 vs. 39,600 pg/ml employing MC38/B7-1/ICAM-1/LFA-3).

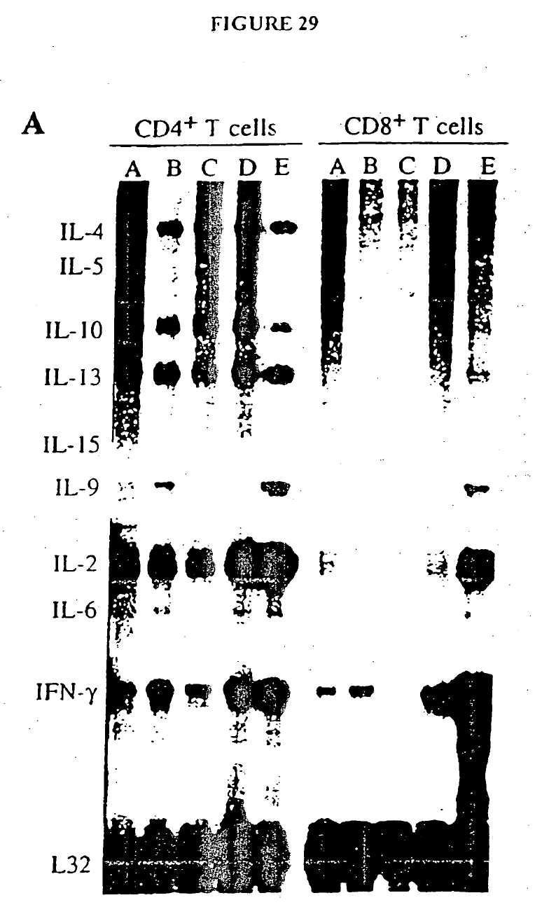

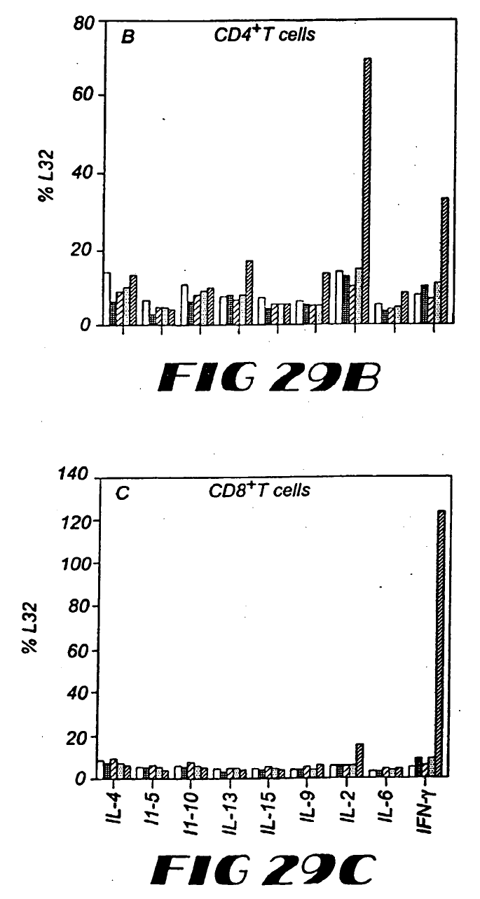

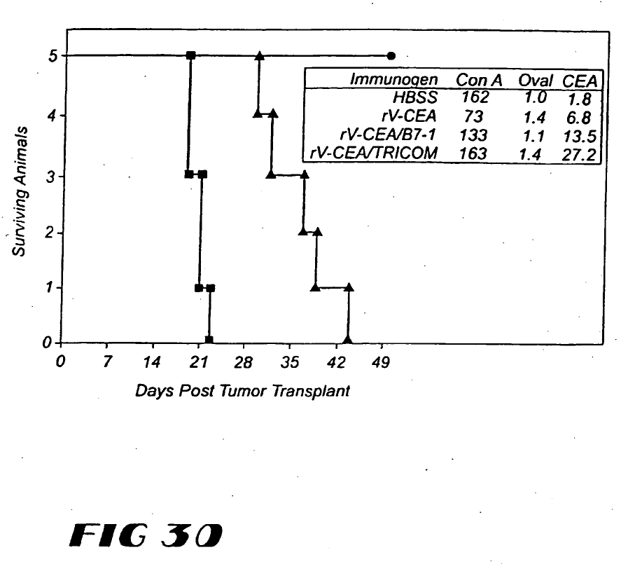

Cytokine expression from CD4⁺ and CD8⁺ T cells stimulated with single or multiple costimulatory molecules was also analyzed at the RNA level utilizing the multiprobe RNAse protection assay (mRPA). A representative radiographic profile and quantitative analysis from two independent experiments are depicted (FIG. 29). Levels of IL-4, IL-5, IL-10, IL-15, and IL-6 were similar in CD4⁺ T cells stimulated with MC38/V-Wyeth, MC38/B7-1, MC38/ICAM-1, MC38/LFA-3, or MC38/B7-1/ICAM-1/LFA-3 (FIG. 29, panel B histogram). IL-2 and IFN-.gamma. expression levels were highest in CD4⁺ T cells stimulated with MC38/B7-1/ICAM-1/LFA-3 when compared with CD4⁺ cells stimulated with MC38 cells expressing any single costimulatory molecule (FIG. 29B). Slightly higher levels of IL-13, IL-9, and IL-6 were also noted in CD4⁺ cells stimulated with MC38/B7-1/ICAM-1/LFA-3. Expression of cytokine genes was also analyzed in stimulated CD8⁺ T cells. Of the cytokine RNAs analyzed, IL-2 and particularly IFN-.gamma. levels were significantly higher when these cells were stimulated with MC38/B7-1/ICAM-1/LFA-3, compared to T cells stimulated with MC38 cells expressing any single costimulatory molecule. Thus, the predominant synergistic effect of the triad of costimulatory molecules in cytokine production was IL-2 in CD4⁺ cells and IFN-.gamma. in CD8⁺ T cells.

### Example 28

Effect of TRICOM costimulation on Apoptosis of Stimulated T Cells

Apoptosis Studies

To determine if stimulation of T cells with signal 1 and rV-TRICOM would lead to cell survival or programmed cell death (PCD), CD8⁺ T cells were activated with Con A for signal 1, cultured with either V-WT, rV-B7-1 or rV-TRICOM-infected MC38 cells for 48 hr, and replated for 24 hr in medium to measure apoptosis. Apoptosis was assessed using the TUNEL assay, as described by Gavrieli, Y et al. J. Cell Biol 119: 493-501, 1992. T cells activated by the combination of MC38 and Con A or MC38/V-WT and Con A in the absence of costimulatory signals exhibited high levels of spontaneous apoptosis (82.9.+−. 1, respectively). T cells activated by Con A and MC38/B7-1 or Con A and MC38/TRICOM exhibited substantially less spontaneous apoptosis (31.3.+−.3.8 and 30.7.+−.0.1, respectively).

The results clearly demonstrate apoptosis in T cells stimulated with MCB8 cells in the presence of Con A with or without V-WT infection (i.e., in the absence of signal 2). While Con A with MC38/TRICOM clearly stimulated CD8⁺ cells to far greater levels than Con A with MC38/B7-1 and resulted in the production of higher levels of IFN-.gamma. and IL-2, this did not result in any greater degree of apoptosis.

### Example 29

Anti-Tumor Effect of rV-CEA/TRICOM In Vivo

Studies were conducted to determine if an antigen-specific immune response could be enhanced using a TRICOM vector. A four-gene vaccinia recombinant was constructed that contained the human CEA gene and the B7-1. ICAM-1 and LFA-3 genes, designated rV-CEA/TRICOM, as disclosed herein. Six to eight-week-old female C57 BL/6 mice (Taconic Farms) or C57BL/6 mice transgenic for human CEA (Kass, E et al Cancer Res. 59: 676-683, 1999) were vaccinated by tail scarification with either Hank's Balanced Salt Solution (HBSS) or one time with, 10⁷ pfu rV-CEA, rV-CEA/B7-1 or rV-CEA/TRICOM, and spleens were harvested 22 days later. Lymphoproliferative activity of splenocytes was analyzed as described previously (5).

As seen in FIG. 30 (insert), splenic T cells of mice vaccinated with rV-TRICOM showed higher levels of CEA-specific stimulation compared with T cells obtained from mice vaccinated with rV-CEA; Ovalbumin and Con A were used as controls. An experiment was then conducted to determine if rV-CEA/TRICOM could induce long-term immunity. Mice (5/group) were vaccinated one time with either V-WT, rV-CEA, or rV-CEA/TRICOM. One hundred days later, mice were challenged with a high dose (1.times.10⁶) of MC38 colon carcinoma cells expressing CEA (5). All mice receiving V-WT and rV-CEA succumbed to tumors, while all mice vaccinated with rV-TRICOM were alive 50 days post-challenge (FIG. 30).

CEA-transgenic mice (Kass 1999, ibid; Thompson, J. A. et al., J. Clin. Lab. Anal. 5:344-366, 1999) in which the human CEA gene is expressed in normal adult gastrointestinal tissue, and whose serum is CEA-positive, were employed to determine if the rV-CEA/TRICOM vector could enhance T-cell responses to a self-antigen. CEA transgenic mice were separated into 5 mice/group. Two mice were vaccinated once with 10⁷ pfu rV-CEA, rV-CEA/B7-1, rV-CEA/TRICOM or buffer and were euthanized on day 30 to analyze CEA-specific T-cell responses. T-cell responses obtained after vaccination with rV-CEA/TRICOM were substantially greater than those obtained with rV-CEA (Table 2). Responses to ovalbumin and Con A were used as controls. The remaining 3 CEA-transgenic mice in each group were used to determine if anti-tumor responses to a CEA-expressing tumor could be enhanced employing a TRICOM vector. These mice were first inoculated s.c. with 4.times.10⁵ MC38 carcinoma cells expressing the CEA gene (5). Four days later, mice were vaccinated one time at a distal site with 10⁷ pfu viral recombinant or buffer. No tumors grew in mice vaccinated with rV-CEA/TRICOM, whereas tumors continued to grow in mice vaccinated with buffer, rV-CEA and rV-CEA/B7-1 (Table 2). These results support the in vivo activity of TRICOM vectors.

TABLE 2
Enhanced Immune Response and Anti-Tumor Response
of rV-CEA/TRICOM in CEA Transgenic Mice

| | Stimulation Index (SI) | | | | | |
|---|---|---|---|---|---|---|
| | Con A | Oval | CEA | CEA | Tumor Value | |
| Immunogen | (5 μg/ml) | (100 μg/ml) | (100 μg/ml) | (25 μg/ml) | Day 14 | Day 35 |
| HBSS | 109 | 1.0 | 1.3 | 2.0 | 698 ± 928 | 3,674 ± 3,107 |
| rV-CEA | 123 | 0.9 | 4.9 | 4.0 | 259 ± 0 | 1,112 ± 1,685 |
| rV-CEA/B7-1 | 93 | 1.3 | 7.1 | 4.3 | 150 ± 236 | 2,696 ± 1,936 |
| rV-CEA/TRICOM | 111 | 1.1 | 19.2 | 15.9 | 0 ± 0 | ±0 |
| C57BL/6 CEA-transgenic mice (5 per group) were vaccinated via skin scarification with buffer or vaccinia recombinant (10⁷ pfu) one time on Day 0. On Day 30, 2 mice were | | | | | | |
| # killed and splenic T cells were analyzed for T-cell proliferative responses. Each value represents the S [symbol] of the mean CPM of triplicate samples versus [symbol] din. Standard | | | | | | |
| # deviation never exceeded 10%. On Day -4, 3 mice per group were given 4 × 10⁵ MC38 colon | | | | | | |
| # carcinoma cells expressing CEA. Tumor volume is given at Days 14 and 35 post-vaccination. | | | | | | |

### Example 30

Costimulation of CD4⁺ and CD8⁺ T Cells by Progenitor Dendritic Cells and Dendritic Cells Infected with rV-B7/ICAM-1/LFA-3

Fresh CD34⁺ bone marrow cells (dendritic cell precursors) were obtained from C57BL/6 mice by the method of Inaba et al (41). These precursor cells were either used immediately or cultured for 6 days in GM-CSF and IL4 (42) to generate mature dendritic cells (DC). CD34⁺ precursor cells and DC were infected for 18 hours with the recombinant vaccinia virus encoding multiple costimulatory molecules rV-B7/ICAM-1/LFA-3 (rV-Tricom), 10 MOI. After 5 hours of infection, a sample of cells were harvested and a phenotypic analysis was performed. Dendritic cells are though of in the art as the ‘ultimate’ APC, expressing a large array of costimulatory molecules at high levels. Table 3 shows that murine DC indeed express the costimulatory molecules B7-1, B7-2, ICAM-1, and LFA-3 at relatively high levels (mean fluorescent intensity, MFI; depicted in parenthesis). However, when DC were infected with rV-B7/ICAM-1/LFA-3, there was a significant increase in both the level of costimulatory molecule expression as well as the percentage of cell expressing the multiple costimulatory molecules. The percentage of cells expressing B7-1 increased from 65% to 86%, while the MFI increased 4-fold; the percentage of cells expressing ICAM-1 increased from 32% to 68%, while the MFI increased 2.5 fold; the percentage of cells expressing LFA-3 increased from 44% to 75%.

TABLE 3
Phenotypic Analysis of Progenitor DC Pre and Post Infection¹ with rV-COS²

| | Marker | | | | | | | |
|---|---|---|---|---|---|---|---|---|
| Infection | H2-K^{b} | I-A^{b} | CD11b | CD11c | B7-2 | B7-1 | ICAM-1 | LFA-3 |
| None | 90³ (994)⁴ | 64 (621) | 63 (397) | 29 (223) | 38 (319) | 65 (300) | 32 (336) | 44 (378) |
| V-Wyeth | 75 (554) | 60 (633) | 59 (398) | 27 (218) | 36 (317) | 65 (311) | 33 (296) | 43 (322) |
| rV-B7 | 76 (516) | 67 (755) | 70 (419) | 34 (213) | 41 (320) | 83 (661) | 43 (363) | 51 (333) |
| rV-B7/ICAM/LFA-3 | 79 (579) | 63 (696) | 63 (408) | 30 (203) | 42 (360) | 86 (1253) | 68 (810) | 75 (484) |

¹5 hour infection at 10 MOI
²rV-COS = recombinant vaccinia encoding a foreign costimulatory molecule.
³= % cells expressing marker
⁴= mean fluorescent intensity

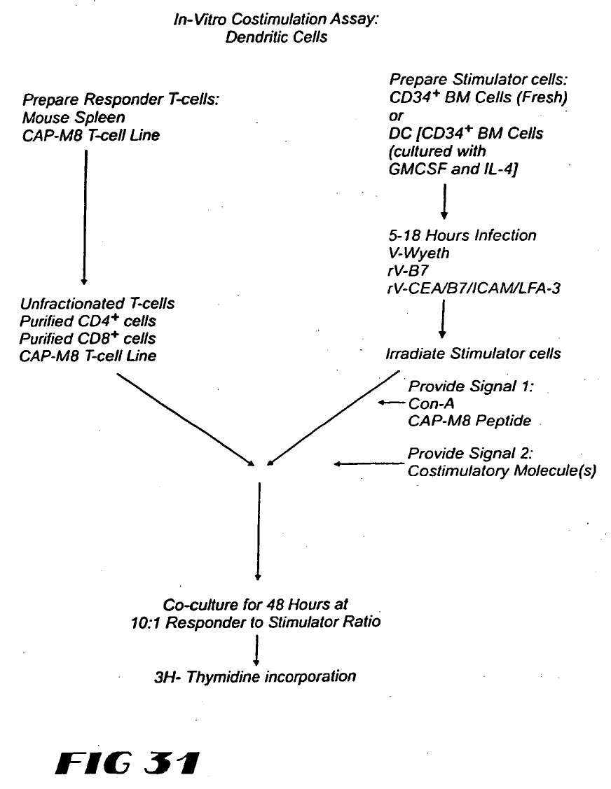

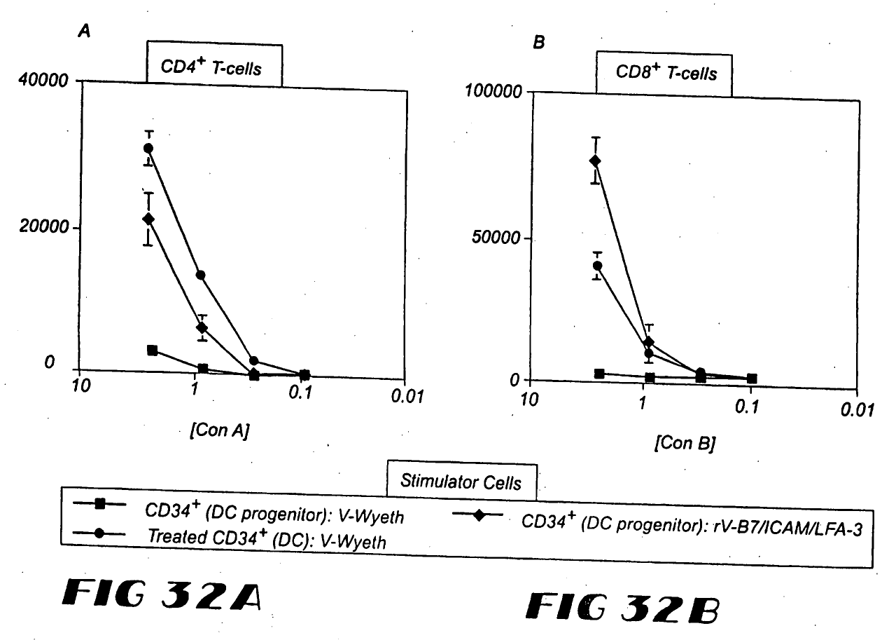

For use as stimulator cells, the infected CD34⁺ precursor cells and DC were irradiated (2000 rad) and used to stimulate naive CD4⁺ and CD8⁺ T-cells in the presence of Con A as outlined in FIG. 31.

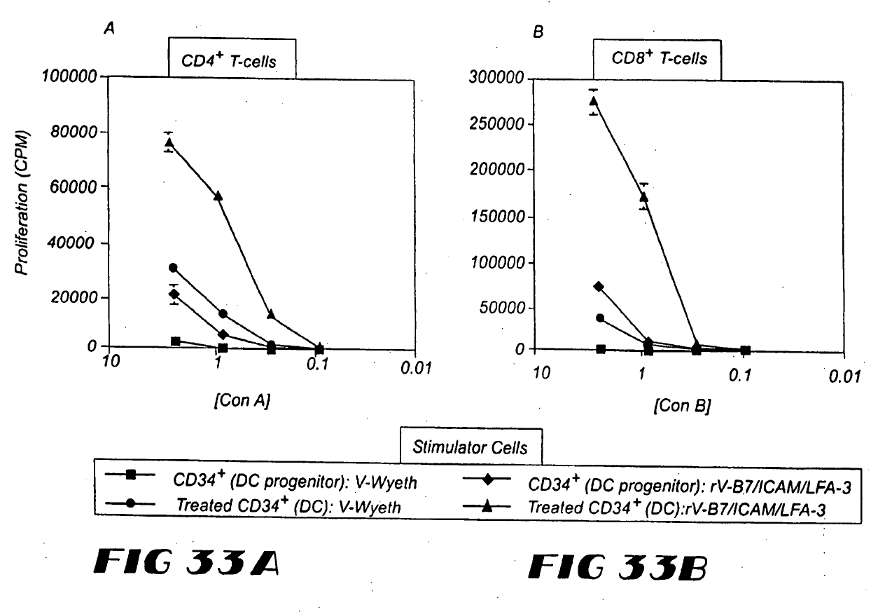

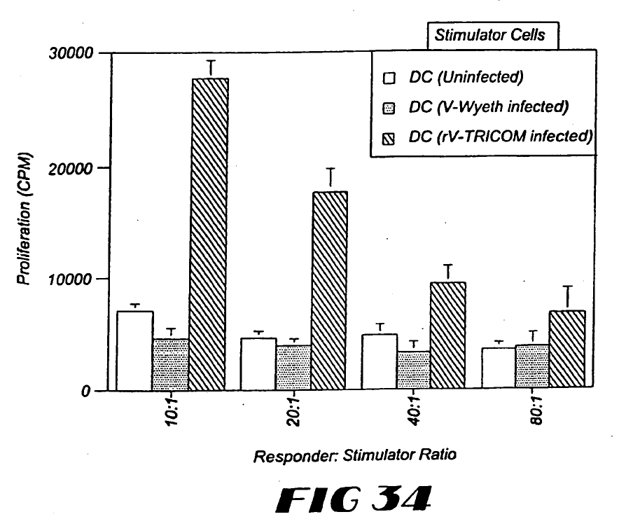

Progenitor dendritic cells infected with recombinant poxvirus encoding B7.1, ICAM-1, and LFA-3 were able to stimulate both CD4⁺ and CD8⁺ T cells. The stimulation of CD8⁺ T cells by the B7.1, ICAM-1 LFA-3 expressing progenitor dendritic cells was greater than that achieved using non-infected mature CD34⁺ dendritic cell (FIG. 32). Moreover, infection and expression of the three costimulatory molecules in mature CD34⁺ dendritic cells (pretreated with IL-4 and GM-CSF) resulted in a dramatic increase in stimulation of both CD4⁺ and CD8⁺ T cells (FIG. 33).

One skilled in the art can also measure the quality of a dendritic cell population by its ability to support an alloreactive response (mixed lymphocyte reaction, MLR) (43). FIG. 34 shows the results of a mixed lymphocyte culture using dendritic cells infected with rV-TRICOM. The mixed lymphocyte reaction uses DCs from C57BL/6 mice which are stimulating T lymphocytes from Balb/c. (i.e. an anti-allotype reaction).

These data show that the degree of proliferation in a mixed lymphocyte reaction is dramatically higher using DCs infected with rV-TRICOM as compared to uninfected DCs or DCs infected with wild-type vaccinia.

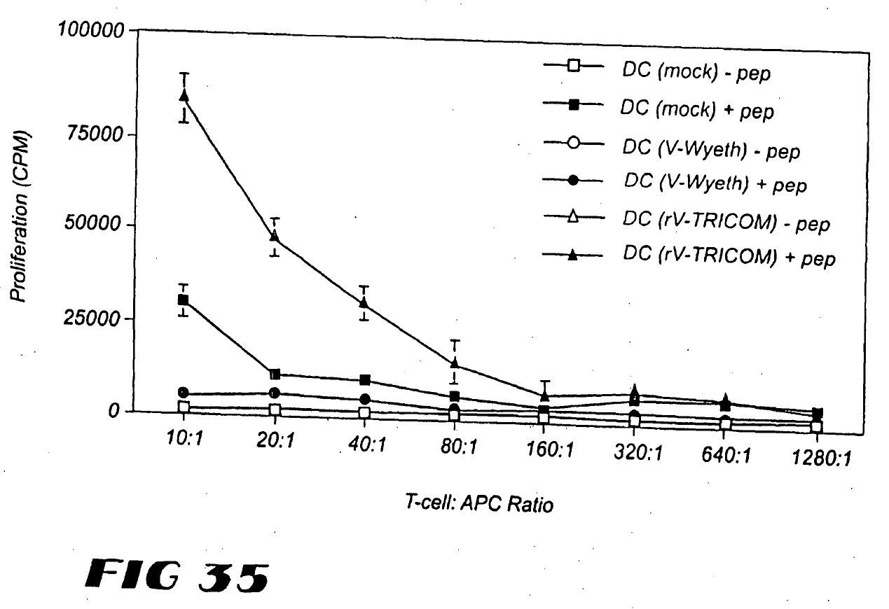

FIG. 35 demonstrates that DCs infected with rV-TRICOM are far superior than standard DCs in stimulating a CEA peptide-specific murine T cell line. This T-cell line is CD8⁺ and is specific for the CEA D^{b} Class-I restricted epitope EAQNTTYL (CAP-M8). The combination of DCs pulsed with the CEA peptide (1 .mu.g/ml) and previously infected with rV-TRICOM is clearly superior in stimulating CEA-specific T cell responses, especially at low T-cell to DC ratios.

### Example 31

Murine T Cell Stimulation In Vitro and In Vivo Using rV- or rF-TRICOM Infected Murine Bone Marrow-Derived Dendritic Cells

Experimental Protocol

Peptides

The H-2k^{b}-restricted peptides OVA (ovalbumin₂₅₇₋₂₆₄, SIINFEKL)⁴¹ and VSVN (vesicular stomatitis virus N₅₂₋₅₉, RGYVYQGL)⁴², and the H-2 D^{b} restricted peptides CAP-M8 (CEA₅₂₆₋₅₃₃, EAQNTTYL) and FLU-NP(NP₃₆₆₋₃₇₄, ASNENMDAM)⁴³ were either purchased (Multiple Peptide Systems, San Diego, Calif.) or synthesized in-house (Applied Biosystems 432A Synergy Peptide Synthesizer, Foster City, Calif.).

Cell Lines and Cell Cultures

The OVA and Cap-M8 CD8⁺ cytotoxic T-cell lines were generated in-house from C57BL/6 mice and recognize the OVA and Cap-M8 peptides, respectively. The CTL lines were maintained by weekly in vitro stimulation cycles with irradiated nave splenocytes in complete medium (CM) [RPMI 1640 with fetal calf serum (10%); glutamine (2 mM), sodium pyruvate (1 mM), Hepes (7 mM), gentamicin (50 .mu.g/ml), 2-mercaptoethanol (50 .mu.M), and non-essential amino acids (0.1 mM), (Biofluids, Rockville, Md.)], supplemented with 1 .mu.g/ml specific peptide and 10 U/ml murine IL-2 (Boehringer Mannheim, Indianapolis, Ind.). Twenty-four hours prior to using these cells as responders in antigen-specific proliferation assays, the cells were purified by centrifugation over a Ficoll-Hypaque gradient (density=1.119 g/ml, Sigma Chemical Co., St. Louis, Mo.) and replated in six-well culture plates (10⁶ cells/ml, 5 ml/well) in CM supplemented with 10 U/ml murine IL-2 only. For cytotoxicity assays, the target tumor-cell line used was EL-4 (C57BL/6, H-2^{b}, thymoma, ATCC TIB-39).

DC Preparation

Bone marrow was derived from six- to eight-week-old female C57BL/6 mice (Taconic Farms, Germantown, N.Y.). The procedure used in this study was a slightly modified version of that described by Inaba et al.⁴¹ Briefly, bone marrow was flushed from the long bones of the limbs and passed over a Ficoll-Hypaque gradient. Bone-marrow cells were depleted of lymphocytes and Ia⁺ cells using a cocktail of magnetic beads specific for CD4, CD8, and anti-MHC Class-II (MiniMACS, Miltenyi Biotec, Auburn, Calif.). Cells were plated in six-well culture plates (10⁶ cells/ml, 5 ml/well) in CM supplemented with 10 ng/ml GM-CSF and 10 ng/ml IL-4 (R&D Systems, Minneapolis, Minn.). Cells were replated in fresh cytokine-supplemented media on days 2 and 4. At 6 days of culture, cells were harvested for infection, analysis and immunizations. For specified experiments, DC were treated with murine TNF-.alpha. (100 ng/ml, Boehringer Mannheim, Indianapolis, Ind.) or CD40 mb (5 .mu.g/ml. PharMingen, San Diego, Calif.) during the final 24 h of culture.

Recombinant Poxviruses

The rV virus containing the gene that encodes the murine costimulatory molecule B7-1 (CD80) under control of the synthetic early/late (sE/L) promoter (designated rV-B7-1) has been described herein. The rV virus containing the murine LFA-3 gene (CD48) under control of the vaccinia 30K (M2L) promoter, the murine ICAM-1 (CD54) gene under control of the vaccinia I3 promoter, and the murine B7-1 gene under control of the synthetic early/late (sE/L) promoter has been designated rV-TRICOM. The vectors rF-B7-1 and rF-B7-1/ICAM-1/LFA-3 (designated rF-TRICOM) are rF viruses that were constructed similarly to rV-B7-1 and rV-TRICOM, respectively. A fowlpox-TRICOM construct containing a reporter gene, human CEA, was used in certain experiments. Non-recombinant wild-type vaccinia virus (Wyeth strain) was designated V-WT, while wild-type fowlpox virus was designated FP-WT.

Infection of DC

DC were harvested on day 6 and washed with Opti-Mem (Gibco-BRL, Gaithersburg, Md.). The cells were then either mock-infected with HBSS; infected with V-WT, rV-B7, or rV-TRICOM at 25 MOI (multiplicity of infection, PFU/cell); or infected with FP-WT, rF-B7-1, or rF-TRICOM at 50 MOI in Opti-Mem for 5 h. Warm CM was added after infection, and the cells were incubated at 37 C. overnight. After infection, the cells were harvested for immunostaining, in vitro costimulation analysis, and in vivo administration.

Flow Cytometric Analysis

Cell-surface staining utilized three-color immunofluorescence. Staining was performed with primary FITC-labeled antibodies CD11c, CD11b, H-2K^{b}, H-2D^{b}, CD19, Pan-NK; primary PE-labeled antibodies 1A^{b}, CD48 (mLFA-3), CD86 (B7-2), CD3, CD14; and the biotin-labeled antibodies CD80 (B7-1), CD57 (ICAM-1), CD40. Biotin-labeled antibodies were subsequently labeled with Cychrome-streptavidin. All antibodies were purchased from PharMingen. Cell fluorescence was analyzed and compared with the appropriate isotype matched controls (PharMingen) with a FACSCAN cytometer (Becton Dickinson, Mountain View, Calif.) using the Lysis II software.

In vitro Costimulation Analysis: Pharmacological Signal-1

Female, six- to eight-week-old C57BL/6 mice were obtained (Taconic Farms, Germantown, N.Y.), and naive T cells were isolated as previously described⁵. T cells were added at 10⁵/well in 96-well, flat-bottomed plates (Costar, Cambridge, Mass.). Stimulator cells consisted of either uninfected DC, mock-infected DC, or DC infected with vaccinia vectors (V-WT, rV-B7-1, rV-TRICOM) or fowlpox vectors (FP-WT, rF-B7-1 or rF-TRICOM) irradiated (20 Gy) and added at 10⁴/well. Cells in all wells were cultured in a total volume of 200 .mu.l of CM in the presence of several concentrations (2.5 to 0.9 .mu.g/ml) of Con A (Sigma) for 2 days. Cells were labeled for the final 12-18 hr of the incubation with 1 .mu.Ci/well ³H-Thymidine (New England Nuclear, Wilmington, Del.) and harvested with a Tomtec cell harvester (Wallac Incorporated, Gaithersburg, Md.). The incorporated radioactivity was measured by liquid scintillation counting (Wallac 1205 Betaplate, Wallac, Inc.). The results from triplicate wells were averaged and are reported as mean CPM.+−.SEM.

Mixed-Lymphocyte Reaction

MLR was used to assess the stimulator, function of DC for allogeneic and syngeneic nave T cells. T cells were isolated from Balb/C or C57BL/6 mice as before. Stimulator cells consisted of DC that were either uninfected; mock infected; or infected with V-WT, rV-137-1, rV-TRICOM, FP-WT, rF-B7-1 or rF-TRICOM and irradiated (20 Gy). T cells (5.times.10⁴/well) were co-cultured with graded numbers of stimulator cells in CM in flat-bottom 96-well culture plates and incubated at 37 C., 5% CO₂ for 4 days, labeled for the final 12-18 hr of the incubation with I .mu.Ci/well ³H-Thymidine, harvested, and analyzed as before.

In vitro Costimulation Analysis: Peptide-Specific Signal

Rested OVA or CAP-M8 T cells (responders) were added at 5.times.10⁴/well in 96-well, flat-bottomed plates. Stimulator cells consisted of DC that were either uninfected, or infected with V-WT, rV-137-1, or rV-TRICOM and irradiated (20 Gy). Cells in all wells were cultured in a total volume of 200 .mu.l of CM. The costimulation assay was carried out using two sets of conditions: (1) a 10:1 fixed ratio of responder:stimulator cells that were cultured in the presence of several concentrations of specific peptide or appropriate control peptide or (2) a fixed concentration of specific peptide or control peptide cultured at various responder:stimulator cell ratios. Cells were cultured for 72 h, labeled for the final 12-18 h of incubation with 1 .mu.Ci/well ³H-Thymidine, harvested, and analyzed as before.

CTL Induction In Vivo and Cytotoxic Analysis

DC (1.times.10⁶) that were either uninfected or infected with V-WT or rV-TRICOM were washed twice in Opti-Mem and resuspended in 1 ml of the same medium containing 10 .mu.M of either OVA or CAP-T48 peptides. After 2 h incubation at 37 C., cells were washed twice in HBSS and resuspended in HBSS for injections. Peptide-pulsed DC (1.times.10⁵ cell/mouse) were injected 1-3 times intravenously at 7-day intervals. Control mice were immunized subcutaneously with 100 .mu.g indicated peptide in Ribi/Detox adjuvant (Ribi ImmunoChem Research, Hamilton, Mont.). Fourteen days following the final inoculation, spleens from two animals per group were removed, dispersed into single-cell suspensions, pooled, and co-incubated with 10 .mu.g/ml of appropriate peptide for six days. Bulk lymphocytes were recovered by centrifugation through a density gradient (LSM, Organon Teknika. West Chester, Pa.). EL-4 cells were prepared for use as targets in a standard cytolytic assay using ¹¹¹In, as previously⁴⁵. Target cells were pulsed with 10 .mu.M specific peptide for 1 hour at 37 C., while a second group of target cells was pulsed with control peptide. Lymphocytes and peptide-pulsed targets (5.times.10³ cells/well) were suspended in CM, combined at effector:target ratios of 80:1 to 10:1 in 96-well U-bottomed plates (Costar) and incubated for 5 h at 37 C. with 5% CO₂. After incubation, supernatants were collected using a Supernatant Collection System (Skantron, Sterling, Va.), and radioactivity was quantified using a gamma counter (Cobra Autogamma, Packard. Downers Grove. Ill.). The percentage of specific release of ¹¹¹In was determined by the standard equation: % specific lysis=[(experimental-spontaneous)/(maximum−spontaneous)].times.100. Where indicated, CTL activity was converted to lactic units (LU) as described by Wunderlich et al, 1994.

Anti-Vaccinia Antibody Analysis

V-WT was added at 5.times.10⁵/well to polyvinyl chloride plates (Dynatech, Chantilly, Va.), dried overnight at 37 C. and blocked with 5% BSA. Graded dilutions of sera from immunized mice was added in triplicate and incubated for 1 h at 37 C. Plates were washed and incubated with peroxidase labeled goat anti-mouse IgG (Kirkegaard and Perry Laboratories, Gaithersburg, Md.) for an additional hour. Wells were developed with o-phenylenediamine dihydrochloride (Sigma, St. Louis. Mo.) and H_{2O2}. Reactions were slopped with H₂SO₄. The absorbance of each well was read at 405 nm using a Bio-Tek EL312e microplate ELISA reader (Winooski, Vt.).

Results

Increased Expression of Costimulatory Molecules on DC

To determine the efficiency of poxvirus infection of DC, these cells were infected with either a rV virus encoding B7-1, ICAM-1 and LFA-3 (designated rV-TRICOM) or a rF virus encoding B7-1, ICAM-1, LFA-3 and human carcinoembryonic antigen (CEA) (designated rF-CEA/TRICOM). In the latter case, CEA was used as a reporter gene since fowlpox structural proteins are not expressed in infected cells. After 18 h, cells were analyzed for the expression of cell-surface markers associated with the particular viral infection. Uninfected control DC expressed CD11b (97%) and were negative for the expression of vaccinia proteins. After infection with rV-TRICOM, 94% of DC co-expressed both CD11b and vaccinia proteins. DC infected with rF-CEA/TRICOM co-expressed both CD11b and CEA (87%). These DC failed to express fowlpox proteins as detected by polyclonal rabbit anti-fowlpox sera (data not shown), which is in agreement with reports stating that fowlpox does not replicate in mammalian cells. Taken together, these data indicate that DC are efficiently infected by both rV and rF vectors.

The cardinal characteristics of DC are high expression levels of both histocompatibility antigens and costimulatory molecules. To further characterize the phenotype of DC after virus infection, cells were infected with wild-type vaccinia virus (V-WT), rV-B7-1, rV-TRICOM, wild-type fowlpox (FP-T) or rF-TRICOM and analyzed for the expression of cell-surface markers associated with the DC phenotype (Table 4). As expected, uninfected and mock-infected DC expressed high levels of MHC Class I and II, CD11b, B7-2 and CD40 molecules, as well as high levels of B7-1, ICAM-1 and LFA-3. DC infected with V-WT expressed lower cell-surface densities (as determined by MFI) of several molecules, while DC infected with rV-B7-1 expressed 5-fold more B7-1 than uninfected DC (MFI from 329 to 1689). Infection of DC with rV-TRICOM substantially increased MFI and the percentage of cells positive for B7-1, ICAM-1, and LFA-3. DC infected with FP-WT had a similar phenotypic profile to that of uninfected DC. Infection of DC with rF-TRICOM also substantially increased MF1 and the percentage of cells positive for B7-1, ICAM-1, and LFA-3. All DC populations remained negative for T-cell (CD3), B-cell (CD19), monocyte/neutrophil (CD14), and NK-cell (Pan NK) markers both before and after infection with rF or N vectors (Table 4).

TABLE 4

| | DC Panel [(% positive cells (MFI)] | | | | | | |
|---|---|---|---|---|---|---|---|
| Infection | I-A^{b} | H-2K^{b}/D^{b} | CD11b | CD11c | B7-2 | CD40 | B7-1 |

Infection of BMDC with rV-TRICOM or FP-TRICOM Increases the Expression Level of B7-1, ICAM-1, and LFA-3

| | | | | | | | |
|---|---|---|---|---|---|---|---|
| None | 88 (1124) | 89 (125) | 93 (935) | 20 (74) | 68 (490) | 68 (82) | 91 (329) |
| Mock | 87 (989) | 88 (125) | 90 (1129) | 25 (29) | 77 (432) | 71 (99) | 85 (330) |
| V-V-WT | 87 (890) | 86 (83) | 86 (588) | 29 (54) | 61 (274) | 79 (98) | 90 (197) |
| rV-B7-1 | 85 (856) | 35 (104) | 81 (693) | 25 (62) | 73 (304) | 73 (103) | 94 (1669) |
| rV-TRICOM | 87 (901) | 78 (103) | 77 (558) | 22 (54) | 66 (298) | 71 (81) | 96 (1442) |
| FP-WT | 91 (935) | 93 (126) | 89 (889) | 24 (69) | 65 (487) | 71 (90) | 94 (382) |
| rF-B7*1 | 88 (987) | 99 (114) | 36 (793) | 28 (72) | 74 (404) | 68 (90) | 95 (1559) |
| rF-TRICOM | 90 (900) | 99 (115) | 84 (789) | 27 (66) | 76 (499) | 73 (93) | 99 (1824) |

| | DC Panel [(% positive | | | | | |
|---|---|---|---|---|---|---|
| | cells (MFI)] | | Non-DC Markers | | | |
| Infection | ICAM-1 | LFA-3 | CD14 | CD19 | CD3 | Pan NK |

Infection of BMDC with rV-TRICOM or FP-TRICOM Increases the Expression Level of B7-1, ICAM-1, and LFA-3

| | | | | | | |
|---|---|---|---|---|---|---|
| *None | 96 (595) | 88 (153) | 2 (20) | 2 (26) | 0.3 (33) | 2 (56) |
| Mock | 97 (519) | 86 (189) | 1 (42) | 4 (30) | 0.6 (37) | 2 (29) |
| V*V*WT | 95 (241) | 70 (196) | 3 (20) | 3 (45) | 1 (59) | 5 (50) |
| rV-B7-1 | 97 (364) | 72 (131) | 3 (42) | 3 (40) | 0.8 (67) | 4 (37) |
| rV-TRICOM | 94 (1528) | 92 (304) | 3 (32) | 4 (29) | 0.9 (70) | 4 (31) |
| FP-WT | 97 (464) | 91 (130) | 2 (18) | 2 (10) | 1 (60) | 2 (31) |
| rF-B7*1 | 98 (437) | 90 (183) | 2 (30) | 2 (29) | 1 (45) | 2 (54) |
| rF-TRICOM | 98 (1697) | 95 (530) | 3 (22) | 3 (13) | 1 (50) | 3 (33) |

DC were uninfected, mock infected, or infected with 25 MOI of either V-WT, rV-B7, or rV-TRICOM or 50 MOI of either WT-FP or FP-TRICOM for 5 h. After 18 h incubation, cells were phenotyped by 3-color flow cytometric analysis. Bold numbers indicate a >100% change in cell-surface expression (MFI).

DC Infected with TRICOM Vectors Exhibit Enhanced Capacity to Stimulate Nave T Cells

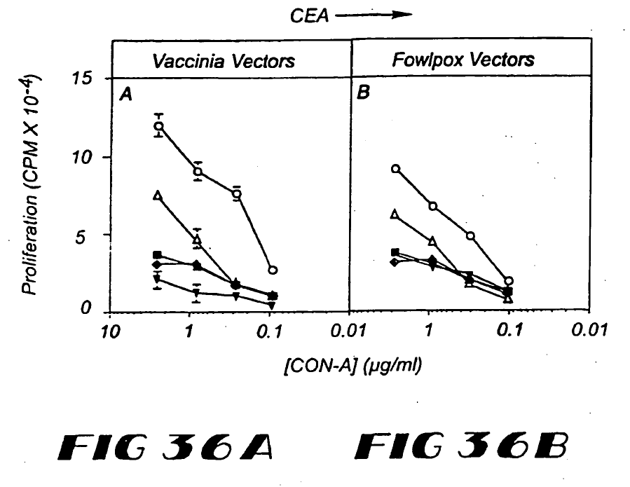

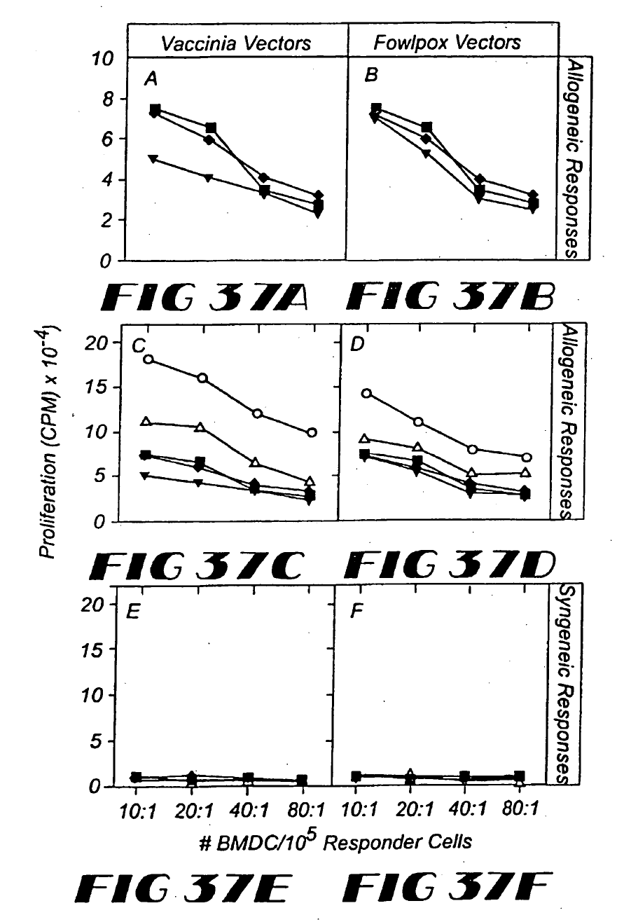

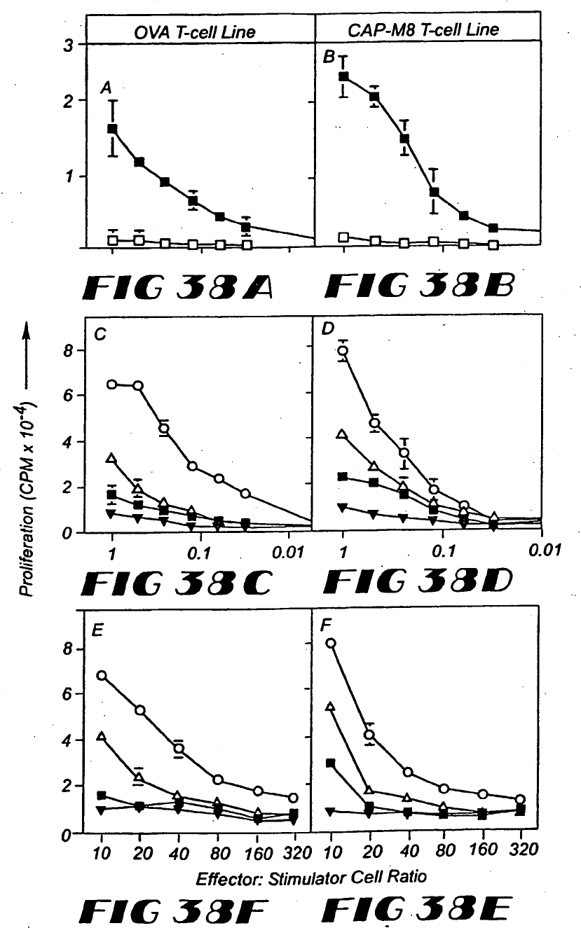

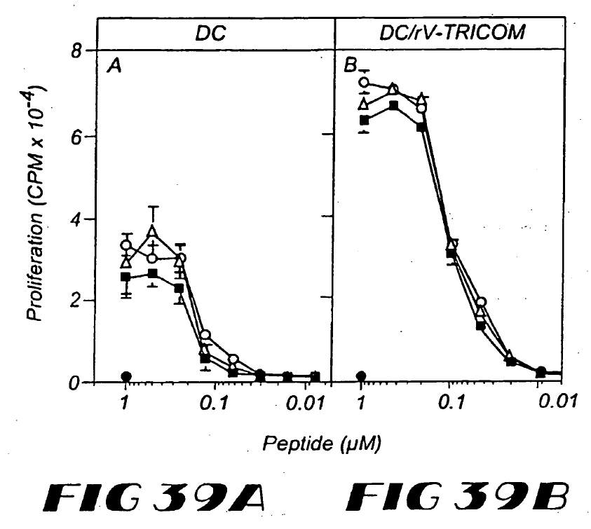

An in vitro model was used to analyze how increased levels of B7-1, ICAM-1 and LFA-3 expression help induce nave T-cell proliferation. In this model, the first signal for T-cell activation was delivered via a pharmacological reagent (Con A) and the additional, or costimulatory, signal was delivered to the T-cell via DC or DC expressing higher levels of TRICOM as a consequence of recombinant poxvirus infection. In these and all subsequent studies reported here, V-WT and FP-WT were also used to rule out effects due to the vector alone. As shown in FIG. 36A, both uninfected and mock-infected DC induced proliferation of T-cells. DC infected with V-WT (designated DCN-TRICOM) induced less T-cell proliferation than uninfected DC. Delivery of additional costimulatory signals via DC infected with rV-B7-1 (designated DC/rV-B7-1) increased proliferation compared with uninfected DC. However, DC infected with rV-TRICOM (designated DC/rV-TRICOM) induced further increases in T-cell proliferation at all concentrations of Con A. In addition, when T-cells were stimulated with DC/rV-TRICOM, 28-fold less Con A was needed to induce proliferation to levels comparable to that of uninfected DC. When these experiments were repeated using fowlpox vectors, DC/rF-TRICOM induced increases in T-cell proliferation at all Con A concentrations, unlike DC or DC/rF-B7-1 (FIG. 36B). These experiments were repeated 4 times with similar results.

Enhanced Allostimulatory Activity by DC Infected with TRICOM Vectors

The effect of rV-TRICOM (FIGS. 37A, C, E) or rF-TRICOM (FIGS. 37B, D, E) infection on DC stimulatory capacity was assessed in an allospecific mixed-lymphocyte reaction. Both uninfected DC and mock-infected DC populations induced a strong proliferation (78,000 CPM) of allogeneic T cells (FIGS. 37A, B). The stimulatory capacity of DC was increased after infection with rV-B7-1 (FIG. 37C). Infection of DC with rV-TRICOM increased the stimulatory capacity over DC and DC/rV-B7-1 at all DC/responder ratios (FIG. 37C). Importantly, DC populations infected with rV-TRICOM vectors failed to stimulate syngeneic T cells (FIG. 37E). When these experiments were repeated using fowlpox vectors (FIGS. 37B, D), DC/rF-TRICOM induced larger increases in allogeneic T-cell proliferation than DC and DC/rF-B7-1, DC/rF-TRICOM, however, failed to stimulate syngeneic T cells (FIG. 37F). These experiments were repeated 3 times with similar results.

In Vitro Costimulation Analysis: Presentation of Peptides to Effector T Cells

Studies were undertaken to determine if the stimulatory capacity of peptide-pulsed DC could be enhanced by infecting DC with rV-TRICOM. To that end, the H-2K^{b}-restricted OVA (ovalbumin₅₇₂₋₂₆₄, SIINFEKL) peptide and an OVA-specific CD8⁺ effector T-cell line were used. DC were exposed to different concentrations of OVA peptide and incubated in the presence of the OVA T-cell line (FIGS. 38A-38F). The conventional (i.e., uninfected) DC induced a strong proliferation of OVA-specific T cells when incubated with the OVA peptide (FIG. 38A). These DC did not induce proliferation of OVA-specific T cells when incubated with the control peptide VSVN (vesicular stomatitis virus N₅₂-59 RGYVYQGL) (FIG. 38A, open squares). DC/rV-B7-1 increased the overall peptide-specific proliferation of these cells 1.8-fold (FIG. 38C). In addition, DC/rV-B7-1 induced similar proliferation to that of uninfected or mock-infected DC in the presence of 4-fold less peptide. In contrast, DC/rV-TRICOM increased the overall proliferation of these T-cells several-fold, and in the presence of 32-fold less OVA peptide, induced proliferation comparable to that of uninfected DC (FIG. 38C). To further evaluate the capacity of vaccinia-infected DC to present peptide, DC were pulsed with a single concentration of OVA peptide (1 .mu.M) and incubated in the presence of several ratios of T cells (FIG. 38E). On a per-cell basis, 4-fold fewer DC/rV-B7-1 were required to induce proliferation levels comparable to that of DC (open triangles vs. closed squares). The greatest stimulatory effect was that of DC/rV-TRICOM, which induced proliferation levels comparable to that of DC with 32-fold less cells (open circles vs. closed squares).

A second peptide system employing peptide-pulsed DC and an established T-cell line were employed to determine if results similar to those obtained with the OVA peptide could be noted. These experiments were conducted using the H-2 D^{b}-restricted peptide CAP-M8 (CEA₅₂₆₋₅₃₃, EAQNTTYL) and a CAP-M8-specific CD8⁺ effector T-cell line; similar results were noted (FIGS. 38B, D, F). These experiments were repeated 5 additional times with the same results.

Effect of rV-TRICOM Infection on TNF.alpha. or CD40-Matured DC

Since the functional maturation of DC is believed to correlate with the upregulation of T-cell costimulatory molecules, experiments were conducted to examine the effect of RV-TRICOM infection on DC that had been matured by co-culture with either TNF-.alpha. or CD40 mAb. Treatment of DC with TNF-.alpha. during the final 24 h of culture resulted in some upregulation of MHC-II, B7-2, and ICAM-1 as determined by flow cytometric analysis (Table 5), while treatment of DC with CD40 mAb resulted in the upregulation of ICAM-1 expression and a slight upregulation of MHC-II. Functionally, treatment of DC with TNF-.alpha. or CD40 mAb culminated in a 28% and 16% increase, respectively, in peptide-specific proliferation over that of unmanipulated DC (FIG. 39A). Similar data were also obtained after treating DC with lipopolysaccharide (LPS). Infection of untreated DC with rV-TRICOM resulted in a substantial increase in T-cell proliferation (FIGS. 39A vs. 39B). Pretreatment with TNF-.alpha. or CD40 mAb followed by infection with rV-TRICOM, however, conferred only a slight stimulatory capacity in excess of that seen with rV-TRICOM infection alone (FIG. 39B). These experiments were repeated 3 additional times with similar results.

TABLE 5
Effect of Pretreatment of DC with TNF-α or CD40 mAB Prior to rV-TRICOM Infection
DC Panel [% positive cells (MFI)]

| Infection | Pretreatment | I-A^{b} | H-2K^{b}/D^{b} | CD11b | CD11c | B7-2 | CD40 | B7-1 | ICAM-1 | LFA-3 |
|---|---|---|---|---|---|---|---|---|---|---|
| DC(Uninfected) | None | 90 (924) | 93 (225) | 90 (835) | 26 (174) | 63 (340) | 62 (182) | 93 (389) | 96 (415) | 90 (263) |
| DC(Uninfected) | TNF-α | 95 (1189) | 91 (195) | 84 (729) | 20 (149) | 71 (412) | 66 (199) | 85 (320) | 97 (421) | 87 (249) |
| DC(Uninfected) | CD40 | 91 (990) | 89 (183) | 87 (788) | 22 (154) | 68 (374) | 69 (198) | 90 (297) | 95 (690) | 86 (216) |
| | mAb | | | | | | | | | |
| DC/rV-TRICOM | None | 87 (756) | 89 (214) | 85 (684) | 24 (93) | 69 (301) | 66 (103) | 96 (1989) | 98 (1487) | 93 (413) |
| DC/rV-TRICOM | TNF-α | 92 (991) | 90 (230) | 79 (558) | 21 (62) | 72 (308) | 68 (81) | 96 (1442) | 94 (1998) | 90 (394) |
| DC/rV-TRICOM | CD40 | 91 (905) | 90 (216) | 81 (614) | 23 (69) | 65 (337) | 71 (120) | 94 (1382) | 97 (1444) | 89 (310) |
| | mAb | | | | | | | | | |

DC were treated with TNF-α (100 ng/ml) or CD40 mAb (1 μg/ml) during the final 24 h of culture. DC or treated DC were then infected
with 25 MOI of rV-TRICOM for 5 h. After 18 h incubation, cells were phenotyped by 3-color flow cytometric analysis.

DC Infected with W-TRICOM are More Efficient at Priming CTL Responses In Vivo

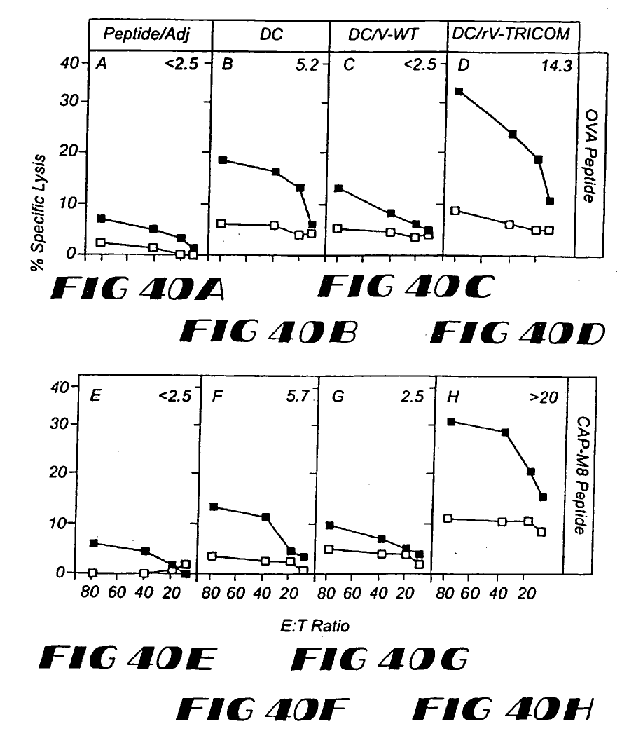

Experiments were conducted to determine if the enhanced stimulatory capacity of DC/rV-TRICOM noted in vitro using Con A (FIGS. 36E-F), mixed-lymphocyte reactions (FIG. 37) and two effector T-cell models (FIG. 38) would translate to enhanced efficacy in priming naive T-cell responses in vivo. To that end, DC, DC/V-WT, and DC/rV-TRICOM were pulsed with 10 .mu.M OVA peptide and administered intravenously to C57BL/6 mice. Control mice were immunized with OVA peptide in Ribi/Detox adjuvant subcutaneously. Splenocytes were harvested 14 days following vaccination, restimulated in vitro for 6 days, and assessed for their peptide-specific lytic ability against OVA-pulsed EL-4 cells. EL-4 cells pulsed with VSVN peptide were used as control target cells. As seen in FIG. 40A, CTL generated from mice immunized with peptide/adjuvant exhibited modest levels of CTL activity (FIG. 40A). Mice immunized with peptide-pulsed DC exhibited a greater peptide-specific CTL response (FIG. 40B). The induced CTL response was somewhat blunted in mice immunized with DC/v-WT (FIG. 40C, <2.5 lytic units (LU) vs. 5.2 LU). In contrast, mice immunized with peptide-pulsed DC/rV-TRICOM (FIG. 40D) exhibited a CTL response that was significantly stronger than that of DC (LU=14.3, p=0.001). Similar experiments were then conducted using a second model peptide, CEA peptide CAP-M8 (FIGS. 40E-H). Again, peptide-pulsed DC elicited much greater CTL activity than that educed by peptide/adjuvant (5.7 LU vs. <2.5 LU). In addition, mice immunized with peptide-pulsed DC/rV-TRICOM (FIG. 40H) exhibited a strong CTL response (>20 LU) compared with that induced by peptide-pulsed DC (5.7 LU, p=<0.001; FIG. 40F).

Efficacy of Multiple Vector-Infected DC Vaccinations

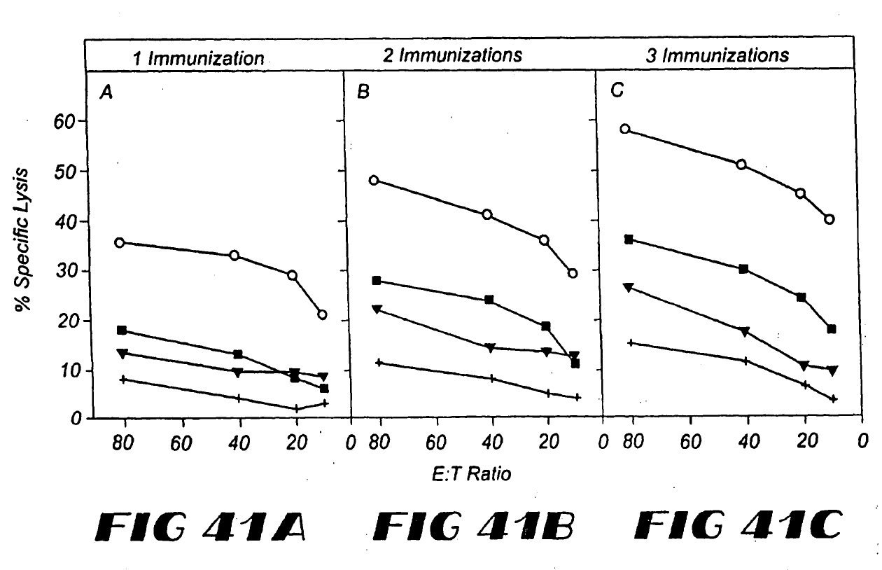

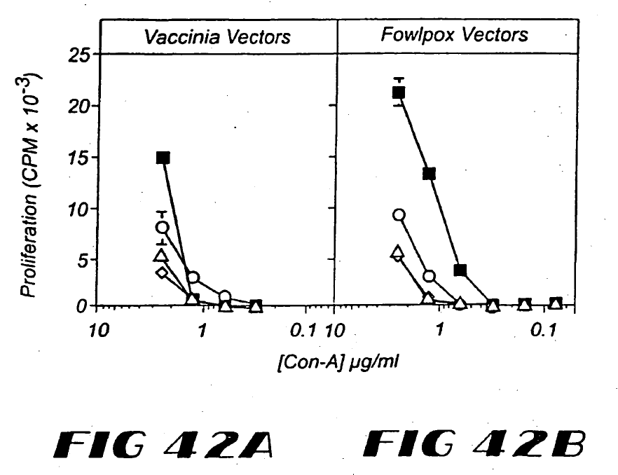

It is generally believed that the generation of anti-vaccinia antibodies can prevent the repeated use of vaccinia virus as immunogens. However, little is known about the repeated use of vaccinia-infected cells as immunogen. To address this issue, an immunization scheme was carried out in which CAP-M8 peptide-pulsed DC immunogens were administered one, two, or three times, at 7-day intervals. As before, splenocytes were harvested 14 days following the final immunization, restimulated in vitro for 6 days, and assessed for their peptide-specific lytic ability against CAP-M8-pulsed EL-4 cells. As seen in FIG. 41A, peptide-pulsed DC/rV-TRICOM induced higher levels of CTL activity when compared with peptide-pulsed DC. These data are similar to those seen in FIGS. 40E-H. This single administration of DC/V-WT or DC/rV-TRICOM induced significant anti-vaccinia IgG antibody titers, with values ranging from 1:4,000 to 1:9,000 as determined by qualitative ELISA. These titers, however, had no effect on the capacity of these immunogens to boost CTL activity upon subsequent immunizations (FIGS. 41B and 41C). While anti-vaccinia virus liters after the second vaccination ranged from 1:12,000 to 1:50,000, a boost in the induction of peptide-specific CTL was seen in all groups. Again, the CTL activity observed employing DC/rV-TRICOM-pulsed cells was greater than that observed with peptide-pulsed DC.

### Example 32

Splenocytes or Bone Marrow Progenitor Cells Infected with TRICOM Vectors Induce T-cell Activation Comparable to Dendritic Cells Materials and Methods

Generation of Bone Marrow Progenitor Cells and Dendritic Cell Cultures.

The procedure used for generation of bone marrow-derived DC was that described by Inaba et al. with minor modifications. Briefly, the femurs were taken from 6-8 week old female C57BL/6 mice (Taconic Farms, Germantown, N.Y.) and the bone marrow was flushed and passed over a Ficoll-Hypaque gradient. Bone marrow cells were depleted of lymphocytes and Ia⁺ cells using a cocktail of magnetic beads specific for CD4, CD8, and MHC Class II (MiniMACS, Miltenyi Biotec, Auburn, Calif.). Designated as dendritic cell progenitors, these depleted bone marrow cells were then prepared for infection, or for dendritic cell cultures depleted bone marrow cells were plated in six-well culture plates (106 cells/ml 5 ml/well) in CM supplemented with 10 ng/ml GM-CSF and 10 ng/ml IL-4 (R&D Systems, Minneapolis, Minn.). DC cultures were replated in fresh cytokine-supplemented CM on days 2 and 4, and split to new plates on day 4. At day 7 of culture, cells were harvested for analysis, in vitro assays, and in vivo immunizations.

Generation of Splenocyte Stimulator Cells.

Spleens were harvested from nave female C57BL/6 mice, crushed into a single-cell suspension, and passed over a Ficoll-Hypaque gradient. Splenocytes were depleted of lymphocytes and Ia⁺ cells using a cocktail of magnetic beads specific for CD90, and MHC Class II. Purified splenocytes were then washed twice with Opti-Mem (Gibco-BRL) and prepared for infection with the recombinant poxviruses.

Infection of Stimulator Cells.

Bone marrow-derived dendritic cell progenitor and splenocyte cells were washed twice with Opti-Mem and mock infected or infected with either 25 MOI V-WT, rV-B7-1, rV-TRICOM, or 50 MOI FP-WT, rF-B7-1 or rF-TRICOM at 25 MOI (multiplicity of infection. PFU/cell) in 1 ml final volume of Opti-Mem for 5 hours. After infection, warm (37 degree) CM was added and the cells were incubated at 37 C. overnight. After infection the cells were harvested for immunostaining, in vitro costimulation analysis, and in vivo administration.

Costimulation Analysis

Rested CAP-M8 T-cells (responders) were added at 5.times.10⁴/well in a 96-well flat-bottomed plates (Costar, Cambridge. Mass.). Stimulator cells consisted of BMDC, splenocytes, or bone marrow progenitors, either uninfected, mock infected, or infected with either V-WT, rV-B7-1, rV-TRICOM, FP-WT, or rF-TRICOM and irradiated (20 Gy). Cells in wells were cultured in a total volume of 200 ml of CM. The costimulation assay was carried out using two sets of conditions: a) fixed ratio of responder:stimulator cell of 2.5:1 for non-BMDC stimulators, and 10:1 for BMDC, cultured in the presence of several concentrations of Con-A as signal one, specific peptide, or appropriate control peptide, or b) a fixed concentration of Con-A as signal one, specific peptide, or control peptide, cultured at various responder:stimulator cell ratios. Cells were cultured for 48 or 72 hours for Con-A and peptide-specific assays, respectively, and labeled for the final 12-18 hours of the incubation with 1 mCi/well 3H-Thymidine, harvested, and analyzed as described above.

Table 6 shows splenocyte and bone marrow (BM) cell surface expression of costimulatory molecules after infection with recombinant vectors. Purified murine splenocytes or bone marrow cells were infected for 5 hours with 25 MOI of vaccinia vectors or 50 MOI of fowlpox vectors. Cell phenotype was compared with that of DC. All cells were immunostained with costimulatory molecule-specific mAbs labeled with fluorescein isothiocyanate, phycoerythrin, or biotin/streptavidin-cychrome. Isotype control were negative (data no shown). Numbers indicate percent positive cells and mean fluorescence intensity in parentheses.

TABLE 6
Infection of BMDC, Splenocytes, and BMDC Progenitors with either rV-TRICOM
or rF-TRICOM increases the level of expression of B7-1, ICAM-1, and LFA-3²

| | | I-A^{b} | H-2K^{b}/D^{b} | CD11b | CD11c | CD40 | B7-2 | B7-1 | ICAM-1 | LFA-3 | CD19 |
|---|---|---|---|---|---|---|---|---|---|---|---|
| DC² | Unin- | 88 (1124) | 89 (125) | 93 (935) | 20 (74) | 68 (82) | 93 (329) | 91 (329) | 96 (595) | 88 (153) | 2 (26) |
| | fected | | | | | | | | | | |
| Spleno- | Unin- | 92 (102) | 96 (389) | 3 (136) | 1 (54) | 87 (494) | 49 (61) | 46 (540) | 85 (258) | 77 (40) | 42 (25) |
| cytes³ | fected | | | | | | | | | | |
| | V-WT | 91 (124) | 94 (400) | 3 (182) | 0.7 (82) | 75 (408) | 63 (92) | 55 (490) | 76 (257) | 47 (33) | 25 (24) |
| | rV-B7 | 91 (123) | 95 (402) | 3 (89) | 1 (159) | 81 (369) | 61 (89) | 87 (1134) | 85 (315) | 45 (29) | 23 (27) |
| | rV-Tricom | 93 (283) | 93 (433) | 3 (41) | 3 (93) | 81 (327) | 49 (69) | 87 (1104) | 92 (788) | 97 (192) | 27 (33) |
| | FP-WT | 90 (104) | 90 (420) | 2 (162) | 0.9 (92) | 79 (418) | 60 (72) | 55 (460) | 70 (257) | 49 (32) | 53 (29) |
| | rF-B7-1 | 91 (133) | 86 (422) | 1 (81) | 1 (149) | 85 (399) | 55 (96) | 83 (830) | 83 (215) | 51 (29) | 52 (31) |
| | rF-Tricom | 89 (238) | 96 (399) | 3 (91) | 2 (80) | 86 (387) | 51 (99) | 86 (1001) | 92 (588) | 99 (292) | 48 (33) |
| BM⁴ | Unin- | 9 (289) | 99 (389) | 80 (114) | 1 (909) | 26 (136) | 28 (72) | 79 (115) | 68 (144) | 37 (89) | 2 (147) |
| | fected | | | | | | | | | | |
| | V-WT | 8 (228) | 98 (236) | 66 (144) | 1 (2132) | 19 (161) | 19 (98) | 75 (131) | 63 (151) | 33 (64) | 5 (50) |
| | rV-A7 | 8 (292) | 97 (259) | 71 (144) | 2 (394) | 25 (233) | 22 (125) | 89 (1117) | 56 (204) | 31 (65) | 2 (106) |
| | rV-Tricom | 7 (242) | 92 (183) | 70 (129) | 1 (875) | 26 (172) | 26 (91) | 92 (880) | 80 (490) | 38 (112) | 3 (62) |
| | FP-WT | 8 (318) | 98 (298) | 64 (133) | 1 (1101) | 23 (175) | 22 (88) | 74 (121) | 60 (112) | 35 (69) | 2 (30) |
| | rF-B7-1 | 7 (292) | 99 (259) | 75 (229) | 2 (1001) | 26 (245) | 26 (101) | 91 (652) | 50 (104) | 34 (72) | 1 (86) |
| | rF-Tricom | 8 (233) | 96 (233) | 72 (228) | 2 (984) | 25 (121) | 13 (99) | 96 (1880) | 79 (310) | 39 (109) | 1 (52) |

¹cells were uninfected or infected with 25 MOI of V-WT, rV-B7-1, rV-Tricom or 50 MOI FP-WT, rF-B7-1, or rF-TRICOM for five hours. After an eighteen hour incubation period, cells were phenotyped by 3-color flow cytometric analysis. Values are given as [ [symbol] positive cells (Main Fluorescence Intensity)]
Bold numbers indicate a >2-fold change in cell surface expression (MFI).
²BMDC: day 6 bone marrow derived dendritic cells (cultured in 10 ng/ml GM-CSF/IL-4).
³Splenocytes depleted of T-cells via α-CD90 (Thy 1.2) magnetic beads.
⁴BMDC Progenitors bone marrow cells were depleted of T-cells and MHCII cells via [symbol] magnetic cells.

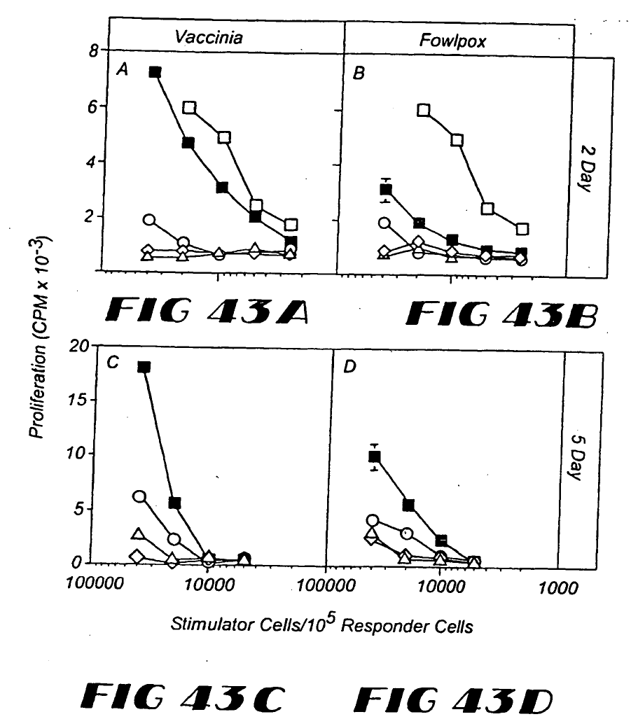

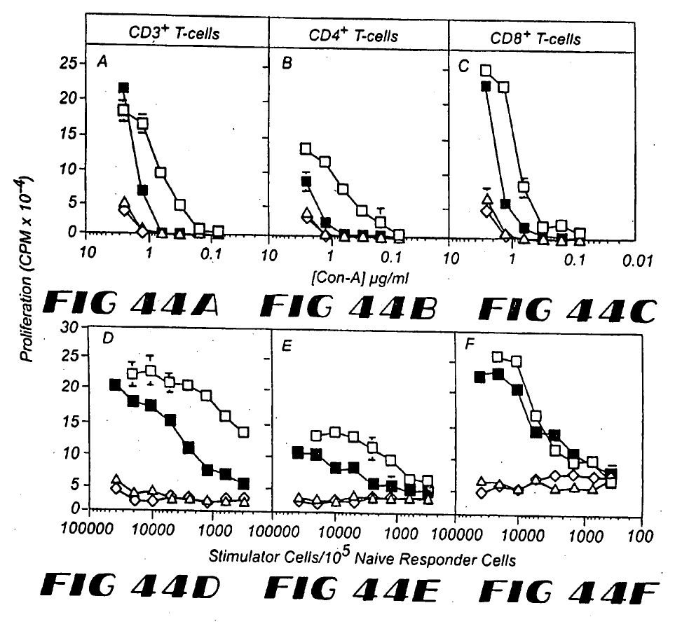

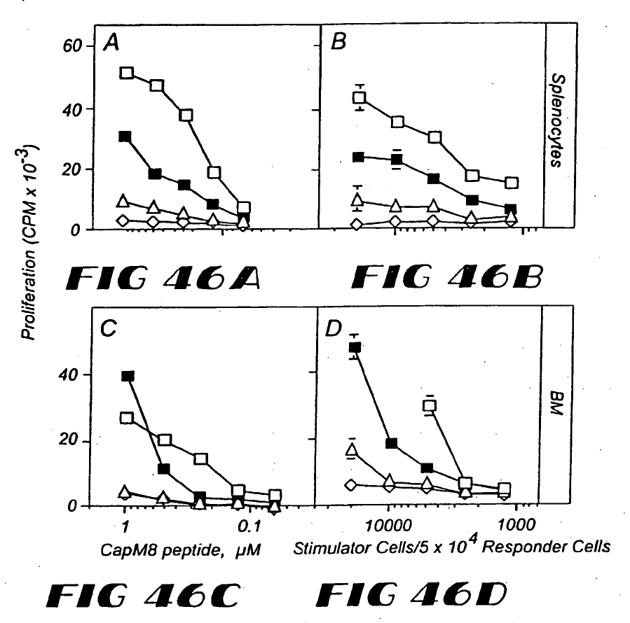

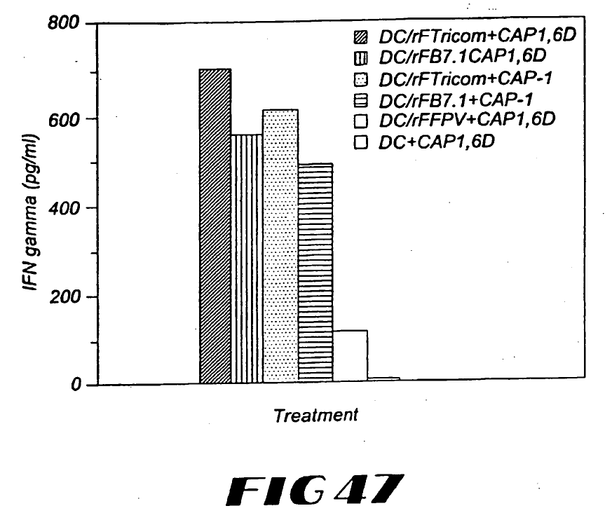

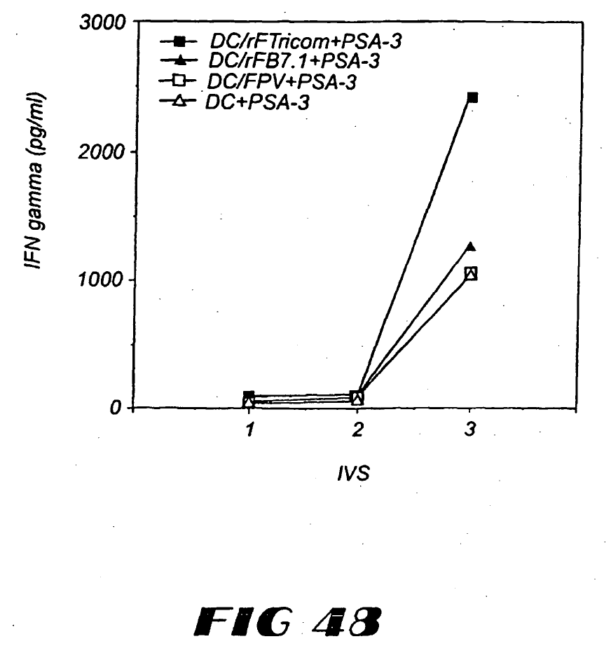

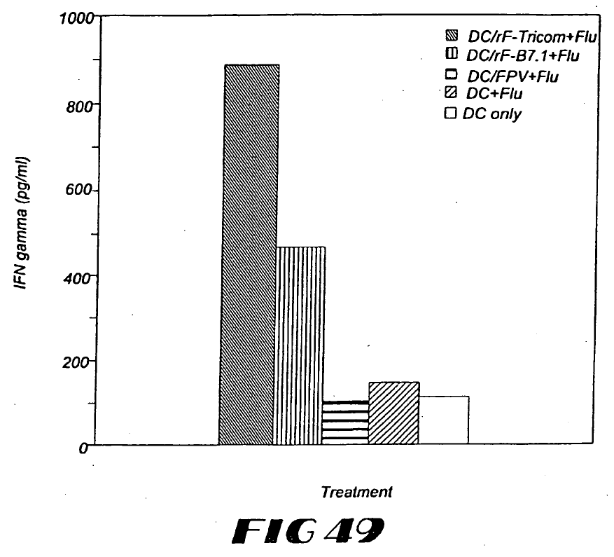

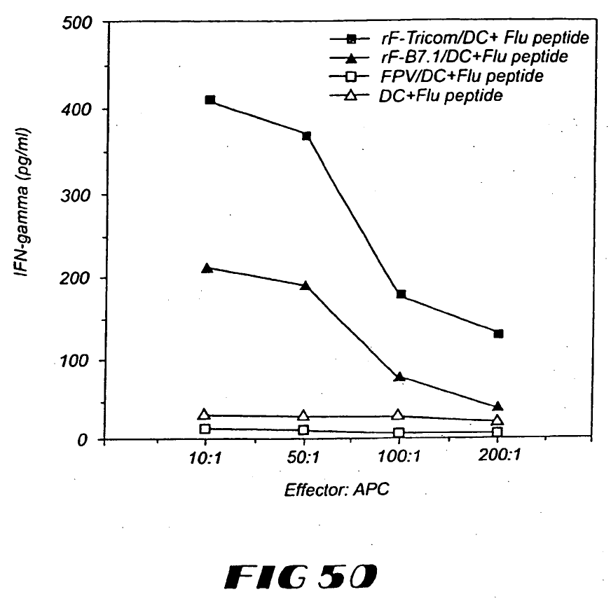

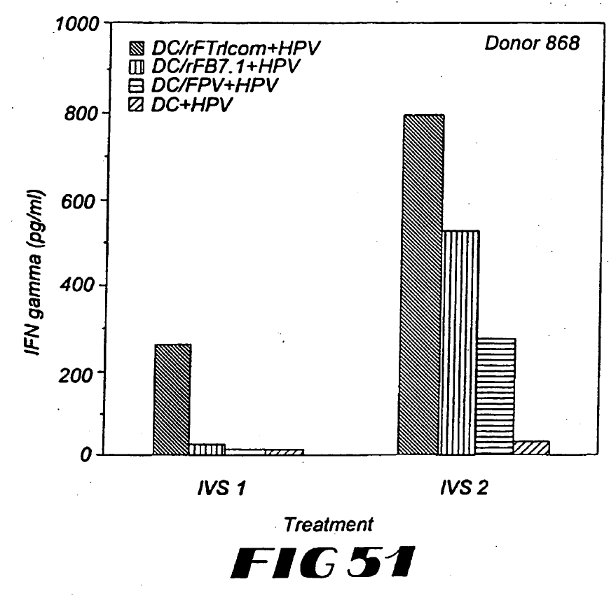

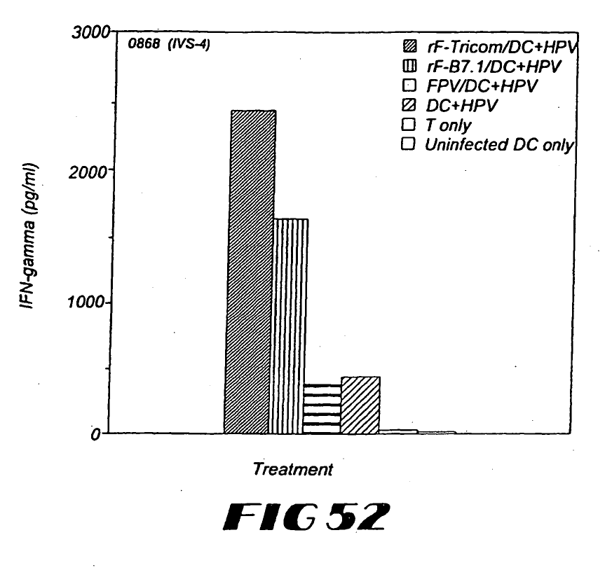

FIGS. 42A through 46 demonstrate that TRICOM-infected splenocytes are comparable to TRICOM infected bone marrow cells in stimulating T cell responses.

### Example 33

Human T Cell Stimulation Using Allogeneic rF-TRICOM Infected Human Dendritic Cells Pulsed with Peptides

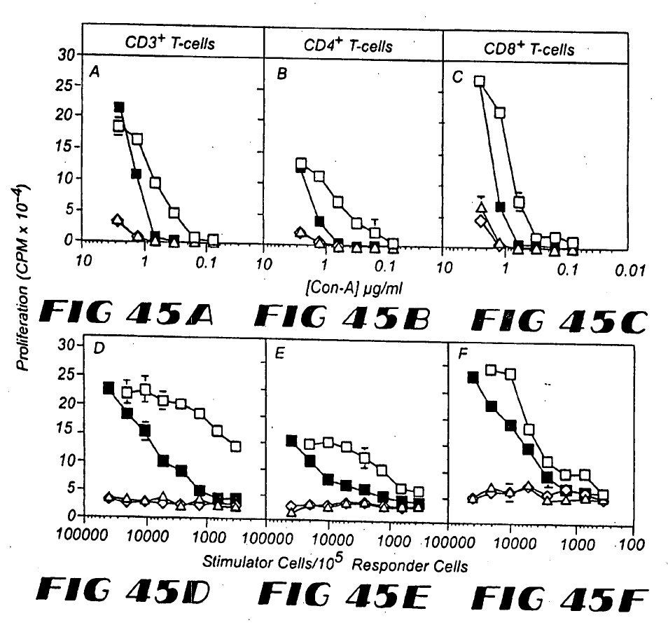

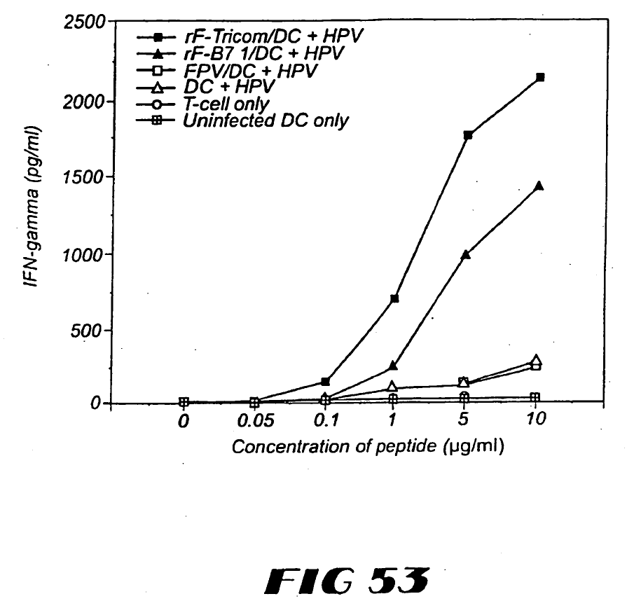

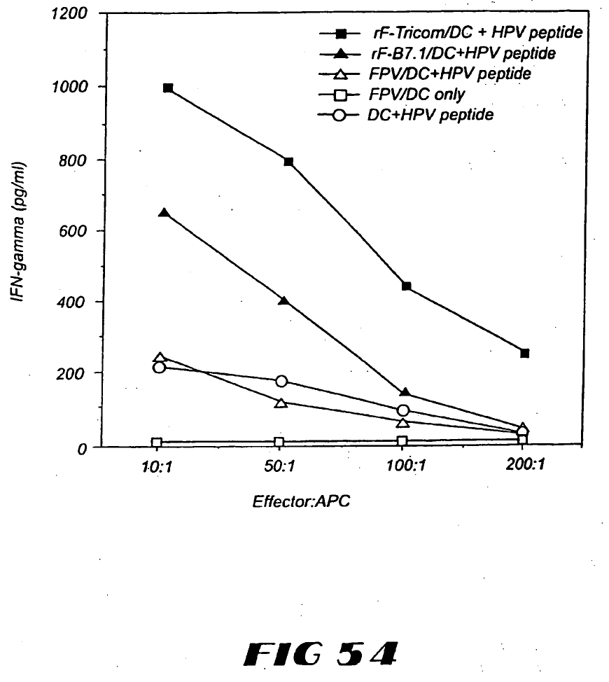

Human dendritic cells were isolated for a normal, healthy individual by leucophoresis. The human dendritic cells were cultured in the presence of GM-CSF and IL4 for 6-9 days, followed by the addition of rF-TRICOM or rF-Controls for infection of the dendritic cells. The rF-TRICOM-infected dendritic cells were pulsed with a CEA peptide (CAP-1 or CAP 1, 6D) (FIG. 47); a PSA peptide (PSA-3) (FIG. 48); an influenza peptide (Flu peptide 58-66) (FIGS. 49 and 50); or an HPV peptide (11-20) (FIGS. 51-45) for 1 hour. Human T cells isolated from peripheral blood mononuclear cells (PBMC) were cultured in the presence of the peptide-pulsed rF-TRICOM-infected dendritic cells and production of IFN-.alpha. by the T cells determined. FIGS. 47-54 show that peptide-pulsed rF-TRICOM infected human dendritic cells stimulated T cells to a greater extent than the controls. FIGS. 47-54, as well as Table 7 demonstrate that allogeneic human dendritic cells infected with rF-TRICOM can efficiently present any antigenic peptide to T cells for enhancement of an immune response.

TABLE 7
CTL activity of T cell lines by using
DC pulsed with HPV E7(11-20) peptide

| | T cell lines established by using | | | |
|---|---|---|---|---|
| | A | | | B |
| Target | rF-Tricom + P | rF-B7.1 + P | rF-FPV + P | DC + P |
| C1R-A2 + | 39.6 (3.1) | 24.7 (0.4) | 19.9 (2.9) | 7.3 (0.4) |
| HPV | | | | |
| C1R-A2 | 5.1 (3.0) | 6.9 (4.0) | 7.6 (2.0) | 8.0 (0.2) |

B:T ratio - 25:1
An 6 hour 111-In release assay was performed C1R-A2 cells were pulsed with HPV E7 peptide (11-20) YMDLQPETT at a concentration of 10 μg/ml.

The results presented in Table 7 demonstrate that DC infected with rF-TRICOM (A), are better as APC to generate CTL than are standard DC (B) when both are pulsed with peptide.

### Example 34

Human Clinical Trials of a rV-huTRICOM, rV-CEA huTRICOM Vaccine and rF-CEA TRICOM

The objective of the human clinical trial is to determine the optimum tolerated dose (OTD) of the recombinant rV-huTRICOM and rV-CEA-huTRICOM vaccine that elicits a host anti-tumor immune response and is associated with acceptable toxicity in patients with advanced CEA-expressing adenocarcinomas.

The rV-huTRICOM and rV-CEA-huTRICOM vaccines are produced under conditions suitable for Phase I and Phase II human clinical trial. In an initial trial, escalating doses of recombinant rV or rF CEA-huTRICOM live virus vaccine or rV-huTRICOM plus rV-CEA vaccine is provided at an initial dose of 10⁶ pfu virus, I.M., followed by a dose of 10⁷ pfu virus, I.M., which is followed later by of 10⁸ pfu virus, or 10⁹ S.C. or by scarification.

The anti-tumor response to each recombinant vaccine is determined using clinical, laboratory and radiologic evidence of tumor size, extent and growth using accepted standard criteria for measuring response of tumors to new forms of therapy as are known in the art.

The patient's immune response to the recombinant vaccine is assessed using a variety of immunological assays including anti-CEA antibody assay, anti-poxvirus antibody assay, immune complex assay, CEA-specific lymphoproliferative assay, CEA-specific cytotoxic T-lymphocyte assays, precursor frequency of CEA-reactive T cells in gamma-interferon release T-cell assay, a ELISPOT, Fast Immune, Tetramere assays for T-cell responses (Scheibenhogen et al Int. J. Cancer 71:932-936, 1997), HLA assays and the like. A comparison of pre-treatment and post-treatment samples are made to document development of humoral and cellular immune responses directed against the CEA tumor antigen.

### Example 35

Clinical Trials of an Recombinant Fowlpox-CEA-huTRICOM In an initial trail, escalating doses of recombinant fowlpox-CEA huTRICOM vaccine of 10⁶ pfu virus, 10⁷ pfu virus and 10⁸ pfu virus is injected directly into a tumor mass of a patient with advanced CEA-expressing adeno carcinomas.

The specific anti-tumor and immune response to the recombinant vaccine is determined as described in Example 34.

### Example 36

Human Clinical Trial of T Lymphocytes Activated by Multiple Costimulatory Molecule-Overexpressing Dendritic Cells

Peripheral blood lymphocytes and dendritic cells are obtained from a patient with advanced prostate cancer. The peripheral blood lymphocytes are enriched for CD8+ lymphocytes. The dendritic cells are infected with rV-PSA epitope QVHIPQKVTK/B7.1/ICAM-1/LFA-3 for a period of time sufficient to allow expression of the PSA epitope and overexpression of the multiple costimulatory molecules. PSA epitope-specific CD8⁺ lymphocytes are activated and expanded in the presence of these treated dendritic cells. The activated PSA epitope-specific CD8⁺ autologous T lymphocytes are injected into the patient alone and in combination with the PSA epitope. The specific anti-tumor and PSA-specific immune response to the treatment is determined by methods comparable to those described in Example 34.

Similar human clinical trials may be conducted for treatment of patients with other TAA-expressing cancers, by replacement of the gene encoding CEA with a gene encoding another TAA into the recombinant vector of the present invention.

### Example 37

Screen for Immunogenic Peptides and/or Human T Cells Immunoreactive with a Specific Peptide Using DC Infected with rF-TRICOM

The present invention encompasses anticancer therapies using ex vivo engineering of DC with viral vectors carrying a tumor associated antigen gene to activate tumor-specific CTL. DC infected with rF-CEA in combination with TRICOM costimulatory molecules are used to augment CEA-specific immune responses. The CTL induction capacity of DC infected with rF-CEA/TRICOM and rF-TRICOM are evaluated. Tetrameric MHC class I CAP-1 complex are used to visualize CAP-1 specific CTL. This protocol is not limited to the tumor associate antigen, CEA, but may be modified to elicit antigen-specific immune responses for any antigenic peptide or immunogenic epitope thereof for immunotherapy against cancer, pathogenic bacteria, virus, protozoans, yeast and the like. Moreover, the method may be modified to screen for and identify immunogenic peptides from a source such as a natural protein, recombinant protein, synthetic protein, or fragments from each, combinatorial libraries, and the like.

Materials and Methods

Cell Cultures

Colorectal carcinoma cell lines SW1463 (HLA-A1, 2), LS174T (HLA-A2,−), were purchased from American Type Culture Collection (Manassas. Md.). The cultures were free of mycoplasma and were maintained in complete medium [DMEM (Life Technologies, Inc. Grand Island, N.Y.) supplemented with 10% fetal bovine serum, 2 mM glutamine, 100 units/ml penicillin, and 100 .mu.g/ml streptomycin (Life Technologies, Inc.)]. The CIR cell line is a human plasma leukemia cell line that does not express endogenous HLA-A or B antigens (Storkus, W. J. et al, J. Immunol. 138(6):1657-1659, 1987). CIR-A2 cells are CIR cells that express a transfected genomic clone of HLA-A2.1 (Hogan, K. T. et al., J. Exp. Med. 168(2):725-736, 1988). These cells were obtained from Dr. William E. Biddison (National Institute of Neurological Disorders and Stroke. NIH, Bethesda, Md.). CIR-A2 culture was mycoplasma free and was maintained in RPMI 1640 complete medium (Life Technologies, Inc.). The V8T cell line, a CTL line directed against the CAP-1 epitope, was established from a patient with metastatic colon carcinoma who was enrolled in a Phase I trial using rV-CEA (Tsang, K. Y. et al., Clin. Cancer Res. 3(12):2439-2449, 1997). 18T cells were cultured in RPMI 1640 complete medium containing 10% human AB serum and IL-2 (provided bad the National Cancer Institute, Surgery Branch, 20 units/ml). V8T cells were restimulated with CAP-1 peptide (25 .mu.g/ml) on day 16 after prior restimulation at an effector cell-to-APC ratio of 1:3. Irradiated (23,000 rads) autologous EBBS transformed B cells were used as APC.

Culture of DC from Peripheral Blood Mononuclear Cells

Peripheral blood mononuclear cells (PBMC) were obtained from heparinized blood from a patient (#15) with metastatic pelvic carcinoma who was enrolled in a Phase I trial using a combination of rV-CEA and ALVAC-CEA. All experiments involving patient materials were conducted according to NIH guidelines, and written, informed consent was obtained from all individuals. PBMC were separated using lymphocyte separation medium gradient (Organon Teknika, Durham, N.C.) as described previously (Boyum. A. Scand J Clin Lab Invest Suppl. 97:51-76, 1968). DC were prepared using a modification of the procedure described by Sallusto et al. (Sallusto, F. et al., J. Exp. Med. 179(4):1109-1118, 1994). PBMC (1.5.times.10⁸) were resuspended in AIM-V medium containing 2 mM glutamine, 50 .mu.g/ml streptomycin, 10 .mu.g/ml gentamycin (Life Technologies, Inc.) and allowed to adhere to a T-150 flask (Corning Costar Corp., Cambridge, Mass.). After 2 hours at 37 C., the non-adherent cells were removed with a gentle rinse. The adherent cells were cultured for 6-7 days in AIM-V medium containing 50 ng/ml of recombinant human GM-CSF (rhGM-CSF) and 0.5 ng/ml of recombinant human IL-4 (rhIL-4). The culture medium was replenished every three days.

Recombinant Virus and Infection of DC with Avipox Virus Containing CEA, CEA/TRICOM and TRICOM

A 2109 bp DNA fragment encoding the entire open reading frame of CEA was obtained as described by Kaufman et al (Kaufman, F. et al. Int. J. Cancer 48(6):900-907, 1991). The recombinant CEA avipox virus (fowlpox CEA; vCP248) was supplied by Therion Corp using methods described by Taylor et al (Taylor, J. et al, Virology 187(1):321-328, 1992), Cox et al (Cox, W. I. et al, Virology 187(1):321-328, 1992) and Perkus et al (Perkus, M. E. et al, J. Virol. 63(9):3829-3836). The recombinant avipox virus encoding CEA and human Tricom gene (designated rF-CEA-Tricom) and the recombinant human fowlpox-TRICOM (rF-Tricom) were made as disclosed herein. Wild type fowlpox (FP-WT) was used as a negative control in selected experiments. DC (1.times.106) were incubated in 1 ml of Optim-MEM medium (Life Technologies. Inc.) at 37 C. with rF TRICOM, rF-CEA, rF-CEA/TRICOM, FP-WT. Titration experiments indicated that 2.times.10⁷ plaque-forming units/ml, equal to a multiplicity of infection (MOI) of 40:1 for 2 hours, were able to consistently induce expression of CEA in approximately 75% of the infected DC. The infected DC were suspended in 10 ml of fresh, warm RPMI-1640 complete medium containing 50 ng/ml of rhGM-CSF and 0.5 ng/ml rhIL-4 cultured for 24 hours, and then subsequently used as stimulators.

Peptide

CAP-1 (Tsang, K. Y. et al, J. Natl Cancer Inst. 87(13):982-990, 1995), CEA amino acid position 571-579 YLSGANLNL, CAP1-6D (Zaremba, S. et al., Cancer Res. 57(20):4570-4577, 1997) YLSGADLNL and Flu peptide, influenza matrix protein peptide 58-66 GILGFVTL greater than 96% pure, were made by Multiple Peptide System (San Diego, Calif.).

Generation of T-Cell Lines

Modification of the protocol described by Tsang et al (Tsang, K. Y. et al, J. Natl Cancer Inst. 87(13):982-990, 1995) was used to generate CEA-specific CTL. Uninfected DC and DC infected with rF-TRICOM, rF-CEA, or rF-CEA/TRICOM were used as APC. CAP-1 peptide was added to the uninfected or rF-TRICOM infected DC at a final concentration of 25 .mu.g/ml. Autologous non adherent cells were then added to APC at an APC-to-effector ratio of 1:10. Cultures were then incubated for 3 days at 37 C. in a humidified atmosphere containing 5% CO₂. After removal of the peptide-containing medium, the cultures were then supplemented with recombinant human IL-2 at a concentration of 20 units/ml for 7 days, with IL-2 containing medium was replenished every 3 days. The 3-day incubation with peptide and 7 day IL-2 supplement constituted one IVS cycle. Primary cultures were restimulated with CAP-1 peptide (25 .mu.g/ml) on day 11 to begin the next IVS cycle. Irradiated (23,000 rads) autologous EBV-transformed B cells were used as APC. A similar procedure was employed for CTL generation when DC infected with rF-CEA or rF-CEA/TRICOM were used as APC, with the exception that no CAP-1 peptide was in the stimulation.

Construction of Peptide MHC Tetramers

Peptide-MHC complexes were synthesized as described by Altman et al (Altman, J. D. et al Science 274(5284):94-95, 1996). In brief, the β₂ microglobulin (beta.₂M) clone was obtained from Dr. Garboczi (Harvard University, Cambridge, Mass.) (Garboczi, D. N. et al, Proc Natl Acad Sci USA 89(8):3429-3433, 1992) and the HLA-A2 construct was obtained from Immunotech (Beckman-Coulter. Marseille, France). The soluble HLA-A2 molecules containing the 15 amino acid substrate peptide for BirA-dependent biotinylation to the COOH-terminus of the HLA-A2 heavy chain and β₂M were grown separately in E. coli and isolated as inclusion bodies. HLA-A2 and β₂M were solubilized and renatured in the presence of CAP-1 or Flu-M1 58-66 peptide. The complex was purified by FPLC on Superdex 200 (Pharmacia, Piscataway, N.J.). Purified peptide-MHC complex was biotinylated using the BirA enzyme (Avidity, Denver. Colo.). Tetramers were produced by mixing the biotinylated peptide-MHC complex with phycoerythrin-labeled UltraAvidin (Leinco Technologies. Inc. Ballwin, Mo.) at a molar ratio of 4:1.

Flow Cytometry

Staining and sorting of T-cells: CAP-1-MHC tetramer-PE was used for flow cytometric analysis and sorting of T-cells. Similar procedure as described above was used for tetramer staining. CAP-1-MHC tetramer-PE was used at a concentration of 0.33 .mu.g/2.times.10⁵ cells. Cells were stained with CAP-1 MHC tetramer-PE for 1 hour at 4 C. and then stained with anti-CD8 FITC for an additional hour. Cells were washed and analyzed on a Vantage Cell sorter (Becton Dickinson) or a FACScan (Becton Dickinson) using Cell Quest software (Becton Dickinson). Sorter cells were cultured and expanded as described previously. Cells stained with UltrAvidin-PE and Flu-MHC tetramer were used as negative controls.

Cytotoxic Assay

Target cells were labeled with 50 .mu.Ci of ¹¹¹Indium-labeled oxyquinoline (Medi-Physics Inc., Arlington, Ill.) for 15 min at room temperature. Target cells (0.3.times.10⁴) in 100 .mu.l of RPMI-1640 complete medium were added to each of 96 wells in flat-bottomed assay plates (Coming Costar, Corp.). The labeled target cells were incubated with peptides for 60 min at 37 C. in 5% C₂ before adding effector cells. No peptide was used when carcinoma cell lines were used as targets. Effector cells were suspended in 100 .mu.l of RPMI-1640 complete medium supplemented with 10% pooled human AB serum and added to the target cells. The plates were then incubated at 37 C. in 5% CO₂ for 4 or 16 hours. Supernatant was harvested for gamma counting with the use of harvester frames (Skatron, Inc., Sterling, Va.). Determinations were carried out in triplicate, and standard deviations were calculated. Specific lysis was calculated with the use of the following formula (all values in cpm): 1% lysis=Observed release−Spontaneous release .times. 100 Total release−Spontaneous release

Spontaneous release was determined from wells to which 100 .mu.l of RPMI-1640 complete medium was added. Total releasable radioactivity was obtained after treatment of targets with 2.5% Triton x-100.

BLA Typing

The HLA phenotyping was performed by the Blood Bank of the National Institutes of Health using a standard antibody-dependent microcytotoxicity assay and a defined panel of anti-HLA antisera. The class I phenotypes of V8T cell line and patient #15 were HLA-A2, −; B18 (W6), 44 (12, W4) and HLA-A2, 28; B13 (BW4), B51 (BW4); CW6, respectively.

Detection of Cytokine

Supernatant of T cells exposed for 24 hours to DC infected with rF-CEA, rF-CEA/TRICOM or to peptide pulsed uninfected DC and rF-TRICOM-infected DC in IL-2-free medium at various responder:stimulator ratio were screened for secretion of IFN.gamma. using an ELISA kit (R&D Systems, Minneapolis, Minn.). The results were expressed in pg/ml.

ELISPOT Assay

A modification of the method described by Scheibenbogen et al (Scheibenbogen, C. et al, Clin Cancer Res 3(2):221-226, 1997) was used to measure IFN.gamma. production to determine CAP-1 specific T cells. Briefly, 96-well Milliliter HA plates (Millipore Corporation, Bedford, Mass.) were coated with 100 .mu.l of capture antibody against human IFN.gamma. at a concentration of 10 .mu.g/ml. After 24 hours incubation at room temperature, plates were blocked for 30 min with RPMI-1640 containing 10% human pool AB serum. 1.times.10⁵ cells to be assayed were added to each well. CAP-1-6D-pulsed CIR-A2 cells were added into each well as APC at an effector:APC ratio of 1:3. Unpulsed C1R-A2 cells were used as negative control. HLA-A2 binding Flu Matrix peptide 58-66 (GILGFVFTL) were also used as control. The responding cells were determined by the use of a Domino Image Analyzer (Otpomax, Hollis, N.H.).

Statistical Analysis

Statistical Analysis of Differences Between Means was Done Using a Two-Tailed T Test.

Discussion

When a nave T cell encounters antigen, several distinct outcomes are possible including proliferation, cytokine secretion, and differentiation into effector cells, as well as inactivation, death, and unresponsiveness (anergy). The predominant outcome under physiologic conditions may be determined by whether appropriate costimulatory signals are delivered to the responding T cell (26). At least three distinct molecules normally found on the surface of professional APC have been thought to be capable of providing the signals critical for T-cell activation: B7-1, ICAM-1, and LFA-3. Here, the role of costimulatory molecules in nave T-cell activation was examined by utilizing vectors engineered to express either B7-1, ICAM-1, LFA-3, or a combination of all three molecules.

Several groups have investigated the cooperation of two of these molecules in T-cell costimulation. Dubey et al. have reported that costimulation by both B7-1 and ICAM-1 is a prerequisite for naive T-cell activation (26), while Cavallo et al. determined that B7-1 and ICAM-1 must by coexpressed by tumor cells to establish an antitumor memory response (27). In addition, costimulation by B7-1 and LFA-3 has been shown to act additively both upon T-cell proliferation and cytokine production (6, 23, 24). These previous studies were carried out using two costimulatory molecules and retroviral vectors. One gene was transduced into the target cell line, drug selected, and then transduced again with a second recombinant retroviral construct followed by selection with a different agent. This process often requires weeks or months. Utilizing recombinant poxvirus vectors, one is able to achieve the coexpression of three costimulatory molecules 5 hours post-infection. In vitro MC38 cells infected with either rV-B7-1/ICAM-1/LFA-3 or rF-CEA/B7-1/ICAM-1/LFA-3 were shown to enhance proliferation of T cells to a much greater extent than MC38 cells infected with vectors containing the gene for any single costimulatory molecule. In addition, the relative strength of the second signal delivered to the T-cell by the combination of costimulatory molecules appeared to be several-fold (>6) greater than that delivered by MC38 cells expressing any single costimulatory molecule. Dubey et al. have demonstrated that at low stimulator to T-cell ratios, moderate to strong synergy was noted with B7-1 and ICAM-1 (26). Our studies confirm these findings. However, at very low stimulator cell to T-cell ratios or weak signal-1 (0.625 .mu.g/ml Con A), the two-gene construct (rV-B7-1/ICAM-1) had little if any effect on proliferation; in contrast, stimulation via the triad construct (rV-B7-1/ICAM-1/LFA-3) had a substantial and statistically significant effect on proliferation. The predominant effect of stimulation via the multi-gene construct (rV-B7-1, ICAM-1, LFA-3) was IL-2 elaboration from CD4⁺ cells and IFN-.gamma. elaboration from CD8⁺ T cells, while few, if any, type 2 cytokines were produced. Cytokine expression analysis by RNAse protection provided a profile compatible with the in vitro cytokine assay, manifested by significantly higher expression of IL-2 and IFN-.gamma. in both CD4⁺ and CD8⁺ T cells stimulated with all three costimulatory molecules, as compared to stimulation by any single costimulatory molecule. These data are in accordance with previous studies which demonstrated that in the context of low CD28 costimulation, T cells produced low levels of IL-1, whereas strong CD28 costimulation supported production of IL-2, IFN-.gamma. and IL-13 (28). Furthermore, it has been reported that IL-13 synergizes with IL-2 in regulating IFN-.gamma. synthesis in T cells p29). Interestingly, our results further support this observation in that stimulation of CD4⁺ T cells with MC38/B7-1/ICAM-1/LFA-3 results in a high level of IL-2 and IFN-.gamma. expression, with some increased expression of IL-13. Moreover, it was noted that IL-9 expression was further enhanced in CD4⁺ T cells upon stimulation with MC38/B7-1/ICAM-1/LFA-3. The increased expression of IL-9 in conjunction with upregulation of IL-2 noted in our studies is in agreement with previous studies which demonstrated that optimal production of IL-9 is regulated by IL-2 (30). Taken together, these studies suggest that optimal naive T-cell responses require a higher level of costimulation than was previously thought, and that this could be provided by the combined action of three costimulatary molecules.

Perhaps the most studied T-cell costimulatory molecule is B7-1. This molecule's ability to enhance T-cell activation using retroviral vectors, anti-CTLA-4 antibodies, and poxvirus vectors is well established. The studies reported here rank the order of T-cell stimulation by a single costimulatory molecule as B7-1>ICAM-1>LFA-3-. However, the employment of three costimulatory molecules was far superior to B7-1 alone or in B7 in combination with a second costimulatory molecule in both T-cell proliferation and cytokine production.

While not being bound by theory, there are several possible mechanisms for efficient cooperation between B7-1, ICAM-1 and LFA-3. The ICAM-1/LFA-3 interaction reportedly costimulates the TCR-mediated activation of T cells by sustaining the increase in the same intracellular second messengers as generated by TCR engagement. This observation suggests that the ligation of LFA-1 by ICAM-1 costimulates T cells by enhancing the signal delivered via the CD3/TCR complex (6). The ICAM-1/LFA-1 interaction is necessary to upregulate expression of the IL-2R-alpha chain and CD28 on T cells, which is required to render them competent to respond to IL-2 and B7-1 costimulation. On the other hand, the B7-1/CD28 interaction delivers a TCR-independent costimulatory signal that increases both transcriptionally and post-transcriptionally the expression of IL-2 and other immunoregulatory lymphokines. The LFA-3/CD2 interaction induces tyrosine phosphorylation of several intracellular second messengers, Ca²⁺ mobilization, and cAMP production, resulting in elaboration of a variety of cytokines, notably IL-2 and IFN-.gamma. (6). Thus, it appears that the three costimulatory molecules could be cooperating by enhancing the antigen-dependent activation of T cells, as well as their production of and response to autocrine and paracrine growth factors.

In conclusion, this invention demonstrates for the first time the ability of vectors to introduce three or more costimulatory molecules into a cell, and to rapidly and efficiently activate both CD4⁺ and CD8⁺ T-cell populations to levels far greater than those achieved when one or two of these costimulatory molecules is used. This new threshold of T-cell activation has broad implications in vaccine design and development.

The effect of the triad of costimulatory molecules on DCs was completely unexpected. DCs are known by those skilled in the art as the most potent APC. The data presented in this invention demonstrates that when DCs are infected with the “Tricom” vector, their ability to activate T-cells increases dramatically. These studies demonstrate for the first time that a DC is not the most potent APC.

### Third Set of Examples

### Example 1

Generation of a Modified CEA Gene T-cell epitopes within the native CEA protein include CAP-1 said epitope comprising the amino acid sequence YLSGANLNL (Tsang et al. (1995) J. Natl

Cancer Inst 87: 982-990). As previously noted, the immunogenicity of this epitope (in the form of a peptide) was increased by changing position six of the CAP-1 epitope from N (asparagine) to D (aspartic acid) (Zaremba et al. (1997) Cancer Res 57: 4570-4577). Using standard techniques of in vitro mutagenesis (Ausubel et al.

(1997) Curr. Protocols in Mol. Blot), this modification was introduced into the full length CEA gene via mutation of the codon AAC (which encodes asparagine) to

GAC (which encodes aspartic acid). The resulting modified CEA gene now comprises the modified epitope YLSGADLNL (the change from N to D is shown in bold). This modified CEA gene, encoding for a protein with aspartic acid (D) in place of asparagine (N) in the sixth amino acid position of the CAP-1 epitope, is designated CEA (6D) (for example, see SEQ ID NO: 1 (FIG. 62)).

### Example 2

Generation of Recombinant Fowlpox. Vaccinia and MVA Constructs. The generation of recombinant poxviruses is accomplished via homologous recombination between poxvirus genomic DNA and a plasmid vector that carries the heterologous sequences to be inserted. Plasmid vectors for the insertion of foreign sequences into poxviruses are constructed by standard methods of recombinant DNA technology (for example, Ausubel et al. (1997) Curr. Protocols. Mol. Biol).

The plasmid vectors contain one or more chimeric genes, each comprising a poxvirus promoter linked to a protein coding sequence, flanked by viral sequences from a non-essential region of the poxvirus genome. The plasmid is transfected into cells infected with the parental poxvirus, and recombination between poxvirus sequences on the plasmid and the corresponding DNA in the viral genome results in the insertion into the viral genome of the chimeric genes on the plasmid.

Recombinant viruses are selected and purified using any of a variety of selection or screening systems (Mazzara et al. (1993) Meth. Enzymol. 217: 557-581; Jenkins et al. (1991) AIDS Res. Hum. Retroviruses 7: 991-998, Sutter et al. (1994) Vaccine 12: 1032-1040). Insertion of the foreign genes into the vaccinia genome is typically confirmed by polymerase chain reaction (PCR) analysis. Expression of the foreign genes is demonstrated by Western analysis and/or via immunoprecipitation of expressed products.

Generation of Recombinant Poxviruses

For the generation of recombinant fowlpox virus rF-CEA (6D), a plasmid vector designated pT5071 was constructed to direct insertion of the foreign sequences into the BamHI J region of the fowlpox genome. The CEA (6D) gene is under the control of the vaccinia 40K promoter (Gritz et al. (1990) J. Virol. 64:5948-5957). In addition, the E. coli lacZ gene, under the control of the fowlpox virus C1 promoter (Jenkins et al. (1991) AIDS Res. Hune. Retroviruses 7: 991-998), is included as a screen for recombinant progeny. These foreign sequences are flanked by DNA sequences from the BamHI J region of the fowlpox genome. A plaque purified isolate from the POXVAC-TC strain of fowlpox was used as the parental virus for this recombinant vaccine. The generation of recombinant fowlpox virus was accomplished via homologous recombinant between fowlpox sequences in the fowlpox genome and the corresponding sequences in pT5071 in fowlpox-infected primary chick embryo dermal cells transfected with pT5071. Recombinant virus was identified using a chromogenic assay preformed on spiral plaques in situ. This assay detects expression of the lacZ gene product in the presence of halogenated indolyl-beta-D-galactoside (Bluo-gal) (Chakrabarti et al. (1985) Mol. Cell Bio 3403-3409). Viral plaques expressing lacZ appear blue against a clear background.

Positive plaques, designated vT233, were picked from the cell monolayer and their progeny replated. Seven rounds of plaque isolation and replating in the presence of

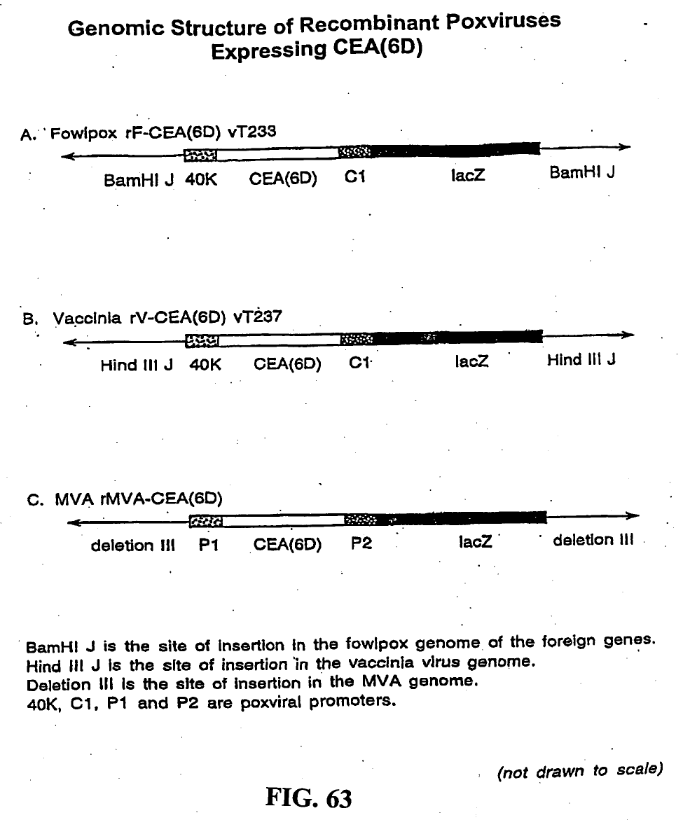

Bluo-Gal resulted in the purification of the desired recombinant. A schematic representation of the genomic structure of vT233 is depicted in FIG. 63 (A).

For the generation of recombinant vaccinia rV-CEA (6D), a plasmid vector designated pT2 146 was constructed to direct insertion of the foreign sequences into the thymidine kinase gene (located in the Hind III J region of the vaccinia genome).

The CEA (6D) gene is under the transcriptional control of the vaccinia 40K promoter and the E. coli. lacZ gene is under the control of the fowlpox virus Ci promoter.

These foreign sequences are flanked by DNA sequences from the Hind IIIJ region of the vaccinia genome. A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT2146 in vaccinia infected cells transfected with pT2146. Recombinant virus was identified using the chromogenic assay for the lacZ gene product (previously described). Positive plaques, designated vT237, were picked from the cell monolayer and their progeny replated. Five rounds of plaque isolation and replating in the presence of Bluo-Gal resulted in the purification of the desired recombinant. A schematic representation of the genomic structure of vT237 is depicted in FIG. 63 (6).

For the generation of a recombinant MVA that expressed CEA (6D), a plasmid vector was constructed to direct insertion of the foreign sequences into the MVA genome. The CEA (6D) gene and the E. coli lacZ gene were each under the control of a poxviral promoter. These foreign sequences were flanked by DNA sequences from the MVA genome into which the foreign sequences were to be inserted (for example, deletion III (Sutter et at (1994) Vaccine 12: 1032-1040)). The generation of recombinant MVA was accomplished via homologous recombination between MVA sequences in the MVA genome and the corresponding sequences in the plasmid vector in MVA-infected cells transfected with the plasmid vector.

Recombinant plaques were picked from the cell monolayer under selective conditions and their progeny further propagated. Additional rounds of plaque isolation and replating resulted in the purification of the desired recombinant virus.

A schematic representation of the genomic structure of the MVA recombinant construct is depicted in FIG. 63 (C).

### Example 3

Generation of axial VAC(2)-modified CEA-B7.1 (Human) RecombinantConstructfrCP1586)

To generate ALVAC (2)-CEAmod/hB7.1, a coding sequence containing the H6 (Perkins et al. (1989) J: Izirol. 63: 3829-3936) promoted modified CEA and the human 67.1 gene (under the control of synthetic Early/Late promoter) was subcloned into a generic ALVAC donor plasmid specific for the C5 insertion site (see below). The donor plasmid was then used to insert the expression cassette into the CS insertion locus in the ALVAC (2) genome by in vitro recombination (Piccini et al. (1987) Meth. Enzyniol. 153: 545).

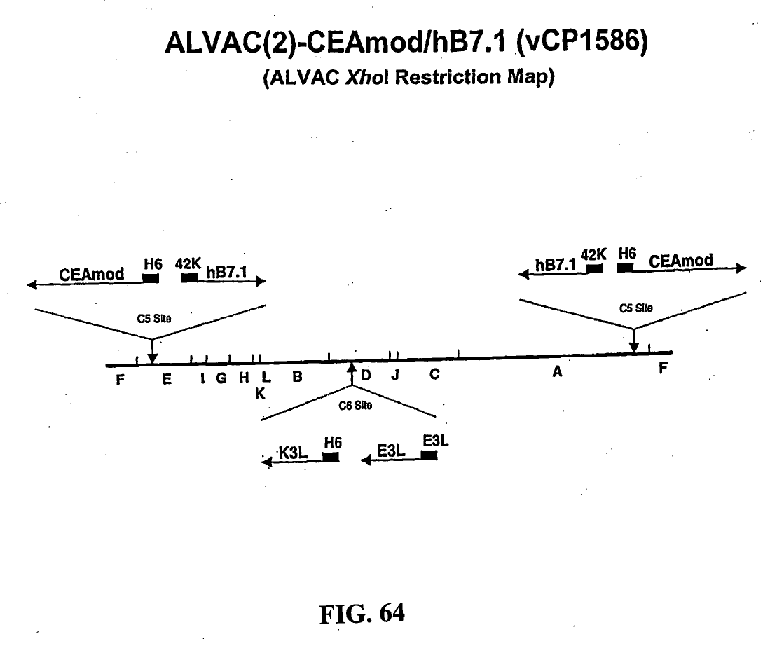

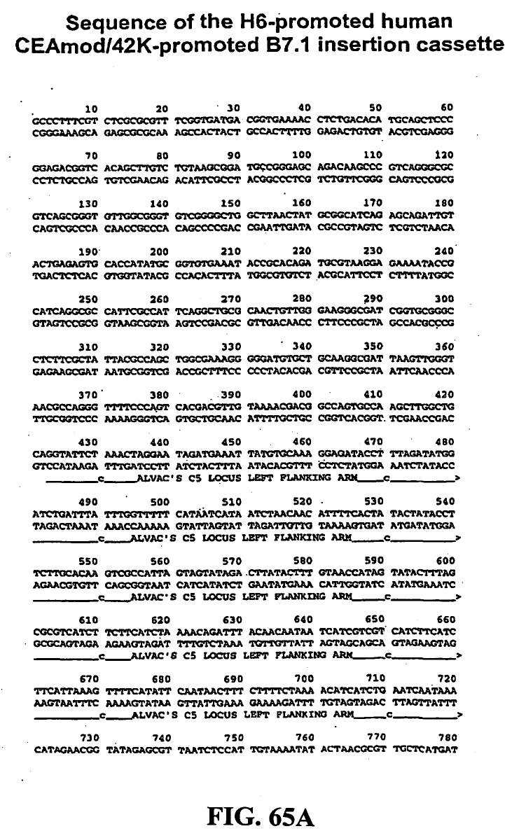

The locus designated CS was used for the insertion of the modified CEA and human B7.1 coding sequences into the ALVAC (2) vector. By virtue of the C5 locus existing within the extensive inverted terminal repetitions (ITRs) of the virus genome, insertion into this locus results in the occurrence of two copies of the inserted sequence. A schematic of the recombinant construct is depicted in FIG. 64.

Presently, no function has been ascribed to the CS encoded polypeptide nor does the deduced amino acid open reading frame encoded in this region share significant homology to any entry in the existing protein databases.] Particulars of the Geiieration of the vCPIS86 ALVA C recombinant (1) Modified CEA: sequence (up to the Bg/II site) by PCR amplification from plasmid pT2147 (comprising a full length sequence of the modified CEA (6D)) using the following PCR primers:

HM102:

(Sequence ID No. 3)

5′-TTG-TCC-GAG-CTC-TCG-CGA-TAT-CCG-TTA-AGT-TTG
TAT CGT-AAT-GGA-GTC-TCC-CTC-GGC-CCC-3′
HM103:

(Sequence ID NO. 4)

5′-CCG-GAA-TTC-TCA-CAA-GAT-CTG-ACT-TTA-TGA-C-3′

The PCR product was digested with SacI and EcoRI to generate compatible ends for cloning into Sacs EcoRI and Shrimp Alkaline Phosphatase digested pBSK+ vector (DH5a cells). The resulting plasmid (designated pF102. 103) was sequenced to confirm the fidelity of the insert. The remainder of the CEA (6D) sequence was isolated by digestion of plasmid pT2147 with Bg/II and Sa/I. The resulting 1.7 Kbp fragment contained the 3′end of CEA (6D). This fragment was ligated to Bg/II, SalI and Shrimp Alkaline Phosphatase digested pF102. 103 plasmid and transformed into DH5a cells. The resulting plasmid was designated p3′B6MCEA.

An ALVAC donor plasmid was constructed by transferring the 3′H6modified CEA sequence fragment from p3′H6MCEA to ALVAC insertion plasmid

NVQH6MC5. NVQH6MC5 was initially made via digestion of CS donor plasmid NVQH6C5LSP-18 within its polylinker region with BamHI. It was subsequently treated with alkaline phosphatase and ligated to kinased and annealed oligonucleotides SPCSPL1 (5′-GAT-CGT-CGA-CGA-GCT-CGA-ATT-CG-3′) (Sequence ID No. 5) and

SPC5PL2 (5′-GAT-CCG-AATTCG-ACC-TCG-TCG-AC-3′) (Sequence ID No. 6) thus generating plasmid NVQH6MC5.

Plasmid p3′H6MCEA was digested with NruI and SalI the fragment containing the 3′H6 modified CEA sequence purified and subsequently ligated to NruI, XhoI and Shrimp Alkaline Phosphatase digested NVQH6MC5 vector. The resulting donor plasmid, containing the modified CEA coding sequences under the control of a full length H6 promoter, was designated pAH6MCEA.

(ii) B7.1

The following primers were used to fuse the 42K promoter and PCR out the entire B7.1 gene from plasmid pT2147:

H M1 04:

(Sequence ID No. 7)

5′-TTG-TCC-GAG-CTC-GAA-TTC-TTT-ATT-G GG-AAG-AAT-
ATG ATA-ATA-TTT-TGG-GAT-TTC-AAA-ATT-GAA-AAT-ATA-
TAA-TTACATAT-AAA-ATG-GGC-CAC-ACA-CGG-AGG-CAG-3′.
HM105:

(Sequence ID No. 8)

5′-ACG-GCA-GTC-GAC-TTA-TAC-AGG-GCG-TAC-ACT-3′.

Primer HM104 contained the entire sequence of the 42K promoter. The resulting PCR product (designated F104. 105) was digested with EcoRI and SalI, and subsequently ligated into EcoRI, SalI and Shrimp Alkaline Phosphatase digested pBSK+ vector (SURE cells), The resulting plasmid was designated pF104. 105 and was sequenced to confirm the fidelity of the insert.

(iii) Donor Plasmid pA2147

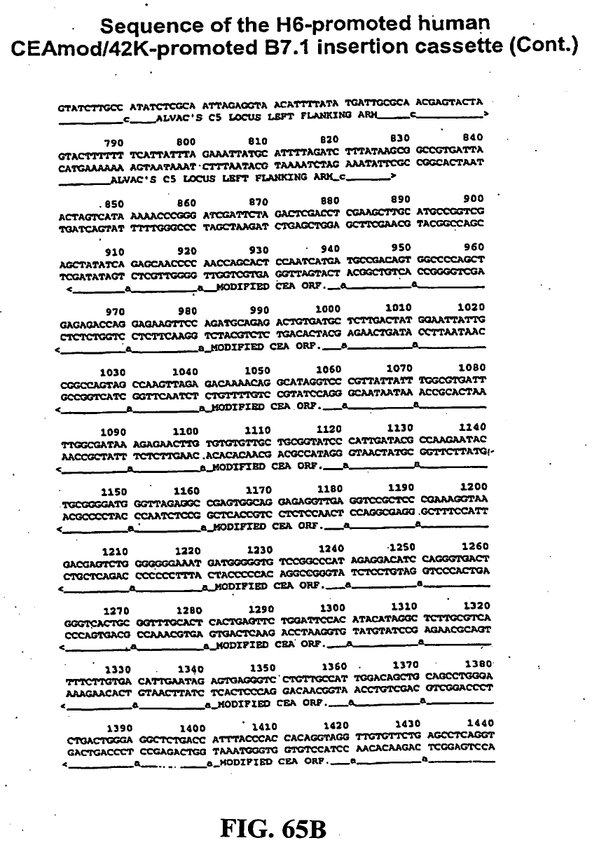

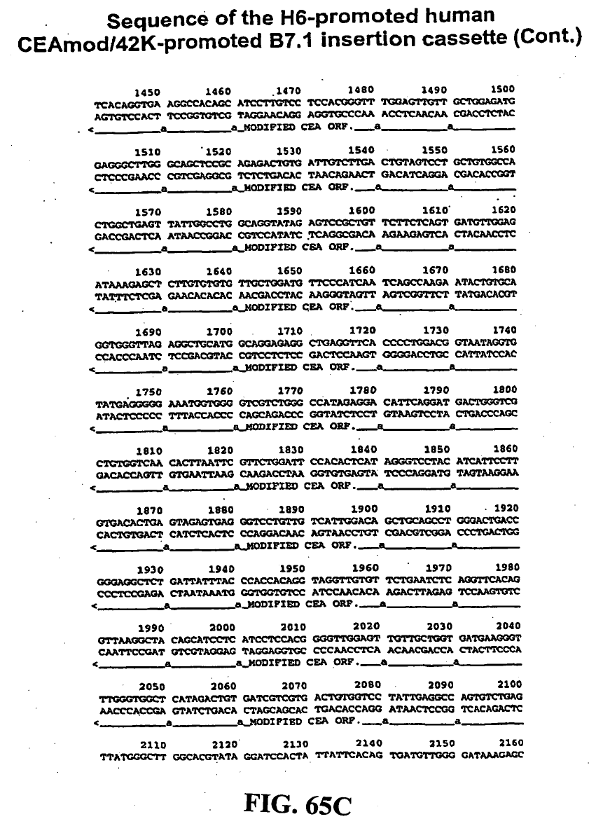

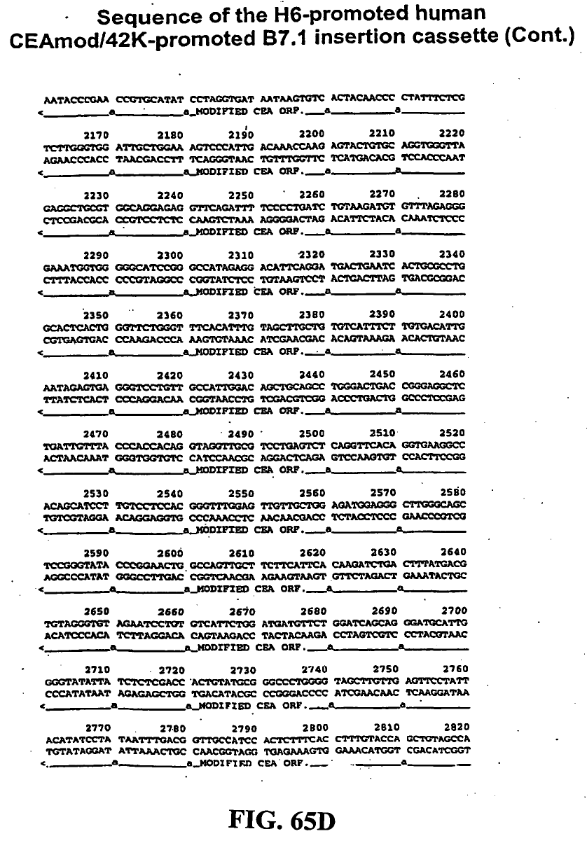

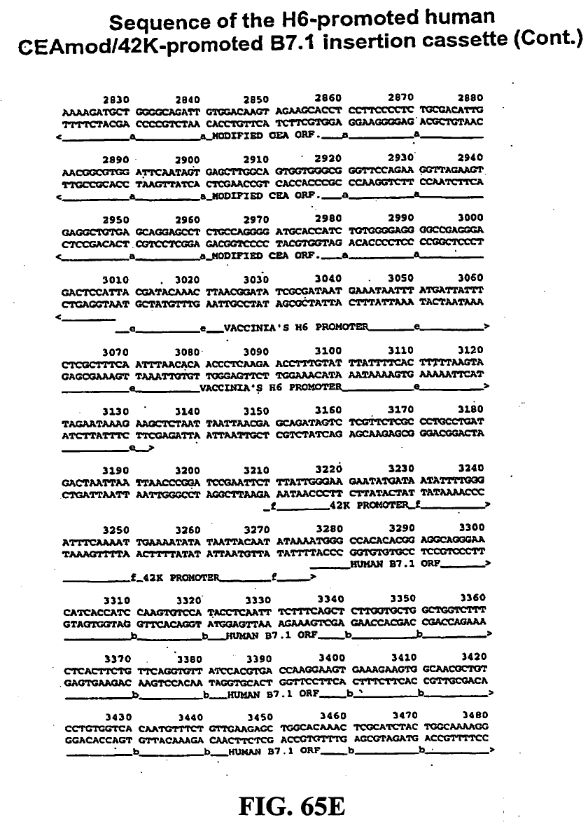

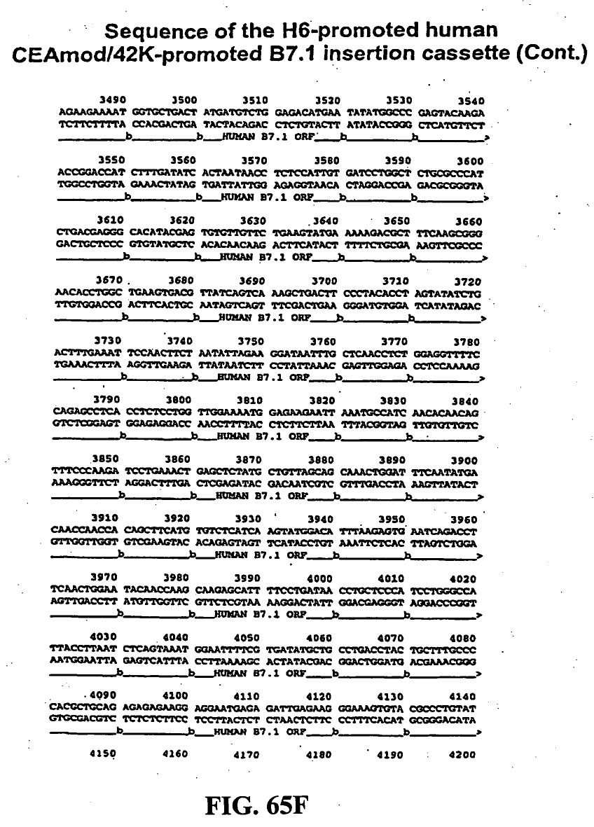

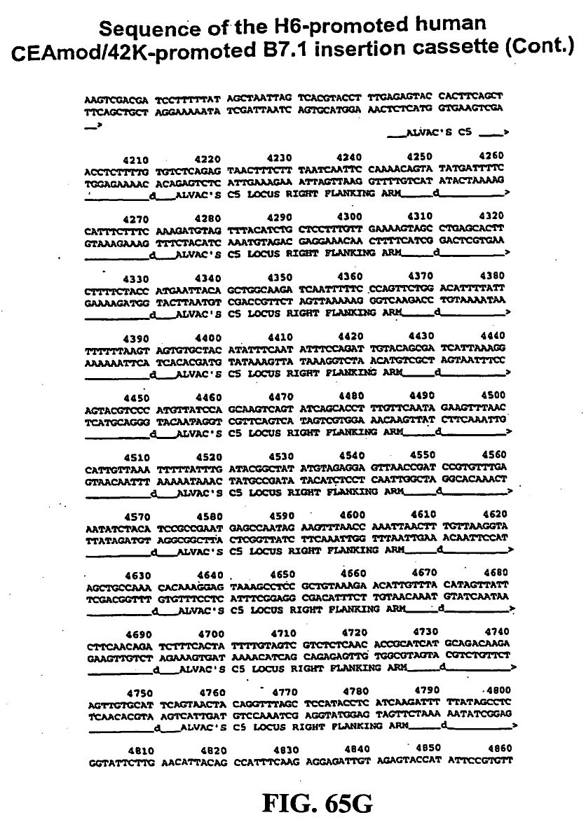

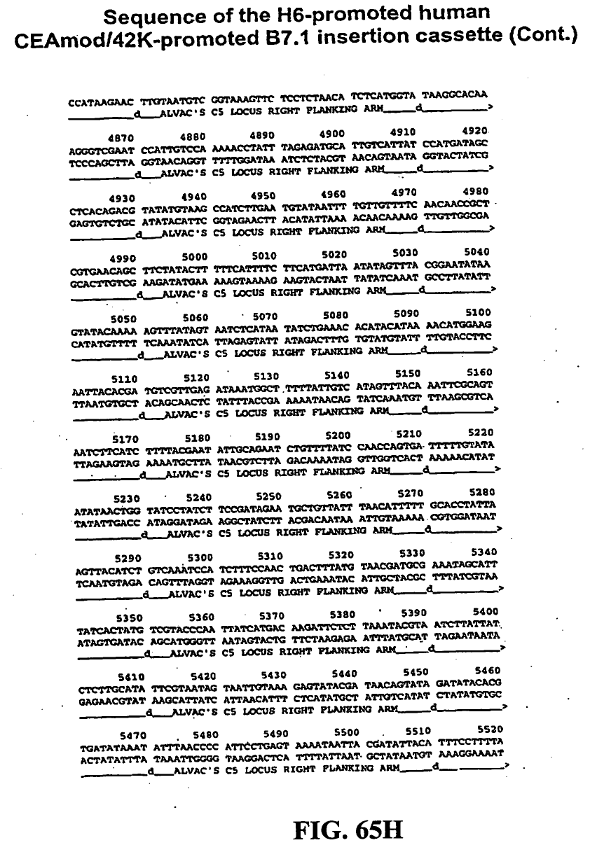

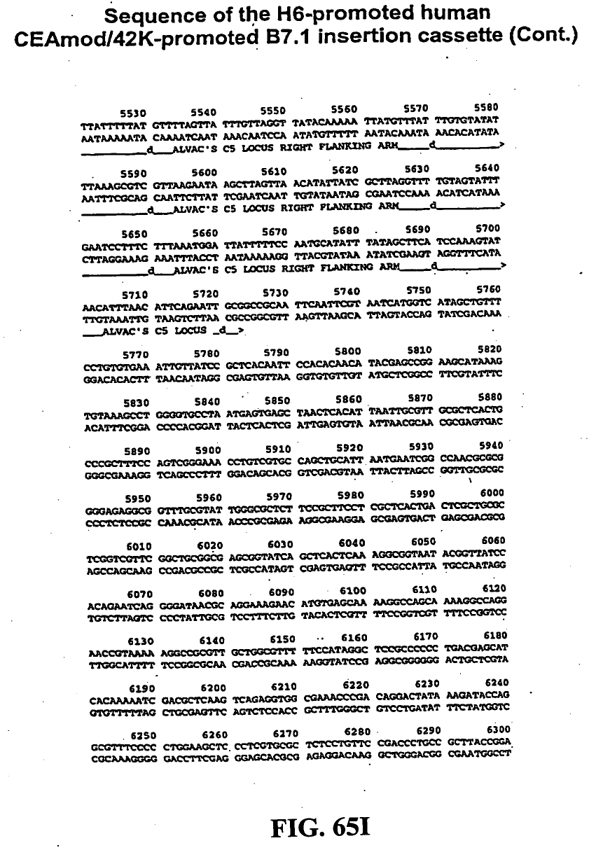

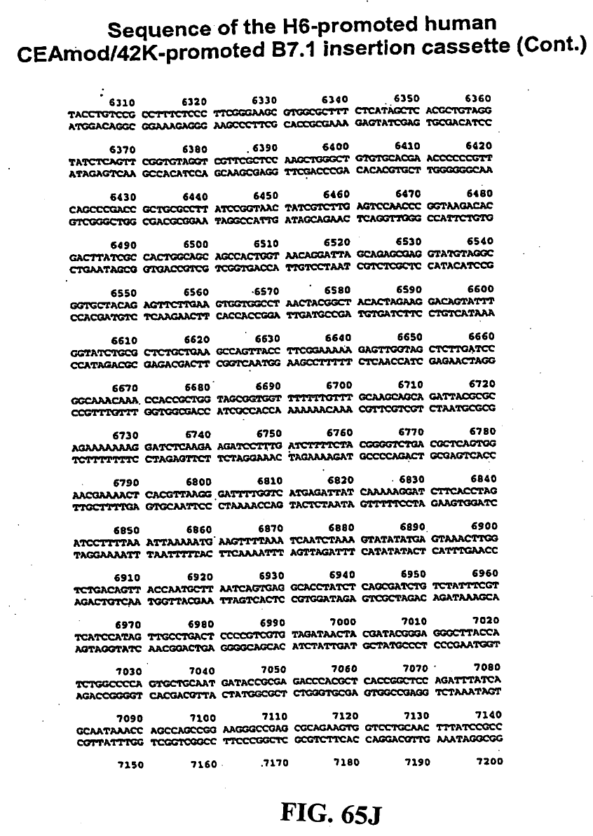

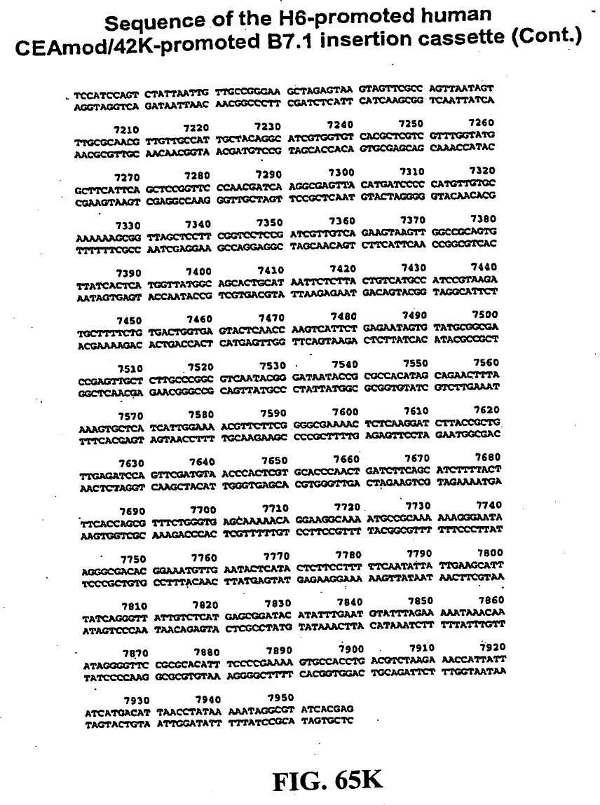

The fragment containing the 42K-67.1 coding sequences was excised from pF104. 105 by digestion with EcoRI and Sa/I, and subsequently purified. This fragment was ligated to EcoRI, SalI, Shrimp Alkaline Phosphatase digested pAH6MCEA plasmid and transformed into SURE cells. The resulting donor plasmid was designated pA2147. (See FIG. 65 for the sequence of the H6-promoted human modified CEA/42K promoted B7.1 cassette of pA2147 for insertion into ALVAC.) (iv) Generation of VCP1586

Recombination between donor plasmid pA2147 and ALVAC (2) rescuing virus generated recombinant virus vCP1586. This recombinant viral construct comprised the vaccinia H6 promoted modified human CEA gene sequence and the 42K promoted human 67.1 gene sequence in the C5 locus (for ALVAC insertion and inserted DNA sequence particulars, see FIGS. 64 and 65). Recombination was performed utilizing procedures described in the art and known to skilled artisans (Piccini et al. (1987), supra other procedures can also be utilized as described above and/or as described, for example, in U.S. Pat. Nos. 4,769,330, 4,722,848, 4,603,112, 5,174,993, 5,110,587, all of which are incorporated herein by reference).

Verification of insertion (1) Restriction Enzyme Analysis

Viral genomic DNA was isolated from cells infected with vCP1586 pursuant to methods well known to those skilled in the art (for example, as taught in Current Protocols in Molecular Biology, F. M. Ausubel et al. (Eds.), Jolm Wiley and Sons, Inc., N.Y., U.S.A. (1998); Molecular Cloning: A Laboratory Manual (2nd Ed.), J. Sambrook, E. F. Fritsch and T. Maniatis (Eds.), Cold Spring Harbor Laboratory Press, N.Y., U.S.A. (1989)). The viral genomic DNA was digested with restriction endonucleases (Hi72dIII, Pst I, BamHI or XhoI). The resultant DNA fragments were fractionated by electrophoresis through an agarose gel and visualized by ethidium bromide staining. The insertion of the modified CEA and B7.1 expression cassette at the CS locus was confirmed (for example, see FIG. 64 for a schematic representation of the XhoI restriction map of vCP1586).

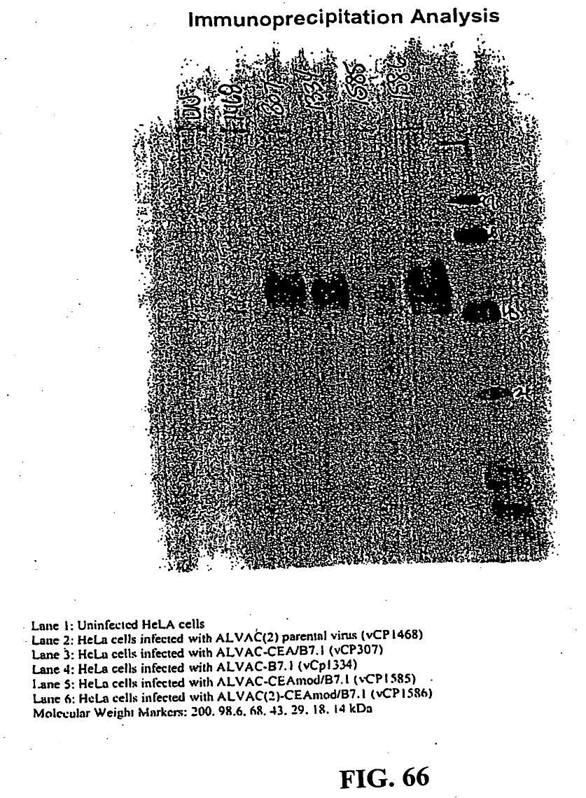

(ii) Immunoprecipitation Immunoprecipitation analyses were performed using radiolabeled lysates derived from uninfected HeLa cells or cells infected with either ALVAC (2) parental virus (vCP1486), ALVAC-CEA/B7 (vCP307), ALVAC-hB7. 1 (vCP11334), ALVAC-CEAmod/IIB7. 1 (vCP1585) or ALVAC (2)-CEAmod/hB7.1 (vCP1586) as previously described (Taylor et al. (1990) J. Virol. 64: 1441-1450). Briefly, HeLa cell cultures were infected at a multiplicity of infection (m.o.i.) of 10 pfu/cell in methionine and cystine-free media supplemented with [35S]-methionine/cysteine (30 uCi/ml). Cells were lysed at 18 hours post-infection. Immunoprecipitation was performed using a B7.1 specific monoclonal antibody (HB71; ATCC&num;80991). immunoprecipitates were fractionated on a 10% SDS-Polyacrylamide gel. The gel was fixed and treated for fluorography with 1 M Na-salicylate for ½ hr. The dried gel was exposed to Kodak XAR-2 film to visualize the protein species. Results with anti-hB7. 1 demonstrate expression of human B7 in HeLa cells infected with ALVAC (2)-CEAmod/hB7.1 (vCP1586) and the other ALVAC-based recombinants expressing hB7.1, but not in parentally infected cells (FIG. 66).

(iii) Western Blot Analysis

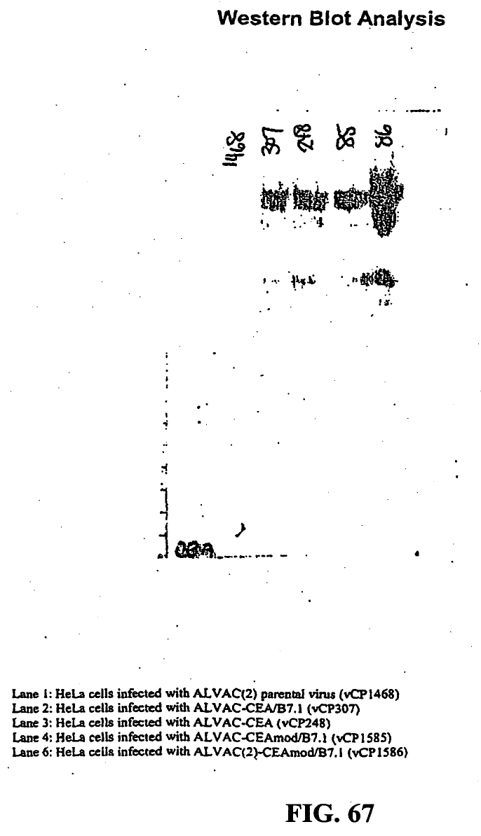

HeLa cells were infected for 18 hours at a multiplicity of 10 pfu/cell with either ALVAC (2) parental virus (vCP1486), ALVAC-CEA/B7 (vCP307), ALVAC CEA (vCP248), ALVAC-CEAmod/hB7.1 (vCP1585) or ALVAC (2) CEAmod/hB7.1 (vCP1586). Cell lysates were separated by SDS-PAGE and transferred to nitrocellulose utilizing standard techniques. The blot was incubated with anti-CEA monoclonal antibody Col-1 (1/1 000 dilution), followed by HRP conjugated rabbit anti-mouse utilizing HRP peroxide substrate with DAB/Metal (Pierce). The results obtained demonstrate the expression of full length CEA in HeLa cells infected with ALVAC (2)-CEAmod/hB7.1 (vCP1586) and other ALVAC-based recombinants expressing CEA (FIG. 67).

### FIRST SET OF REFERENCES

1. Muraro, R., Wunderlich, D., Thor, A., Lundy, J., Noguchi, P., Cunningham, R., and Schlom, J. Definition by monoclonal antibodies of a repertoire of epitopes of carcinoembryonic antigen differentially expressed in human colon carcinoma versus normal adult tissues. Cancer Res., 45: 5769-5780 1985.
2. Steward, A. M., Nixon, D., Zamcheck, N., and Aisenberg, A. Carcinoembryonic antigen in breast cancer patients: serum levels and disease progress. Cancer, 33: 1246-1252, 1974.
3. Vincent, R. G. and Chu, T. M. Carcinoembryonic antigen in patients with carcinoma of the lung. J. Thor. Cardiovas. Surg., 66: 320-328, 1978.
4. Gold, J. M., Freedman, S. O., and Gold, P. Human anti CEA antibodies detected by radioimmunoelectrophoresis. Nature New Biology, 239: 60-62. 1973.
5. Pompecki, R. Presence of immunoglobulin G in human sera binding to carcinoembryonic antigen (CEA) and nonspecific crossreacting antigen (NCA). Eur. J Cancer, 16: 973-974, 1980.
6. Ura, Y., Ochi, Y., Hamazu, M., Ishida, M., Nakajima, K., and Watanabe, T. Studies on circulating antibodies against CEA and CEA like antigen in cancer patients. Cancer Lett., 25: 283-295.1985.
7. Fuchs, C., Krapf, F., Kern, P., Hoferichter, S., Jager, W., and Kalden, J. R. CEA-containing immune complexes in sera of patients with colorectal and breast cancer-analysis of complexed immunoglobulin classes. Cancer Immunol. Immunother., 26:180-184, 1988.
8. LoGerfo, P., Herter, F. P., and Bennett, S. J. Absence of circulating antibodies to carcinoembryonic antigen in patients with gastrointestinal malignancies. Int. J. Cancer, 9: 344-348. 1972.
9. MacSween, J. M. The antigenicity of carcinoembryonic antigen in man. Int. J. Cancer, 15: 246-252. 1975.
10. Chester, K. A. and Begent, H. J. Circulating immune complexes (CIC), carcinoembryonic-antigen (CEA) and CIC containing CEA as markers for colorectal cancer. Clin. Exp. Immunol. 58: 685-693, 1984.
11. Tsang, K. Y., Zaremba, S., Nieroda, C. A., Zhu, M. Z., Hamilton, J. M., and Schlom, J. Generation of human cytotoxic T cells specific for human carcinoembryonic antigen epitopes from patients immunized with recombinant vaccinia-CEA vaccine. J. Natl Cancer Inst., 87: 982-990. 1995.
12. Gadea, J., Brunette, E., Philip, M., Lyeriy, H. K., Philip, R., and Alters, S. Generation of antigen specific CTL using peptide and gene modified dendritic cells. Proc. Am. Assoc. Cancer Res. [abstr], 3154, 1996.
13. Marshall, J. L., Hawkins, M. J., Richmond, E., Tsang, K., and Schlom, J. A study of recombinant ALVAC-CEA in patients with advanced CEA-bearing cancers. J. Immunol. [abstr]. 19: 461, 1996.
14. Foon, K. A., Chakraborty, M., John, W. J., Sherraft, A., Kohler, H., and Bhattacharya-Chatterjee, M. Immune response to the carcinoembryonic antigen in patients treated with an anti-idiotype antibody vaccine. J. Clin. Invest., 96: 334-342, 1995.
15. Fagerberg, J., Samanci, A., Yi, Q., Strigard, K. Ryden, U., Wahren, B., and Mellstedt, H. Recombinant carcinoembryonic antigen and granulocyte-macrophage colony-stimulating factor for active immunization of colorectal carcinoma patients. J. Immunol. [abstr]. 19: 461, 1996.
16. Conry, R. M., Saleh, M. N., Schlom, J., and LoBuglio, A. F. Human immune response to carcinoembryonic antigen tumor vaccines. J. Immunother. [abstr], 18: 137, 1995.
17. Parkhurst, M. R., Salgaller, M. L, Southwood, S., Robbins, P. F., Sette, A., Rosenberg, S A., and Kawakami, Y. Improved induction of melanoma-reactive CTL with peptides from the melanoma antigen gp100 modified at HLA-A*0201-binding residues. J. Immunol., 157: 2539-2548, 1996.
18. Salgaller, M. L, Marincola, F. M., Cormier, J. N., and Rosenberg, S. A. Immunization against epitopes in the human melanoma antigen gp100 following patient immunization with synthetic peptides. Cancer Res. 56: 4749-4757, 1996.
19. Bakker, A. B. H., van der Burg. S. H., Huubens, R. J. F., Drijfhout, J. W., Melief, C. J., Adema, G. J., and Figdor, C. G. Analogues of CTL epitopes with improved MHC class-I binding capacity elicit anti-melanoma CTL recognizing the wild type epitope. Int. J. Cancer. 70: 302-309, 1997.
20. Pogue, R. R., Eron, J., Frelinger, J. A., and Matsui, M. Amino-terminal alteration of the HLA-A*0201-restricted human immunodeficiency virus pol peptide increases complex stability and in vitro immunogenicity. Proc. Nat. Acad. Sci. 92: 8166-8170, 1995.
21. Lipford, G., Bauer, S., Wagner, H., and Heeg, K. Peptide engineering allows cytotoxic T cell vaccination against human papilloma virus tumor antigen E6. Immunol., 84: 298-303, 1995.
22. Grey, H. M., Ruppert, J., Vitiello, A., Sidney, J., Kast, W. M., Kubo, R. T., and Sette, A. Class I MHC-peptide interactions: structural requirements and functional implications. Cancer Sur. 22: 37-49, 1995.
23. De Magistris, M. T., Alexander, J., Coggeshall, M., Altman, A., Gaeta, F. C A., Grey, H. M., and Sette, A. Antigen analog-major histocompatibility complexes act as antagonists of the T cell receptor. Cell, 68: 625-634, 1992.
24. Bertoletti, A., Sette, A., Chissari, F. V., Penna, A., Levrero, M., Carli, M. D., Fiaccadori, F., and Ferrari, F. Natural variants of cytotoxic epitopes are T cell receptor antagonists for antiviral cytotoxic T cells. Nature, 369: 407-410, 1994.
25. Klenerman, P., Rowland-Jones, S., McAdam, S., Edwards, J., Daenke, S., Lalloo, D., Koppe, B., Rosenberg, W., Boyd, D., Edwards; A., Giangrande, P., Phillips, R. E., and McMichael, A. J. Cytotoxic T cell activity antagonized by naturally occurring HIV-1 gag variants. Nature. 369: 403-407. 1994.
26. Kuchroo, V. K., Greer, J. M., Kaul, D., Ishioka, G., Franco, A., Sette, A., Sobel, R A., and Lees, M. B. A single TCR antagonist peptide inhibits experimental allergic encephalomyelitis mediated by a diverse T cell repertoire. J., Immunol., 153: 3326-3336. 1994.
27. Jameson, S. C. and Bevan, M. J. T cell receptor antagonists and partial agonists. Immunity, 2: 1-11, 1995.
28. Meier, U-C., Klenerman, P., Griffin, P., James, W., Koppe, B., Larder, B., McMichael, A., and Phillips, R. Cytotoxic T lymphocyte lysis inhibited by viable HIV-mutants. Science. 270: 1360-1362. 1995.
29. Chen, Y., Matsushita, S., and Nishimura, Y. Response of a human T cell clone to a large panel of altered peptide ligands carrying single residue substitutions in an antigenic peptide: characterization and frequencies of TCR agonist and TCR antagonism with or without partial activation. J. Immunol., 157: 3783-3790. 1996.
30. Nijman, H. W., Houbiers, J. G., Vierboom, M. P., van der Hurg, S. H., Drijfhout, J. W., D'Amaro, J., Kenemans, P., Melief, C. J., and Kast, W. M. Identification of peptide sequences that potentially trigger HLA-A2.1-restricted cytotoxic T lymphocytes. Eur. J. Immunol., 23: 1215-1219, 1993.
31. Rammensee, H-G., Friede, T., and Stevanovic, S. MHC ligands and peptide motifs: first listing. Immunogenetics, 41: 178-228, 1995.
32. Ruppert, J., Sidney, J., Celis, E., Kubo, R. T., Grey, B. M., and Sette, A. Prominent role of secondary anchor residues in peptide binding to HLA-A2.1 molecules. Cell, 74: 929-937. 1993.
33. Madden, D. R., Garboczi, D. N., and Wiley, D. C. The antigenic identity of peptide-MHC complexes: a comparison of the conformations of five viral peptides presented by HLA-A2. Cell, 75: 693-708, 1993.
34. Hamilton, J. M., Chen, A. P., Nguyen, B., Grem, J., Abrams, S., Chung, Y., Kantor, J., Phares, J. C., Bastian, A., Brooks, C., Morrison, G., Allegra, C. J., and Schlom, J. Phase I study of recombinant vaccinia virus (rV) that expresses human carcinoembryonic antigen (CEA) in adult patients with adenocarcinomas. Proc. Am. Soc. Clin. Oncol. [abstr], 961. 1994.
35. Abrams, S. I., Horan Hand, P., Tsang, K. Y., and Schlom, J. Mulant ras epitopes as targets for cancer vaccines. Semin. Oncol., 23: 118-134, 1996.
36. Elas, A. V., Nijman, H. W., Van Minne, C. E., Mourer, J. S., Kast, W. M., Melief, C. J. M., and Schrier, P. I. Induction and characterization of cytotoxic T lymphocytes recognizing a mutated p21 RAS peptide presented by HLA-A2010. Int. J. Cancer, 61: 389-396, 1995.
37. Houbiers, J. G. A., Nijman, B. W., van der Burg, S. H., Drijfhout, J. W., Kenemans, P., van de Velde, C. J. H., Brande, A., Momburg, F., Kasi, W. M., and Melief, C. J. M. In vitro induction of human.cytotoxic T lymphocyte responses against peptides of mutant and wild type p53. Eur. J. Immunol., 23:2072-2077, 1993.
38. Ropke, M., Regner, M., and Claesson, M. H. T cell mediated cytotoxicity against p53-protein derived peptides in bulk and limiting dilution cultures of healthy donors. Scand. J. Immunol., 42: 98-103, 1995.
39. Robbins, P. F., El-Gamil, M., Li, Y. F., Kawakami, Y., Loftus, D., Appella, E., and Rosenberg, S. A mutated β-catenin gene encodes a melanoma-specific antigen recognized by tumor infiltrating lymphocytes. J. Exp. Med. 183: 1185-1192, 1996.
40. Skipper, J. C. A., Hendrickson, R. C., Guiden, P. H., Brichard, V., van Pel, A., Chen, Y., Shabanowitz, J., Wolfelii, T., Slingluff, C. L., Jr., Boon, T., Hunt, D. F., and Engelhard, V. H. An HLA-A2-restricted tyrosinase antigen on melanoma cells results from post-translational modification and suggests a novel pathway for processing of membrane proteins. J. Exp. Med., 183: 527-534, 1996.
41. Thompson, J. A., Grunert, F., and Zimmerman, W. Carcinoembryonic antigen gene family: molecular biology and clinical perspectives. J. Clin. Lab. Anal., 5: 344-366, 1991.
42. Van der Bruggen, P., Traversari, C., Chomez, P., Lurquin, C., De Plaen, E., Van den Eynde, B., Knuth, A., and Boon, T. A gene encoding an antigen recognized by cytotoxic T lymphocytes on a human melanoma. Science, 254: 1643-1647, 1991.
43. Kawakari, Y., Eliyahu, S., Delgaldo, C. H., Robbins, P. F., Rivoltini, L., Topalian, S. L, Miki, T., and Rosenberg, S. A. Cloning of the gene coding for a shared human melanocyte antigen recognized by autologous T cells infiltrating into tumor. Proc. Natl. Acad. Sci., 91: 3515-3519. 1994.
44. Peoples, G. E., Goedegebuure, P. S., Smith, R. Linehan, D. C., Yoshino, I., and Eberlein, T. J. Breast and ovarian cancer-specific cytotoxic T lymphocytes recognize the same HER2/neu-derived peptide. Proc. Nat. Acad. Sci. 92: 432-436, 1995.
45. Matsuoka, T., Kohrogi, H., Ando, M., Nishimura, Y., and Matsushita, S. Altered TCR ligands affect antigen-presenting cell responses: up regulation of IL-12 by analogue peptide. J. Immunol., 157:4837-4843. 1996.
46. Ikagawa, S., Matsushita, S., Chen, Y-Z, Ishikawa, T., and Nishimura, Y. Single amino acid substitutions on a Japanese cedar pollen allergen (Cry j 1)-derived peptide induced alterations in human T cell responses and T cell receptor antagonism. J. Aller. Clin. Immunol., 97: 53-64, 1996.
47. England, R. D., Kullberg, M. C., Cornete, J. L., and Berzofsky, J. A. Molecular analysis of a heteroclitic T cell response to the immunodominant epitope of sperm whale myoglobin: Implications for peptide partial agonists. J. Immunol., 155: 4295-4306, 1995.
48. Ropke, M., Hald, J., Guldberg, P., Zeuthen, J., Norgaard, L, Fugger, L., Svejgaard, A., Van Der Burg, S., Nijman, H. W., Melief, C. J. M., and Claesson, M. H. Spontaneous human squamous cell carcinomas are killed by a human cytotoxic T lymphocyte clone recognizing a wild-type p53-derived peptide. Proc. Nat. Acad. Sci., 93: 14704-14707, 1996.
49. Correalei P., Walmsley, K., Nieroda, C., Zaremba, S., Zhu, M., Schlom, J., and Tsang, K. Y. In vitro generation of human cytotoxic T lymphocytes specific for peptides derived from prostate-specific antigen. J. Natl. Cancer Inst., 89: 293-300, 1997.
50. Wucherpfennig, K. W. and Strominger, J. L. Molecular mimicry in T cell-mediated autoimmunity: viral peptides activate human T cell clones specific for myelin basic protein. Cell, 80: 695-705, 1995.
51. Chen, W., Ede, N J. Jackson, D. C., McCluskey, J., and Purcell, A. W. CTL recognition of an altered peptide associated with asparagine bond rearrangement: Implications for immunity and vaccine design. J. Immunol., 157: 1000-1005, 1996.
52. Kersh, G. J. and Allen, P. M. Structural basis for T cell recognition of altered peptide ligands: A single T cell receptor can productively recognize a large continuum of related ligands. J. Exp. Med 184: 1259-1268, 1996.
53. Tsang, K. Y. Zhu, M. Z. Nieroda, C. A, Correale, P., Zaremba, S., Hamilton, J. M., Cole, D., Lam, C., and Schlom, J. Phenotypic Stability of a Cytotoxic T Cell Line Directed Against an Immunodominant Epitope of Human Carcinoembryonic Antigen (accepted by Clinical Cancer Res.).

### SECOND SET OF REFERENCES

A1. Hellstrom, K. E., Chen, L. & Hellstrom, I. (1996) Cancer Chemother Pharmacol 38, S40-1.
A2. Damle, N. K., Klussman, K., Linsley, P. S. & Aruffo, A. (1992) J Immunol 148, 1985-92.
A3. Green, J. M., Zheng, X. G., Shimizu, Y., Thompson, C. B. & Turka, L. A. (1994) Eur J Immunol 24, 265-72.
A4. Guinan, E. C., Gribben, J. G., Boussiotis, V. A., Freeman, G. J. & Nadler, L. M. (1994) Blood 84, 3261-82.
A5. Hodge, J. W., McLaughlin, J. P., Abrams, S. I., Shupert, W. L., Schlom, J. & Kantor, J. A. (1995) Cancer Res 55, 3598-603.
A6. Wingren, A. G., Parra, E., Varga, M., Kalland, T., Sjogren, H. O., Hedlund, G. & Dohlsten, M. (1995) Crit Reiv Immunol 15, 235-53.
A7. Paria, E., Wingren, A. G., Hedlund C., Sjosren, H. O., Kalland, T., Sansom, D. & Dohlsten, M. (1993) Scand J Immunol 38, 508-14.
A8. Harding, F. A. & Allison, J. P. (1993) J Exp Med 177, 1791-6.
A9. Hellstrom, K. E., Hellstrom, I., Linsley, P. & Chen, L. (1993) Ann NY Acad Sci 690, 225-30.
A10. Hodge, J. W., Abrams, S., Schlom, J. & Kantor, J. A. (1994) Cancer Res 54, 5552-5.
A11. Uzendoski, K., Kantor, J. A., Abrams, S. I., Schlom, J. & Hodge, J. W. (1997) Hum Gene Ther 8, 851-60.
A12. Lorenz, M. G. O., Kantor, J. A., Schlom, J. & Hodge, J. W. (1998) Human Gene Therapy In Press.
A13. Gritz, L., Destree, A., Cormier, N., Day, E., Stallard, V., Caiazzo, T., Mazzara, G. & Panicali, D. (1990) J Virol 64, 5948-57.
A14. Mazzara, G. P., Destree, A. & Mahr, A. (1993) Methods Enzymol 217, 557-81.
A15. Jenkins, S., Gritz, L., Fedor, C. H., O'Neill, E. M., Cohen, L. K. & Panicali, D. L. (1991) AIDS Res Hum Retroviruses 7, 991-8.
A16. Chakrabarti, S., Sisler, J. R. & Moss, B. (1997) BioTechniques 23, 1094-7.
A17. Perkus, M. E., Piccini, A., Lipinskas, B. R. & Paoletti, E. (1985) Science 229, 981-4.
A18. Schmitt, J. F. & Stunnenberg, H. G. (1988) J Virol 62, 1889-97.
A19. Venkatesan, S., Baroudy, B. M. & Moss, B. (1981) Cell 25, 805-13.
A20. Fox, B. A., Spiess, P. J., Kasid, A., Puri, R., Mule, J. J., Weber, J. S. & Rosenberg, S. A. (1990) J Biol Response Mod 9, 499-511.
A21. Abrams, S. I., Dobrzanski, M. J., Wells, D. T., Stanziale, S. F., Zaremba, S., Masuelli, L., Kantor, J. A., Schoom, J. & Masuelle, L. (1995) Eur J Immunol 25, 2588-97.
A22. Sabzevari, B., Propp, S., Kono, D. B. & Theofilopoulos, A. N. (1997) Eur J Immunol 27, 1901-10.
A23. Parra, E., Wingren, A. G., Hedlund, G., Bjorklund, M., Sjogren. H. O., Kalland, T., Sansom, D. & Dohlsten, M. (1994) J Immunol 153, 2479-87.
A24. Parra, E., Wingren, A. G., Hedlund, G., Kalland, T. & Dohlsten, M. (1997) J Immunol 158, 637-42.
A25. Sperling, A. I., Auger, J. A., Ehst, B. D., Rulifson, I. C., Thompson, C. B. & Bluestone, J. A. (1996) J Immunol 157, 3909-17.
A26. Dubey, C., Croft, M. & Swain, S. L. (1995) J Immunol 155, 45-57.
A27. Cavallo, F., Martin-Fontecha, A., Bellone, M., Heltai, S., Gatti, E., Toraghi, P., Freschi, M., Formi, G., Dellabona, P. & Casorati, G. (1995) Eur J Immunol 25, 1154-62.
A28. Delespesse, G., Yang, L. P., Ohshima, Y., Demeure, C., Shu, U., Byun, D. G. & Sarfati, M. (1998) Vaccine 16, 1415-9.
A29. Minty, A., Chalon, P., Derocq, J. M., Dumont, X., Guillemol, J. C., Kaghad, M., Labit, C., Leplatois, P., Liauzun, P., Miloux, B. & et al., (1993) Nature 362, 248-50.
A30. Schmitt, E., Germann, T., Goederi, S., Hoehn, P., Huels, C., Koelsch, S., Kuhn, R., Muller, W., Palm, N. & Rude, E. (1994) J Immunol 153, 3989-96.
A31. Chakrabarti, S., Brechling, K. and Moss, B (1985) Mol. Cell. Biol. 5:3403-3409.
A32. Chakrabarti, S., Sisler, J. R., and Moss, B. (1997) BioTechniques 23:1094-1097.
A33. Gillard, S., Spehner, D., Drillien, R., and Kim, A. (1986) Proc. Natl. Acad. Sci. USA 83:5573-5577.
A34. Morgan, J. R, and Roberts, B. E. (1994) J. Virol. 51:283-297.
A35. Panicali, D., Grzelecki, A., and Huang, C. (1986) Gene 47:193-199.
A36. Sambrook, J., Fritsch, E. F., and Maniatis, T., eds. Molecular Cloning, Cold Spring Harbor Laboratory, 1989.
A37. Schmitt, J. F. C. and Stunnenberg, H. G. 1988) J. Virol. 62:1889-1897.
A38. Smith, K., Stallard, V., Ross, J., Hart, C. Cormier, N., Cohen, L., Roberts, B., and Payne, L. (1993) Vaccine 11:43-53.
A39. Venkatesan, S., Baroudy, B. M., and Moss, B. (1981) Cell 125:805-813.
A40. Bacchetti, S. and Graham, F. L. Proc. Nat'l. Acad. Sci. USA 74:1590-1594, 1977.
A41. Inaba, K., Inaba, M., Romani, N. Aya, H., Deguchi, M. Ikehara, S., Muramatsu, S. & Steinman, R. M. (1992) J. Exp. Med. 176, 1693-702.
A42. Sallusto, F. & Lanzavecchia, A. (1994) J. Exp. Med. 179, 1109-8.
A43. Fields, R. C., Osterholzer, J. J., Fuller, J. A., Thomas, E. K., Geraghty, P. J. & Mule, J. J. (1998) J. Immunother. 21, 323-39.
A44. Zaremba et al Cancer Res. 57:4570-4577, 1997.
A45. Gong, J. et al Proc. Natl. Acad. Sci. USA 95:6279-6283, 1998.
A46. Correale, P. et al 1998, J. Immunol. 161(6):3186-94.

Having illustrated and described the principles of the invention in particular and/or preferred embodiments, it should be appreciated by those skilled in the art that the invention can be modified in arrangement and detail without departure from such principles.

The patent applications to which this application claims priority and all publications, patents and patent applications referred to herein, are herein incorporated by reference in their entirety to the same extent as if each individual publication, patent or patent application was specifically and individually indicated to be incorporated by reference in its entirely.

1. A CEA agonist polypeptide comprising a modified epitope of CEA, wherein said modified epitope contains the sequence YLSGADLNL (SEQ ID NO: 24).

2. The CEA agonist polypeptide of claim 1 having the sequence of SEQ ID NO: 46 (FIG. 62).

3. A nucleic acid comprising a nucleic acid sequence which encodes the CEA agonist polypeptide of claim 1.

4. The nucleic acid of claim 2 comprising the sequence of SEQ ID NO: 45 (FIG. 62).

5. The nucleic acid of claim 3 wherein the nucleic acid is selected from the group consisting of viral nucleic acid, bacterial DNA, plasmid DNA, naked/free DNA, and RNA.

6. The nucleic acid of claim 5 wherein the viral nucleic acid is selected from the group consisting of adenoviral, alphaviral, and poxviral nucleic acid.

7. The poxviral nucleic acid of claim 6, wherein said nucleic acid is selected from the group consisting of avipox, orthopox, and suipox nucleic acid.

8. The poxviral nucleic acid of claim 7, wherein said nucleic acid is selected from the group consisting of vaccinia, fowlpox, canary pox and swinepox.

9. The poxviral nucleic acid of claim 7, wherein said nucleic acid is selected from the group consisting of TROVAC, NYVAC, ALVAC, MVA, Wyeth, and Poxvac-TC.

10. The nucleic acid of any one of claim 1, further comprising a nucleic acid sequence encoding at least one member selected from the group comprising cytokines, lymphokines, and co-stimulatory molecules.

11. A vector comprising the nucleic acid of claim 1.

12. The vector of claim 11, wherein said vector is selected from the group consisting of recombinant viruses and bacteria.

13. The recombinant virus vector of claim 12, selected from the group consisting of adenovirus, alphavirus and poxvirus.

14. The poxvirus of claim 13, selected from the group consisting of avipox, orthopox, and suipox.

15. The poxvirus of claim 13, selected from the group consisting of vaccinia, fowlpox, canary pox, and swinepox.

16. The poxvirus of claim 14, selected from the group consisting of TROVAC, NYVAC, ALVAC, MVA, Wyeth, and Poxvac-TC.

18. The vector of claim 11 further comprising a nucleic acid sequence encoding at least one member selected from the group consisting of cytokines, lymphokines, and co-stimulatory molecules.

19. A cell comprising a nucleic acid according to claim 1 wherein the cell expresses a CEA agonist polypeptide.

20. A method of inhibiting a CEA epitope expressing carcinoma cell in a patient comprising administering to said patient an effective amount of the CEA agonist polypeptide according to claim 1.

21. The polypeptide of claim 1, wherein said polypeptide is purified.

22. The polypeptide of claim 21, wherein said polypeptide is comprised in a sterile pharmaceutical composition.

23. The vectore of claim 11, wherein said vector is a CEA/TRICOM vector.


SEQUENCE LISTING (64 sequences)

SEQ ID NO: 1 - protein, 9 aa, Artificial Sequence
  Description of Artificial Sequence Synthetic Peptide
KTWGQYWZY

SEQ ID NO: 2 - protein, 9 aa, Artificial Sequence
  Description of Artificial Sequence Synthetic Peptide
ITDQVPPSV

SEQ ID NO: 3 - protein, 9 aa, Artificial Sequence
  Description of Artificial Sequence Synthetic Peptide
YLEPGPVTA

SEQ ID NO: 4 - protein, 10 aa, Artificial Sequence
  Description of Artificial Sequence Synthetic Peptide
LLDGTATLRL

SEQ ID NO: 5 - protein, 10 aa, Artificial Sequence
  Description of Artificial Sequence Synthetic Peptide
VLYRYGSFSV

SEQ ID NO: 6 - protein, 9 aa, Artificial Sequence
  Description of Artificial Sequence Synthetic Peptide
AAGIGILTV

SEQ ID NO: 7 - protein, 9 aa, Artificial Sequence
  Description of Artificial Sequence Synthetic Peptide
ILTVILGVL

SEQ ID NO: 8 - protein, 9 aa, Artificial Sequence
  Description of Artificial Sequence Synthetic Peptide
MSLQRQFLR

SEQ ID NO: 9 - protein, 9 aa, Artificial Sequence
  Description of Artificial Sequence Synthetic Peptide
MLLAVLYCL

SEQ ID NO: 10 - protein, 9 aa, Artificial Sequence
  Description of Artificial Sequence Synthetic Peptide
YMNGTMSQV

SEQ ID NO: 11 - protein, 9 aa, Artificial Sequence
  Description of Artificial Sequence Synthetic Peptide
SEIWRDIDF

SEQ ID NO: 12 - protein, 9 aa, Artificial Sequence
  Description of Artificial Sequence Synthetic Peptide
AFLPWHRLF

SEQ ID NO: 13 - protein, 15 aa, Artificial Sequence
  Description of Artificial Sequence Synthetic Peptide
QNILLSNAPLGPQFP

SEQ ID NO: 14 - protein, 13 aa, Artificial Sequence
  Description of Artificial Sequence Synthetic Peptide
SYLQDSDPDSFQD

SEQ ID NO: 15 - protein, 9 aa, Artificial Sequence
  Description of Artificial Sequence Synthetic Peptide
EADPTGHSY

SEQ ID NO: 16 - protein, 9 aa, Artificial Sequence
  Description of Artificial Sequence Synthetic Peptide
SAYGEPRKL

SEQ ID NO: 17 - protein, 9 aa, Artificial Sequence
  Description of Artificial Sequence Synthetic Peptide
EVDPIGHLY

SEQ ID NO: 18 - protein, 9 aa, Artificial Sequence
  Description of Artificial Sequence Synthetic Peptide
FLWGPRALV

SEQ ID NO: 19 - protein, 9 aa, Artificial Sequence
  Description of Artificial Sequence Synthetic Peptide
AARAVFLAL

SEQ ID NO: 20 - protein, 8 aa, Artificial Sequence
  Description of Artificial Sequence Synthetic Peptide
YRPRPRRY

SEQ ID NO: 21 - protein, 9 aa, Artificial Sequence
  Description of Artificial Sequence Synthetic Peptide
VLPDVFIRC

SEQ ID NO: 22 - protein, 9 aa, Artificial Sequence
  Description of Artificial Sequence Synthetic Peptide
AYGLDFYIL

SEQ ID NO: 23 - protein, 9 aa, Artificial Sequence
  Description of Artificial Sequence Synthetic Peptide
YLSGANLNL

SEQ ID NO: 24 - protein, 9 aa, Artificial Sequence
  Description of Artificial Sequence Synthetic Peptide
YLSGADLNL

SEQ ID NO: 25 - protein, 9 aa, Artificial Sequence
  Description of Artificial Sequence Synthetic Peptide
SYLDSGIHF

SEQ ID NO: 26 - protein, 9 aa, Artificial Sequence
  Description of Artificial Sequence Synthetic Peptide
EEKLIVVLF

SEQ ID NO: 27 - protein, 10 aa, Artificial Sequence
  Description of Artificial Sequence Synthetic Peptide
ACDPHSGHFV

SEQ ID NO: 28 - protein, 9 aa, Artificial Sequence
  Description of Artificial Sequence Synthetic Peptide
IISAVVGIL

SEQ ID NO: 29 - protein, 9 aa, Artificial Sequence
  Description of Artificial Sequence Synthetic Peptide
KIFGSLAFL

SEQ ID NO: 30 - protein, 10 aa, Artificial Sequence
  Description of Artificial Sequence Synthetic Peptide
YMLDLQPETT

SEQ ID NO: 31 - protein, 20 aa, Artificial Sequence
  Description of Artificial Sequence Synthetic Peptide
PDTRPAPGSTAPPAHGVTSA

SEQ ID NO: 32 - protein, 30 aa, Artificial Sequence
  Description of Artificial Sequence Synthetic Peptide
FLTPKKLQCVDLHVISNDVCAQVHPQKVTK

SEQ ID NO: 33 - protein, 10 aa, Artificial Sequence
  Description of Artificial Sequence Synthetic Peptide
FLTPKKLQCV

SEQ ID NO: 34 - protein, 9 aa, Artificial Sequence
  Description of Artificial Sequence Synthetic Peptide
KLQCVDLHV

SEQ ID NO: 35 - protein, 10 aa, Artificial Sequence
  Description of Artificial Sequence Synthetic Peptide
VISNDVCAQV

SEQ ID NO: 36 - protein, 9 aa, Artificial Sequence
  Description of Artificial Sequence Synthetic Peptide
QVHPQKVTK

SEQ ID NO: 37 - protein, 8 aa, Artificial Sequence
  Description of Artificial Sequence Synthetic Peptide
EAQNTTYL

SEQ ID NO: 38 - protein, 8 aa, Artificial Sequence
  Description of Artificial Sequence Synthetic Peptide
SIINFEKL

SEQ ID NO: 39 - protein, 8 aa, Artificial Sequence
  Description of Artificial Sequence Synthetic Peptide
RGYVYQGL

SEQ ID NO: 40 - protein, 9 aa, Artificial Sequence
  Description of Artificial Sequence Synthetic Peptide
ASNENMDAM

SEQ ID NO: 41 - DNA, 2297 bp, Artificial Sequence
  Description of Artificial Sequence Synthetic DNA Vector
atgaccatgattacgccaagcgcgcaattaaccctcactaaagggaacaaaagctgggtaccgggccccccctcgaggtcgacggtatcgataagcttgatatcgaatttcgaggtcgacatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctgtgagtatgacaagcttgatatcgaattccggtgtccggggctccaccgcccccccagcccacggtgtcacctcggccccggacaccaggccggccccgggcagtactgcaccaccggcacatggcgtaacatcagcacctgatacaagacctgcacctggatccaccgcgccgcctgcgcacggagtgacgtcggcgcccgacacgcgccccgctcccgggtcaacagctcctcccgctcatggggttacttctgctccagatactcgcccagctccaggttcgacggccccccctgctcacggtgtaacatccgccccggataccagaccggcccctggcagcaccgcaccgcccgcccatggagttacaagtgcacccgatacccggccggcacccggaagtaccgctccacctgcacacggggtcacaagcgcgccagacactcgacctgcgccagggtcgactgcccctccggcgcatggtgtgacctcagctcctgacacaaggccagccccaggttcaacggcacctccagcacacggagtcacgtctgcacccgacacccgtccagctccgggtagtacagcgccacccgcacatggcgtcacgagcgctccggatacgagaccggcgcctgctagcactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatcccagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagacccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaagaggactggcccagagagccctgagatagcggggatccactagttctagagcggcgccaccgcggtggagctccaattcgcctaatagtgagtcgtattacgcgcgctcactggccgtcgttttacaacgtcgtgactgggaaaacctggcgttaccaacttaatcgcttgcaacacatcccctttcgcagctggcgtaatacgaagaggccgcacgatcgcccttccaacagttgcgcacctgaatggcaatgga

SEQ ID NO: 42 - protein, 9 aa, Homo sapiens
YLSGADINL

SEQ ID NO: 43 - protein, 9 aa, Homo sapiens
YLSGANINL

SEQ ID NO: 44 - protein, 9 aa, Homo sapiens
YLSGACLNL

SEQ ID NO: 45 - DNA, 2106 bp, Artificial Sequence
  Description of Artificial Sequence Synthetically modified CEA
  CDS (1)..(2103)
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 46 - protein, 701 aa, Artificial Sequence
  Description of Artificial Sequence Synthetically modified CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 47 - DNA, 7958 bp, Artificial Sequence
  Description of Artificial Sequence Synthetic DNA construct
gccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggctgcaggtattctaaactaggaatagatgaaattatgtgcaaaggagatacctttagatatggatctgatttatttggtttttcataatcataatctaacaacattttcactatactataccttcttgcacaagtcgccattagtagtatagacttatactttgtaaccatagtatactttagcgcgtcatcttcttcatctaaaacagatttacaacaataatcatcgtcgtcatcttcatcttcattaaagttttcatattcaataactttcttttctaaaacatcatctgaatcaataaacatagaacggtatagagcgttaatctccattgtaaaatatactaacgcgttgctcatgatgtactttttttcattatttagaaattatgcattttagatctttataagcggccgtgattaactagtcataaaaacccgggatcgattctagactcgacctcgaagcttgcatgccggtcgagctatatcagagcaaccccaaccagcactccaatcatgatgccgacagtggccccagctgagagaccaggagaagttccagatgcagagactgtgatgctcttgactatggaattattgcggccagtagccaagttagagacaaaacaggcataggtcccgttattatttggcgtgattttggcgataaagagaacttgtgtgtgttgctgcggtatcccattgatacgccaagaatactgcggggatgggttagaggccgagtggcaggagaggttgaggtccgctcccgaaaggtaagacgagtctgggggggaaatgatgggggtgtccggcccatagaggacatccagggtgactgggtcactgcggtttgcactcactgagttctggattccacatacataggctcttgcgtcatttcttgtgacattgaatagagtgagggtcctgttgccattggacagctgcagcctgggactgactgggaggctctgaccatttacccaccacaggtaggttgtgttctgagcctcaggttcacaggtgaaggccacagcatccttgtcctccacgggtttggagttgttgctggagatggagggcttgggcagctccgcagagactgtgattgtcttgactgtagtcctgctgtggccactggctgagttattggcctggcaggtatagagtccgctgttcttctcagtgatgttggagataaagagctcttgtgtgtgttgctggatgttcccatcaatcagccaagaatactgtgcaggtgggttagaggctgcatggcaggagaggctgaggttcacccctggacggtaataggtgtatgagggggaaatggtggggtcgtctgggccatagaggacattcaggatgactgggtcgctgtggtcaacacttaattcgttctggattccacactcatagggtcctacatcattccttgtgacactgagtagagtgagggtcctgttgtcattggacagctgcagcctgggactgaccgggaggctctgattatttacccaccacaggtaggttgtgttctgaatctcaggttcacaggttaaggctacagcatcctcatcctccacggggttggagttgttgctggtgatgaagggtttgggtggctcatagactgtgatcgtcgtgactgtggtcctattgaggccagtgtctgagttatgggcttggcacgtataggatccactattattcacagtgatgttgggcataaagagctcttgggtggattgctggaaagtcccattgacaaaccaagagtactgtgcaggtgggttagaggctgcgtggcaggagaggttcagattttcccctgatctgtaagatgtgtttagaggggaaatggtgggggcatccgggccatagaggacattcaggatgactgaatcactgcgcctggcactcactgggttctgggtttcacatttgtagcttgctgtgtcatttcttgtgacattgaatagagtgagggtcctgttgccattggacagctgcagcctgggactgaccgggaggctctgattgtttacccaccacaggtaggttgcgtcctgagtctcaggttcacaggtgaaggccacagcatccttgtcctccacgggtttggagttgttgctggagatggagggcttgggcagctccgggtatacccggaactggccagttgcttcttcattcacaagatctgactttatgacgtgtagggtgtagaatcctgtgtcattctggatgatgttctggatcagcagggatgcattggggtatattatctctcgaccactgtatgcgggccctggggtagcttgttgagttcctattacatatcctataatttgacggttgccatccactctttcacctttgtaccagctgtagccaaaaagatgctggggcagattgtggacaagtagaagcacctccttcccctctgcgacattgaacggcgtggattcaatagtgagcttggcagtggtgggcgggttccagaaggttagaagtgaggctgtgagcaggagcctctgccaggggatgcaccatctgtggggaggggccgagggagactccattacgatacaaacttaacggatatcgcgataatgaaataatttatgattatttctcgctttcaatttaacacaaccctcaagaacctttgtatttattttcactttttaagtatagaataaagaagctctaattaattaacgagcagatagtctcgttctcgccctgcctgatgactaattaattaacccggatccgaattctttattgggaagaatatgataatattttgggatttcaaaattgaaaatatataattacaatataaaatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggagacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaattttcgtgatatgctgcctgacctactgctttgccccacgctgcagagagagaaggaggaatgagagattgagaagggaaagtgtacgccctgtataagtcgacgatcctttttatagctaattagtcacgtacctttgagagtaccacttcagctacctcttttgtgtctcagagtaactttctttaatcaattccaaaacagtatatgattttccatttctttcaaagatgtagtttacatctgctcctttgttgaaaagtagcctgagcacttcttttctaccatgaattacagctggcaagatcaatttttcccagttctggacattttattttttttaagtagtgtgctacatatttcaatatttccagattgtacagcgatcattaaaggagtacgtcccatgttatccagcaagtcagtatcagcacctttgttcaatagaagtttaaccattgttaaatttttatttgatacggctatatgtagaggagttaaccgatccgtgtttgaaatatctacatccgccgaatgagccaatagaagtttaaccaaattaactttgttaaggtaagctgccaaacacaaaggagtaaagcctccgctgtaaagaacattgtttacatagttattcttcaacagatctttcactattttgtagtcgtctctcaacaccgcatcatgcagacaagaagttgtgcattcagtaactacaggtttagctccatacctcatcaagatttttatagcctcggtattcttgaacattacagccatttcaagaggagattgtagagtaccatattccgtgttagggtcgaatccattgtccaaaaacctatttagagatgcattgtcattatccatgatagcctcacagacgtatatgtaagccatcttgaatgtataattttgttgttttcaacaaccgctcgtgaacagcttctatactttttcattttcttcatgattaatatagtttacggaatataagtatacaaaaagtttatagtaatctcataatatctgaaacacatacataaaacatggaagaattacacgatgtcgttgagataaatggctttttattgtcatagtttacaaattcgcagtaatcttcatcttttacgaatattgcagaatctgttttatccaaccagtgatttttgtataatataactggtatcctatcttccgatagaatgctgttatttaacatttttgcacctattaagttacatctgtcaaatccatctttccaactgactttatgtaacgatgcgaaatagcatttatcactatgtcgtacccaattatcatgacaagattctcttaaatacgtaatcttattatctcttgcatattcgtaatagtaattgtaaagagtatacgataacagtatagatatacacgtgatataaatatttaaccccattcctgagtaaaataattacgatattacatttccttttattatttttatgttttagttatttgttaggttatacaaaaattatgtttatttgtgtatatttaaagcgtcgttaagaataagcttagttaacatattatcgcttaggttttgtagtatttgaatcctttctttaaatggattatttttccaatgcatatttatagcttcatccaaagtataacatttaacattcagaattgcggccgcaattcaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 48 - DNA, 27 bp, Homo sapiens
tacctttcgggagcgaacctcaacctc

SEQ ID NO: 49 - DNA, 27 bp, Homo sapiens
tacctttcgggagcggacctcaacctc

SEQ ID NO: 50 - DNA, 27 bp, Homo sapiens
tacctttcgggagcggacatcaacctc

SEQ ID NO: 51 - protein, 9 aa, Homo sapiens
YLNVQDLNL

SEQ ID NO: 52 - protein, 9 aa, Homo sapiens
YLHDPEFNL

SEQ ID NO: 53 - protein, 9 aa, Human papillomavirus
YMDLQPETT

SEQ ID NO: 54 - protein, 9 aa, Influenza virus
GILGFVFTL

SEQ ID NO: 55 - DNA, 60 bp, Artificial Sequence
  Description of Artificial Sequence Synthetic oligonucleotide
ttgtccgagctctcgcgatatccgttaagtttgtatcgtaatggagtctccctcggcccc

SEQ ID NO: 56 - DNA, 31 bp, Artificial Sequence
  Description of Artificial Sequence Synthetic oligonucleotide
ccggaattctcacaagatctgactttatgac

SEQ ID NO: 57 - DNA, 23 bp, Artificial Sequence
  Description of Artificial Sequence Synthetic oligonucleotide
gatcgtcgacgagctcgaattcg

SEQ ID NO: 58 - DNA, 23 bp, Artificial Sequence
  Description of Artificial Sequence Synthetic oligonucleotide
gatccgaattcgacctcgtcgac

SEQ ID NO: 59 - DNA, 104 bp, Artificial Sequence
  Description of Artificial Sequence Synthetic oligonucleotide
ttgtccgagctcgaattctttattgggaagaatatgataatattttgggatttcaaaattgaaaatatataattacatataaaatgggccacacacggaggcag

SEQ ID NO: 60 - DNA, 30 bp, Artificial Sequence
  Description of Artificial Sequence Synthetic oligonucleotide
acggcagtcgacttatacagggcgtacact

SEQ ID NO: 61 - protein, 10 aa, Homo sapiens
QVHIPQKVTK

SEQ ID NO: 62 - DNA, 27 bp, Homo sapiens
tacctttcgggagcgaacatcaacctc

SEQ ID NO: 63 - DNA, 27 bp, Homo sapiens
tacctttcgggagcgtgtctcaacctc

SEQ ID NO: 64 - DNA, 105 bp, Artificial Sequence
  Description of Artificial Sequence Synthetic oligonucleotide
ttgtccgagctcgaattctttattgggaagaatatgataatattttgggatttcaaaattgaaaatatataattacaatataaaatgggccacacacggaggcag